(19) United States
(12) Patent Application Publication
(10) Pub. No.: US 2014/0315929 A1
(43) Pub. Date: Oct. 23, 2014

(54) HSP90 COMBINATION THERAPY

(71) Applicant: Gabriela Chiosis, New York, NY (US)

(72) Inventor: Gabriela Chiosis, New York, NY (US)

(73) Assignee: SLOAN-KETTERING INSTITUTE FOR CANCER RESEARCH, New York, NY (US)

(21) Appl. No.: 14/113,779

(22) PCT Filed: Apr. 27, 2012
(86) PCT No.: PCT/US12/35690

§ 371 Date: Jun. 12, 2014

Related U.S. Application Data
(60) Provisional application No. 61/480,198, filed on Apr. 28, 2011.

(51) Int. Cl. G01N 33/50; A61K 45/06; G01N 33/574; A61K 31/52

(52) U.S. Cl. CPC G01N 33/5011; A61K 31/52; A61K 45/06; G01N 33/5748
USPC 514/263.24; 435/7.1; 435/7.4; 435/7.92; 506/9; 435/7.23

(57) ABSTRACT

This invention concerns a method for selecting an inhibitor of a cancer-implicated pathway or of a component of a cancer-implicated pathway for coadministration, with an inhibitor of HSP90, to a subject suffering from a cancer which comprises the following steps:
(a) contacting a sample containing cancer cells from a subject with an inhibitor of HSP90 or an analog, homolog or derivative of an inhibitor of HSP90 under conditions such that one or more cancer pathway components present in the sample bind to the HSP90 inhibitor or the analog, homolog or derivative of the HSP90 inhibitor;
(b) detecting pathway components bound to the HSP90 inhibitor or to the analog, homolog or derivative of the HSP90 inhibitor;
(c) analyzing the pathway components detected in step (b) so as to identify a pathway which includes the components detected in step (b) and additional components of such pathway; and
(d) selecting an inhibitor of the pathway or of a pathway component identified in step (c).

This invention further concerns a method of treating a cancer patient by coadministering an inhibitor of HSP90 and an inhibitor of a cancer-implicated pathway or component thereof.

The inventions described herein were made, at least in part, with support from Grant No. ROI CA 155226 from the National Cancer Institute, Department of Health and Human Services; and the U.S. Government has rights in any such subject invention.

Throughout this application numerous public documents including issued and pending patent applications, publications, and the like are identified. These documents in their entireties are hereby incorporated by reference into this application to help define the state of the art as known to persons skilled therein.

### BACKGROUND OF THE INVENTION

There is a great need to understand the molecular aberrations that maintain the malignant phenotype of cancer cells. Such an understanding would enable more selective targeting of tumor-promoting molecules and aid in the development of more effective and less toxic anti-cancer treatments. Most cancers arise from multiple molecular lesions, and likely the resulting redundancy limits the activity of specific inhibitors of signaling molecules. While combined inhibition of active pathways promises a better clinical outcome, comprehensive identification of oncogenic pathways is currently beyond reach.

Application of genomics technologies, including large-scale genome sequencing, has led to the identification of many gene mutations in various cancers, emphasizing the complexity of this disease (Ley et al., 2008; Parsons et al., 2008). However, whereas these genetic analyses are useful in providing information on the genetic make-up of tumors, they intrinsically lack the ability to elucidate the functional complexity of signaling networks aberrantly activated as a consequence of the genetic defect(s). Development of complementary proteomic methodologies to identify molecular lesions intrinsic to tumors in a patient- and disease stage-specific manner must thus follow.

Most proteomic strategies are limited to measuring protein expression in a particular tumor, permitting the identification of new proteins associated with pathological states, but are unable to provide information on the functional significance of such findings (Hanash & Taguchi, 2010). Some functional information can be obtained using antibodies directed at specific proteins or post-translational modifications and by activity-based protein profiling using small molecules directed to the active site of certain enzymes (Kolch & Pitt, 2010; Nomura et al., 2010; Brehme et al., 2009; Ashman & Villar, 2009). Whereas these methods have proven useful to query a specific pathway or post-translational modification, they are not as well suited to capture more global information regarding the malignant state (Hanash & Taguchi, 2010). Moreover, current proteomic methodologies are costly and time consuming. For instance, proteomic assays often require expensive SILAC labeling or two-dimensional gel separation of samples.

Accordingly, there exists a need to develop simpler, more cost effective proteomic methodologies that capture important information regarding the malignant state. As it is recognized that the molecular chaperone protein heat shock protein (Hsp90) maintains many oncoproteins in a pseudo-stable state (Zuehlke & Johnson, 2010; Workman et al., 2007), Hsp90 may be an important protein in the development of new proteomic methods.

In support of this hypothesis, heat shock protein (Hsp90), a chaperone protein that functions to properly fold numerous proteins to their active conformation, is recognized to play important roles in maintaining the transformed phenotype (Zuehlke & Johnson, 2010; Workman et al., 2007). Hsp90 and its associated co-chaperones assist in the correct conformational folding of cellular proteins, collectively referred to as “client proteins”, many of which are effectors of signal transduction pathways controlling cell growth, differentiation, the DNA damage response, and cell survival. Tumor cell addiction to deregulated proteins (i.e. through mutations, aberrant expression, improper cellular translocation etc) can thus become critically dependent on Hsp90 (Workman et al., 2007). While Hsp90 is expressed in most cell types and tissues, work by Kamal et at demonstrated an important distinction between normal and cancer cell Hsp90 (Kamal et al, 2003). Specifically, they showed that tumors are characterized by a multi-chaperone complexed Hsp90 with high affinity for certain Hsp90 inhibitors, while normal tissues harbor a latent, uncomplexed Hsp90 with low affinity for these inhibitors.

Many of the client proteins of Hsp90 also play a prominent role in disease onset and progression in several pathologies, including cancer. (Whitesell and Lindquist, Nat Rev Cancer 2005, 5, 761; Workman et al., Ann NY Acad Sci 2007, 1113, 202; Luo et al., Mol Neurodegener 2010, 5, 24.) As a result there is also significant interest in the application of Hsp90 inhibitors in the treatment of cancer. (Taldone et al., Opin Pharmacol 2008, 8, 370; Janin, Drug Discov Today 2010, 15, 342.)

Based on the body of evidence set forth above, we hypothesize that proteomic approaches that can identify key oncoproteins associated with Hsp90 can provide global insights into the biology of individual tumor and can have widespread application towards the development of new cancer therapies. Accordingly, the present disclosure provides tools and methods for identifying oncoproteins that associate with Hsp90. Moreover, the present disclosure provides methods for identifying treatment regimens for cancer patient.

### SUMMARY OF THE INVENTION

The present disclosure relates to the discovery that small molecules able to target tumor-enriched Hsp90 complexes (e.g., Hsp90 inhibitors) can be used to affinity-capture Hsp90-dependent oncogenic client proteins. The subsequent identification combined with bioinformatic analysis enables the creation of a detailed molecular map of transformation-specific lesions. This map can guide the development of combination therapies that are optimally effective for a specific patient. Such a molecular map has certain advantages over the more common genetic signature approach because most anti-cancer agents are small molecules that target proteins and not genes, and many small molecules targeting specific molecular alterations are currently in pharmaceutical development.

Accordingly, the present disclosure relates to Hsp90 inhibitor-based chemical biology/proteomics approach that is integrated with bioinformatic analyses to discover oncogenic proteins and pathways. We show that the method can provide a tumor-by-tumor global overview of the Hsp90-dependent proteome in malignant cells which comprises many key signaling networks and is considered to represent a significant fraction of the functional malignant proteome.

The disclosure provides small-molecule probes that can affinity-capture Hsp90-dependent oncogenic client proteins. Additionally, the disclosure provides methods of harnessing the ability of the molecular probes to affinity-capture Hsp90-dependent oncogenic client proteins to design a proteomic approach that, when combined with bioinformatic pathway analysis, identifies dysregulated signaling networks and key oncoproteins in different types of cancer.

In one aspect, the disclosure provides small-molecule probes derived from Hsp90 inhibitors based on purine and purine-like (e.g., PU-H71, MPC-3100, Debio 0932), isooxazole (e.g., NVP-AUY922) and indazol-4-one (e.g., SNX-2112) chemical classes (see FIG. 3). In one embodiment, the Hsp90 inhibitor is PU-H71 8-(6-Iodo-benzo[1,3]dioxol-5-ylsulfanyl)-9-(3-isopropylamino-propyl)-9H-purin-6-ylamine, (see FIG. 3). The PU-H71 molecules may be linked to a solid support (e.g., bead) through a tether or a linker. The site of attachment and the length of the tether were chosen to ensure that the molecules maintain a high affinity for Hsp90. In a particular embodiment, the PU-H71-based molecular probe has the structure shown in FIG. 30. Other embodiments of Hsp90 inhibitors attached to solid support are shown in FIGS. 32-35 and 38. It will be appreciated by those skilled in the art that the molecule maintains higher affinity for the oncogenic Hsp90 complex species than the housekeeping Hsp90 complex. The two Hsp90 species are as defined in Moulick et al, Nature chemical biology (2011). When bound to Hsp90, the Hsp90 inhibitor traps Hsp90 in a client-protein bound conformation.

In another aspect, the disclosure provides methods of identifying specific oncoproteins associated with Hsp90 that are implicated in the development and progression of a cancer. Such methods involve contacting a sample containing cancer cells from a subject suffering from cancer with an inhibitor of Hsp90, and detecting the oncoproteins that are bound to the inhibitor of Hsp90. In particular embodiments, the inhibitor of Hsp90 is linked to a solid support, such as a bead. In these embodiments, oncoproteins that are harbored by the Hsp90 protein bound to the solid support can be eluted in a buffer and submitted to standard SDS-PAGE, and the eluted proteins can be separated and analyzed by traditional means. In some embodiments of the method the detection of oncoproteins comprises the use of mass spectroscopy. Advantageously, the methods of the disclosure do not require expensive SILAC labeling or two-dimensional separation of samples.

In certain embodiments of the invention the analysis of the pathway components comprises use of a bioinformatics computer program, for example, to define components of a network of such components.

The methods of the disclosure can be used to determining oncoproteins associated with various types of cancer, including but not limited to a breast cancer, a lung cancer including a small cell lung cancer and a non-small cell lung cancer, a cervical cancer, a colon cancer, a choriocarcinoma, a bladder cancer, a cervical cancer, a basal cell carcinomachoriocarcinoma, a colon cancer, a colorectal cancer, an endometrial cancer esophageal cancer, a gastric cancer, a head and neck cancer, a acute lymphocytic cancer (ACL), a myelogenous leukemia including an acute myeloid leukemia (AML) and a chronic myeloid chronic myeloid leukemia (CML), a multiple myeloma, a T-cell leukemia lymphoma, a liver cancer, lymphomas including Hodgkin's disease, lymphocytic lymphomas neuroblastomas follicular lymphoma and a diffuse large B-cell lymphoma, an oral cancer, an ovarian cancer, a pancreatic cancer, a prostate cancer, a rectal cancer, sarcomas, skin cancers such as melanoma, a testicular cancer, a thyroid cancer, a renal cancer, myeloproliferative disorders, gastrointestinal cancers including gastrointestinal stromal tumors, an esophageal cancer, a stomach cancer, a gallbladder cancer, an anal cancer, brain tumors including gliomas, lymphomas including a follicular lymphoma and a diffuse large B-cell lymphoma. Additionally, the disclosure provides proteomic methods to identify dysregulated signaling networks associated with a particular cancer. In addition, the approach can be used to identify new oncoproteins and mechanisms.

In another aspect, the methods of the disclosure can be used to provide a rational basis for designing personalized therapy for cancer patients. A personalized therapeutic approach for cancer is based on the premise that individual cancer patients will have different factors that contribute to the development and progression of the disease. For instance, different oncogenic proteins and/or cancer-implicated pathways can be responsible for the onset and subsequent progression of the disease, even when considering patients with identical types at cancer and at identical stages of progression, as determined by currently available methods. Moreover, the oncoproteins and cancer-implicated pathways are often altered in an individual cancer patient as the disease progresses. Accordingly, a cancer treatment regimen should ideally be targeted to treat patients on an individualized basis. Therapeutic regimens determined from using such an individualized approach will allow for enhanced anti-tumor activity with less toxicity and with less chemotherapy or radiation.

Hence, in one aspect, the disclosure provides methods of identifying therapeutic regimens for cancer patients on an individualized basis. Such methods involve contacting a sample containing cancer cells from a subject suffering from cancer with an inhibitor of Hsp90, detecting the oncoproteins that are bound to the inhibitor of Hsp90, and selecting a cancer therapy that targets at least one of the oncoproteins bound to the inhibitor of Hsp90. In certain aspects, a combination of drugs can be selected following identification of oncoproteins bound to the Hsp90. The methods of the disclosure can be used to identify a treatment regimen for a variety of different cancers, including, but not limited to a breast cancer, a lung cancer, a brain cancer, a cervical cancer, a colon cancer, a choriocarcinoma, a bladder cancer, a cervical cancer, a choriocarcinoma, a colon cancer, an endometrial cancer an esophageal cancer, a gastric cancer, a head and neck cancer, an acute lymphocytic cancer (ACL), a myelogenous leukemia, a multiple myeloma, a T-cell leukemia lymphoma, a liver cancer, lymphomas including Hodgkin's disease and lymphocytic lymphomas neuroblastomas, an oral cancer, an ovarian cancer, a pancreatic cancer, a prostate cancer, a rectal cancer, sarcomas, a skin cancer, a testicular cancer, a thyroid cancer and a renal cancer.

In another aspect, the methods involve contacting a sample containing cancer cells from a subject suffering from cancer with an inhibitor of Hsp90, detecting the oncoproteins that are bound to the inhibitor of Hsp90, determining the protein network(s) associated with these oncoproteins and selecting a cancer therapy that targets at least one of the molecules from the networks of the oncoproteins bound to the inhibitor of Hsp90.

In certain aspects, a combination of drugs can be selected following identification of oncoproteins bound to the Hsp90. In other aspects, a combination of drugs can be selected following identification of networks associated with the oncoproteins bound to the Hsp90. The methods of the disclosure can be used to identify a treatment regimen for a variety of different cancers, including, but not limited to a breast cancer, a lung cancer, a brain cancer, a cervical cancer, a colon cancer, a choriocarcinoma, a bladder cancer, a cervical cancer, a choriocarcinoma, a colon cancer, an endometrial cancer an esophageal cancer, a gastric cancer, a head and neck cancer, an acute lymphocytic cancer (ACL), a myelogenous leukemia, a multiple myeloma, a T-cell leukemia lymphoma, a liver cancer, lymphomas including Hodgkin's disease and lymphocytic lymphomas neuroblastomas, an oral cancer, an ovarian cancer, a pancreatic cancer, a prostate cancer, a rectal cancer, sarcomas, a skin cancer, a testicular cancer, a thyroid cancer and a renal cancer.

In one embodiment of the present invention, after a personalized treatment regimen for a cancer patient is identified using the methods described above, the selected drugs or combination of drugs is administered to the patient. After a sufficient amount of time taking the selected drug or drug combination, another sample can be taken from the patient and the an assay of the present can be run again to determine if the oncogenic profile of the patient changed. If necessary, the dosage of the drug(s) can be changed or a new treatment regimen can be identified. Accordingly, the disclosure provides methods of monitoring the progress of a cancer patient over time and changing the treatment regimen as needed.

In another aspect, the methods of the disclosure can be used to provide a rational basis for designing personalized combinatorial therapy for cancer patients built around the Hsp90 inhibitors. Such therapeutic regimens may allow for enhanced anti-tumor activity with less toxicity and with less chemotherapy. Targeting Hsp90 and a complementary tumor-driving pathway may provide a better anti-tumor strategy since several lines of data suggest that the completeness with which an oncogenic target is inhibited could be critical for therapeutic activity, while at the same time limiting the ability of the tumor to adapt and evolve drug resistance.

Accordingly this invention provides a method for selecting an inhibitor of a cancer-implicated pathway, or of a component of a cancer-implicated pathway, for coadministration with an inhibitor of Hsp90, to a subject suffering from a cancer which comprises the following steps:
(a) contacting a sample containing cancer cells from the subject with (i) an inhibitor of Hsp90 which binds to Hsp90 when such Hsp90 is bound to cancer pathway components present in the sample; or (ii) an analog, homolog, or derivative of such Hsp90 inhibitor which binds to Hsp90 when such Hsp90 is bound to such cancer pathway components in the sample;
(b) detecting pathway components bound to Hsp90;
(c) analyzing the pathway components detected in step (b) so as to identify a pathway which includes the components detected in step (b) and additional components of such pathway; and
(d) selecting an inhibitor of the pathway or of a pathway component identified in step (c).

In connection with the invention a cancer-implicated pathway is a pathway involved in metabolism, genetic information processing, environmental information processing, cellular processes, or organismal systems including any pathway listed in Table 1.

In the practice of this invention the cancer-implicated pathway or the component of the cancer-implicated pathway is involved with a cancer selected from the group consisting of colorectal cancer, pancreatic cancer, thyroid cancer, a leukemia including acute myeloid leukemia and chronic myeloid leukemia, basal cell carcinoma, melanoma, renal cell carcinoma, bladder cancer, prostate cancer, a lung cancer including small cell lung cancer and non-small cell lung cancer, breast cancer, neuroblastoma, myeloproliferative disorders, gastrointestinal cancers including gastrointestinal stromal tumors, esophageal cancer, stomach cancer, liver cancer, gallbladder cancer, anal cancer, brain tumors including gliomas, lymphomas including follicular lymphoma and diffuse large B-cell lymphoma, and gynecologic cancers including ovarian, cervical, and endometrial cancers. For example the component of the cancer-implicated pathway and/or the pathway may be any component identified in FIG. 1.

In a preferred embodiment involving personalized medicine in step (a) the subject is the same subject to whom the inhibitor of the cancer-implicated pathway or the component of the cancer-implicated pathway is to be administered although the invention in step (a) also contemplates the subject is a cancer reference subject.

In the practice of this invention in step (a) the sample comprises any tumor tissue or any biological fluid, for example, blood.

Suitable samples for use in the invention include, but are not limited to, disrupted cancer cells, lysed cancer cells, and sonicated cancer cells.

In connection with the practice of the invention the inhibitor of Hsp90 to be administered to the subject may be the same as or different from the (a) inhibitor of Hsp90 used, or (b) the inhibitor of Hsp90, the analog, homolog or derivative of the inhibitor of Hsp90 used, in step (a).

In one embodiment, wherein the inhibitor of Hsp90 to be administered to the subject is PU-H71 or an analog, homolog or derivative of PU-H71 having the biological activity of PU-H71.

In another embodiment PU-H71 is the inhibitor of Hsp90 used, or is the inhibitor of Hsp90, the analog, homolog or derivative of which is used, in step (a). Alternatively, the inhibitor of Hsp90 may be selected from the group consisting of the compounds shown in FIG. 3.

In one embodiment in step (a) the inhibitor of Hsp90 or the analog, homolog or derivative of the inhibitor of Hsp90 is preferred immobilized on a solid support, such as a bead.

In certain embodiments in step (b) the detection of pathway components comprises the use of mass spectroscopy, and in step (c) the analysis of the pathway components comprises use of a bioinformatics computer program.

In one example of the invention the cancer is a lymphoma, and in step (c) the pathway component identified is Syk. In another example, the cancer is a chronic myelogenous leukemia (CML) and in step (c) the pathway or the pathway component identified is a pathway or component shown in any of the Networks shown in FIG. 15, for example one of the following pathway components identified in FIG. 15, i.e. mTOR, IKK, MEK, NFκB, STAT3, STAT5A, STAT5B, Raf-1, bcr-abl, Btk, CARM1, or c-MYC. In one such example in step (c) the pathway component identified is mTOR and in step (d) the inhibitor selected is PP242. In another such example in step (c) the pathway identified is a pathway selected from the following pathways: PI3K/mTOR-, NFκB-, MAPK-, STAT-, FAK-, MYC and TGF-β mediated signaling pathways. In yet another example the cancer is a lymphoma, and in step (c) the pathway component identified is Btk. In a still further example the cancer is a pancreatic cancer, and in step (c) the pathway or pathway component identified is a pathway or pathway component shown in any of Networks 1-10 of FIG. 16 and in those of FIG. 24. In another example, in step (c) the pathway and pathway component identified is mTOR and in an example thereof in step (d) the inhibitor of mTOR selected is PP242. This invention further provides a method of treating a subject suffering from a cancer comprises coadministering to the subject (A) an inhibitor of Hsp90 and (B) an inhibitor of a component of a cancer-implicated pathway which in (B) need not be but may be selected by the method described herein. Thus this invention provides a treatment method wherein coadministering comprises administering the inhibitor in (A) and the inhibitor in (B) simultaneously, concomitantly, sequentially, or adjunctively. One example of the method of treating a subject suffering from a cancer comprises coadministering to the subject (A) an inhibitor of Hsp90 and (B) an inhibitor of Btk. Another example of the method of treating a subject suffering from a cancer which comprises coadministering to the subject (A) an inhibitor of Hsp90 and (B) an inhibitor of Syk. In such methods the cancer may be a lymphoma. Another example of the method of treating a subject suffering from a chronic myelogenous leukemia (CML) comprises coadministering to the subject (A) an inhibitor of Hsp90 and (B) an inhibitor of any of mTOR, IKK, MEK, NFκB, STAT3, STAT5A, STAT5B, Raf-1, bcr-abl, CARM1, CAMKII, or c-MYC. In an embodiment of the invention the inhibitor in (B) is an inhibitor of mTOR. In a further embodiment of the method described above in (a) binding of the inhibitor of Hsp90 or the analog, homolog, or derivative of such Hsp90 inhibitor traps Hsp90 in a cancer pathway components-bound state. Still further the invention provides a method of treating a subject suffering from a pancreatic cancer which comprises coadministering to the subject (A) an inhibitor of Hsp90 and (B) an inhibitor of the pathway or of a pathway component shown in any of the Networks shown in FIGS. 16 and 24. This invention also provides a method of treating a subject suffering from a breast cancer which comprises coadministering to the subject (A) an inhibitor of Hsp90 and (B) an inhibitor of the pathway or of a pathway component shown in any of the Networks shown in FIG. 22. Still further this invention provides a method of treating a subject suffering from a lymphoma which comprises coadministering to the subject (A) an inhibitor of Hsp90 and (B) an inhibitor of the pathway or of a pathway component shown in any of the Networks shown in FIG. 23. In the immediately preceeding methods the inhibitor in (B) may be an inhibitor of mTOR, e.g. PP242. Still further this invention provides a method of treating a subject suffering from a chronic myelogenous leukemia (CML) which comprises administering to the subject an inhibitor of CARM1. In another embodiment this invention provides a method for identifying a cancer-implicated pathway or one or more components of a cancer-implicated pathway in a subject suffering from cancer which comprises:
(a) contacting a sample containing cancer cells from the subject with (i) an inhibitor of Hsp90 which binds to Hsp90 when such Hsp90 is bound to cancer pathway components present in the sample; or (ii) an analog, homolog, or derivative of such Hsp90 inhibitor which binds to Hsp90 when such Hsp90 is bound to such cancer pathway components in the sample;
(b) detecting pathway components bound to Hsp90,
   so as to thereby identify the cancer-implicated pathway or said one or more pathway components. In this embodiment the cancer-implicated pathway or the component of the cancer-implicated pathway may be involved with any cancer selected from the group consisting of colorectal cancer, pancreatic cancer, thyroid cancer, a leukemia including acute myeloid leukemia and chronic myeloid leukemia, basal cell carcinoma, melanoma, renal cell carcinoma, bladder cancer, prostate cancer, a lung cancer including small cell lung cancer and non-small cell lung cancer, breast cancer, neuroblastoma, myeloproliferative disorders, gastrointestinal cancers including gastrointestinal stromal tumors, esophageal cancer, stomach cancer, liver cancer, gallbladder cancer, anal cancer, brain tumors including gliomas, lymphomas including follicular lymphoma and diffuse large B-cell lymphoma, and gynecologic cancers including ovarian, cervical, and endometrial cancers. Further in step (a) the sample may comprise a tumor tissue or a biological fluid, e.g., blood. In certain embodiments in step (a) the sample may comprise disrupted cancer cells, lysed cancer cells, or sonicated cancer cells. However, cells in other forms may be used.

In the practice of this method the inhibitor of Hsp90 may be PU-H71 or an analog, homolog or derivative of PU-H71 although PU-H71 is currently a preferred inhibitor. In the practice of the invention, however the inhibitor of Hsp90 may be selected from the group consisting of the compounds shown in FIG. 3. In an embodiment in step (a) the inhibitor of Hsp90 or the analog, homolog or derivative of the inhibitor of Hsp90 is immobilized on a solid support, such as a bead; and/or in step (b) the detection of pathway components comprises use of mass spectroscopy; and/or in step (c) the analysis of the pathway components comprises use of a bioinformatics computer program.

In one desirable embodiment of the invention in (a) binding of the inhibitor of Hsp90 or the analog, homolog, or derivative of such Hsp90 inhibitor traps Hsp90 in a cancer pathway components-bound state.

This invention further provides a kit for carrying out the method which comprises an inhibitor of Hsp90 immobilized on a solid support such as a bead. Typically, such a kit will further comprise control beads, buffer solution, and instructions for use. This invention further provides an inhibitor of Hsp90 immobilized on a solid support wherein the inhibitor is useful in the method described herein. One example is where the inhibitor is PU-H71. In another aspect this invention provides a compound having the structure:

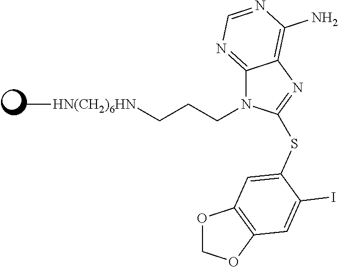

Still further the invention provides a method for selecting an inhibitor of a cancer-implicated pathway or a component of a cancer-implicated pathway which comprises identifying the cancer-implicated pathway or one or more components of such pathway according to the method described and then selecting an inhibitor of such pathway or such component. In addition, the invention provides a method of treating a subject comprising selecting an inhibitor according to the method described and administering the inhibitor to the subject alone or in addition to administering the inhibitor of the pathway component. More typically said administering will be effected repeatedly. Still further the methods described for identifying pathway components or selecting inhibitors may be performed at least twice for the same subject. In yet another embodiment this invention provides a method for monitoring the efficacy of treatment of a cancer with an Hsp90 inhibitor which comprises measuring changes in a biomarker which is a component of a pathway implicated in such cancer. For example, the biomarker used may be a component identified by the method described herein. In addition, this invention provides a method for monitoring the efficacy of a treatment of a cancer with both an Hsp90 inhibitor and a second inhibitor of a component of the pathway implicated in such cancer which Hsp90 inhibits which comprises monitoring changes in a biomarker which is a component of such pathway. For example, the biomarker used may be the component of the pathway being inhibited by the second inhibitor. Finally, this invention provides a method for identifying a new target for therapy of a cancer which comprises identifying a component of a pathway implicated in such cancer by the method described herein, wherein the component so identified has not previously been implicated in such cancer.

### BRIEF DESCRIPTION OF THE FIGURES

FIG. 1 depicts exemplary cancer-implicated pathways in humans and components thereof.

FIG. 2 shows several examples of protein kinase inhibitors.

FIG. 3 shows the structure of PU-H71 and several other known Hsp90 inhibitors.

FIG. 4. PU-H71 interacts with a restricted fraction of Hsp90 that is more abundant in cancer cells. (a) Sequential immuno-purification steps with H9010, an anti-Hsp90 antibody, deplete Hsp90 in the MDA-MB-468 cell extract. Lysate=control cell extract. (b) Hsp90 from MDA-MB-468 extracts was isolated through sequential chemical- and immuno-purification steps. The amount of Hsp90 in each pool was quantified by densitometry and values were normalized to an internal standard. (c) Saturation studies were performed with ¹³¹I-PU-H71 in the indicated cells. All the isolated cell samples were counted and the specific uptake of ¹³¹I-PU-H71 determined. These data were plotted against the concentration of ¹³¹I-PU-H71 to give a saturation binding curve. Representative data of four separate repeats is presented (lower). Expression of Hsp90 in the indicated cells was analyzed by Western blot (upper).

FIG. 5. PU-H71 is selective for and isolates Hsp90 in complex with onco-proteins and co-chaperones. (a) Hsp90 complexes in K562 extracts were isolated by precipitation with H9010, a non-specific IgG, or by PU-H71- or Control-beads. Control beads contain ethanolamine, an Hsp90-inert molecule. Proteins in pull-downs were analyzed by Western blot. (b,c) Single or sequential immuno- and chemical-precipitations, as indicated, were conducted in K562 extracts with H9010 and PU-beads at the indicated frequency and in the shown sequence. Proteins in the pull-downs and in the remaining supernatant were analyzed by WB. NS=non-specific. (d) K562 cell were treated for 24 h with vehicle (−) or PU-H71 (+), and proteins analyzed by Western blot. (e) Expression of proteins in Hsp70-knocked-down cells was analyzed by Western blot (left) and changes in protein levels presented in relative luminescence units (RLU) (right). Control=scramble siRNA. (f) Sequential chemical-precipitations, as indicated, were conducted in K562 extracts with GM-, SNX- and NVP-beads at the indicated frequency and in the shown sequence. Proteins in the pull-downs and in the remaining supernatant were analyzed by Western blot. (g) Hsp90 in K562 cells exists in complex with both aberrant, Bcr-Abl, and normal, c-Abl, proteins. PU-H71, but not H9010, selects for the Hsp90 population that is Bcr-Abl onco-protein bound.

FIG. 6. PU-H71 identifies the aberrant signalosome in CML cells. (a) Protein complexes were isolated through chemical precipitation by incubating a K562 extract with PU-beads, and the identity of proteins was probed by MS. Connectivity among these proteins was analyzed in IPA, and protein networks generated. The protein networks identified by the PU-beads (Networks 1 through 13) overlap well with the known canonical myeloid leukemia signaling (provided by IPA). A detailed list of identified protein networks and component proteins is shown in Table 5f and FIG. 15. (b) Pathway diagram highlighting the PU-beads identified CML signalosome with focus on Networks 1 (Raf-MAPK and PI3K-AKT pathway), 2 (NF-κB pathway) and 8 (STAT5-pathway). Key nodal proteins in the identified networks are depicted in yellow. (c) MS findings were validated by Western blot. (left) Protein complexes were isolated through chemical precipitation by incubating a K562 extract with PU- or control-beads, and proteins analyzed by Western blot. No proteins were detected in the Control-bead pull-downs and those data are omitted for simplicity of presentation. (right) K562 cell were treated for 24 h with vehicle (−) or PU-H71 (+), and proteins were analyzed by WB. (d) Single chemical-precipitations were conducted in primary CML cell extracts with PU- and Control-beads. Proteins in the pull-downs were analyzed by WB.

FIG. 7. PU-H71 identified proteins and networks are those important for the malignant phenotype. (a) K562 cells were treated for 72 h with the indicated inhibitors and cell growth analyzed by the Alamar Blue assay. Data are presented as means±SD (n=3). (b) Sequential chemical-precipitations, as indicated, were conducted in K562 extracts with the PU-beads at the indicated frequency. Proteins in the pull-downs and in the remaining supernatant were analyzed by WB. (c) The effect of CARM1 knock-down on cell viability using Tryptan blue (left) or Acridine orange/Ethidium bromide (right) stainings was evaluated in K562 cells. (d) The expression of select potential Hsp90-interacting proteins was analyzed by WB in K562 leukemia and Mia-PaCa-2 pancreatic cancer cells. (e) Select proteins isolated on PU-beads from K562 and Mia-PaCa-2 cell extracts, respectively, and subsequently identified by MS were tabulated. +++, very high; ++, high; +, moderate and −, no identifying peptides were found in MS analyses. (f) Single chemical-precipitations were conducted in Mia-PaCa-2 cell extracts with PU- and Control-beads. Proteins in the pull-downs were analyzed by WB. (g) The effect of select inhibitors on Mia-PaCa-2 cell growth was analyzed as in panel (a).

FIG. 8. Hsp90 facilitates an enhanced STAT5 activity in CML. (a) K562 cells were treated for the indicated times with PU-H71 (5 μM), Gleevec (0.5 μM) or DMSO (vehicle) and proteins analyzed by WB. (b) Sequential chemical-precipitations were conducted in K562 cells with PU- and Control-beads, as indicated. Proteins in the pull-downs and in the remaining supernatant were analyzed by WB. (c) STAT5 immuno-complexes from cells pre-treated with vehicle or PU-H71 were treated for the indicated times with trypsin and proteins analyzed by WB. (d) K562 cells were treated for the indicated times with vanadate (1 mM) in the presence and absence of PU-H71 (5 μM). Proteins were analyzed by WB (upper), quantified by densitometry and graphed against treatment time (lower). Data are presented as means±SD (n=3). (e) The DNA-binding capacity of STAT5a and STAT5b was assayed by an ELISA-based assay in K562 cells treated for 24 h with indicated concentrations of PU-H71. (f) Quantitative chromatin immunoprecipitation assays (QChIP) performed with STAT5 or Hsp90 antibodies vs. IgG control for two known STAT5 target genes (CCND2 and MYC). A primer that amplifies an intergenic region was used as negative control. Results are expressed as percentage of the input for the specific antibody (STAT5 or Hsp90) over the respective IgG control. (g) The transcript abundance of CCND2 and MYC was measured by QPCR in K562 cells exposed to 1 μM of PU-H71. Results are expressed as fold change compared to baseline (time 0 h) and were normalized to RPL13A. HPRT was used as negative control. Experiments were carried out in biological quintuplicates with experimental duplicates. Data are presented as means±SEM. (h) Proposed mechanism for and Hsp90-facilitated increased STAT5 signaling in CML. Hsp90 binds to and influences the conformation of STAT5 and maintains STAT5 in an active conformation directly within STAT5-containing transcriptional complexes.

FIG. 9. Schematic representation of the chemical-proteomics method for surveying tumor oncoproteins. Hsp90 forms biochemically distinct complexes in cancer cells. A major fraction of cancer cell Hsp90 retains “house keeping” chaperone functions similar to normal cells (green), whereas a functionally distinct Hsp90 pool enriched or expanded in cancer cells specifically interacts with oncogenic proteins required to maintain tumor cell survival (yellow). PU-H71 specifically interacts with Hsp90 and preferentially selects for onco-protein (yellow)/Hsp90 species but not WT protein (green)/Hsp90 species, and traps Hsp90 in a client binding conformation. The PU-H71 beads therefore can be used to isolate the onco-protein/Hsp90 species. In an initial step, the cancer cell extract is incubated with the PU-H71 beads (1). This initial chemical precipitation step purifies and enriches the aberrant protein population as part of PU-bead bound Hsp90 complexes (2). Protein cargo from PU-bead pull-downs is then eluted in SDS buffer, submitted to standard SDS-PAGE (3), and then the separated proteins are extracted and trypsinized for LC/MS/MS analyses (4). Initial protein identification is performed using the Mascot search engine, and is further evaluated using Scaffold Proteome Software (5). Ingenuity Pathway Analysis (IPA) is then used to build biological networks from the identified proteins (6,7). The created protein network map provides an invaluable template to develop personalized therapies that are optimally effective for a specific tumor. The method may (a) establish a map of molecular alterations in a tumor-by-tumor manner, (b) identify new oncoproteins and cancer mechanisms (c) identify therapeutic targets complementary to Hsp90 and develop rationally combinatorial targeted therapies and (d) identify tumor-specific biomarkers for selection of patients likely to benefit from Hsp90 therapy and for pharmacodynamics monitoring of Hsp90 inhibitor efficacy during clinical trials

FIG. 10. (a,b) Hsp90 from breast cancer and CML cell extracts (120 μg) was isolated through serial chemical- and immuno-purification steps, as indicated. The supernatant was isolated to analyze the left-over Hsp90. Hsp90 in each fraction was analyzed by Western blot. Lysate=endogenous protein content; PU-, GM- and Control-beads indicate proteins isolated on the particular beads. H9010 and IgG indicate protein isolated by the particular Ab. Control beads contain an Hsp90 inert molecule. The data are consistent with those obtained from multiple repeat experiments (n≧2). (c) Sequential chemical- and immuno-purification steps were performed in peripheral blood leukocyte (PBL) extracts (250 μg) to isolate PU-H71 and H9010-specific Hsp90 species. All samples were analyzed by Western blot. (upper). Binding to Hsp90 in PBL was evaluated by flow cytometry using an Hsp90-PE antibody and PU-H71-FITC. FITC-TEG=control for non-specific binding (lower).

FIG. 11. (a) Within normal cells, constitutive expression of Hsp90 is required for its evolutionarily conserved housekeeping function of folding and translocating cellular proteins to their proper cellular compartment (“housekeeping complex”). Upon malignant transformation, cellular proteins are perturbed through mutations, hyperactivity, retention in incorrect cellular compartments or other means. The presence of these functionally altered proteins is required to initiate and maintain the malignant phenotype, and it is these oncogenic proteins that are specifically maintained by a subset of stress modified Hsp90 (“oncogenic complex”). PU-H71 specifically binds to the fraction of Hsp90 that chaperones oncogenic proteins (“oncogenic complex”). (b) Hsp90 and its interacting co-chaperones were isolated in K562 cell extracts using PU- and Control-beads, and H9010 and IgG-immobilized Abs. Control beads contain an Hsp90 inert molecule. (c) Hsp90 from K562 cell extracts was isolated through three serial immuno-purification steps with the H9010 Hsp90 specific antibody. The remaining supernatant was isolated to analyze the left-over proteins. Proteins in each fraction were analyzed by Western blot. Lysate=endogenous protein content. The data are consistent with those obtained from multiple repeat experiments (n≧2).

FIG. 12. GM and PU-H71 are selective for aberrant protein/Hsp90 species. (a) Bcr-Abl and Abl bound Hsp90 species were monitored in experiments where a constant volume of PU-H71 beads (80 μL) was probed with indicated amounts of K562 cell lysate (left), or where a constant amount of lysate (1 mg) was probed with the indicated volumes of PU-H71 beads (right). (b) (left) PU- and GM-beads (80 μL) recognize the Hsp90-mutant B-Raf complex in the SKMel28 melanoma cell extract (300 μg), but fail to interact with the Hsp90-WT B-Raf complex found in the normal colon fibroblast CCD18Co extracts (300 μg). H9010 Hsp90 Ab recognizes both Hsp90 species. (c) In MDA-MB-468 cell extracts (300 μg), PU- and GM-beads (80 μl) interact with HER3 and Raf-1 kinase but not with the non-oncogenic tyrosine-protein kinase CSK, a c-Src related tyrosine kinase, and p38. (d) (right) PU-beads (80 μL) interact with v-Src/Hsp90 but not c-Src/Hsp90 species. To facilitate c-Src detection, a protein in lower abundance than v-Src, higher amounts of c-Src expressing 3T3 cell lysate (1,000 μg) were used when compared to the v-Src transformed 3T3 cell (250 μg), providing explanation for the higher Hsp90 levels detected in the 3T3 cells (Lysate, 3T3 fibroblasts vs v-Src 3T3 fibroblasts). Lysate=endogenous protein content; PU-, GM- and Control-beads indicate proteins isolated on the particular beads. Hsp90 Ab and IgG indicate protein isolated by the particular Ab. Control beads contain an Hsp90 inert molecule. The data are consistent with those obtained from multiple repeat experiments (n≧2).

FIG. 13. Single chemical-precipitations were conducted in Bcr-Abl-expressing CML cell lines (a) and in primary CML cell extracts (b) with PU- and Control-beads. Proteins in the pull-downs were analyzed by Western blot. Several Bcr-Abl cleavage products are noted in the primary CML samples as reported (Dierov et al., 2004). N/A=not available.

FIG. 14. PU-H71 is selective for Hsp90. (a) Coomassie stained gel of several Hsp90 inhibitor bead-pulldowns. K562 lysates (60 μg) were incubated with 25 μL of the indicated beads. Following washing with the indicated buffer, proteins in the pull-downs were applied to an SDS-PAGE gel. (b) PU-H71 (10 μM) was tested in the scanMAX screen (Ambit) against 359 kinases. The TREEspot™ Interaction Map for PU-H71 is presented. Only SNARK (NUAK family SNF 1-like kinase 2) (red dot on the kinase tree) appears as a potential low affinity kinase hit of the small molecule.

FIG. 15. Top scoring networks enriched on the PU-beads and as generated by bioinformatic pathways analysis through the use of the Ingenuity Pathways Analysis (IPA) software. Analysis was performed in the K562 chronic myeloid leukemia cells. (a) Network 1; Score=38; mTOR/PI3K and MAPK pathways. (b) Network 2; Score=36; NFκB pathway. (c) Network 8; Score=14; STAT pathway. (d) Network 12; Score=13; Focal adhesion network. (e) Network 7; Score=22; c-MYC oncogene driven pathway. (f) Network 10; Score=18; TGFβ pathway. Scores of 2 or higher have at least a 99% confidence of not being generated by random chance alone.

Gene expression, cell cycle and cellular assembly Individual proteins are displayed as nodes, utilizing gray to represent that the protein was identified in this study. Proteins identified by IPA only are represented as white nodes. Different shapes are used to represent the functional class of the gene product. Proteins are depicted in networks as two circles when the entity is part of a complex; as a single circle when only one unit is present; a triangle pointing up or down to describe a phosphatase or a kinase, respectively; by a horizontal oval to describe a transcription factor; and by circle to depict “other” functions. The edges describe the nature of the relationship between the nodes: an edge with arrow-head means that protein A acts on protein B, whereas an edge without an arrow-head represents binding only between two proteins. Direct interactions appear in the network diagram as a solid line, whereas indirect interactions as a dashed line. In some cases a relationship may exist as a circular arrow or line originating from one molecule and pointing back at that same molecule. Such relationships are termed “self-referential” and arise from the ability of a molecule to act upon itself

FIG. 16. Top scoring networks enriched on the PU-beads and as generated by bioinformatic pathways analysis through the use of the Ingenuity Pathways Analysis (IPA) software. Analysis was performed in the MiaPaCa2 pancreatic cancer cells.

FIG. 17. The mTOR inhibitor PP242 synergizes with the Hsp90 inhibitor PU-H71 in Mia-PaCa-2 cells. Pancreatic cells (Mia-PaCa-2) were treated for 72 h with single agent or combinations of PP242 and PU-H71 and cytotoxicity determined by the Alamar blue assay. Computerized simulation of synergism and/or antagonism in the drug combination studies was analyzed using the Chou-Talalay method. (a) In the median-effect equation, fa is the fraction of affected cells, e.g. fractional inhibition; fu=(1-fa) which is the fraction of unaffected cells; D is the dose required to produce fa. (b) Based on the actual experimental data, serial CI values were calculated for an entire range of effect levels (Fa), to generate Fa-CI plots. CI<1, =1, and >1 indicate synergism, additive effect, and antagonism, respectively. (c) Normalized isobologram showing the normalized dose of Drug 1 (PU-H71) and Drug2 (PP242). PU=PU-H71, PP=PP242.

Quantitative Analysis of Synergy Between mTOR and Hsp90 Inhibitors: To determine the drug interaction between pp242 (mTOR inhibitor) and PU-H71 (Hsp90 inhibitor), the combination index (CI) isobologram method of Chou-Talalay was used as previously described. This method, based on the median-effect principle of the law of mass action, quantifies synergism or antagonism for two or more drug combinations, regardless of the mechanisms of each drug, by computerized simulation. Based on algorithms, the computer software displays median-effect plots, combination index plots and normalized isobolograms (where non constant ratio combinations of 2 drugs are used). PU-H71 (0.5, 0.25, 0.125, 0.0625, 0.03125, 0.0125 μM) and pp242 (0.5, 0.125, 0.03125, 0.0008, 0.002, 0.001 μM) were used as single agents in the concentrations mentioned or combined in a non constant ratio (PU-H71:pp242; 1:1, 1:2, 1:4, 1:7.8, 1:15.6, 1:12.5). The Fa (fraction killed cells) was calculated using the formulae Fa=1−Fu; Fu is the fraction of unaffected cells and was used for a dose effect analysis using the computer software (CompuSyn, Paramus, N.J., USA).

FIG. 18. Bcl-6 is a client of Hsp90 in Bcl-6 dependent DLBCL cells and the combination of an Hsp90 inhibitor with a Bcl-6 inhibitor is more efficacious than each inhibitor alone. a) Cells were treated for 24 h with the indicated concentration of PU-H71 and proteins were analyzed by Western blot. b) PU-H71 beads indicate that Hsp90 interacts with Bcl-6 in the nucleus. c) the combination of the Hsp90 inhibitor PU-H71 with the Bcl-6 inhibitor RI-BPI is more efficacious in Bcl-6 dependent DLBCL cells than each inhibitor alone

FIG. 19. Several repeats of the method of the invention identify the B cell receptor network as a major pathway in the OCI-Ly1 cells to demonstrate and validate the robustness and accuracy of the method

FIG. 20. Validation of the B cell receptor network as an Hsp90 dependent network in OCI-LY1 and OCI-LY7 DLBCL cells. a) cells were treated with the Hsp90 inhibitor PU-H71 and proteins analyzed by Western blot. b) PU-H71 beads indicate that Hsp90 interacts with BTK and SYK in the OCI-LY1 and OCI-LY7 DLBCL cells. c) the combination of the Hsp90 inhibitor PU-H71 with the SYK inhibitor R406 is more efficacious in the Bcl-6 dependent OCI-LY1, OCI-LY7, Farage and SUDHL6 DLBCL cells than each inhibitor alone

FIG. 21. The CAMKII inhibitor KN93 and the mTOR inhibitor PP242 synergize with the Hsp90 inhibitor PU-H71 in K562 CML cells.

FIG. 22. Top scoring networks enriched on the PU-beads and as generated by bioinformatic pathways analysis through the use of the Ingenuity Pathways Analysis (IPA) software. Analysis was performed in the MDA-MB-468 triple-negative breast cancer cells. Major signaling networks identified by the method were the PI3K/AKT, IGF-IR, NRF2-mediated oxidative stress response, MYC, PKA and the IL-6 signaling pathways. (a) Simplified representation of networks identified in the MDA-MB-468 breast cancer cells by the PU-beads proteomics and bioinformatic method. (b) IL-6 pathway. Key network components identified by the PU-beads method in MDA-MB-468 breast cancer cells are depicted in grey.

FIG. 23. Top scoring networks enriched on the PU-beads and as generated by bioinformatic pathways analysis through the use of the Ingenuity Pathways Analysis (IPA) software. Analysis was performed in the OCI-Ly1 diffuse large B cell lymphoma (DLBCL) cells. In the Diffuse large B-cell lymphoma (DLBCL) cell line OCI-LY1, major signaling networks identified by the method were the B receptor, PKCteta, PI3K/AKT, CD40, CD28 and the ERK/MAPK signaling pathways. (a) B cell receptor pathway. Key network components identified by the PU-beads method are depicted in grey. (b) CD40 signaling pathway. Key network components identified by the PU-beads method are depicted in grey. (c) CD28 signaling pathway. Key network components identified by the PU-beads method are depicted in grey.

FIG. 24. Top scoring networks enriched on the PU-beads and as generated by bioinformatic pathways analysis through the use of the Ingenuity Pathways Analysis (IPA) software. Analysis was performed in the Mia-PaCa-2 pancreatic cancer cells. (a) PU-beads identify the aberrant signalosome in Mia-PaCa-2 cancer cells. Among the protein pathways identified by the PU-beads are those of the PI3K-Akt-mTOR-NFkB-pathway, TGF-beta pathway, Wnt-beta-catenin pathway, PKA-pathway, STAT3-pathway, JNK-pathway and the Rac-cdc42-ras-ERK pathway. (b) Cell cycle-G2/M DNA damage checkpoint regulation. Key network components identified by the PU-beads method are depicted in grey.

FIG. 25. PU-H71 synergizes with the PARP inhibitor olaparib in inhibiting the clonogenic survival of MDA-MB-468 (upper panels) and the HCC1937 (lower panel) breast cancer cells.

FIG. 26. Structures of Hsp90 inhibitors.

FIG. 27. A) Interactions of Hsp90α (PDB ID: 2FWZ) with PU-H71 (ball and stick model) and compound 5 (tube model). B) Interactions of Hsp90α (PDB ID: 2VCI) with NVP-AUY922 (ball and stick model) and compound 10 (tube model). C) Interactions of Hsp90α(PDB ID: 3D0B) with compound 27 (ball and stick model) and compound 20 (tube model). Hydrogen bonds are shown as dotted yellow lines and important active site amino acid residues and water molecules are represented as sticks.

FIG. 28. A) Hsp90 in K562 extracts (250 μg) was isolated by precipitation with PU-, SNX- and NVP-beads or Control-beads (80 μL). Control beads contain 2-methoxyethylamine, an Hsp90-inert molecule. Proteins in pull-downs were analyzed by Western blot. B) In MDA-MB-468 cell extracts (300 μg), PU-beads isolate Hsp90 in complex with its onco-client proteins, c-Kit and IGF-IR. To evaluate the effect of PU-H71 on the steady-state levels of Hsp90 onco-client proteins, cells were treated for 24 h with PU-H71 (5 μM). C) In K562 cell extracts, PU-beads (40 μL) isolate Hsp90 in complex with the Raf-1 and Bcr-Abl onco-proteins. Lysate=endogenous protein content; PU- and Control-beads indicate proteins isolated on the particular beads. The data are consistent with those obtained from multiple repeat experiments (n≧2).

FIG. 29. A) Hsp90-containing protein complexes from the brains of JNPL3 mice, an Alzheimer's disease transgenic mouse model, isolated through chemical precipitation with beads containing a streptavidin-immobilized PU-H71-biotin construct or control streptavidin-immobilized D-biotin. Aberrant tau species are indicated by arrow. c1, c2 and s1, s2, cortical and subcortical brain homogenates, respectively, extracted from 6-month-old female JNPL3 mice (Right). Western blot analysis of brain lysate protein content (Left). B) Cell surface Hsp90 in MV4-11 leukemia cells as detected by PU-H71-biotin. The data are consistent with those obtained from multiple repeat experiments (n≧2).

FIG. 30. Synthesis of PU-H71 beads (6).

FIG. 31. Synthesis of PU-H71-biotin (7).

FIG. 32. Synthesis of NVP-AUY922 beads (11).

FIG. 33. Synthesis of SNX-2112 beads (21).

FIG. 34. Synthesis of SNX-2112.

FIG. 35. Synthesis of purine and purine-like Hsp90 inhibitor beads. Both the pyrimidine and imidazopyridine (i.e X=N or CH) type inhibitors are described. Reagents and conditions: (a) Cs₂CO₃, 1,2-dibromoethane or 1,3-dibromopropane, DMF, rt; (b) NH₂(CH₂)₆NHBoc, DMF, rt, 24 h; (c) TFA, CH₂Cl₂, rt, 1 h; (d) Affigel-10, DIEA, DMAP, DMF.

9-(2-Bromoethyl)-8-(6-(dimethylamino)benzo[d][1,3]dioxol-5-ylthio)-9H-purin-6-amine (2a). 1a (29 mg, 0.0878 mmol), Cs₂CO₃ (42.9 mg, 0.1317 mmol), 1,2-dibromoethane (82.5 mg, 37.8 μL, 0.439 mmol) in DMF (0.6 mL) was stirred for 1.5 h at rt. Then additional Cs₂CO₃ (14 mg, 0.043 mmol) was added and the mixture stirred for an additional 20 min. The mixture was dried under reduced pressure and the residue purified by preparatory TLC (CH₂Cl₂:MeOH:AcOH, 15:1:0.5) to give 2a (24 mg, 63%). ¹H NMR (500 MHz, CDCl₃/MeOH-d₄) δ 8.24 (s, 1H), 6.81 (s, 1H), 6.68 (s, 1H), 5.96 (s, 2H), 4.62 (t, J=6.9 Hz, 2H), 3.68 (t, J=6.9 Hz, 2H), 2.70 (s, 6H); MS (ESI) m/z 437.2/439.1 [M+H]⁺.

tert-Butyl (6-((2-(6-amino-8-((6-(dimethylamino)benzo[d][1,3]dioxol-5-yl)thio)-9H-purin-9-yl)ethyl)amino)hexyl)carbamate (3a). 2a (0.185 g, 0.423 mmol) and tert-butyl 6-aminohexylcarbamate (0.915 g, 4.23 mmol) in DMF (7 mL) was stirred at rt for 24 h. The reaction mixture was concentrated and the residue chromatographed [CHCl₃:MeOH:MeOH—NH₃ (7N), 100:7:3] to give 0.206 g (85%) of 3a; MS (ESI) m/z 573.3 [M+H]⁺.

(4a). 3a (0.258 g, 0.45 mmol) was dissolved in 15 mL of CH₂Cl₂:TFA (4:1) and the solution was stirred at rt for 45 min. Solvent was removed under reduced pressure and the residue dried under high vacuum overnight. This was dissolved in DMF (12 mL) and added to 25 mL of Affi-Gel 10 beads (prewashed, 3×50 mL DMF) in a solid phase peptide synthesis vessel. 225 μL of N,N-diisopropylethylamine and several crystals of DMAP were added and this was shaken at rt for 2.5 h. Then 2-methoxyethylamine (0.085 g, 97 μl, 1.13 mmol) was added and shaking was continued for 30 minutes. Then the solvent was removed and the beads washed for 10 minutes each time with CH₂Cl₂:Et₃N (9:1, 4×50 mL), DMF (3×50 mL), Felts buffer (3×50 mL) and i-PrOH (3×50 mL). The beads 4a were stored in i-PrOH (beads: i-PrOH (1:2), v/v) at −80° C.

9-(3-Bromopropyl)-8-(6-(dimethylamino)benzo[d][1,3]dioxol-5-ylthio)-9H-purin-6-amine (2b). 1a (60 mg, 0.1818 mmol), Cs₂CO₃ (88.8 mg, 0.2727 mmol), 1,3-dibromopropane (184 mg, 93 μL, 0.909 mmol) in DMF (2 mL) was stirred for 40 min. at rt. The mixture was dried under reduced pressure and the residue purified by preparatory TLC (CH₂Cl₂:MeOH:AcOH, 15:1:0.5) to give 2b (60 mg, 73%). ¹H NMR (500 MHz, CDCl₃) δ 8.26 (s, 1H), 6.84 (br s, 2H), 6.77 (s, 1H), 6.50 (s, 1H), 5.92 (s, 2H), 4.35 (t, J=7.0 Hz, 2H), 3.37 (t, J=6.6 Hz, 2H), 2.68 (s, 6H), 2.34 (m, 2H); MS (ESI) m/z 451.1/453.1 [M+H]⁺.

tert-Butyl (6-((3-(6-amino-8-((6-(dimethylamino)benzo[d][1,3]dioxol-5-yl)thio)-9H-purin-9-yl)propyl)amino)hexyl)carbamate (3b). 2b (0.190 g, 0.423 mmol) and tert-butyl 6-aminohexylcarbamate (0.915 g, 4.23 mmol) in DMF (7 mL) was stirred at rt for 24 h. The reaction mixture was concentrated and the residue chromatographed [CHCl₃:MeOH:MeOH—NH₃ (7N), 100:7:3] to give 0.218 g (88%) of 3b; MS (ESI) m/z 587.3 [M+H]⁺.

(4b). 3b (0.264 g, 0.45 mmol) was dissolved in 15 mL of CH₂Cl₂:TFA (4:1) and the solution was stirred at rt for 45 min. Solvent was removed under reduced pressure and the residue dried under high vacuum overnight. This was dissolved in DMF (12 mL) and added to 25 mL of Affi-Gel 10 beads (prewashed, 3×50 mL DMF) in a solid phase peptide synthesis vessel. 225 μL of N,N-diisopropylethylamine and several crystals of DMAP were added and this was shaken at rt for 2.5 h. Then 2-methoxyethylamine (0.085 g, 97 μl, 1.13 mmol) was added and shaking was continued for 30 minutes. Then the solvent was removed and the beads washed for 10 minutes each time with CH₂Cl₂:Et₃N (9:1, 4×50 mL), DMF (3×50 mL), Felts buffer (3×50 mL) and i-PrOH (3×50 mL). The beads 4b were stored in i-PrOH (beads: i-PrOH (1:2), v/v) at −80° C.

1-(2-Bromoethyl)-2-((6-(dimethylamino)benzo[d][1,3]dioxol-5-yl)thio)-1H-imidazo[4,5-c]pyridin-4-amine (5a). 1b (252 mg, 0.764 mmol), Cs₂CO₃ (373 mg, 1.15 mmol), 1,2-dibromoethane (718 mg, 329 μL, 3.82 mmol) in DMF (2 mL) was stirred for 1.5 h at rt. Then additional Cs₂CO₃ (124 mg, 0.38 mmol) was added and the mixture stirred for an additional 20 min. The mixture was dried under reduced pressure and the residue purified by preparatory TLC (CH₂Cl₂:MeOH, 10:1) to give 5a (211 mg, 63%); MS (ESI) m/z 436.0/438.0 [M+H]⁺.

tert-Butyl (6-((2-(4-amino-2-((6-(dimethylamino)benzo[d][1,3]dioxol-5-yl)thio)-1H-imidazo[4,5-c]pyridin-1-yl)ethyl)amino)hexyl)carbamate (6a). 5a (0.184 g, 0.423 mmol) and tert-butyl 6-aminohexylcarbamate (0.915 g, 4.23 mmol) in DMF (7 mL) was stirred at rt for 24 h. The reaction mixture was concentrated and the residue chromatographed [CHCl₃:MeOH:MeOH—NH₃ (7N), 100:7:3] to give 0.109 g (45%) of 6a; MS (ESI) m/z 572.3 [M+H]⁺.

(7a). 6a (0.257 g, 0.45 mmol) was dissolved in 15 mL of CH₂Cl₂:TFA (4:1) and the solution was stirred at rt for 45 min. Solvent was removed under reduced pressure and the residue dried under high vacuum overnight. This was dissolved in DMF (12 mL) and added to 25 mL of Affi-Gel 10 beads (prewashed, 3×50 mL DMF) in a solid phase peptide synthesis vessel. 225 μL of N,N-diisopropylethylamine and several crystals of DMAP were added and this was shaken at rt for 2.5 h. Then 2-methoxyethylamine (0.085 g, 97 μl, 1.13 mmol) was added and shaking was continued for 30 minutes. Then the solvent was removed and the beads washed for 10 minutes each time with CH₂Cl₂:Et₃N (9:1, 4×50 mL), DMF (3×50 mL), Felts buffer (3×50 mL) and i-PrOH (3×50 mL). The beads 7a were stored in i-PrOH (beads: i-PrOH (1:2), v/v) at −80° C.

The beads 7b were prepared in a similar manner as described above for 7a.

FIG. 36. Synthesis of biotinylated purine and purine-like Hsp90 inhibitors. Reagents and conditions: (a) EZ-Link® Amine-PEO₃-Biotin, DMF, rt.

(8a). 2a (3.8 mg, 0.0086 mmol) and EZ-Link® Amine-PEO₃-Biotin (5.4 mg, 0.0129 mmol) in DMF (0.2 mL) was stirred at rt for 24 h. The reaction mixture was concentrated and the residue chromatographed [CHCl₃:MeOH—NH₃ (7N), 10:1] to give 2.3 mg (35%) of 8a. MS (ESI): m/z 775.2 [M+H]⁺.

(9a). 5a (3.7 mg, 0.0086 mmol) and EZ-Link® Amine-PEO₃-Biotin (5.4 mg, 0.0129 mmol) in DMF (0.2 mL) was stirred at rt for 24 h. The reaction mixture was concentrated and the residue chromatographed [CHCl₃:MeOH—NH₃ (7N), 10:1] to give 1.8 mg (27%) of 9a. MS (ESI): m/z 774.2 [M+H]⁺.

Biotinylated compounds 8b and 9b were prepared in a similar manner from 2b and 5b, respectively.

FIG. 37. Synthesis of biotinylated purine and purine-like Hsp90 inhibitors. Reagents and conditions: (a) N-(2-bromoethyl)-phthalimide or N-(3-bromopropyl)-phthalimide, Cs₂CO₃, DMF, rt; (b) hydrazine hydrate, MeOH, CH₂Cl₂, rt; (c) EZ-Link® NHS-LC-LC-Biotin, DIEA, DMF, rt; (d) EZ-Link® NHS-PEG₄-Biotin, DIEA, DMF, rt.

2-(3-(6-Amino-8-(6-(dimethylamino)benzo[d][1,3]dioxol-5-ylthio)-9H-purin-9-yl)propyl)isoindoline-1,3-dione. 1a (0.720 g, 2.18 mmol), Cs₂CO₃ (0.851 g, 2.62 mmol), 2-(3-bromopropyl)isoindoline-1,3-dione (2.05 g, 7.64 mmol) in DMF (15 mL) was stirred for 2 h at rt. The mixture was dried under reduced pressure and the residue purified by column chromatography (CH₂Cl₂:MeOH:AcOH, 15:1:0.5) to give 0.72 g (63%) of the titled compound. ¹H NMR (500 MHz, CDCl₃/MeOH-d₄): δ 8.16 (s, 1H), 7.85-7.87 (m, 2H), 7.74-7.75 (m, 2H), 6.87 (s, 1H), 6.71 (s, 1H), 5.88 (s, 2H), 4.37 (t, J=6.4 Hz, 2H), 3.73 (t, J=6.1 Hz, 2H), 2.69 (s, 6H), 2.37-2.42 (m, 2H); HRMS (ESI) m/z [M+H]⁺ calcd. for C₂₅H₂₄N₇O₄S, 518.1610. found 518.1601.

9-(3-Aminopropyl)-8-(6-(dimethylamino)benzo[d][1,3]dioxol-5-ylthio)-9H-purin-6-amine (10b). 2-(3-(6-Amino-8-(6-(dimethylamino)benzo[d][1,3]dioxol-5-ylthio)-9H-purin-9-yl)propyl)isoindoline-1,3-dione (0.72 g, 1.38 mmol), hydrazine hydrate (2.86 g, 2.78 mL, 20.75 mmol), in CH₂Cl₂:MeOH (4 mL:28 mL) was stirred for 2 h at rt. The mixture was dried under reduced pressure and the residue purified by column chromatography (CH₂Cl₂:MeOH—NH₃ (7N), 20:1) to give 430 mg (80%) of 10b. ¹H NMR (500 MHz, CDCl₃): δ 8.33 (s, 1H), 6.77 (s, 1H), 6.49 (s, 1H), 5.91 (s, 2H), 5.85 (br s, 2H), 4.30 (t, J=6.9 Hz, 2H), 2.69 (s, 6H), 2.65 (t, J=6.5 Hz, 2H), 1.89-1.95 (m, 2H); ¹³C NMR (125 MHz, CDCl₃): δ 154.5, 153.1, 151.7, 148.1, 147.2, 146.4, 144.8, 120.2, 120.1, 109.3, 109.2, 101.7, 45.3, 45.2, 40.9, 38.6, 33.3; HRMS (ESI) m/z [M+H]⁺ calcd. for C₁₇H₂₂N₇O₂S, 388.1556. found 388.1544.

(12b). 10b (13.6 mg, 0.0352 mmol), EZ-Link® NHS-LC-LC-Biotin (22.0 mg, 0.0387 mmol) and DIEA (9.1 mg, 12.3 μL, 0.0704 mmol) in DMF (0.5 mL) was stirred at rt for 1 h. The reaction mixture was concentrated under reduced pressure and the resulting residue was purified by preparatory TLC (CH₂Cl₂:MeOH—NH₃ (7N), 10:1) to give 22.7 mg (77%) of 12b. MS (ESI): m/z 840.2 [M+H]⁺.

(14b). 10b (14.5 mg, 0.0374 mmol), EZ-Link® NHS-PEG₄-Biotin (24.2 mg, 0.0411 mmol) and DIEA (9.7 mg, 13 μL, 0.0704 mmol) in DMF (0.5 mL) was stirred at rt for 1 h. The reaction mixture was concentrated under reduced pressure and the resulting residue was purified by preparatory TLC (CH₂Cl₂:MeOH—NH₃ (7N), 10:1) to give 24.1 mg (75%) of 14b. MS (ESI): m/z 861.3 [M+H]⁺.

Biotinylated compounds 12a, 13a, 13b, 14a, 15a and 15b were prepared in a similar manner as described for 12b and 14b.

FIG. 38. Synthesis of Debio 0932 type beads. Reagents and conditions: (a) Cs₂CO₃, DMF, rt; (b) TFA, CH₂Cl₂, rt; (c) 6-(BOC-amino)caproic acid, EDCI, DMAP, rt, 2 h; (d) Affigel-10, DIEA, DMAP, DMF.

8-((6-Bromobenzo[d][1,3]dioxol-5-yl)thio)-9-(2-(piperidin-4-yl)ethyl)-9H-purin-6-amine (18). 16 (300 mg, 0.819 mmol), Cs₂CO₃ (534 mg, 1.64 mmol), 17 (718 mg, 2.45 mmol) in DMF (10 mL) was stirred for 1.5 h at rt. The reaction mixture was filtered and dried under reduced pressure and chromatographed (CH₂Cl₂:MeOH, 10:1) to give a mixture of Boc-protected N9/N3 isomers. 20 mL of TFA:CH₂Cl₂ (1:1) was added at rt and stirred for 6 h. The reaction mixture was dried under reduced pressure and purified by preparatory HPLC to give 18 (87 mg, 22%); MS (ESI) m/z 477.0 [M+H]⁺.

6-Amino-1-(4-(2-(6-amino-8-((6-bromobenzo[d][1,3]dioxol-5-yl)thio)-9H-purin-9-yl)ethyl)piperidin-1-yl)hexan-1-one (19). To a mixture of 18 (150 mg, 0.314 mmol) in CH₂Cl₂ (5 ml) was added 6-(Boc-amino)caproic acid (145 mg, 0.628 mmol), EDCI (120 mg, 0.628 mmol) and DMAP (1.9 mg, 0.0157 mmol). The reaction mixture was stirred at rt for 2 h then concentrated under reduced pressure and the residue purified by preparatory TLC [CH₂Cl₂:MeOH—NH₃ (7N), 15:1] to give 161 mg (74%) of 19; MS (ESI) m/z 690.1 [M+H]⁺.

(20). 19 (0.264 g, 0.45 mmol) was dissolved in 15 mL of CH₂Cl₂:TFA (4:1) and the solution was stirred at rt for 45 min. Solvent was removed under reduced pressure and the residue dried under high vacuum overnight. This was dissolved in DMF (12 mL) and added to 25 mL of Affi-Gel 10 beads (prewashed, 3×50 mL DMF) in a solid phase peptide synthesis vessel. 225 μL of N,N-diisopropylethylamine and several crystals of DMAP were added and this was shaken at rt for 2.5 h. Then 2-methoxyethylamine (0.085 g, 97 μl, 1.13 mmol) was added and shaking was continued for 30 minutes. Then the solvent was removed and the beads washed for 10 minutes each time with CH₂Cl₂:Et₃N (9:1, 4×50 mL), DMF (3×50 mL), Felts buffer (3×50 mL) and i-PrOH (3×50 mL). The beads 20 were stored in i-PrOH (beads: i-PrOH (1:2), v/v) at −80° C.

FIG. 39. Synthesis of Debio 0932 linked to biotin. Reagents and conditions: (a) EZ-Link® NHS-LC-LC-Biotin, DIEA, DMF, 35° C.; (b) EZ-Link® NHS-PEG₄-Biotin, DIEA, DMF, 35° C.

(21). 18 (13.9 mg, 0.0292 mmol), EZ-Link® NHS-LC-LC-Biotin (18.2 mg, 0.0321 mmol) and DIEA (7.5 mg, 10.2 μL, 0.0584 mmol) in DMF (0.5 mL) was heated at 35° C. for 6 h. The reaction mixture was concentrated under reduced pressure and the resulting residue was purified by preparatory TLC (CH₂Cl₂:MeOH—NH₃ (7N), 10:1) to give 7.0 mg (26%) of 21. MS (ESI): m/z 929.3 [M+H]⁺.

(22). 18 (13.9 mg, 0.0292 mmol), EZ-Link® NHS-PEG₄-Biotin (18.9 mg, 0.0321 mmol) and DIEA (7.5 mg, 10.2 μL, 0.0584 mmol) in DMF (0.5 mL) was heated at 35° C. for 6 h. The reaction mixture was concentrated under reduced pressure and the resulting residue was purified by preparatory TLC (CH₂Cl₂:MeOH—NH₃ (7N), 10:1) to give 8.4 mg (30%) of 22; MS (ESI): m/z 950.2 [M+H]⁺.

FIG. 40. Synthesis of the SNX 2112type Hsp90 inhibitor linked to biotin. Reagents and conditions: (a) EZ-Link® NHS-LC-LC-Biotin, DIEA, DMF, rt; (b) EZ-Link® NHS-PEG₄-Biotin, DIEA, DMF, rt.

(24). 23 (16.3 mg, 0.0352 mmol), EZ-Link® NHS-LC-LC-Biotin (22.0 mg, 0.0387 mmol) and DIEA (9.1 mg, 12.3 μL, 0.0704 mmol) in DMF (0.5 mL) was stirred at rt for 1 h. The reaction mixture was concentrated under reduced pressure and the resulting residue was purified by preparatory TLC (CH₂Cl₂:MeOH, 10:1) to give 26.5 mg (82%) of 24; MS (ESI): m/z 916.4 [M+H]⁺.

(25). 23 (17.3 mg, 0.0374 mmol), EZ-Link® NHS-PEG₄-Biotin (24.2 mg, 0.0411 mmol) and DIEA (9.7 mg, 13 μL, 0.0704 mmol) in DMF (0.5 mL) was stirred at rt for 1 h. The reaction mixture was concentrated under reduced pressure and the resulting residue was purified by preparatory TLC (CH₂Cl₂:MeOH, 10:1) to give 30.1 mg (78%) of 25; MS (ESI): m/z 937.3 [M+H]⁺.

### DETAILED DESCRIPTION OF THE INVENTION

The present disclosure provides methods of identifying cancer-implicated pathways and specific components of cancer-implicated pathways (e.g., oncoproteins) associated with Hsp90 that are implicated in the development and progression of a cancer. Such methods involve contacting a sample containing cancer cells from a subject suffering from cancer with an inhibitor of Hsp90, and detecting the components of the cancer-implicated pathway that are bound to the inhibitor of Hsp90.

As used herein, certain terms have the meanings set forth after each such term as follows:

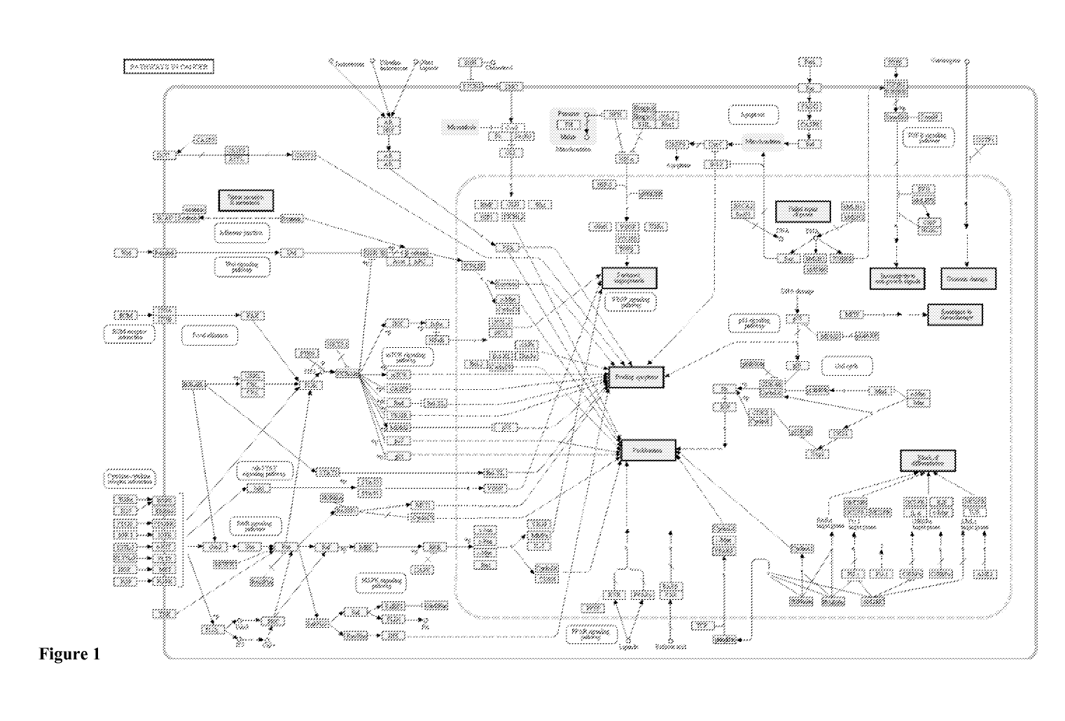

“Cancer-Implicated Pathway” means any molecular pathway, a variation in which is involved in the transformation of a cell from a normal to a cancer phenotype. Cancer-implicated pathways may include pathways involved in metabolism, genetic information processing, environmental information processing, cellular processes, and organismal systems. A list of many such pathways is set forth in Table 1 and more detailed information may be found about such pathways online in the KEGG PATHWAY database; and the National Cancer Institute's Nature Pathway Interaction Database. See also the websites of Cell Signaling Technology, Beverly, Mass.; BioCarta, San Diego, Calif.; and Invitrogen/Life Technologies Corporation, Clarsbad, Calif. In addition, FIG. 1 depicts pathways which are recognized to be involved in cancer.

TABLE 1
Examples of Potential Cancer-Implicated Pathways.

| | |
|---|---|
| 1. Metabolism | 1.1 Carbohydrate Metabolism |
| | Glycolysis/Gluconeogenesis |
| | Citrate cycle (TCA cycle) |
| | Pentose phosphate pathway |
| | Pentose and glucuronate interconversions |
| | Fructose and mannose metabolism |
| | Galactose metabolism |
| | Ascorbate and aldarate metabolism |
| | Starch and sucrose metabolism |
| | Amino sugar and nucleotide sugar metabolism |
| | Pyruvate metabolism |
| | Glyoxylate and dicarboxylate metabolism |
| | Propanoate metabolism |
| | Butanoate metabolism |
| | C5-Branched dibasic acid metabolism |
| | Inositol phosphate metabolism |
| | 1.2 Energy Metabolism |
| | Oxidative phosphorylation |
| | Photosynthesis |
| | Photosynthesis - antenna proteins |
| | Carbon fixation in photosynthetic organisms |
| | Carbon fixation pathways in prokaryotes |
| | Methane metabolism |
| | Nitrogen metabolism |
| | Sulfur metabolism |
| | 1.3 Lipid Metabolism |
| | Fatty acid biosynthesis |
| | Fatty acid elongation in mitochondria |
| | Fatty acid metabolism |
| | Synthesis and degradation of ketone bodies |
| | Steroid biosynthesis |
| | Primary bile acid biosynthesis |
| | Secondary bile acid biosynthesis |
| | Steroid hormone biosynthesis |
| | Glycerolipid metabolism |
| | Glycerophospholipid metabolism |
| | Ether lipid metabolism |
| | Sphingolipid metabolism |
| | Arachidonic acid metabolism |
| | Linoleic acid metabolism |
| | alpha-Linolenic acid metabolism |
| | Biosynthesis of unsaturated fatty acids |
| | 1.4 Nucleotide Metabolism |
| | Purine metabolism |
| | Pyrimidine metabolism |
| | 1.5 Amino Acid Metabolism |
| | Alanine, aspartate and glutamate metabolism |
| | Glycine, serine and threonine metabolism |
| | Cysteine and methionine metabolism |
| | Valine, leucine and isoleucine degradation |
| | Valine, leucine and isoleucine biosynthesis |
| | Lysine biosynthesis |
| | Lysine degradation |
| | Arginine and proline metabolism |
| | Histidine metabolism |
| | Tyrosine metabolism |
| | Phenylalanine metabolism |
| | Tryptophan metabolism |
| | Phenylalanine, tyrosine and tryptophan biosynthesis |
| | 1.6 Metabolism of Other Amino Acids |
| | beta-Alanine metabolism |
| | Taurine and hypotaurine metabolism |
| | Phosphonate and phosphinate metabolism |
| | Selenoamino acid metabolism |
| | Cyanoamino acid metabolism |
| | D-Glutamine and D-glutamate metabolism |
| | D-Arginine and D-ornithine metabolism |
| | D-Alanine metabolism |
| | Glutathione metabolism |
| | 1.7 Glycan Biosynthesis and Metabolism |
| | N-Glycan biosynthesis |
| | Various types of N-glycan biosynthesis |
| | Mucin type O-Glycan biosynthesis |
| | Other types of O-glycan biosynthesis |
| | Glycosaminoglycan biosynthesis - chondroitin sulfate |
| | Glycosaminoglycan biosynthesis - heparan sulfate |
| | Glycosaminoglycan biosynthesis - keratan sulfate |
| | Glycosaminoglycan degradation |
| | Glycosylphosphatidylinositol(GPI)-anchor biosynthesis |
| | Glycosphingolipid biosynthesis - lacto and neolacto series |
| | Glycosphingolipid biosynthesis - globo series |
| | Glycosphingolipid biosynthesis - ganglio series |
| | Lipopolysaccharide biosynthesis |
| | Peptidoglycan biosynthesis |
| | Other glycan degradation |
| | 1.8 Metabolism of Cofactors and Vitamins |
| | Thiamine metabolism |
| | Riboflavin metabolism |
| | Vitamin B6 metabolism |
| | Nicotinate and nicotinamide metabolism |
| | Pantothenate and CoA biosynthesis |
| | Biotin metabolism |
| | Lipoic acid metabolism |
| | Folate biosynthesis |
| | One carbon pool by folate |
| | Retinol metabolism |
| | Porphyrin and chlorophyll metabolism |
| | Ubiquinone and other terpenoid-quinone biosynthesis |
| | 1.9 Metabolism of Terpenoids and Polyketides |
| | Terpenoid backbone biosynthesis |
| | Monoterpenoid biosynthesis |
| | Sesquiterpenoid biosynthesis |
| | Diterpenoid biosynthesis |
| | Carotenoid biosynthesis |
| | Brassinosteroid biosynthesis |
| | Insect hormone biosynthesis |
| | Zeatin biosynthesis |
| | Limonene and pinene degradation |
| | Geraniol degradation |
| | Type I polyketide structures |
| | Biosynthesis of 12-, 14- and 16-membered macrolides |
| | Biosynthesis of ansamycins |
| | Biosynthesis of type II polyketide backbone |
| | Biosynthesis of type II polyketide products |
| | Tetracycline biosynthesis |
| | Polyketide sugar unit biosynthesis |
| | Nonribosomal peptide structures |
| | Biosynthesis of siderophore group nonribosomal peptides |
| | Biosynthesis of vancomycin group antibiotics |
| | 1.10 Biosynthesis of Other Secondary Metabolites |
| | Phenylpropanoid biosynthesis |
| | Stilbenoid, diarylheptanoid and gingerol biosynthesis |
| | Flavonoid biosynthesis |
| | Flavone and flavonol biosynthesis |
| | Anthocyanin biosynthesis |
| | Isoflavonoid biosynthesis |
| | Indole alkaloid biosynthesis |
| | Isoquinoline alkaloid biosynthesis |
| | Tropane, piperidine and pyridine alkaloid biosynthesis |
| | Acridone alkaloid biosynthesis |
| | Caffeine metabolism |
| | Betalain biosynthesis |
| | Glucosinolate biosynthesis |
| | Benzoxazinoid biosynthesis |
| | Penicillin and cephalosporin biosynthesis |
| | beta-Lactam resistance |
| | Streptomycin biosynthesis |
| | Butirosin and neomycin biosynthesis |
| | Clavulanic acid biosynthesis |
| | Puromycin biosynthesis |
| | Novobiocin biosynthesis |
| | 1.11 Xenobiotics Biodegradation and Metabolism |
| | Benzoate degradation |
| | Aminobenzoate degradation |
| | Fluorobenzoate degradation |
| | Chloroalkane and chloroalkene degradation |
| | Chlorocyclohexane and chlorobenzene degradation |
| | Toluene degradation |
| | Xylene degradation |
| | Nitrotoluene degradation |
| | Ethylbenzene degradation |
| | Styrene degradation |
| | Atrazine degradation |
| | Caprolactam degradation |
| | DDT degradation |
| | Bisphenol degradation |
| | Dioxin degradation |
| | Naphthalene degradation |
| | Polycyclic aromatic hydrocarbon degradation |
| | Metabolism of xenobiotics by cytochrome P450 |
| | Drug metabolism - cytochrome P450 |
| | Drug metabolism - other enzymes |
| | 1.12 Overview |
| | Overview of biosynthetic pathways |
| | Biosynthesis of plant secondary metabolites |
| | Biosynthesis of phenylpropanoids |
| | Biosynthesis of terpenoids and steroids |
| | Biosynthesis of alkaloids derived from shikimate pathway |
| | Biosynthesis of alkaloids derived from ornithine, lysine |
| | and nicotinic acid |
| | Biosynthesis of alkaloids derived from histidine and purine |
| | Biosynthesis of alkaloids derived from terpenoid and |
| | polyketide |
| | Biosynthesis of plant hormones |
| 2. Genetic | 2.1 Transcription |
| Information | RNA polymerase |
| Processing | Basal transcription factors |
| | Spliceosome |
| | 2.2 Translation |
| | Ribosome |
| | Aminoacyl-tRNA biosynthesis |
| | RNA transport |
| | mRNA surveillance pathway |
| | Ribosome biogenesis in eukaryotes |
| | 2.3 Folding, Sorting and Degradation |
| | Protein export |
| | Protein processing in endoplasmic reticulum |
| | SNARE interactions in vesicular transport |
| | Ubiquitin mediated proteolysis |
| | Sulfur relay system |
| | Proteasome |
| | RNA degradation |
| | 2.4 Replication and Repair |
| | DNA replication |
| | Base excision repair |
| | Nucleotide excision repair |
| | Mismatch repair |
| | Homologous recombination |
| | Non-homologous end joining |
| 3. Environmental | 3.1 Membrane Transport |
| Information | ABC transporters |
| Processing | Phosphotransferase system (PTS) |
| | Bacterial secretion system |
| | 3.2 Signal Transduction |
| | Two-component system |
| | MAPK signaling pathway |
| | MAPK signaling pathway - fly |
| | MAPK signaling pathway - yeast |
| | ErbB signaling pathway |
| | Wnt signaling pathway |
| | Notch signaling pathway |
| | Hedgehog signaling pathway |
| | TGF-beta signaling pathway |
| | VEGF signaling pathway |
| | Jak-STAT signaling pathway |
| | Calcium signaling pathway |
| | Phosphatidylinositol signaling system |
| | mTOR signaling pathway |
| | Plant hormone signal transduction |
| | 3.3 Signaling Molecules and Interaction |
| | Neuroactive ligand-receptor interaction |
| | Cytokine-cytokine receptor interaction |
| | ECM-receptor interaction |
| | Cell adhesion molecules (CAMs) |
| 4. Cellular Processes | 4.1 Transport and Catabolism |
| | Endocytosis |
| | Phagosome |
| | Lysosome |
| | Peroxisome |
| | Regulation of autophagy |
| | 4.2 Cell Motility |
| | Bacterial chemotaxis |
| | Flagellar assembly |
| | Regulation of actin cytoskeleton |
| | 4.3 Cell Growth and Death |
| | Cell cycle |
| | Cell cycle - yeast |
| | Cell cycle - Caulobacter |
| | Meiosis - yeast |
| | Oocyte meiosis |
| | Apoptosis |
| | p53 signaling pathway |
| | 4.4 Cell Communication |
| | Focal adhesion |
| | Adherens junction |
| | Tight junction |
| | Gap junction |
| 5. Organismal | 5.1 Immune System |
| Systems | Hematopoietic cell lineage |
| | Complement and coagulation cascades |
| | Toll-like receptor signaling pathway |
| | NOD-like receptor signaling pathway |
| | RIG-I-like receptor signaling pathway |
| | Cytosolic DNA-sensing pathway |
| | Natural killer cell mediated cytotoxicity |
| | Antigen processing and presentation |
| | T cell receptor signaling pathway |
| | B cell receptor signaling pathway |
| | Fc epsilon RI signaling pathway |
| | Fc gamma R-mediated phagocytosis |
| | Leukocyte transendothelial migration |
| | Intestinal immune network for IgA production |
| | Chemokine signaling pathway |
| | 5.2 Endocrine System |
| | Insulin signaling pathway |
| | Adipocytokine signaling pathway |
| | PPAR signaling pathway |
| | GnRH signaling pathway |
| | Progesterone-mediated oocyte maturation |
| | Melanogenesis |
| | Renin-angiotensin system |
| | 5.3 Circulatory System |
| | Cardiac muscle contraction |
| | Vascular smooth muscle contraction |
| | 5.4 Digestive System |
| | Salivary secretion |
| | Gastric acid secretion |
| | Pancreatic secretion |
| | Bile secretion |
| | Carbohydrate digestion and absorption |
| | Protein digestion and absorption |
| | Fat digestion and absorption |
| | Vitamin digestion and absorption |
| | Mineral absorption |
| | 5.5 Excretory System |
| | Vasopressin-regulated water reabsorption |
| | Aldosterone-regulated sodium reabsorption |
| | Endocrine and other factor-regulated calcium reabsorption |
| | Proximal tubule bicarbonate reclamation |
| | Collecting duct acid secretion |
| | 5.6 Nervous System |
| | Long-term potentiation |
| | Long-term depression |
| | Neurotrophin signaling pathway |
| | 5.7 Sensory System |
| | Phototransduction |
| | Phototransduction - fly |
| | Olfactory transduction |
| | Taste transduction |
| | 5.8 Development |
| | Dorso-ventral axis formation |
| | Axon guidance |
| | Osteoclast differentiation |
| | 5.9 Environmental Adaptation |
| | Circadian rhythm - mammal |
| | Circadian rhythm - fly |
| | Circadian rhythm - plant |
| | Plant-pathogen interaction |

“Component of a Cancer-Implicated Pathway” means a molecular entity located in a Cancer-Implicated Pathway which can be targeted in order to effect inhibition of the pathway and a change in a cancer phenotype which is associated with the pathway and which has resulted from activity in the pathway. Examples of such components include components listed in FIG. 1.

“Inhibitor of a Component of a Cancer-Implicated Pathway” means a compound (other than an inhibitor of Hsp90) which interacts with a Cancer-Implicated Pathway or a Component of a Cancer-Implicated Pathway so as to effect inhibition of the pathway and a change in a cancer phenotype which has resulted from activity in the pathway. Examples of inhibitors of specific Components are widely known. Merely by way of example, the following U.S. patents and U.S. patent application publications describe examples of inhibitors of pathway components as listed follows:
SYK: U.S. Patent Application Publications US 2009/0298823 A1, US 2010/0152159 A1, US 2010/0316649 A1
BTK: U.S. Pat. No. 6,160,010; U.S. Patent Application Publications US 2006/0167090 A1, US 2011/0008257 A1
EGFR: U.S. Pat. No. 5,760,041; U.S. Pat. No. 7,488,823 B2; U.S. Pat. No. 7,547,781 B2
mTOR: U.S. Pat. No. 7,504,397 B2; U.S. Patent Application Publication US 2011/0015197 A1
MET: U.S. Pat. No. 7,037,909 B2; U.S. Patent Application Publications US 2005/0107391 A1, US 2006/0009493 A1
MEK: U.S. Pat. No. 6,703,420 B1; U.S. Patent Application Publication US 2007/0287737 A1
VEGFR: U.S. Pat. No. 7,790,729 B2; U.S. Patent Application Publications US 2005/0234115 A1, US 2006/0074056 A1
PTEN: U.S. Patent Application Publications US 2007/0203098 A1, US 2010/0113515 A1
PKC: U.S. Pat. No. 5,552,396; U.S. Pat. No. 7,648,989 B2
Bcr-Abl: U.S. Pat. No. 7,625,894 B2; U.S. Patent Application Publication US 2006/0235006 A1

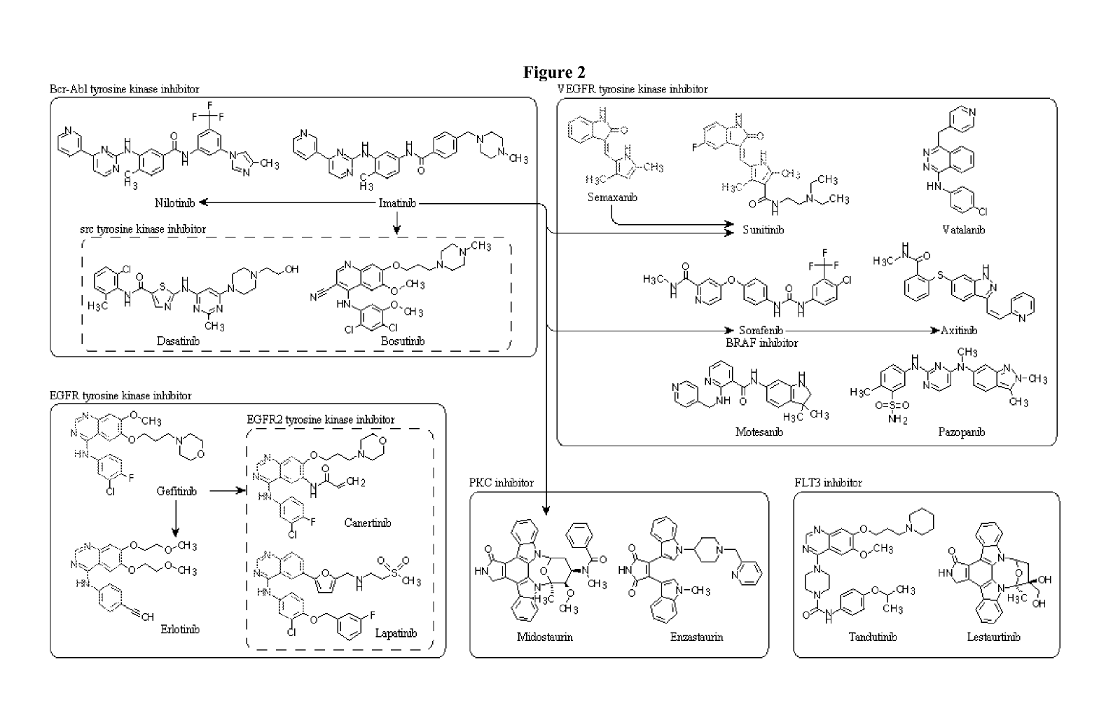

Still further a few examples of inhibitors of protein kinases are shown in FIG. 2.

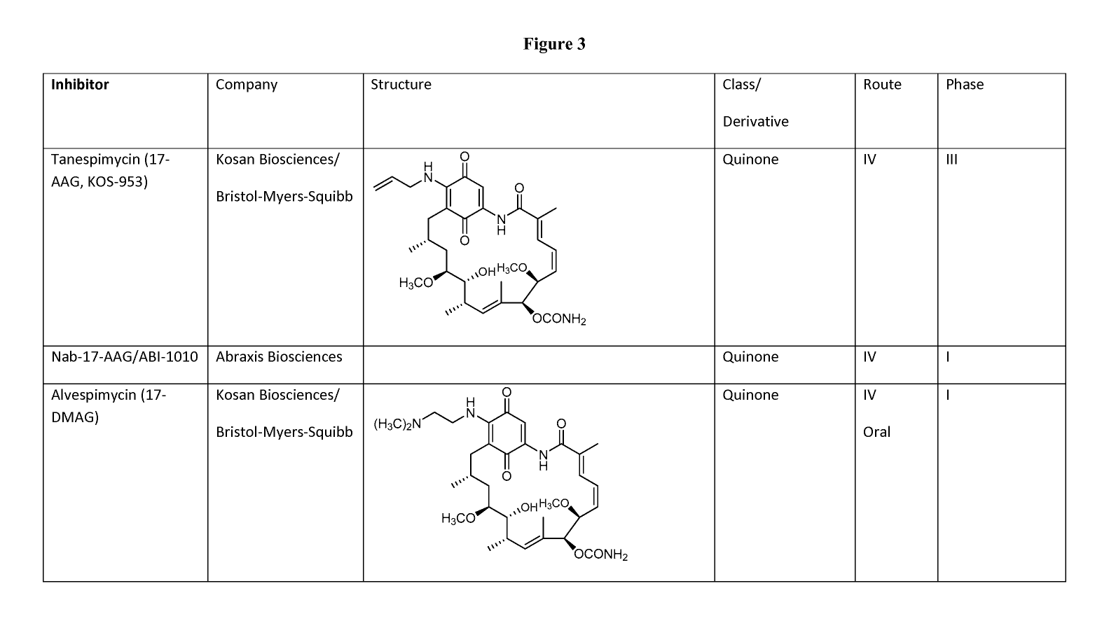

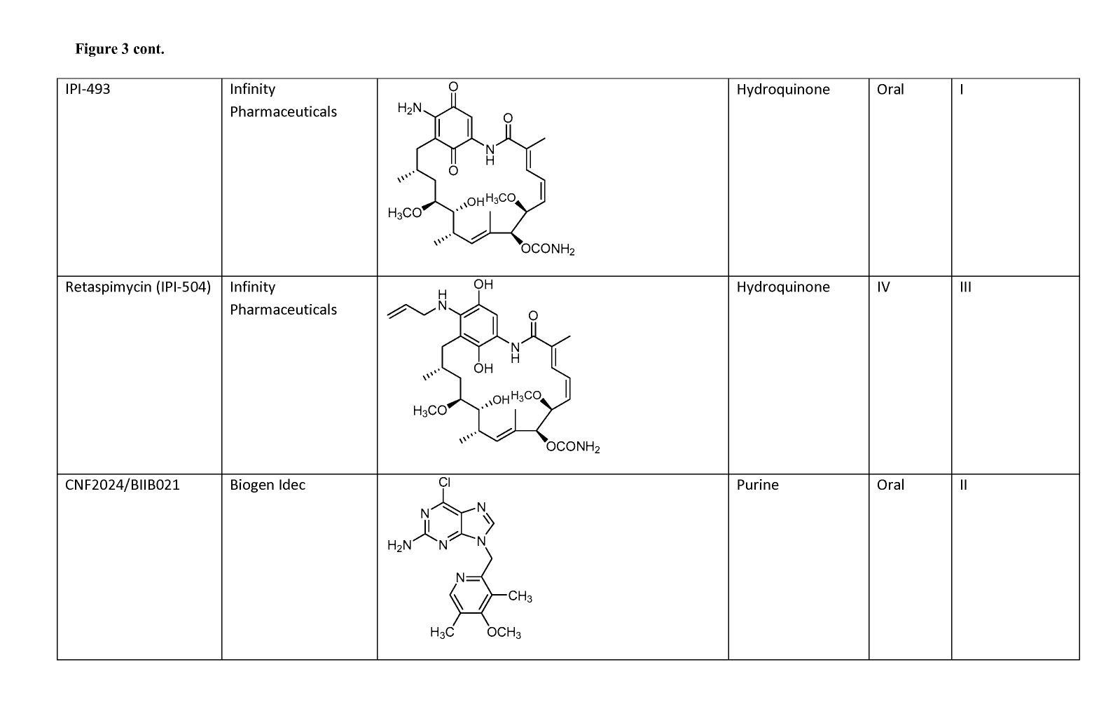

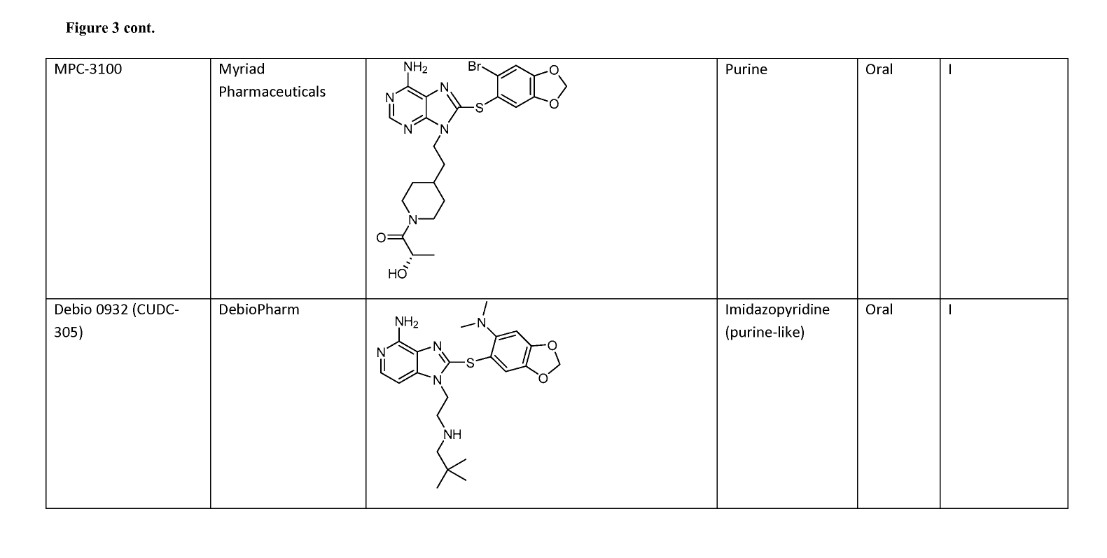

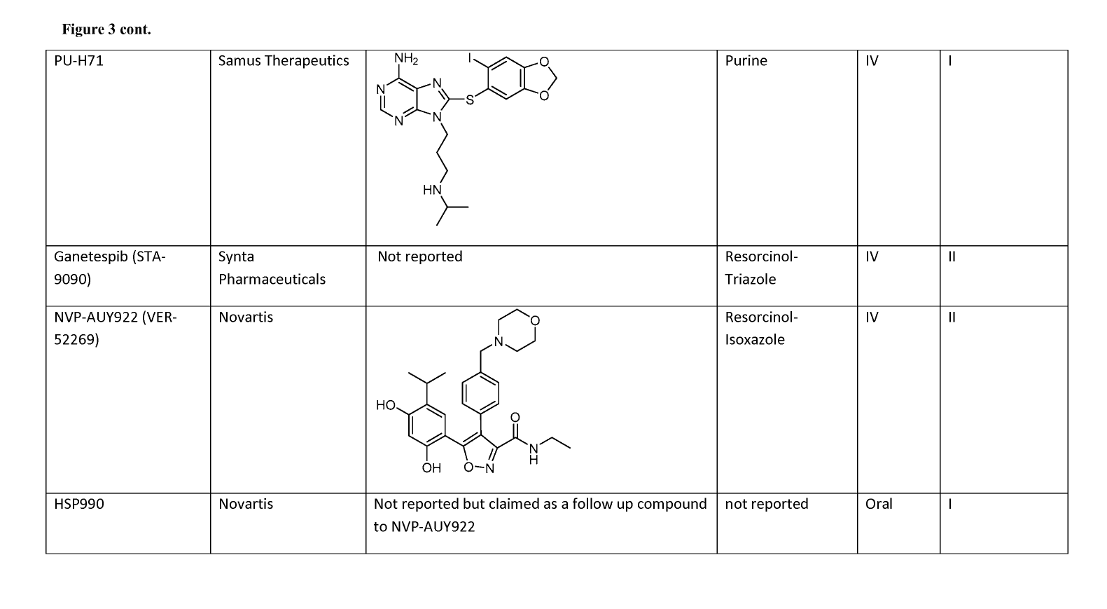

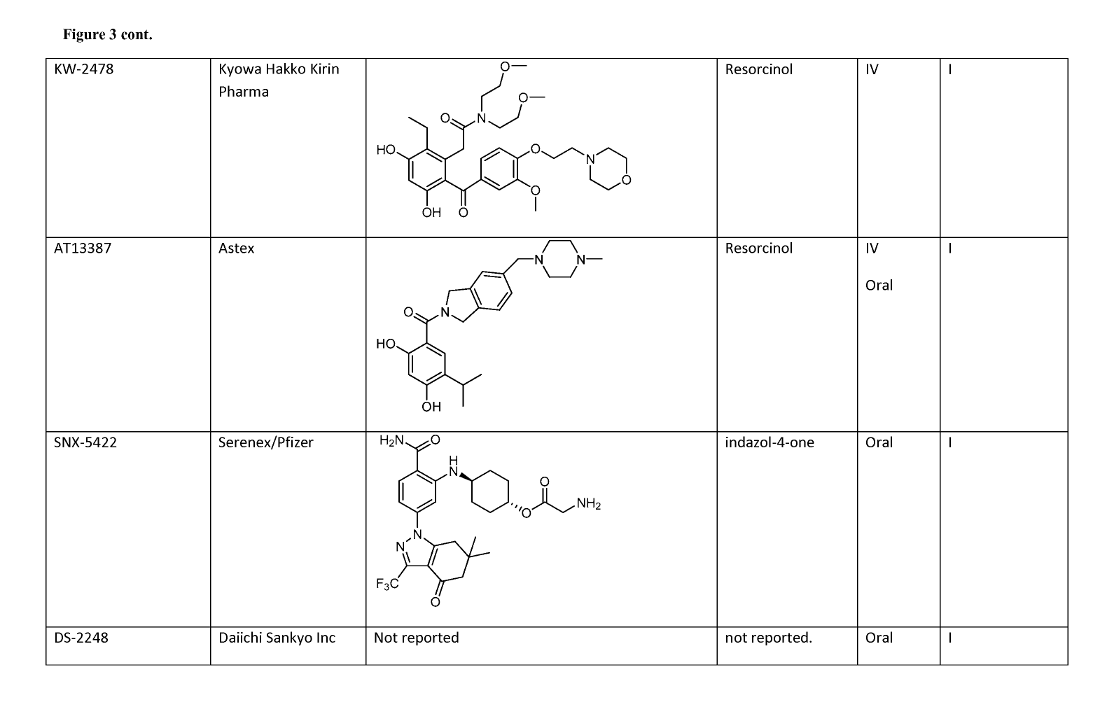

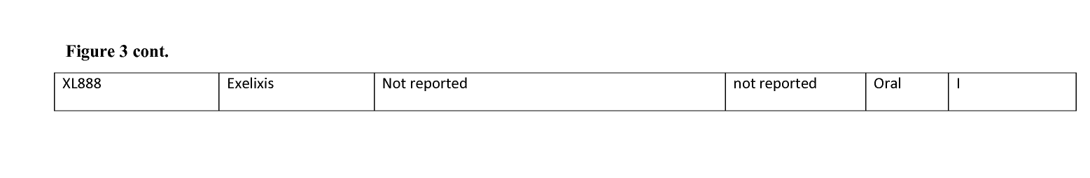

“Inhibitor of Hsp90” means a compound which interacts with, and inhibits the activity of, the chaperone, heat shock protein 90 (Hsp90). The structures of several known Hsp90 inhibitors, including PU-H71, are shown in FIG. 3. Many additional Hsp90 inhibitors have been described. See, for example, U.S. Pat. No. 7,820,658 B2; U.S. Pat. No. 7,834,181 B2; and U.S. Pat. No. 7,906,657 B2. See also the following:
Hardik J Patel, Shanu Modi, Gabriela Chiosis, Tony Taldone. Advances in the discovery and development of heat-shock protein 90 inhibitors for cancer treatment. Expert Opinion on Drug Discovery May 2011, Vol. 6, No. 5, Pages 559-587: 559-587;
Porter J R, Fritz C C, Depew K M. Discovery and development of Hsp90 inhibitors: a promising pathway for cancer therapy. Curr Opin Chem Biol. 2010 June; 14(3): 412-20;
Janin Y L. ATPase inhibitors of heat-shock protein 90, second season. Drug Discov Today. 2010 May; 15(9-10): 342-53;
Taldone T, Chiosis G. Purine-scaffold Hsp90 inhibitors. Curr Top Med Chem. 2009; 9(15): 1436-46; and
Taldone T, Sun W, Chiosis G. Discovery and Development of heat shock protein 90 inhibitors. Bioorg Med Chem. 2009 Mar. 15; 17(6): 2225-35.

### Small Molecule Hsp90 Probes

The attachment of small molecules to a solid support is a very useful method to probe their target and the target's interacting partners. Indeed, geldanamycin attached to solid support enabled for the identification of Hsp90 as its target. Perhaps the most crucial aspects in designing such chemical probes are determining the appropriate site for attachment of the small molecule ligand, and designing an appropriate linker between the molecule and the solid support. Our strategy to design Hsp90 chemical probes entails several steps. First, in order to validate the optimal linker length and its site of attachment to the Hsp90 ligand, the linker-modified ligand was docked onto an appropriate X-ray crystal structure of Hsp90α. Second, the linker-modified ligand was evaluated in a fluorescent polarization (FP) assay that measures competitive binding to Hsp90 derived from a cancer cell extract. This assay uses Cy3b-labeled geldanamycin as the FP-optimized Hsp90 ligand (Du et al., 2007). These steps are important to ensure that the solid-support immobilized molecules maintain a strong affinity for Hsp90. Finally, the linker-modified small molecule was attached to the solid support, and its interaction with Hsp90 was validated by incubation with an Hsp90-containing cell extract.

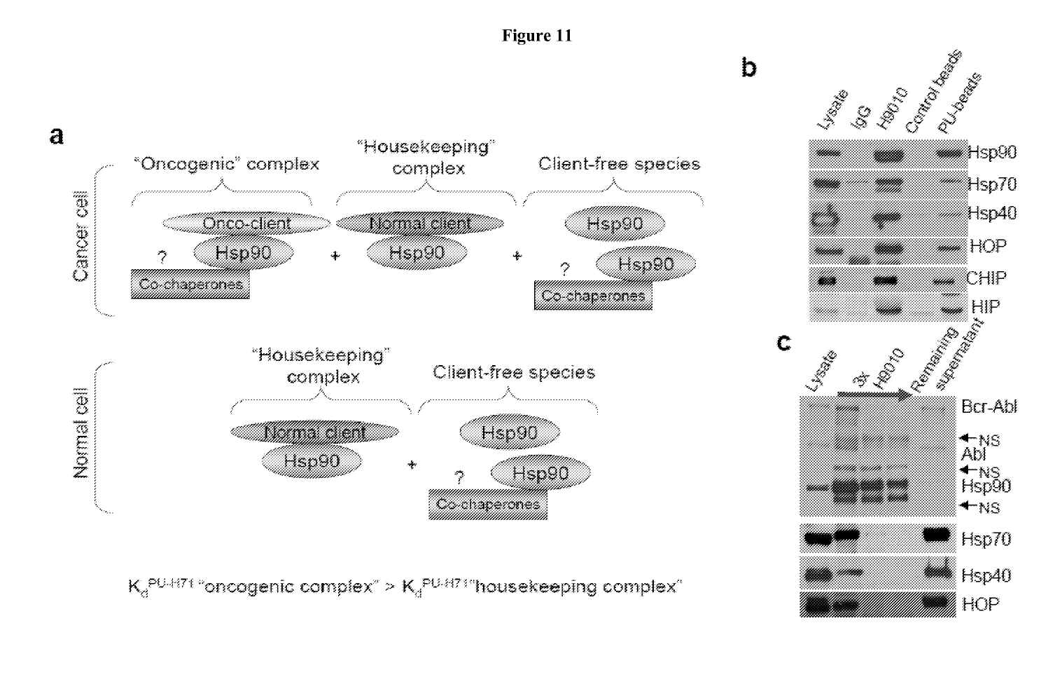

When a probe is needed to identify Hsp90 in complex with its onco-client proteins, further important requirements are (1.) that the probe retains selectivity for the “oncogenic Hsp90 species” and (2.) that upon binding to Hsp90, the probe locks Hsp90 in a client-protein bound conformation. The concept of “oncogenic Hsp90” is further defined in this application as well as in FIG. 11.

When a probe is needed to identify Hsp90 in complex with its onco-client proteins by mass spectrometry techniques, further important requirements are (1.) that the probe isolates sufficient protein material and (2.) that the signal to ratio as defined by the amount of Hsp90 onco-clients and unspecifically resin-bound proteins, respectively, be sufficiently large as to be identifiable by mass spectrometry. This application provides examples of the production of such probes.

We chose Affi-Gel® 10 (BioRad) for ligand attachment. These agarose beads have an N-hydroxysuccinimide ester at the end of a 10C spacer arm, and in consequence, each linker was designed to contain a distal amine functionality. The site of linker attachment to PU-H71 was aided by the co-crystal structure of it bound to the N-terminal domain of human Hsp90α (PDB ID: 2FWZ). This structure shows that the purine's N9 amine makes no direct contact with the protein and is directed towards solvent (FIG. 27A) (Immormino et al., 2006). As well, a previous SAR indicated that this is an attractive site since it was previously used for the introduction of water solubilizing groups (He et al., 2006). Compound 5 (PU-H71-C₆ linker) was designed and docked onto the Hsp90 active site (FIG. 27A). All the interactions of PU-H71 were preserved, and the computer model clearly showed that the linker oriented towards the solvent exposed region. Therefore, compound 5 was synthesized as the immediate precursor for attachment to solid support (see Chemistry, FIG. 30). In the FP assay, 5 retained affinity for Hsp90 (IC₅₀=19.8 nM compared to 22.4 nM for PU-H71, Table 8) which then enabled us to move forward with confidence towards the synthesis of solid support immobilized PU-H71 probe (6) by attachment to Affi-Gel® 10 (FIG. 30).

We also designed a biotinylated derivative of PU-H71. One advantage of the biotinylated agent over the solid supported agents is that they can be used to probe binding directly in cells or in vivo systems. The ligand-Hsp90 complexes can then be captured on biotin-binding avidin or streptavidin containing beads. Typically this process reduces the unspecific binding associated with chemical precipitation from cellular extracts. Alternatively, for in vivo experiments, the presence of active sites (in this case Hsp90), can be detected in specific tissues (i.e. tumor mass in cancer) by the use of a labeled-streptavidin conjugate (i.e. FITC-streptavidin). Biotinylated PU-H71 (7) was obtained by reaction of 2 with biotinyl-3,6,9-trioxaundecanediamine (EZ-Link® Amine-PEO₃-Biotin) (FIG. 31). 7 retained affinity for Hsp90 (IC₅₀=67.1 nM) and contains an exposed biotin capable of interacting with streptavidin for affinity purification.

From the available co-crystal structure of NVP-AUY922 with Hsp90α (PDB ID: 2VCI, FIG. 27B) and co-crystal structures of related 3,4-diarylpyrazoles with Hsp90α, as well as from SAR, it was evident that there was a considerable degree of tolerance for substituents at the para-position of the 4-aryl ring (Brough et al., 2008; Cheung et al., 2005; Dymock et al., 2005; Barril et al., 2006). Because the 4-aryl substituent is largely directed towards solvent and substitution at the para-position seems to have little impact on binding affinity, we decided to attach the molecule to solid support at this position. In order to enable attachment, the morpholine group was changed to the 1,6-diaminohexyl group to give 10 as the immediate precursor for attachment to solid support. Docking 10 onto the active site (FIG. 27B) shows that it maintains all of the interactions of NVP-AUY922 and that the linker orients towards the solvent exposed region. When 10 was tested in the binding assay it also retained affinity (IC₅₀=7.0 nM compared to 4.1 nM for NVP-AUY922, Table 8) and was subsequently used for attachment to solid support (see Chemistry, FIG. 32).

Although a co-crystal structure of SNX-2112 with Hsp90 is not publicly available, that of a related tetrahydro-4H-carbazol-4-one (27) bound to Hsp90α (PDB ID: 3D0B, FIG. 27C) is (Barta et al., 2008). This, along with the reported SAR for 27 suggests linker attachment to the hydroxyl of the trans-4-aminocylohexanol substituent. Direct attachment of 6-amino-caproic acid via an ester linkage was not considered desirable because of the potential instability of such bonds in lysate mixtures due to omnipresent esterases. Therefore, the hydroxyl was substituted with amino to give the trans-1,4-diaminocylohexane derivative 18 (FIG. 33). Such a change resulted in nearly a 14-fold loss in potency as compared to SNX-2112 (Table 8). 6-(Boc-amino)caproic acid was attached to 18 and following deprotection, 20 was obtained as the immediate precursor for attachment to beads (see Chemistry, FIG. 33). Docking suggested that 20 interacts similarly to 27 (FIG. 27C) and that the linker orients towards the solvent exposed region. 20 was determined to have good affinity for Hsp90 (IC₅₀=24.7 nM compared to 15.1 nM for SNX-2112 and 210.1 nM for 18, Table 8) and to have regained almost all of the affinity lost by 18. The difference in activity between 18 and both 20 and SNX-2112 is well explained by our binding model, as compounds 20 (—C═O, FIG. 27C) and SNX-2112 (—OH, Figure not shown) form a hydrogen bond with the side-chain amino of Lys 58. 18 contains a strongly basic amino group and is incapable of forming a hydrogen bond with Lys 58 side chain (NH₂, Figure not shown). This is in good agreement with the observation of Huang et al. that basic amines at this position are disfavored. The amide bond of 20 converts the basic amino of 18 into a non-basic amide group capable of acting as an H-bond acceptor to Lys 58, similarly to the hydroxyl of SNX-2112.

Synthesis of PU-H71 beads (6) is shown in FIG. 30 and commences with the 9-alkylation of 8-arylsulfanylpurine (1) (He et al., 2006) with 1,3-dibromopropane to afford 2 in 35% yield. The low yield obtained in the formation of 2 can be primarily attributed to unavoidable competing 3-alkylation. Five equivalents of 1,3-dibromopropane were used to ensure complete reaction of 1 and to limit other undesirable side-reactions, such as dimerization, which may also contribute to the low yield. 2 was reacted with tert-butyl 6-aminohexylcarbamate (3) to give the Boc-protected amino purine 4 in 90% yield. Deprotection with TFA followed by reaction with Affi-Gel® 10 resulted in 6. Biotinylated PU-H71 (7) was also synthesized by reacting 2 with EZ-Link® Amine-PEO₃-Biotin (FIG. 31).

Synthesis of NVP-AUY922 beads (11) from aldehyde 8 (Brough et al., 2008) is shown in FIG. 32. 9 was obtained from the reductive amination of 8 with 3 in 75% yield with no detectable loss of the Boc group. In a single step, both the Boc and benzyl protecting groups were removed with BCl₃ to give isoxazole 10 in 78% yield, which was then reacted with Affi-Gel® 10 to give 11.

Synthesis of SNX-2112 beads (21) is shown in FIG. 33, and while compounds 17 and 18 are referred to in the patent literature (Serenex et al., 2008, WO-2008130879A2; Serenex et al., 2008, US-20080269193A1), neither is adequately characterized, nor are their syntheses fully described. Therefore, we feel that it is worth describing the synthesis in detail. Tosylhydrazone 14 was obtained in 89% yield from the condensation of tosyl hydrazide (12) with dimedone (13). The one-pot conversion of 14 to tetrahydroindazolone 15 occurs following base promoted cyclocondensation of the intermediate trifluoroacyl derivative generated by treatment with trifluroacetic anhydride in 55% yield. 15 was reacted with 2-bromo-4-fluorobenzonitrile in DMF to give 16 in 91% yield. It is interesting to note the regioselectivity of this reaction as arylation occurs selectively at N1. In computational studies of indazol-4-ones similar to 15, both 1H and 2H-tautomers are known to exist in equilibrium, however, because of its higher dipole moment the 1H tautomer is favored in polar solvents (Claramunt et al., 2006). The amination of 16 with trans-1,4-diaminocyclohexane was accomplished under Buchwald conditions (Old et al., 1998) using tris(dibenzylideneacetone)dipalladium [Pd₂(dba)₃] and 2-dicyclohexylphosphino-2′-(N,N-dimethylamino)biphenyl (DavePhos) to give nitrile 17 (24%) along with amide 18 (17%) for a combined yield of 41%. Following complete hydrolysis of 17, 18 was coupled to 6-(Boc-amino)caproic acid with EDCI/DMAP to give 19 in 91% yield. Following deprotection, 20 was obtained which was then reacted with Affi-Gel® 10 to give 21.

Several methods were employed to measure the progress of the reactions for the synthesis of the final probes. UV monitoring of the liquid was used by measuring a decrease in λₘₐₓ for each compound. In general, it was observed that that there was no further decrease in the λₘₐₓ after 1.5 h, indicating completion of the reaction. TLC was employed as a crude measure of the progress of the reaction whereas LC-MS monitoring of the liquid was used to confirm complete reaction. While on TLC the spot would not disappear since excess compound was used (1.2 eq.), a clear decrease in intensity indicated progress of the reaction.

The synthesis and full characterization of the Hsp90 inhibitors PU-H71 (He et al., 2006) and NVP-AUY922 (Brough et al., 2008) have been reported elsewhere. SNX-2112 had previously been mentioned in the patent literature (Serenex et al., 2008, WO-2008130879A2; Serenex et al., 2008, US-20080269193A1), and only recently has it been fully characterized and its synthesis adequately described (Huang et al., 2009). At the time this research project began specific details on its synthesis were lacking Additionally, we had difficulty reproducing the amination of 16 with trans-4-aminocyclohexanol under conditions reported for similar compounds [Pd(OAc)₂, DPPF, NaOtBu, toluene, 120° C., microwave]. In our hands, only trace amounts of product were detected at best. Changing catalyst to PdCl₂, Pd(PPh₃)₄ or Pd₂(dba)₃ or solvent to DMF or 1,2-dimethoxyethane (DME) or base to K₃PO₄ did not result in any improvement. Therefore, we modified this step and were able to couple 16 to trans-4-aminocyclohexanol tetrahydropyranyl ether (24) under Buchwald conditions (Old et al., 1998) using Pd₂(dba)₃ and DavePhos in DME to give nitrile 25 (28%) along with amide 26 (17%) for a combined yield of 45% (FIG. 34). These were the conditions used to couple 16 to trans-1,4-diaminocyclohexane, and similarly some of 25 was hydrolysed to 26 during the course of the reaction. Because for our purpose it was unnecessary, we did not optimize this reaction for 25. We surmised that a major hindrance to the reaction was the low solubility of trans-4-aminocyclohexanol in toluene and that using the THP protected alcohol 24 at the very least increased solubility. SNX-2112 was obtained and fully characterized (¹H, ¹³C-NMR, MS) following removal of the THP group from 26.

Next, we investigated whether the synthesized beads retained interaction with Hsp90 in cancer cells. Agarose beads covalently attached to either of PU-H71, NVP-AUY922, SNX-2112 or 2-methoxyethylamine (PU-, NVP-, SNX-, control-beads, respectively), were incubated with K562 chronic myeloid leukemia (CML) or MDA-MB-468 breast cancer cell extracts. As seen in FIG. 28A, the Hsp90 inhibitor, but not the control-beads, efficiently isolated Hsp90 in the cancer cell lysates. Control beads contain an Hsp90 inactive chemical (2-methoxyethylamine) conjugated to Affi-Gel® 10 (see Experimental) providing an experimental control for potential unspecific binding of the solid-support to proteins in cell extracts.

Further, to probe the ability of these chemical tools to isolate genuine Hsp90 client proteins in tumor cells, we incubated PU-H71 attached to solid support (6) with cancer cell extracts. We were able to demonstrate dose-dependent isolation of Hsp90/c-Kit and Hsp90/IGF-IR complexes in MDA-MB-468 cells (FIG. 28B) and of Hsp90/Bcr-Abl and Hsp90/Raf-1 complexes in K562 cells (FIG. 28C). These are Hsp90-dependent onco-proteins with important roles in driving the transformed phenotype in triple-negative breast cancers and CML, respectively (Whitesell & Lindquist, 2005; Hurvitz & Finn, 2009; Law et al., 2008). In accord with an Hsp90 mediated regulation of c-Kit and IGF-IR, treatment of MDA-MB-468 cells with PU-H71 led to a reduction in the steady-state levels of these proteins (FIG. 28B, compare Lysate, − and + PU-H71). Using the PU-beads (6), we were recently able to isolate and identify novel Hsp90 clients, such as the transcriptional repressor BCL-6 in diffuse large B-cell lymphoma (Cerchietti et al., 2009) and JAK2 in mutant JAK2 driven myeloproliferative disorders (Marubayashi et al., 2010). We were also able to identify Hsp90 onco-clients specific to a triple-negative breast cancer (Caldas-Lopes et al., 2009). In addition to shedding light on the mechanisms of action of Hsp90 in these tumors, the identified proteins are important tumor-specific onco-clients and will be introduced as biomarkers in monitoring the clinical efficacy of PU-H71 and Hsp90 inhibitors in these cancers during clinical studies.

Similar experiments were possible with PU-H71-biotin (7) (FIG. 29A), although the PU-H71-beads were superior to the PU-H71-biotin beads at isolating Hsp90 in complex with a client protein.

It is important to note that previous attempts to isolate Hsp90/client protein complexes using a solid-support immobilized GM were of little success (Tsaytler et al., 2009). In that case, the proteins bound to Hsp90 were washed away during the preparative steps. To prevent the loss of Hsp90-interacting proteins, the authors had to subject the cancer cell extracts to cross-linking with DSP, a homobifunctional amino-reactive DTT-reversible cross-linker, suggesting that unlike PU-H71, GM is unable to stabilize Hsp90/client protein interactions. We observed a similar profile when using beads with GM directly covalently attached to the Affi-Gel® 10 resin. Crystallographic and biochemical investigations suggest that GM preferentially interacts with Hsp90 in an apo, open-conformation, that is unfavorable for certain client protein binding (Roe et al., 1999; Stebbins et al., 1997; Nishiya et al., 2009) providing a potential explanation for the limited ability of GM-beads to capture Hsp90/client protein complexes. It is currently unknown what Hsp90 conformations are preferred by the other Hsp90 chemotypes, but with the NVP- and SNX-beads also available, as reported here, similar evaluations are now possible, leading to a better understanding of the interaction of these agents with Hsp90, and of the biological significance of these interactions.

In another application of the chemical tools designed here, we show that PU-H71-biotin (7) can also be used to specifically detect Hsp90 when expressed on the cell surface (FIG. 29B). Hsp90, which is mainly a cytosolic protein, has been reported in certain cases to translocate to the cell surface. In a breast cancer for example, membrane Hsp90 is involved in aiding cancer cell invasion (Sidera & Patsavoudi, 2008). Specific detection of the membrane Hsp90 in live cells is possible by the use of PU-H71-biotin (7) because, while the biotin conjugated Hsp90 inhibitor may potentially enter the cell, the streptavidin conjugate used to detect the biotin, is cell impermeable. FIG. 29B shows that PU-H71-biotin but not D-biotin can detect Hsp90 expression on the surface of leukemia cells.

In summary, we have prepared useful chemical tools based on three different Hsp90 inhibitors, each of a different chemotype. These were prepared either by attachment onto solid support, such as PU-H71 (purine), NVP-AUY922 (isoxazole) and SNX-2112 (indazol-4-one)-beads, or by biotinylation (PU-H71-biotin). The utility of these probes was demonstrated by their ability to efficiently isolate Hsp90 and, in the case of PU-H71 beads (6), isolate Hsp90 onco-protein containing complexes from cancer cell extracts. Available co-crystal structures and SAR were utilized in their design, and docking to the appropriate X-ray crystal structure of Hsp90α used to validate the site of attachment of the linker. These are important chemical tools in efforts towards better understanding Hsp90 biology and towards designing Hsp90 inhibitors with most favorable clinical profile.

### Identification of Oncoproteins and Pathways Using Hsp90 Probes

The disclosure provides methods of identifying components of cancer-implicated pathway (e.g., oncoproteins) using the Hsp90 probes described above. In one embodiment of the invention the cancer-implicated pathway is a pathway involved in metabolism, genetic information processing, environmental information processing, cellular processes, or organismal systems. For example, the cancer-implicated pathway may be a pathway listed in Table 1.

More particularly, the cancer-implicated pathway or the component of the cancer-implicated pathway is involved with a cancer such as a cancer selected from the group consisting of a colorectal cancer, a pancreatic cancer, a thyroid cancer, a leukemia including an acute myeloid leukemia and a chronic myeloid leukemia, a basal cell carcinoma, a melanoma, a renal cell carcinoma, a bladder cancer, a prostate cancer, a lung cancer including a small cell lung cancer and a non-small cell lung cancer, a breast cancer, a neuroblastoma, myeloproliferative disorders, gastrointestinal cancers including gastrointestinal stromal tumors, an esophageal cancer, a stomach cancer, a liver cancer, a gallbladder cancer, an anal cancer, brain tumors including gliomas, lymphomas including a follicular lymphoma and a diffuse large B-cell lymphoma, and gynecologic cancers including ovarian, cervical, and endometrial cancers.

The following subsections describe use of the Hsp90 probes of the present disclosure to determine properties of Hsp90 in cancer cells and to identify oncoproteins and cancer-implicated pathways.

### Heterogeneous Hsp90 Presentation in Cancer Cells

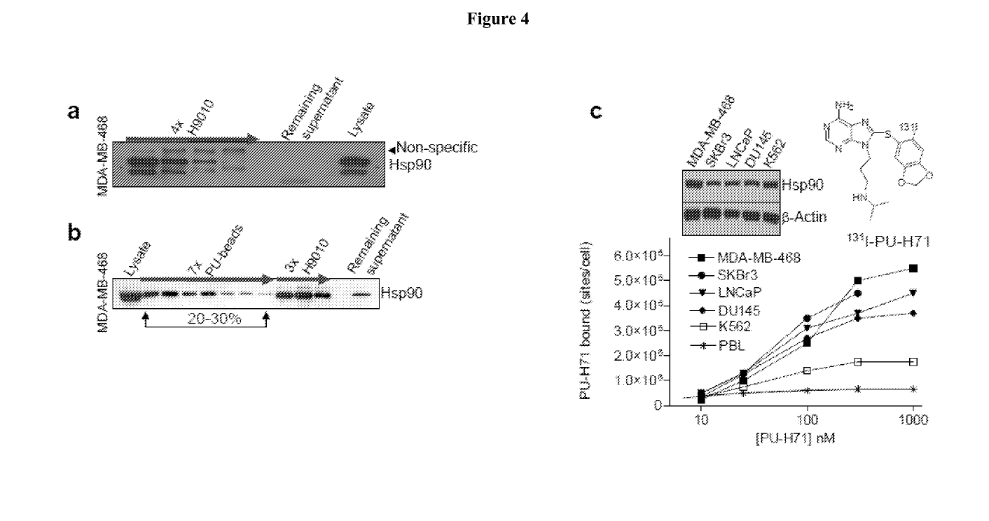

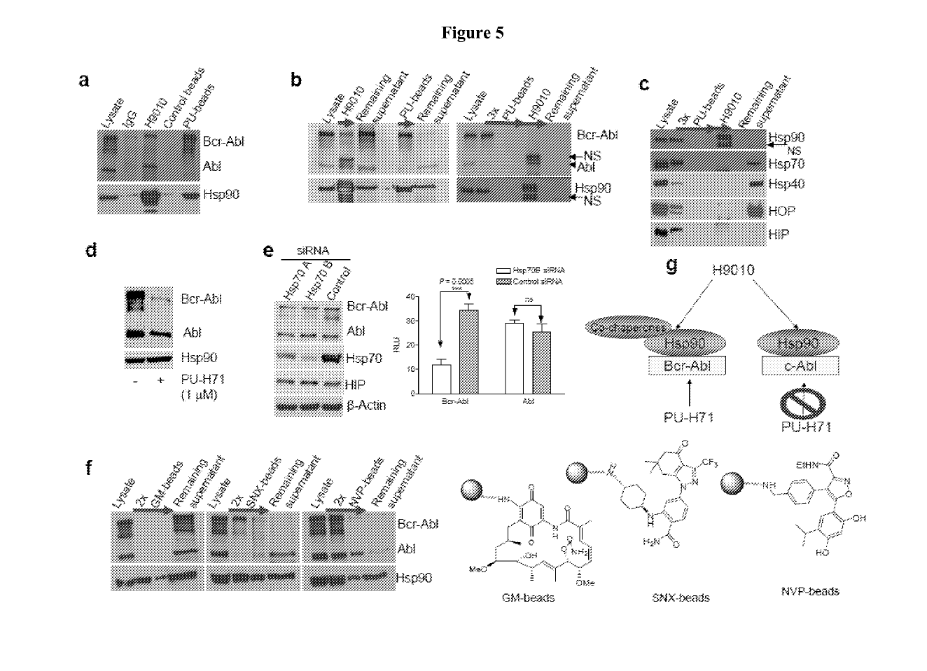

To investigate the interaction of small molecule Hsp90 inhibitors with tumor Hsp90 complexes, we made use of agarose beads covalently attached to either geldanamycin (GM) or PU-H71 (GM- and PU-beads, respectively) (FIGS. 4, 5). Both GM and PU-H71, chemically distinct agents, interact with and inhibit Hsp90 by binding to its N-terminal domain regulatory pocket (Janin, 2010). For comparison, we also generated G protein agarose-beads coupled to an anti-Hsp90 antibody (H9010).

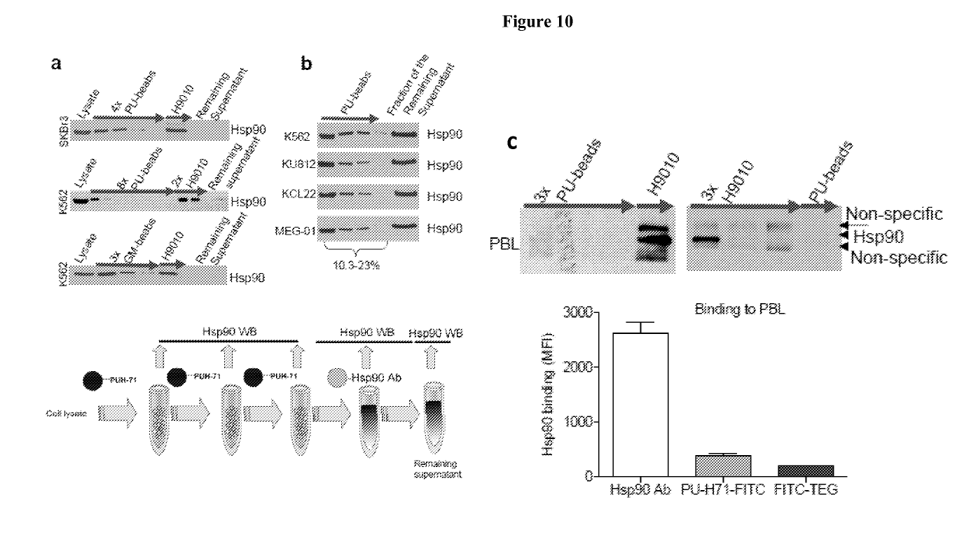

First we evaluated the binding of these agents to Hsp90 in a breast cancer and in chronic myeloid leukemia (CML) cell lysates. Four consecutive immunoprecipitation (IP) steps with H9010, but not with a non-specific IgG, efficiently depleted Hsp90 from these extracts (FIG. 4a, 4×H9010 and not shown). In contrast, sequential pull-downs with PU- or GM-beads removed only a fraction of the total cellular Hsp90 (FIGS. 4b, 10a, 10b). Specifically, in MDA-MB-468 breast cancer cells, the combined PU-bead fractions represented approximately 20-30% of the total cellular Hsp90 pool, and further addition of fresh PU-bead aliquots failed to precipitate the remaining Hsp90 in the lysate (FIG. 4b, PU-beads). This PU-depleted, remaining Hsp90 fraction, while inaccessible to the small molecule, maintained affinity for H9010 (FIG. 4b, H9010). From this we conclude that a significant fraction of Hsp90 in the MDA-MB-468 cell extracts was still in a native conformation but not reactive with PU-H71.

To exclude the possibility that changes in Hsp90 configuration in cell lysates make it unavailable for binding to immobilized PU-H71 but not to the antibody, we analyzed binding of radiolabeled ¹³¹I-PU-H71 to Hsp90 in intact cancer cells (FIG. 4c, lower). The chemical structures of ¹³¹I-PU-H71 and PU-H71 are identical: PU-H71 contains a stable iodine atom (¹²⁷I) and ¹³¹I-PU-H71 contains radioactive iodine; thus, isotopically labeled ¹³¹I-PU-H71 has identical chemical and biological properties to the unlabeled PU-H71. Binding of ¹³¹I-PU-H71 to Hsp90 in several cancer cell lines became saturated at a well-defined, although distinct, number of sites per cell (FIG. 4c, lower). We quantified the fraction of cellular Hsp90 that was bound by PU-H71 in MDA-MB-468 cells. First, we determined that Hsp90 represented 2.66-3.33% of the total cellular protein in these cells, a value in close agreement with the reported abundance of Hsp90 in other tumor cells (Workman et al., 2007). Approximately 41.65×10⁶ MDA-MB-468 cells were lysed to yield 3875 μg of protein, of which 103.07-129.04 μg was Hsp90. One cell, therefore, contained (2.47-3.09)×10⁻⁶ μg, (2.74-3.43)×10⁻¹¹ μmols or (1.64-2.06)×10⁷ molecules of Hsp90. In MDA-MB-468 cells, ¹³¹I-PU-H71 bound at most to 5.5×10⁶ of the available cellular binding sites (FIG. 4c, lower), which amounts to 26.6-33.5% of the total cellular Hsp90 (calculated as 5.5×10⁶/(1.64-2.06)×10⁷*100). This value is remarkably similar to the one obtained with PU-bead pull-downs in cell extracts (FIG. 4b), confirming that PU-H71 binds to a fraction of Hsp90 in MDA-MB-468 cells that represents approximately 30% of the total Hsp90 pool and validating the use of PU-beads to efficiently isolate this pool. In K562 and other established t(9;22)+ CML cell lines, PU-H71 bound 10.3-23% of the total cellular Hsp90 (FIGS. 4c, 10b, 10c).

Collectively, these data suggest that certain Hsp90 inhibitors, such as PU-H71, preferentially bind to a subset of Hsp90 species that is more abundant in cancer cells than in normal cells (FIG. 11a).

### Onco- and WT-Protein Bound Hsp90 Species Co-Exist in Cancer Cells, but PU-H71 Selects for the Onco-Protein/Hsp90 Species

To explore the biochemical functions associated with these Hsp90 species, we performed immunoprecipitations (IPs) and chemical precipitations (CPs) with antibody- and Hsp90-inhibitor beads, respectively, and we analysed the ability of Hsp90 bound in these contexts to co-precipitate with a chosen subset of known clients. K562 CML cells were first investigated because this cell line co-expresses the aberrant Bcr-Abl protein, a constitutively active kinase, and its normal counterpart c-Abl. These two Abl species are clearly separable by molecular weight and thus easily distinguishable by Western blot (FIG. 5a, Lysate), facilitating the analysis of Hsp90 onco- and wild type (WT)-clients in the same cellular context. We observed that H9010, but not a non-specific IgG, isolated Hsp90 in complex with both Bcr-Abl and Abl (FIGS. 5a and 11, H9010). Comparison of immunoprecipitated Bcr-Abl and Abl (FIGS. 5a and 5b, left, H9010) with the fraction of each protein remaining in the supernatant (FIG. 5b, left, Remaining supernatant), indicated that the antibody did not preferentially enrich for Hsp90 bound to either mutant or WT forms of Abl in K562 cells.

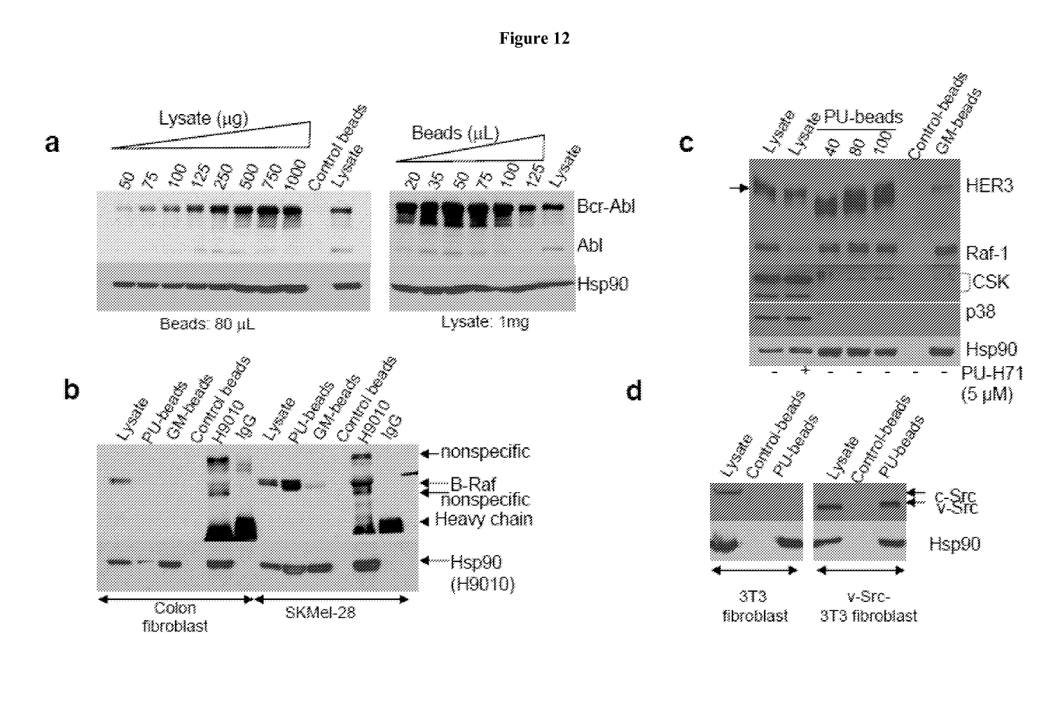

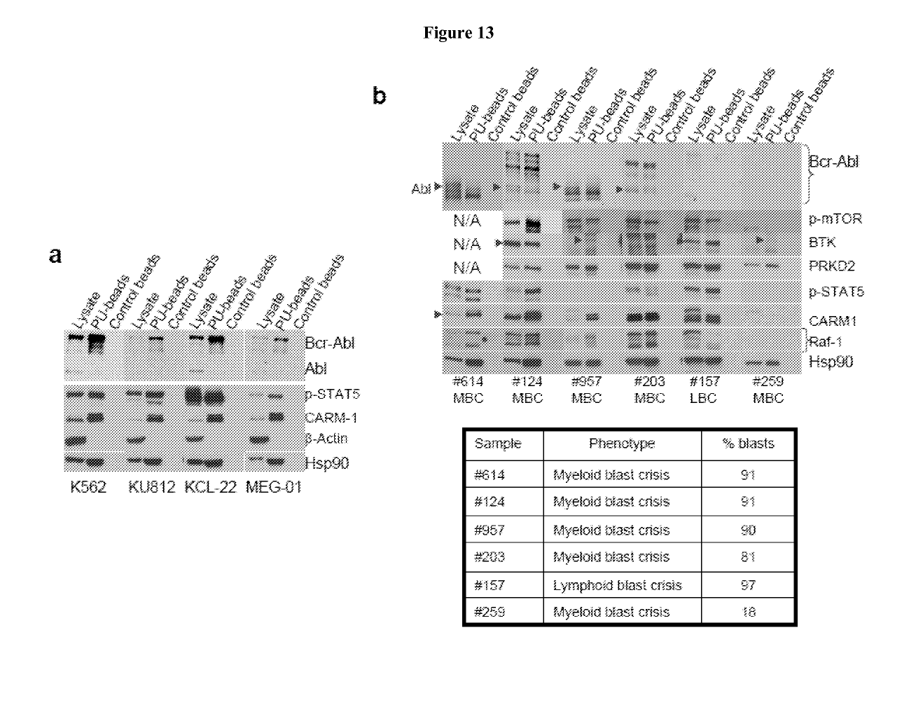

In contrast, PU-bound Hsp90 preferentially isolated the Bcr-Abl protein (FIGS. 5a and 5b, right, PU-beads). Following PU-bead depletion of the Hsp90/Bcr-Abl species (FIG. 5b, right, PU-beads), H9010 precipitated the remaining Hsp90/Abl species (FIG. 5b, right, H9010). PU-beads retained selectivity for Hsp90/Bcr-Abl species at substantially saturating conditions (i.e. excess of lysate, FIG. 12a, left, and beads, FIG. 12a, right). As further confirmation of the biochemical selectivity of PU-H71 for the Bcr-Abl/Hsp90 species, Bcr-Abl was much more susceptible to degradation by PU-H71 than was Abl (FIG. 5d). The selectivity of PU-H71 for the aberrant Abl species extended to other established t(9;22)+ CML cell lines (FIG. 13a), as well as to primary CML samples (FIG. 13b).

### the Onco- but not WT-Protein Bound Hsp90 Species are Most Dependent on Co-Chaperone Recruitment for Client Protein Regulation by Hsp90

To further differentiate between the PU-H71- and antibody-associated Hsp90 fractions, we performed sequential depletion experiments and evaluated the co-chaperone constituency of the two species (Zuehlke & Johnson, 2010). The fraction of Hsp90 containing the Hsp90/Bcr-Abl complexes bound several co-chaperones, including Hsp70, Hsp40, HOP and HIP (FIG. 5c, PU-beads). PU-bead pull-downs were also enriched for several additional Hsp90 co-chaperone species (Tables 5a-d). These findings strongly suggest that PU-H71 recognizes co-chaperone-bound Hsp90. The PU-beads-depleted, remaining Hsp90 pool, shown to include Hsp90/Abl species, was not associated with co-chaperones (FIG. 5c, H9010), although their abundant expression was detected in the lysate (FIG. 5c, Remaining supernatant). Co-chaperones are however isolated by H9010 in the total cellular extract (FIGS. 11b, 11c).

These findings suggest the existence of distinct pools of Hsp90 preferentially bound to either Bcr-Abl or Abl in CML cells (FIG. 5g). H9010 binds to both the Bcr-Abl and the Abl containing Hsp90 species, whereas PU-H71 is selective for the Bcr-Abl/Hsp90 species. Our data also suggest that Hsp90 may utilize and require more acutely the classical co-chaperones Hsp70, Hsp40 and HOP when it modulates the activity of aberrant (i.e. Bcr-Abl) but not normal (i.e. Abl) proteins (FIG. 11a). In accord with this hypothesis, we find that Bcr-Abl is more sensitive than Abl to knock-down of Hsp70, an Hsp90 co-chaperone, in K562 cells (FIG. 5e).

### the Onco-Protein/Hsp90 Species Selectivity and the Complex Trapping Ability of PU-H71 are not Shared by all Hsp90 Inhibitors

We next evaluated whether other inhibitors that interact with the N-terminal regulatory pocket of Hsp90 in a manner similar to PU-H71, including the synthetic inhibitors SNX-2112 and NVP-AUY922, and the natural product GM (Janin, 2010), could selectively isolate similar Hsp90 species (FIG. 5f). SNX-beads demonstrated selectivity for Bcr-Abl/Hsp90, whereas NVP-beads behaved similarly to H9010 and did not discriminate between Bcr-Abl/Hsp90 and Abl/Hsp90 species (see SNX- versus NVP-beads, respectively; FIG. 5f). While GM-beads also recognized a subpopulation of Hsp90 in cell lysates (FIG. 10a), they were much less efficient than were PU-beads in co-precipitating Bcr-Abl (FIG. 5f, GM-beads). Similar ineffectiveness for GM in trapping Hsp90/client protein complexes was previously reported (Tsaytler et al., 2009).

### the Onco-Protein/Hsp90 Species Selectivity and the Complex Trapping Ability of PU-H71 is not Restricted to Bcr-Abl/Hsp90 Species

To determine whether selectivity towards onco-proteins was not restricted to Bcr-Abl, we tested several additional well-defined Hsp90 client proteins in other tumor cell lines (FIGS. 12b-d) (da Rocha Dias et al., 2005; Grbovic et al., 2006). In agreement with our results in K562 cells, H9010 precipitated Hsp90 complexed with both mutant B-Raf expressed in SKMel28 melanoma cells and WT B-Raf expressed in CCD18Co normal colon fibroblasts (FIG. 12b, H9010). PU- and GM-beads however, selectively recognized Hsp90/mutant B-Raf, showing little recognition of Hsp90/WT B-Raf (FIG. 12b, PU-beads and GM-beads). However, as was the case in K562 cells, GM-beads were significantly less efficient than PU-beads in co-precipitating the mutant client protein. Similar results were obtained for other Hsp90 clients (FIGS. 12c, 12d; Tsaytler et al., 2009).

### PU-H71-Beads Identify the Aberrant Signalosome in CML

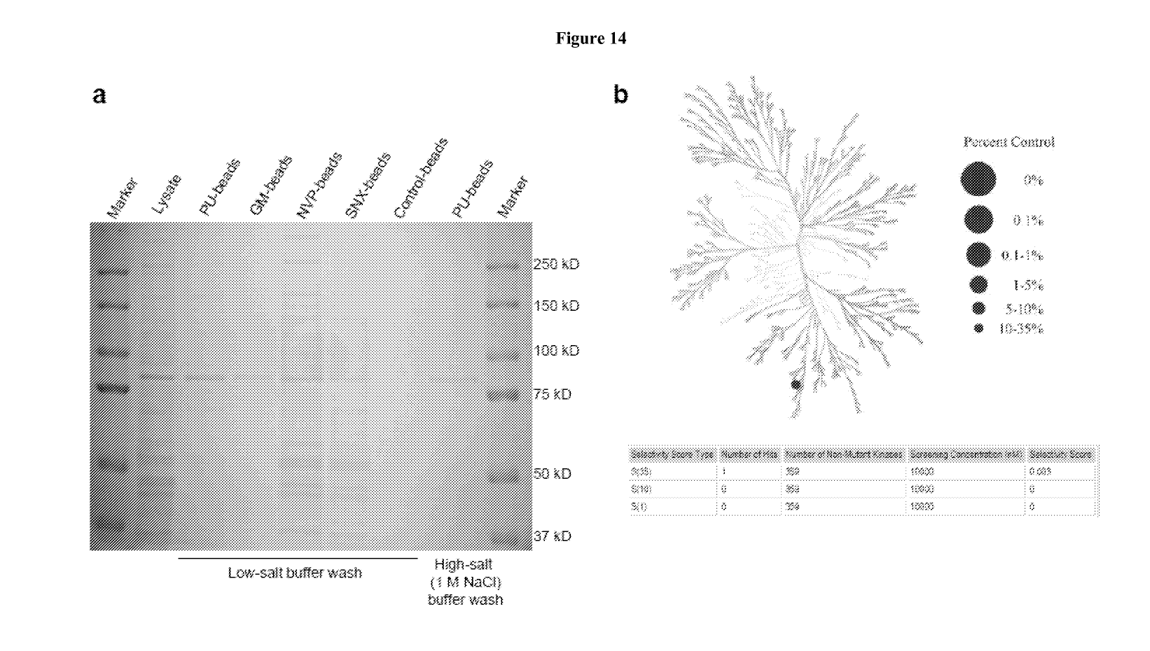

The data presented above suggest that PU-H71, which specifically interacts with Hsp90 (FIG. 14; Taldone & Chiosis, 2009), preferentially selects for onco-protein/Hsp90 species and traps Hsp90 in a client binding conformation (FIG. 5). Therefore, we examined whether PU-H71 beads could be used as a tool to investigate the cellular complement of oncogenic Hsp90 client proteins. Because the aberrant Hsp90 clientele is hypothesized to comprise the various proteins most crucial for the maintenance of the tumor phenotype (Zuehlke & Johnson, 2010; Workman et al., 2007; Dezwaan & Freeman, 2008), this approach could potentially identify critical signaling pathways in a tumor-specific manner. To test this hypothesis, we performed an unbiased analysis of the protein cargo isolated by PU-H71 beads in K562 cells, where at least some of the key functional lesions are known (Ren, 2005; Burke & Carroll, 2010).

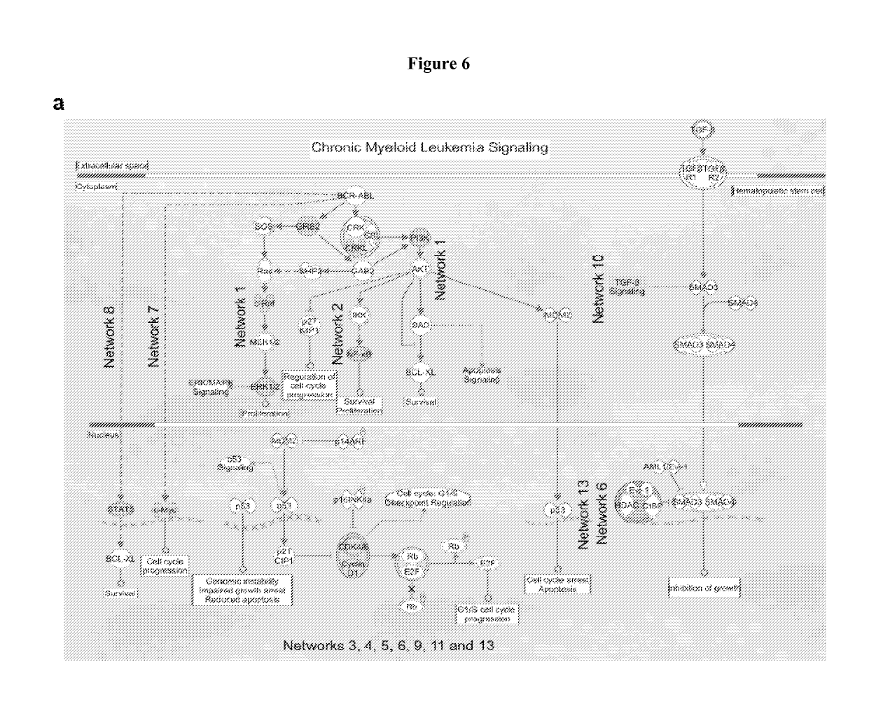

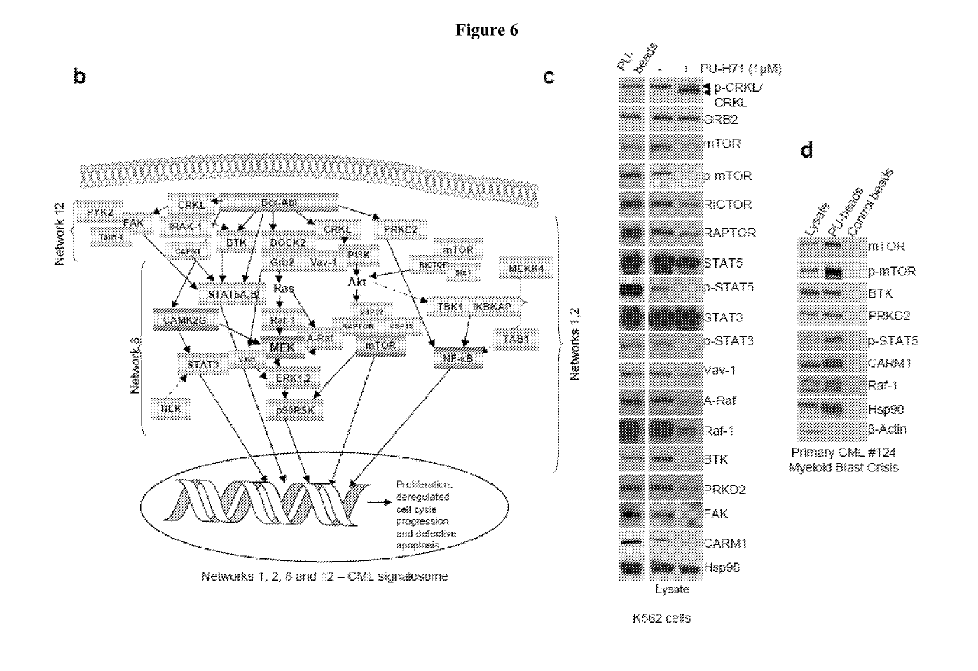

Protein cargo isolated from cell lysate with PU-beads or control-beads was subjected to proteomic analysis by nano liquid chromatography coupled to tandem mass spectrometry (nano LC-MS/MS). Initial protein identification was performed using the Mascot search engine, and was further evaluated using Scaffold Proteome Software (Tables 5a-d). Among the PU-bead-interacting proteins, Bcr-Abl was identified (see Bcr and Abl1, Table 5a and FIG. 6), confirming previous data (FIG. 5).

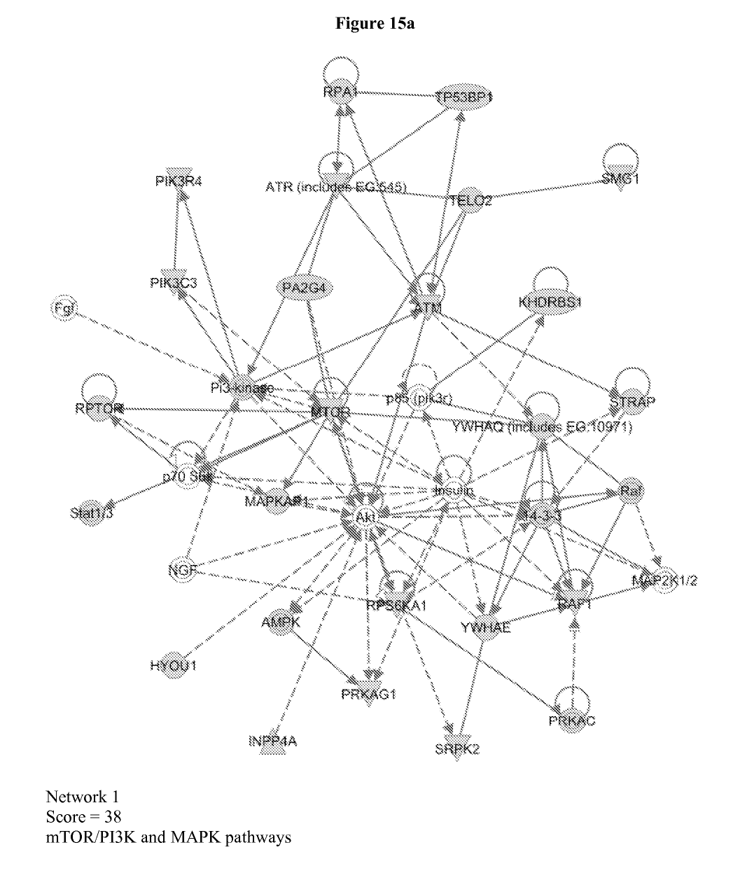

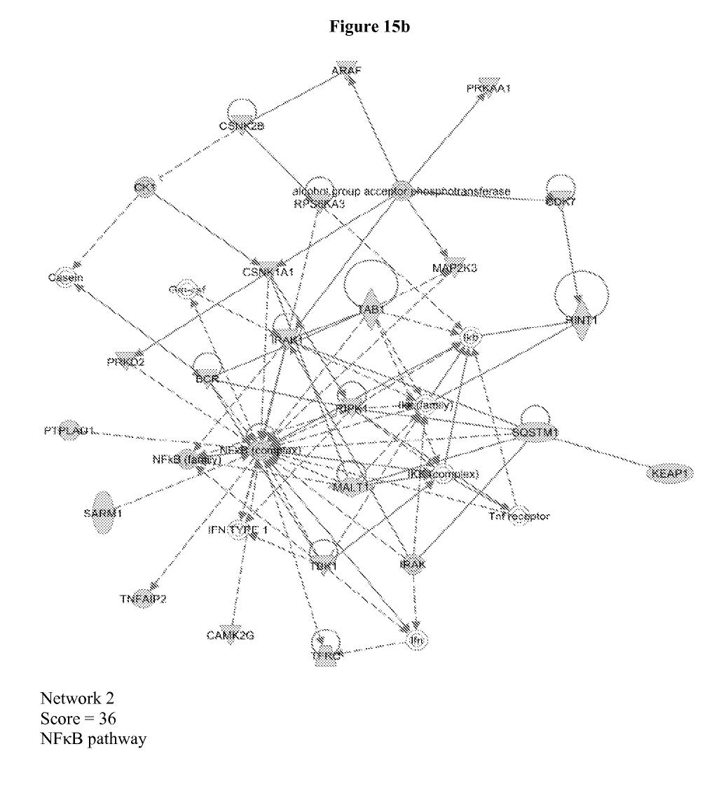

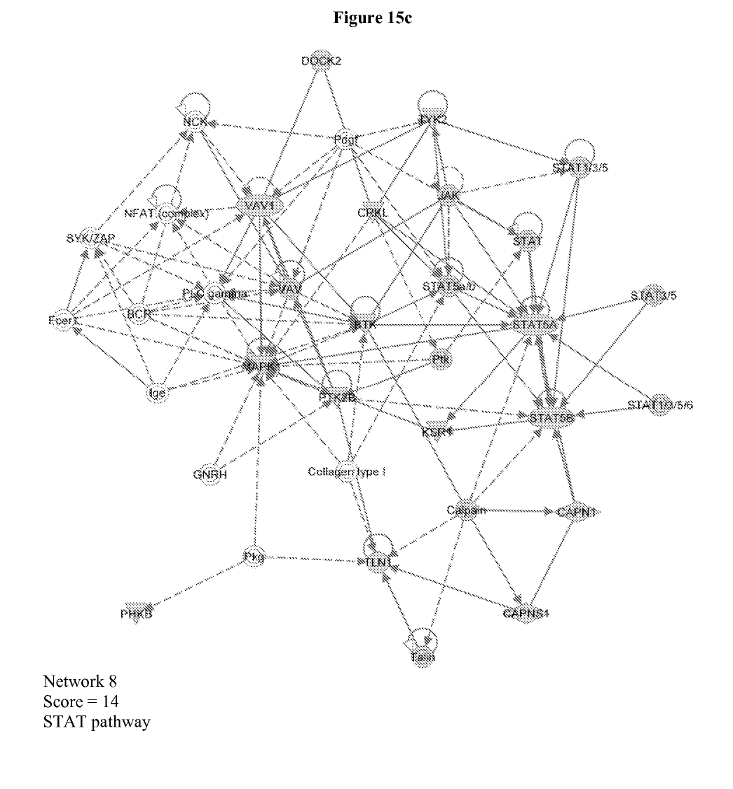

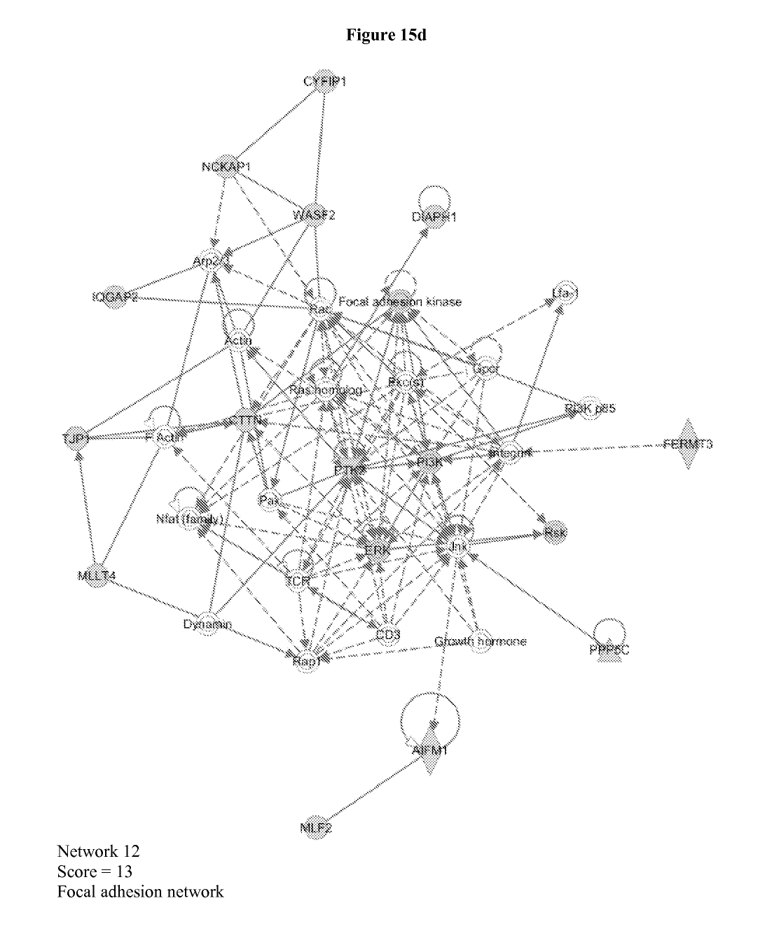

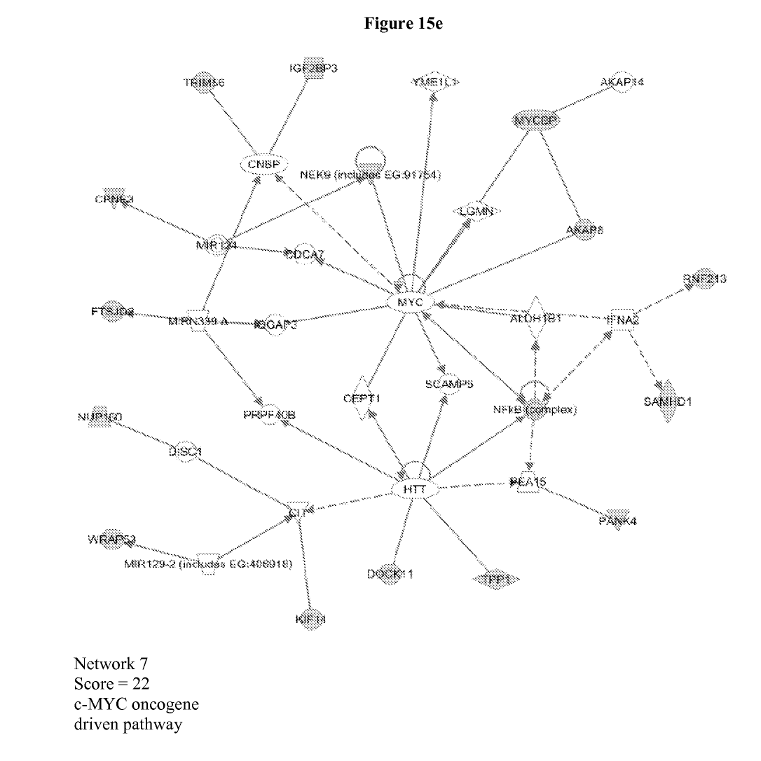

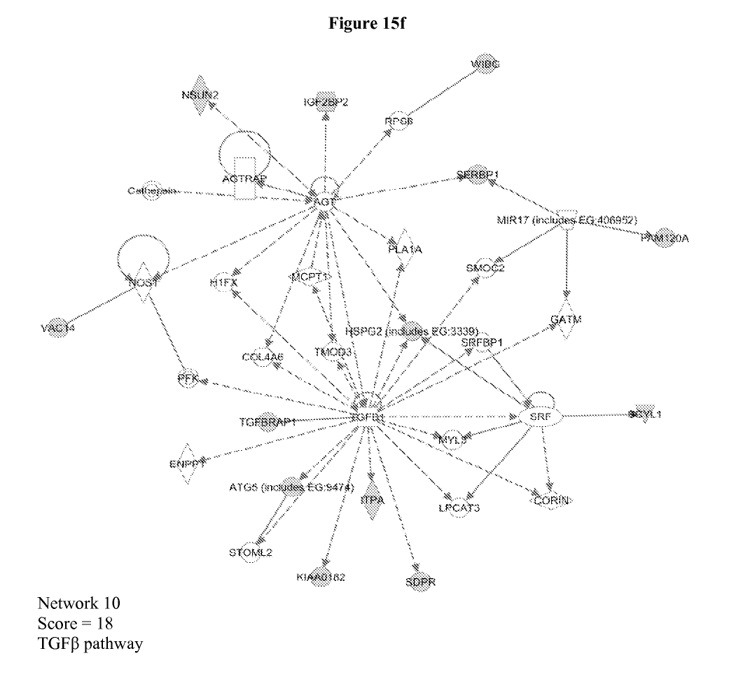

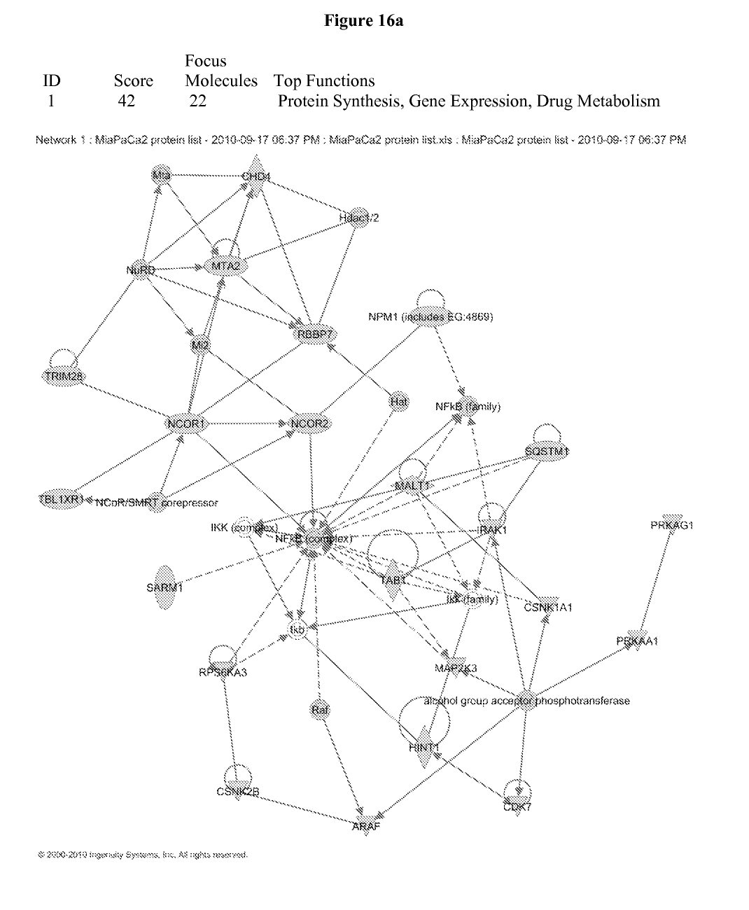

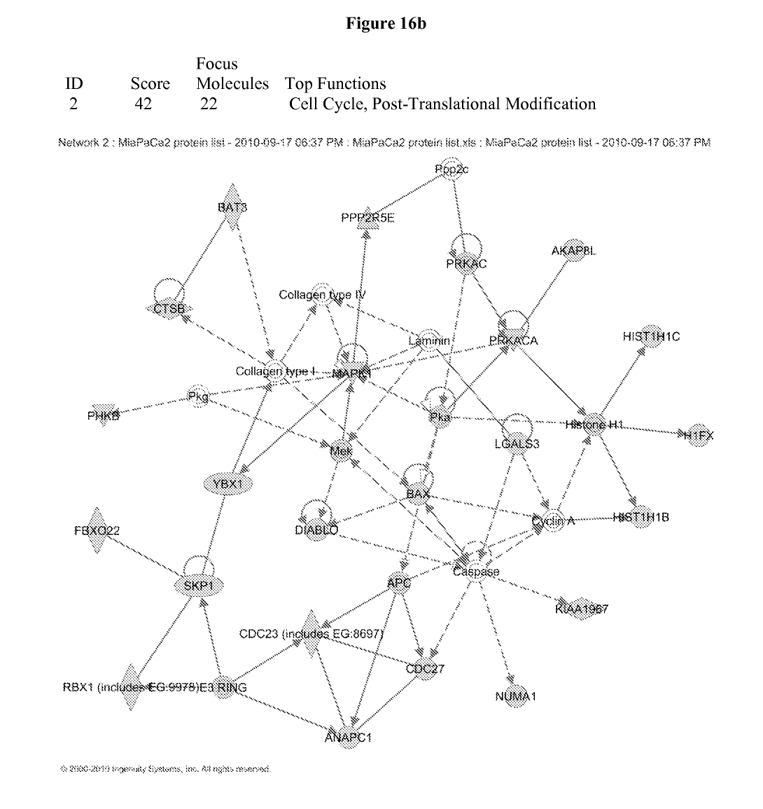

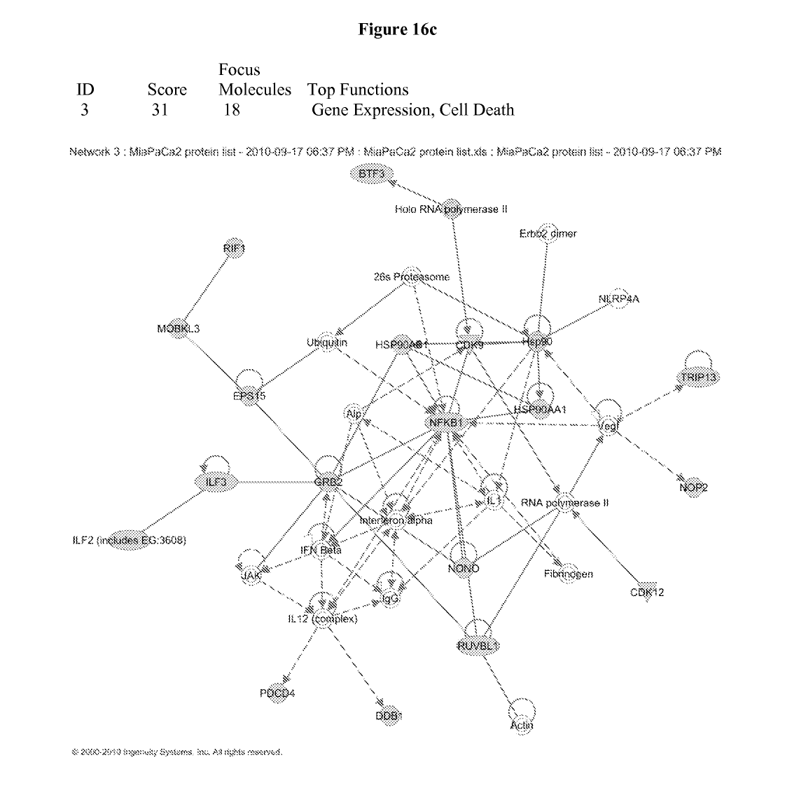

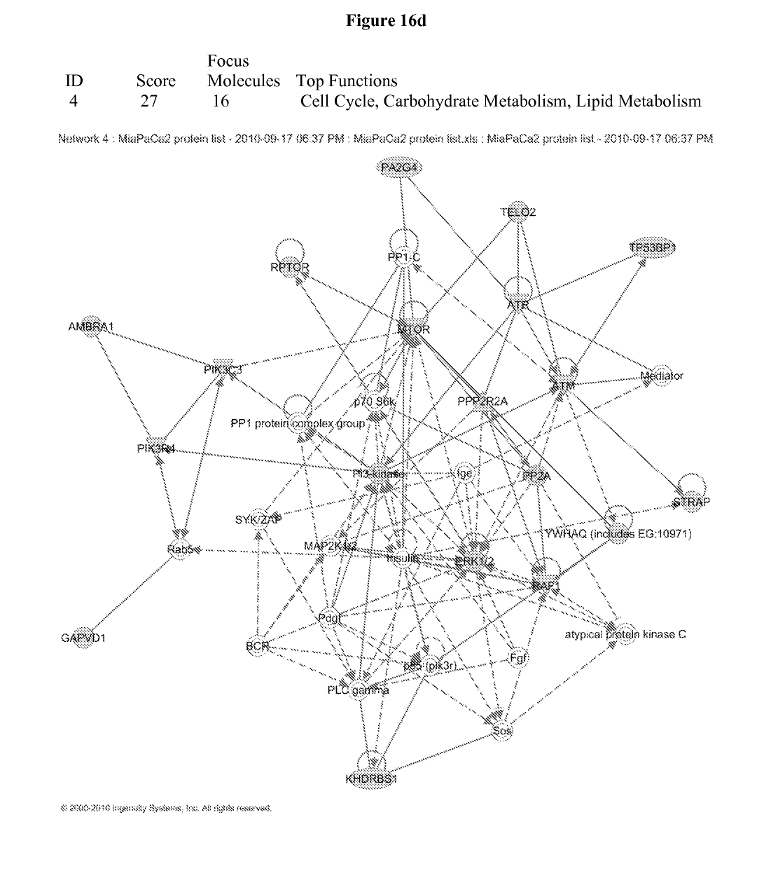

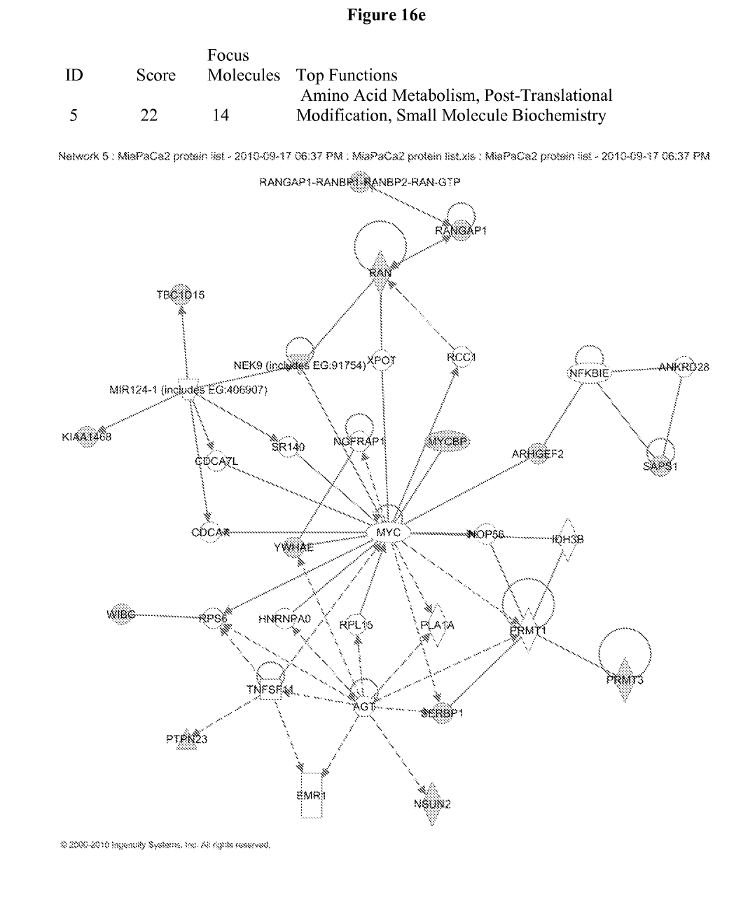

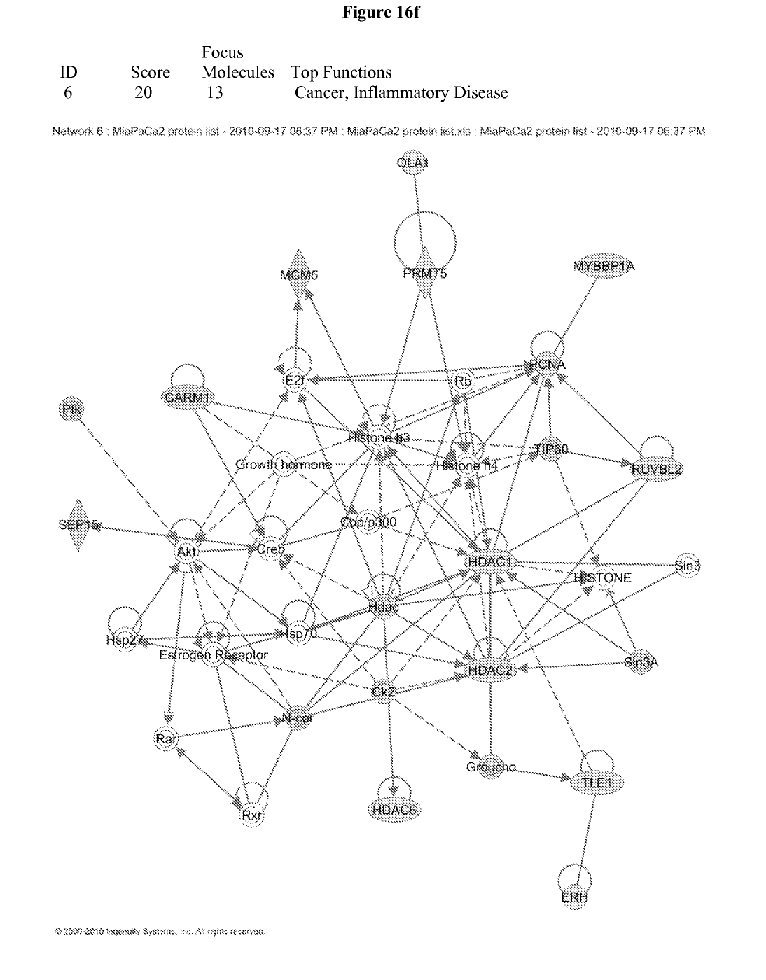

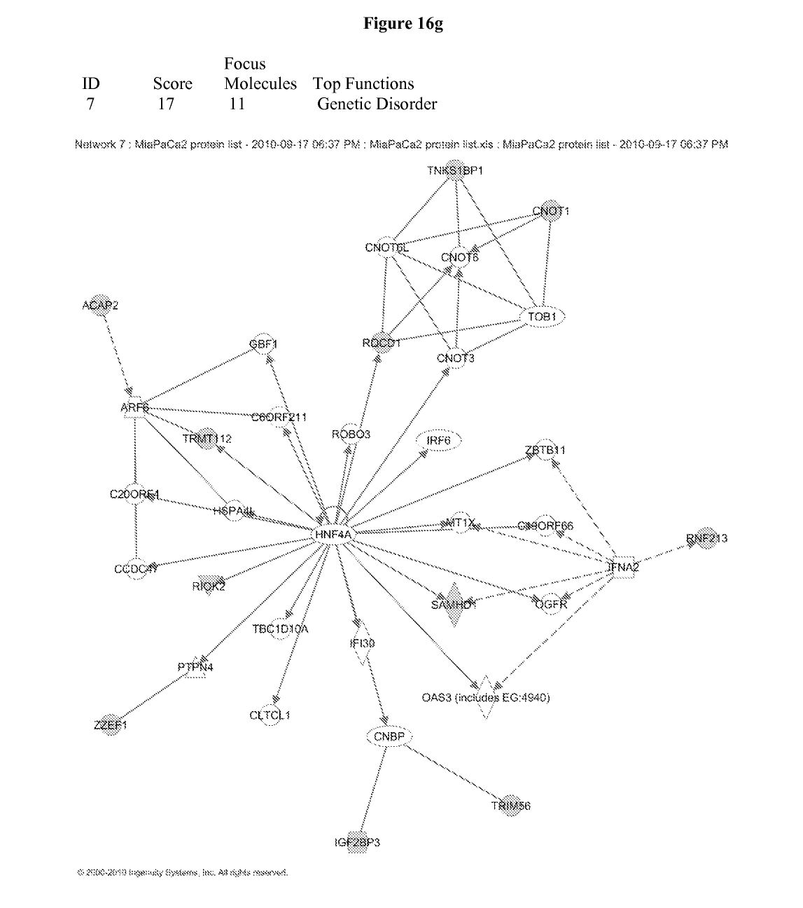

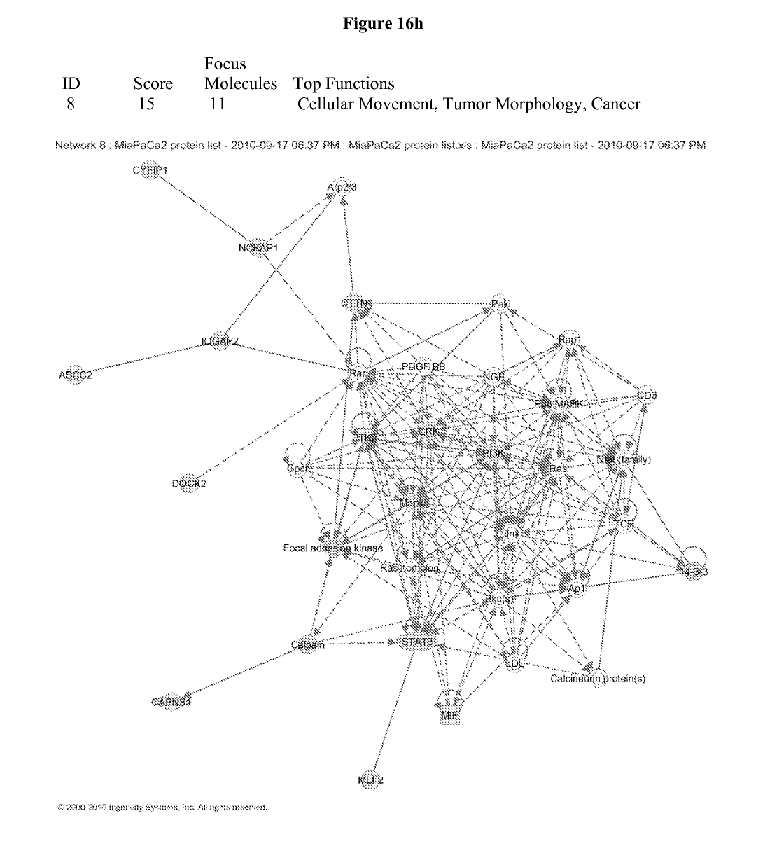

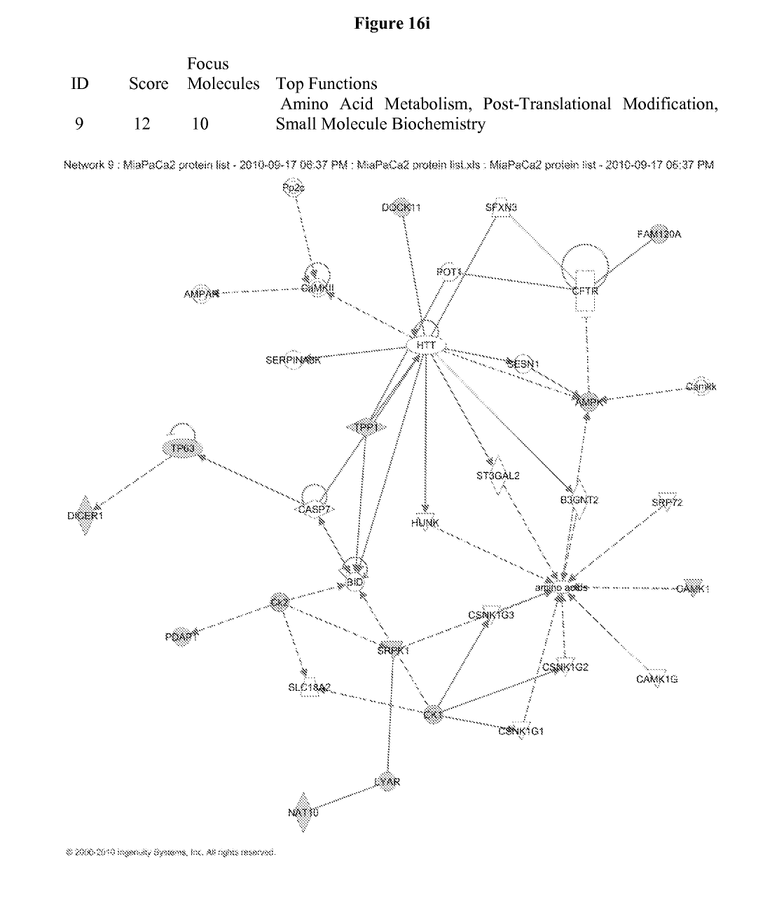

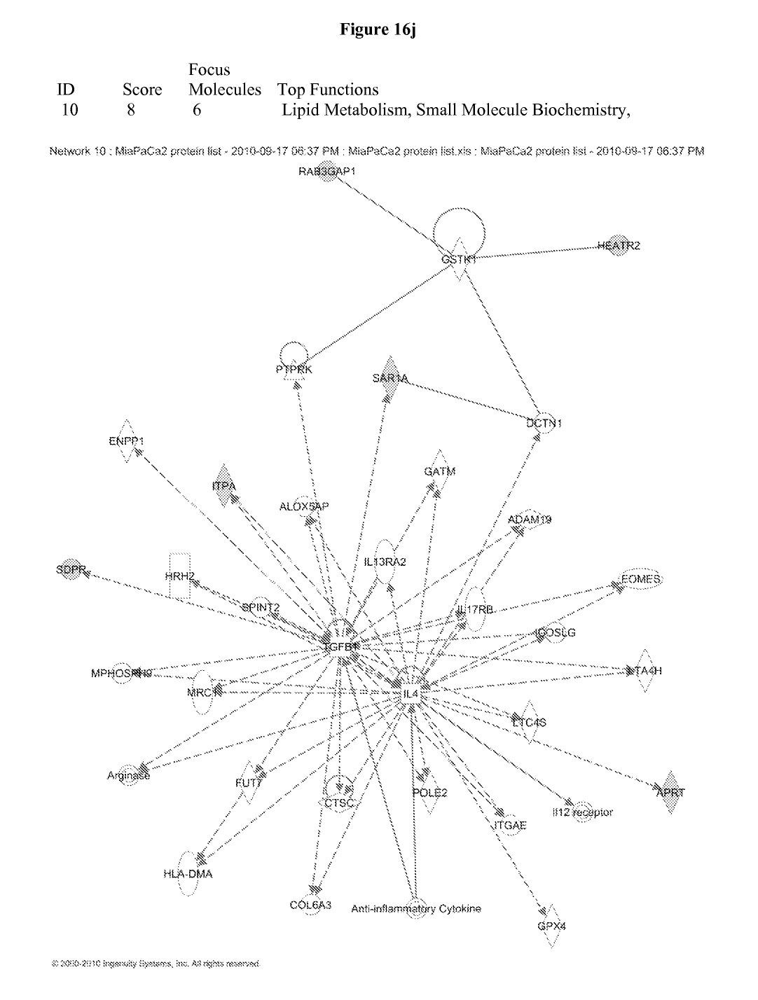

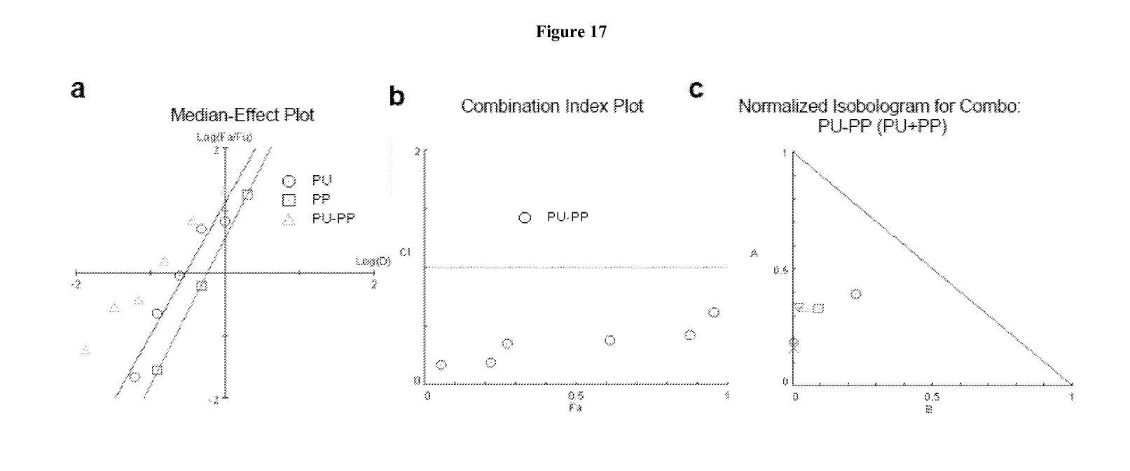

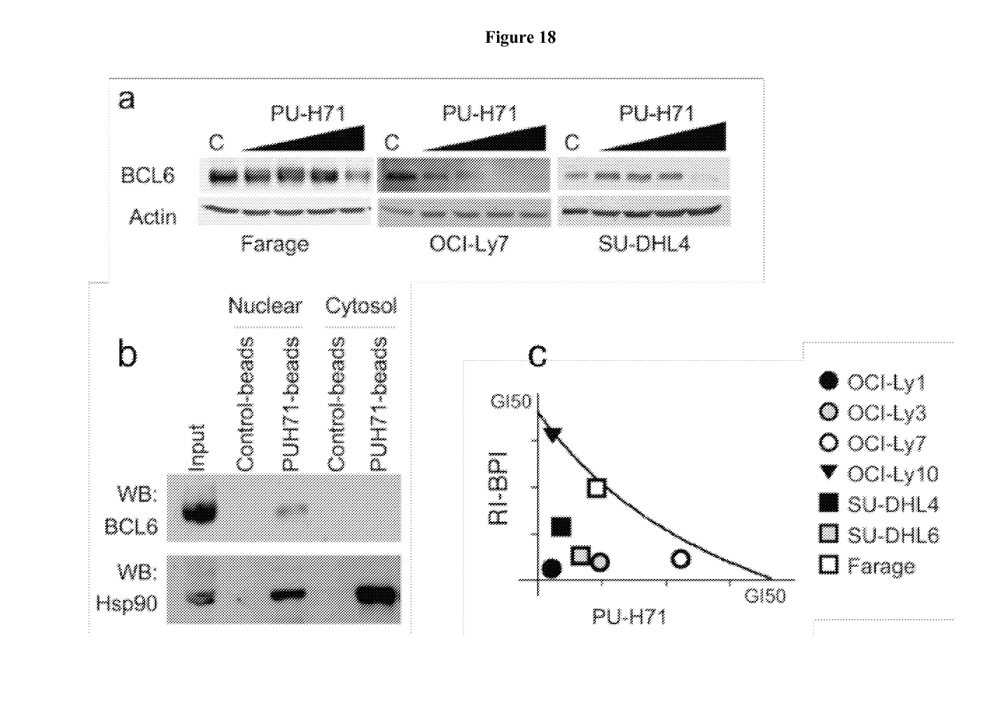

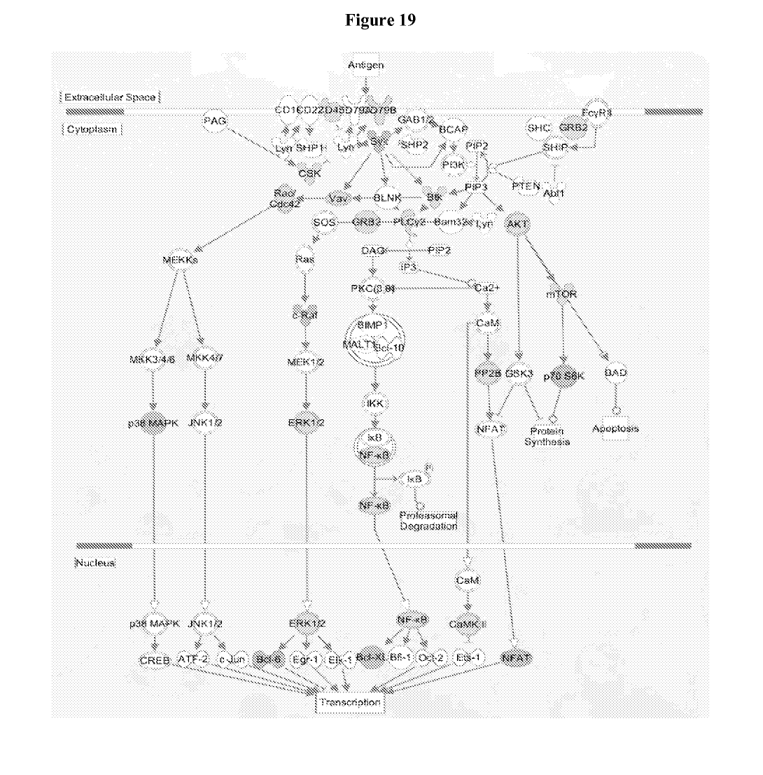

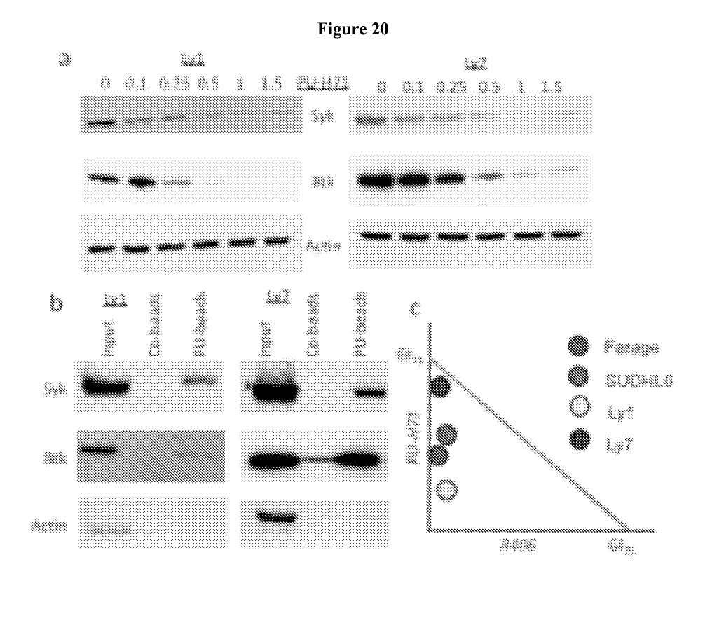

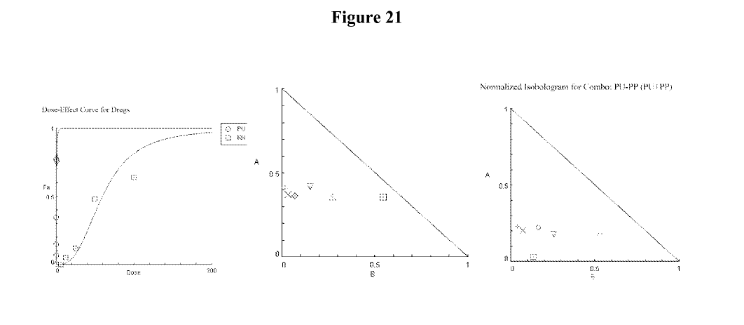

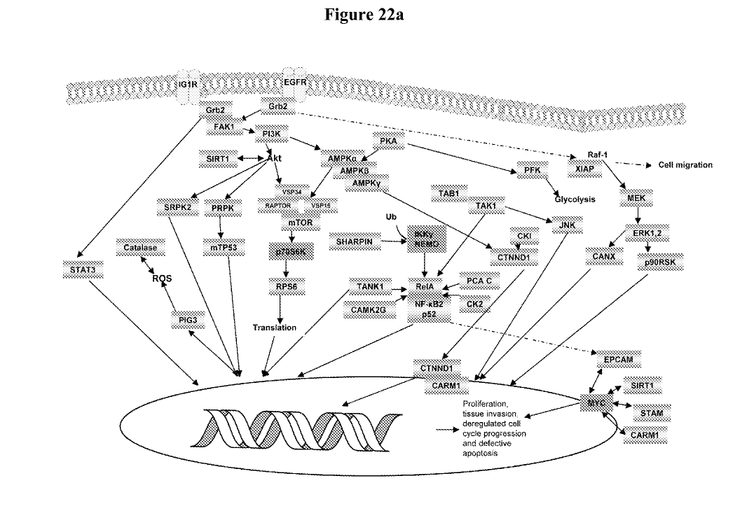

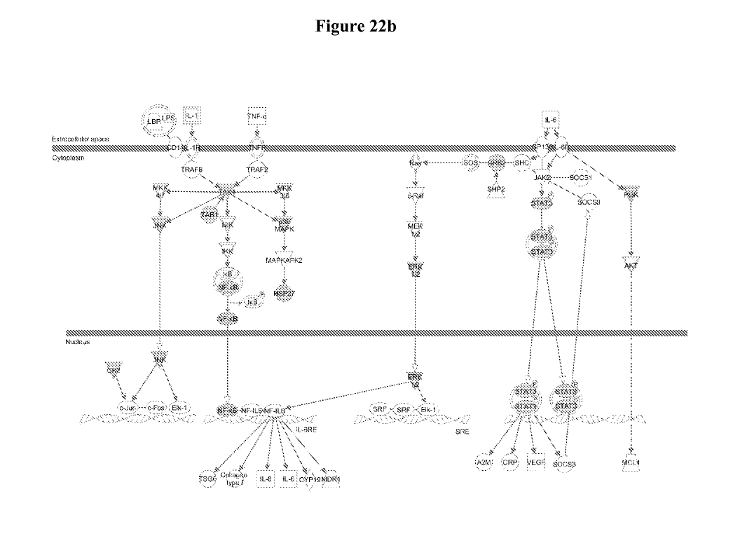

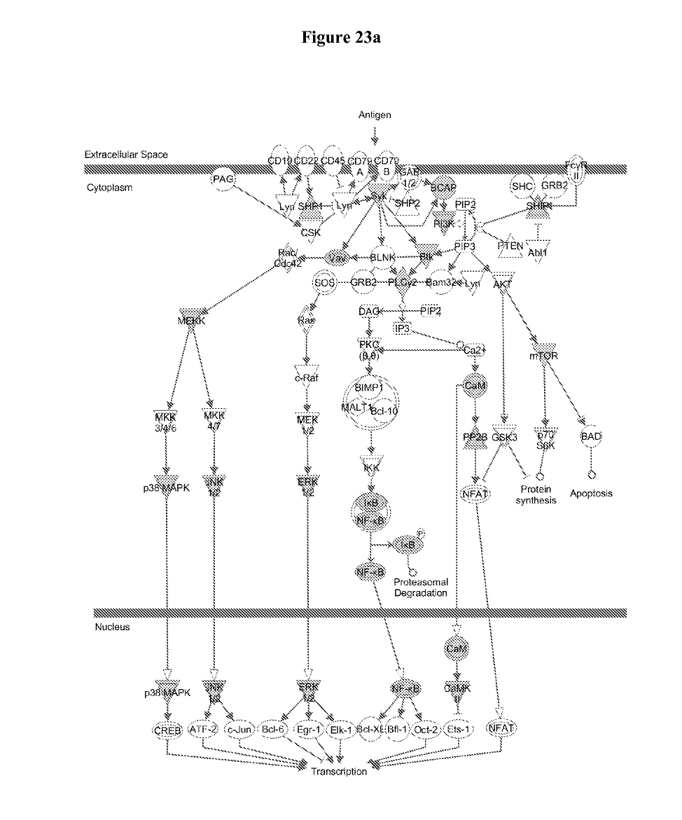

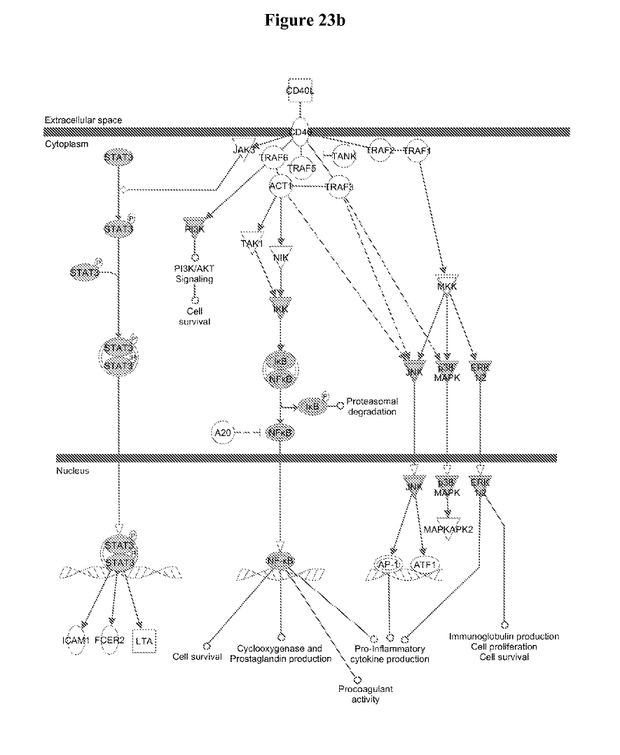

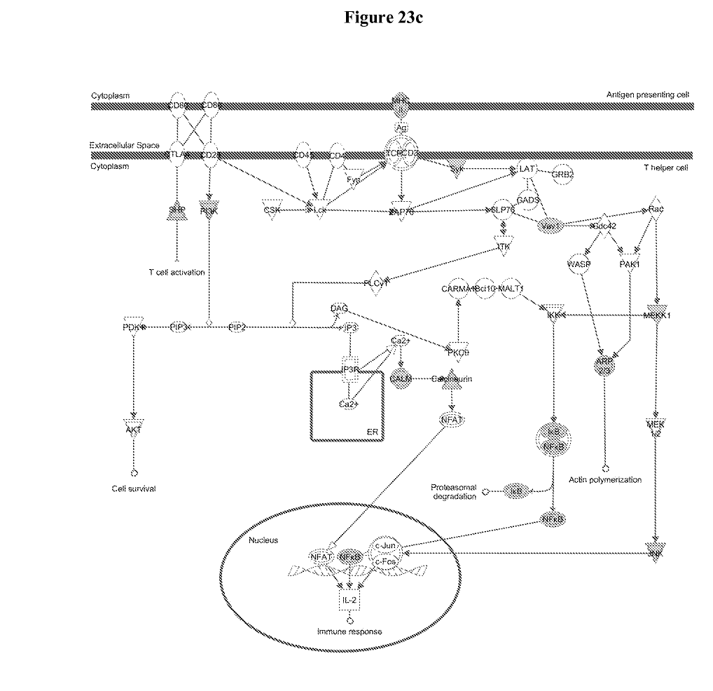

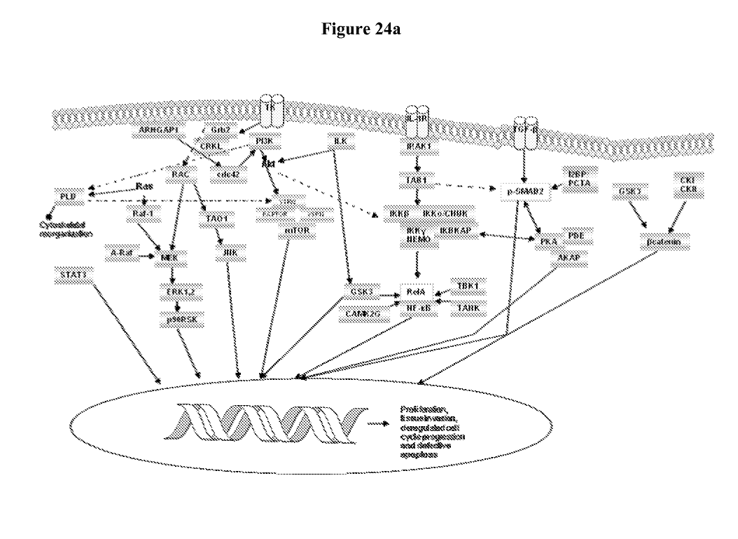

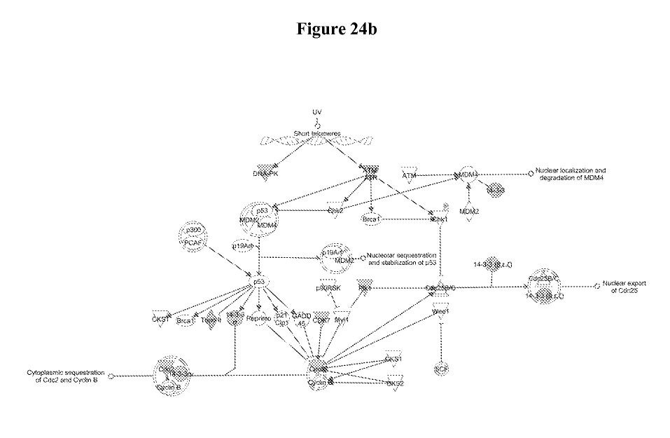

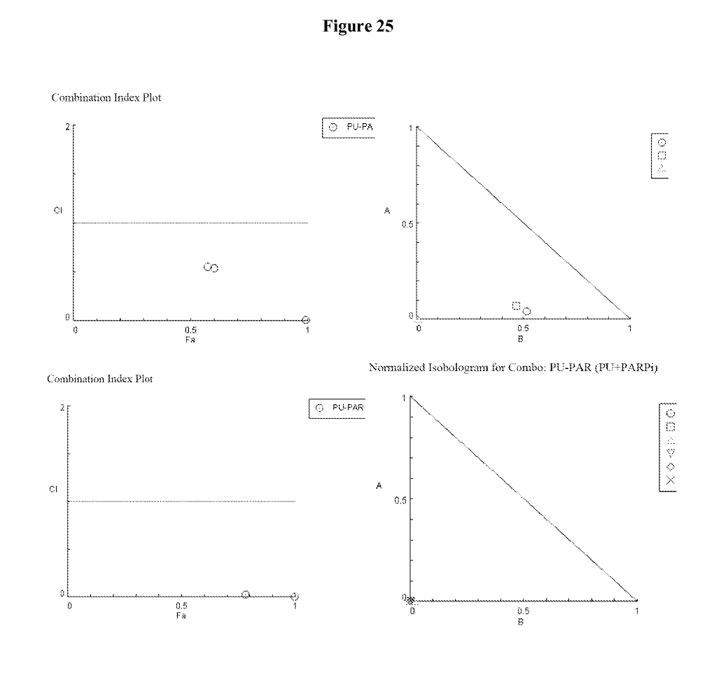

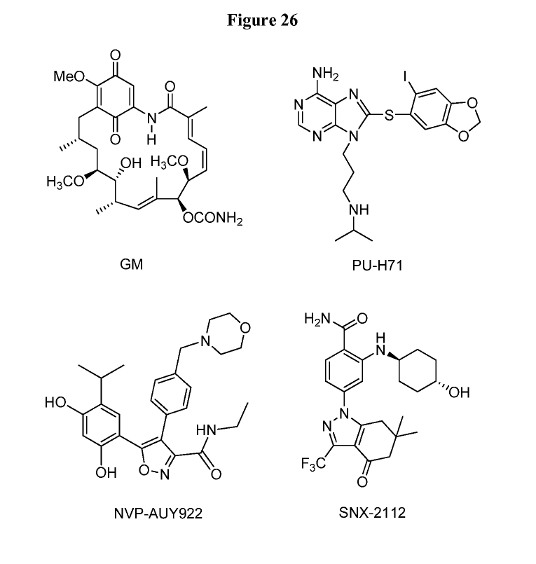

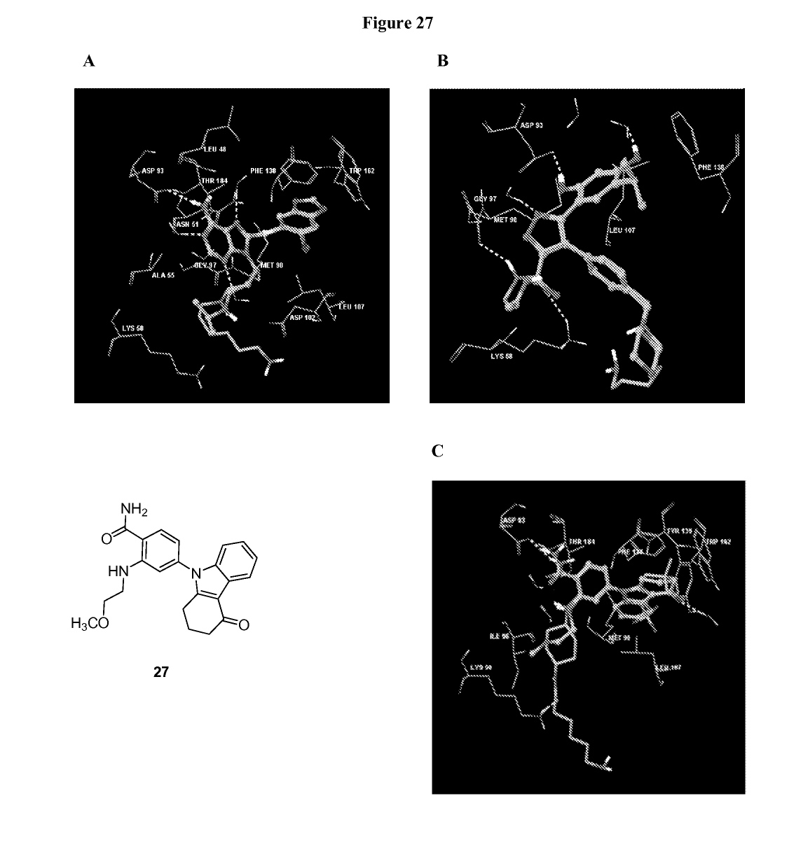

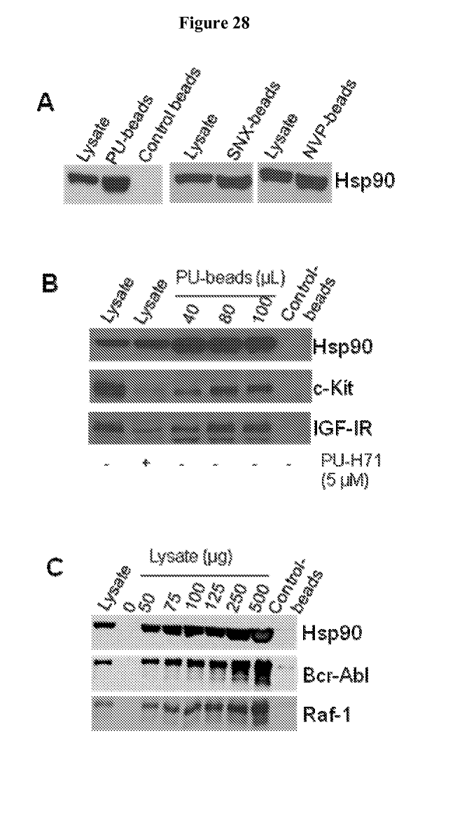

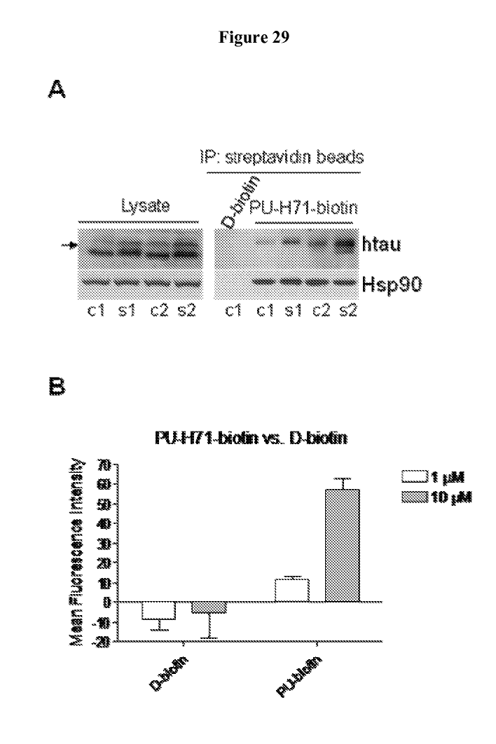

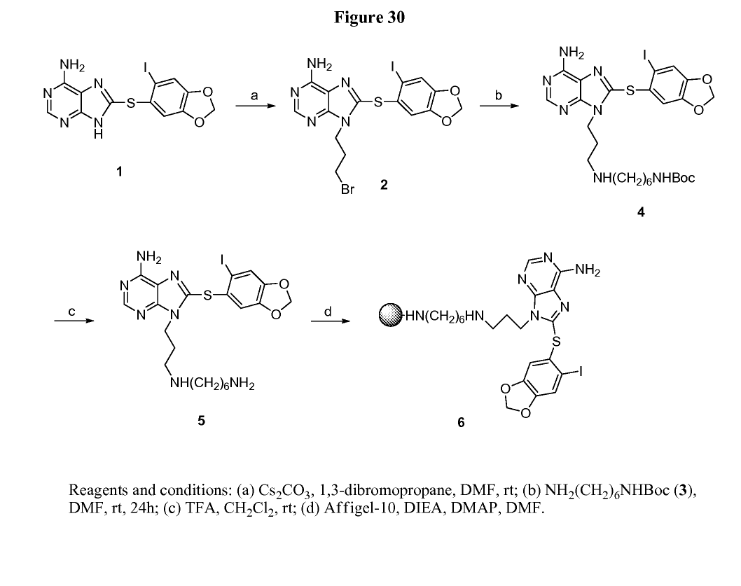

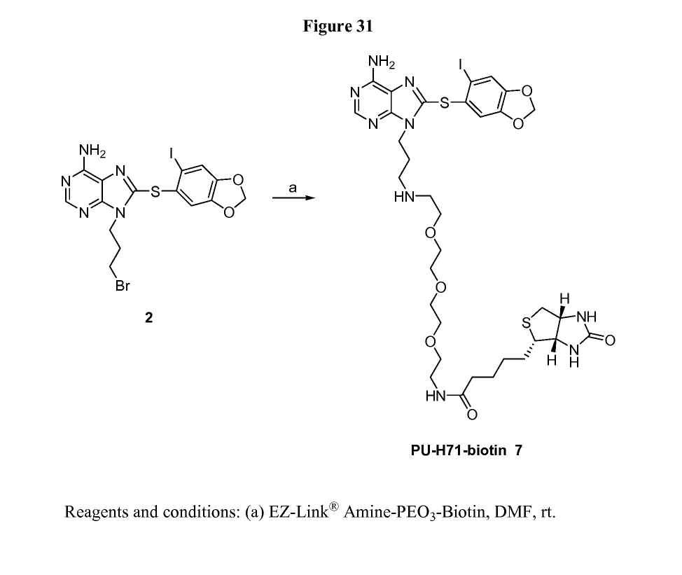

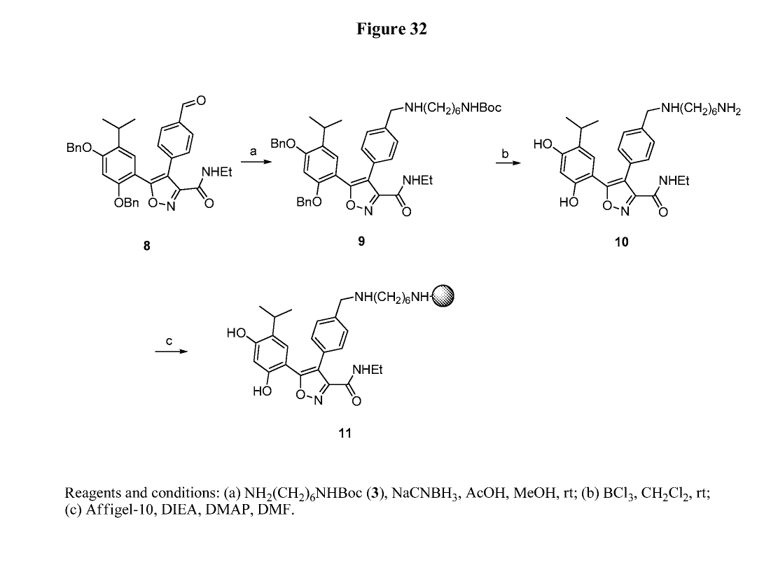

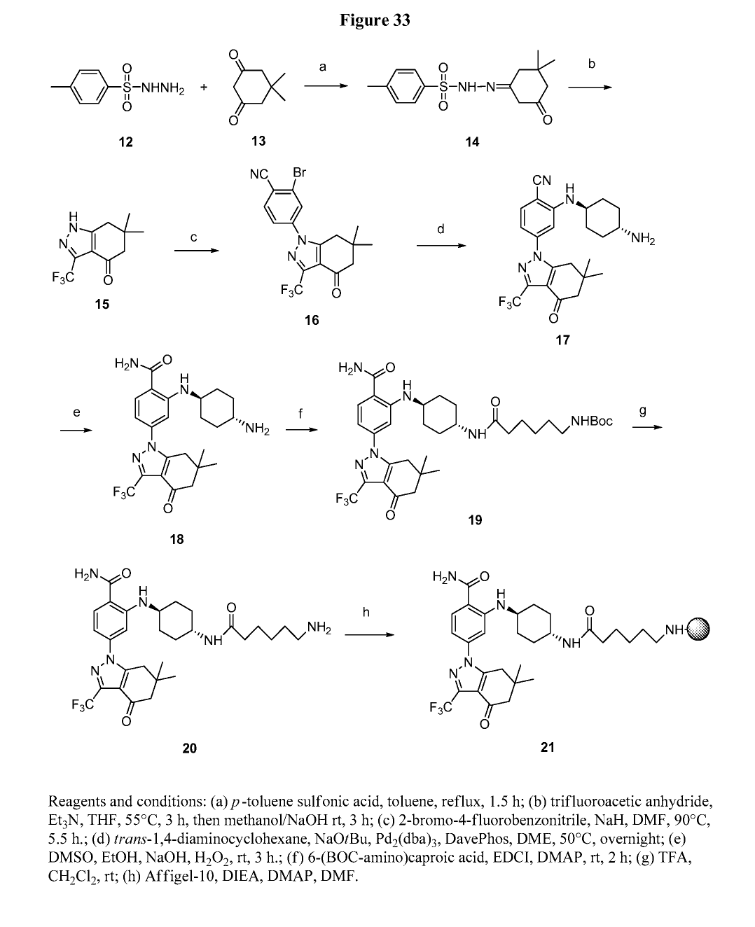

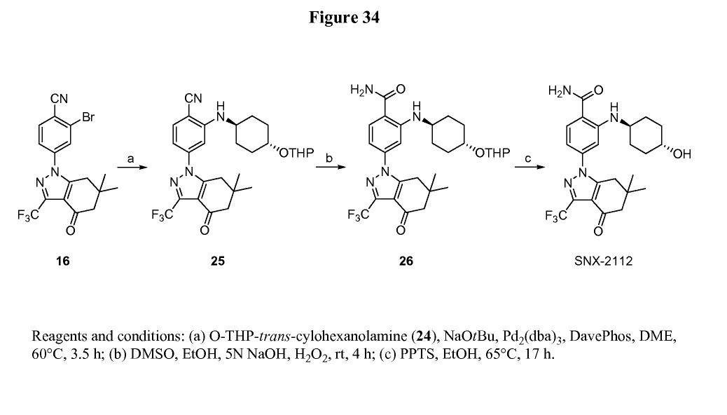

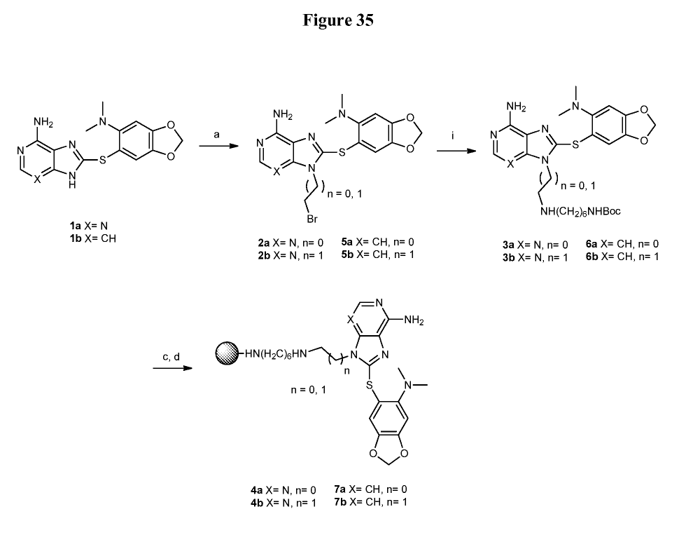

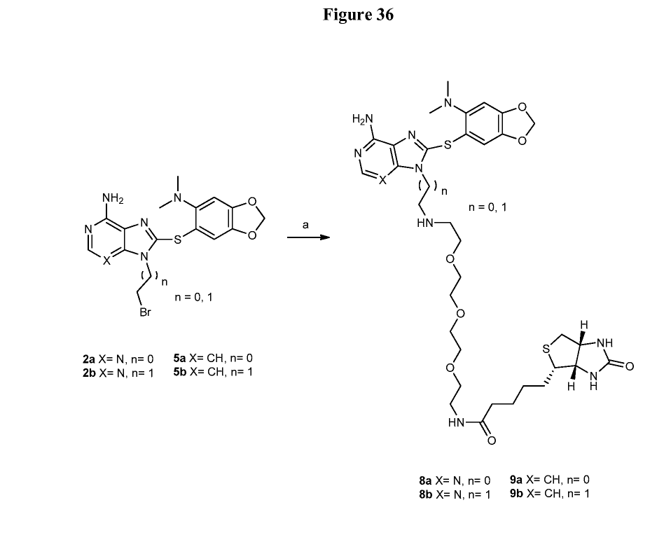

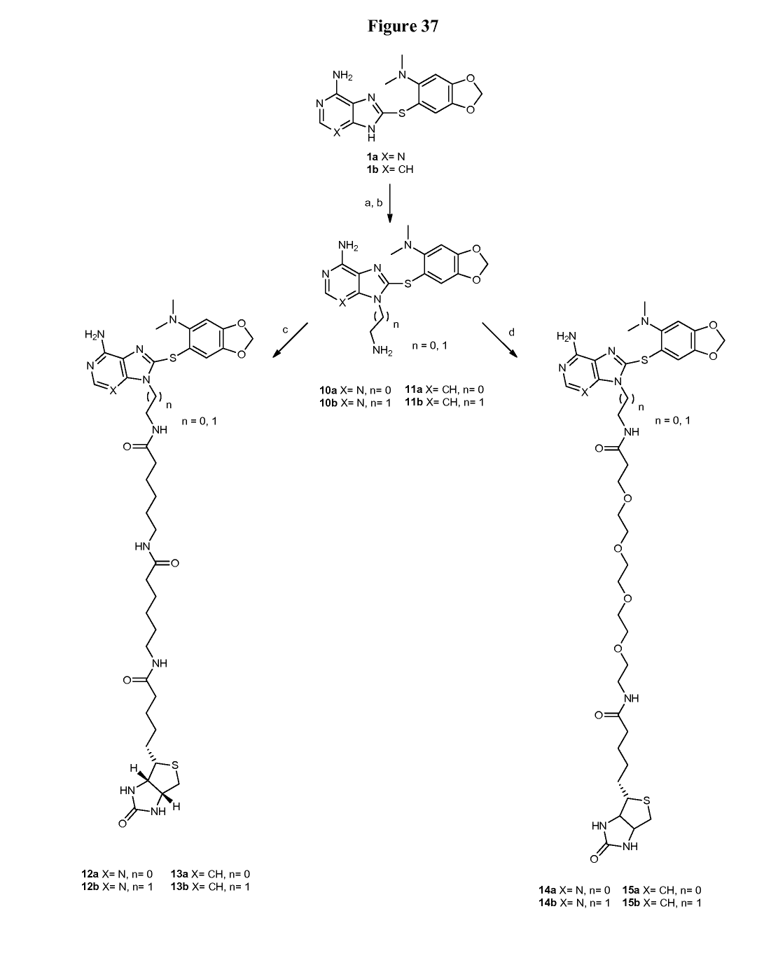

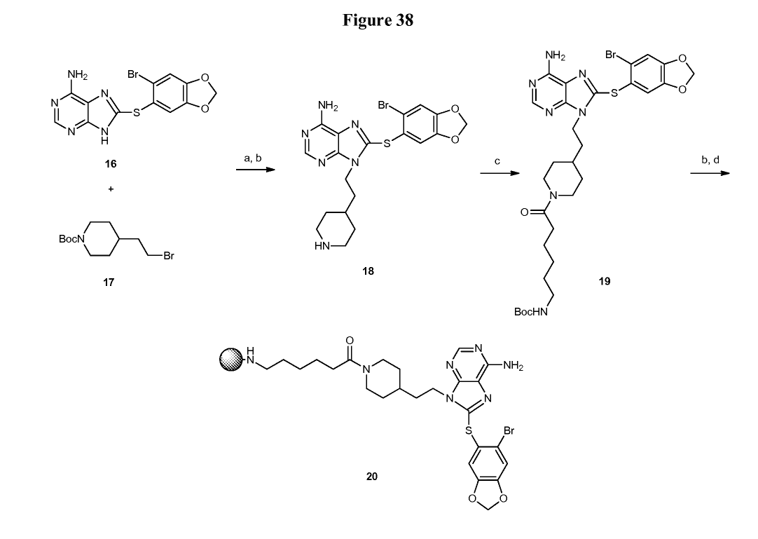

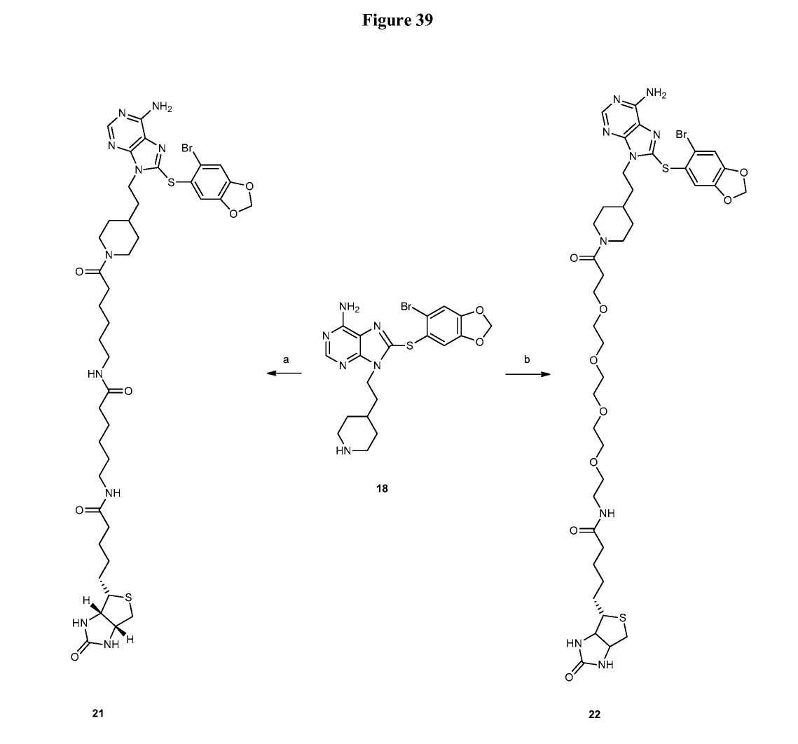

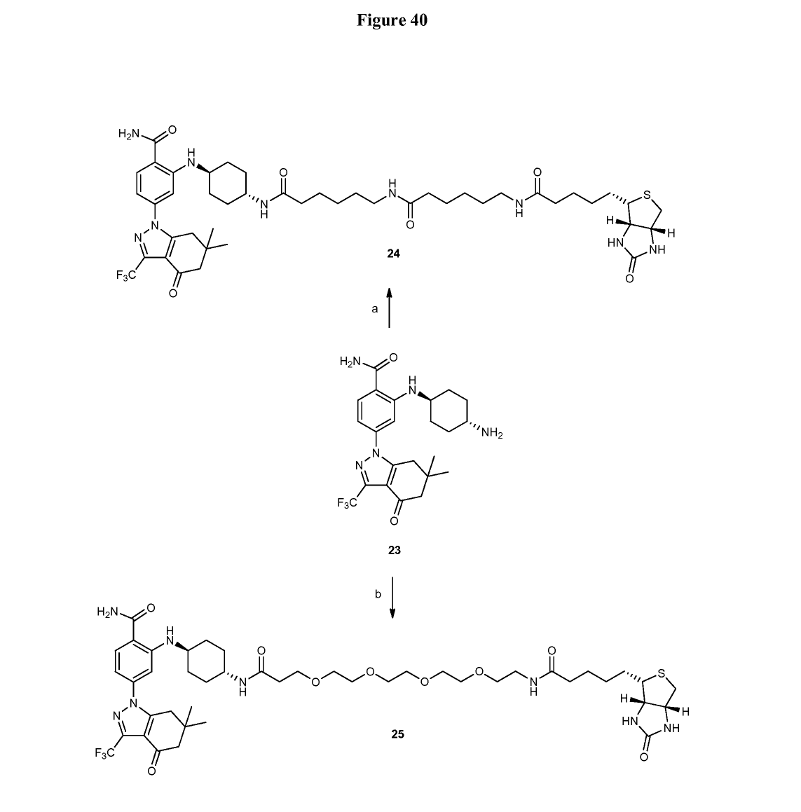

Ingenuity Pathway Analysis (IPA) was then used to build biological networks from the identified proteins (FIGS. 6a, 6b, 15; Tables 5e, 5f). IPA assigned PU-H71-isolated proteins to thirteen networks associated with cell death, cell cycle, cellular growth and proliferation. These networks overlap well with known canonical CML signaling pathways (FIG. 6a).

In addition to signaling proteins, we identified proteins that regulate carbohydrate and lipid metabolism, protein synthesis, gene expression, and cellular assembly and organization. These findings are in accord with the postulated broad roles of Hsp90 in maintaining cellular homeostasis and in being an important mediator of cell transformation (Zuehlke & Johnson, 2010; Workman et al., 2007; Dezwaan & Freeman, 2008; McClellan et al., 2007).

Following identification by MS, a number of key proteins were further validated by chemical precipitation and Western blot, in both K562 cells and in primary CML blasts (FIG. 6c, left, FIGS. 6d, 13a, 13b). The effect of PU-H71 on the steady-state levels of these proteins was also queried to further support their Hsp90-regulated expression/stability (FIG. 6c, right) (Zuehlke & Johnson, 2010).

The top scoring networks enriched on the PU-beads were those used by Bcr-Abl to propagate aberrant signaling in CML: the PI3K/mTOR-, MAPK- and NFκB-mediated signaling pathways (Network 1, 22 focus molecules, score=38 and Network 2, 22 focus molecules, score=36, Table 5f). Connectivity maps were created for these networks to investigate the relationship between component proteins (FIGS. 15a, 15b). These maps were simplified for clarity, retaining only major pathway components and relationships (FIG. 6b).

### the PI3K/mTOR-Pathway

Activation of the PI3K/mTOR-pathway has emerged as one of the essential signaling mechanisms in Bcr-Abl leukemogenesis (Ren, 2005). Of particular interest within this pathway is the mammalian target of rapamycin (mTOR), which is constitutively activated in Bcr-Abl-transformed cells, leading to dysregulated translation and contributing to leukemogenesis. A recent study provided evidence that both the mTORC1 and mTORC2 complexes are activated in Bcr-Abl cells and play key roles in mRNA translation of gene products that mediate mitogenic responses, as well as in cell growth and survival (Carayol et al., 2010). mTOR and key activators of mTOR, such as RICTOR, RAPTOR, Sin1 (MAPKAP1), class 3 PI3Ks PIK3C3, also called hVps34, and PIK3R4 (VSP15) (Nobukuni et al., 2007), were identified in the PU-Hsp90 pull-downs (Tables 5a, 5d; FIGS. 6c, 6d, 13b).

### the NF-κB Pathway

Activation of nuclear factor-κB (NF-κB) is required for Bcr-Abl transformation of primary bone marrow cells and for Bcr-Abl-transformed hematopoietic cells to form tumors in nude mice (McCubrey et al., 2008). PU-isolated proteins enriched on this pathway include NF-κB as well as activators of NF-kB such as IKBKAP, that binds NF-kappa-B-inducing kinase (NIK) and IKKs through separate domains and assembles them into an active kinase complex, and TBK-1 (TANK-binding kinase 1) and TAB1 (TAK1-binding protein 1), both positive regulators of the I-kappaB kinase/NF-kappaB cascade (Häcker & Karin, 2006) (Tables 5a, 5d). Recently, Bcr-Abl-induced activation of the NF-κB cascade in myeloid leukemia cells was demonstrated to be largely mediated by tyrosine-phosphorylated PKD2 (or PRKD2) (Mihailovic et al., 2004) which we identify here to be a PU-H71/Hsp90 interactor (Tables 5a, 5d; FIGS. 6c, 6d, 13b).

### The Raf/MAPK Pathway

Key effectors of the MAPK pathway, another important pathway activated in CML (Ren, 2005; McCubrey et al., 2008), such as Raf-1, A-Raf, ERK, p90RSK, vav and several MAPKs were also included the PU-Hsp90-bound pool (Tables 5a, 5d; FIGS. 6c, 6d, 13b). In addition to the ERK signal transduction cascade, we identify components that act on activating the P38 MAPK pathway, such as MEKK4 and TAB1. IPA connects the MAPK-pathway to key elements of many different signal transduction pathways including PI3K/mTOR-, STAT- and focal adhesion pathways (FIGS. 15a-d, 6b).

### the STAT Pathway

The STAT-pathway is also activated in CML and confers cytokine independence and protection against apoptosis (McCubrey et al., 2008) and was enriched by PU-H71 chemical precipitation (Network 8, 20 focus molecules, score=14, Table 5f, FIG. 15c). Both STAT5 and STAT3 were associated with PU-H71-Hsp90 complexes (Tables 5a, 5d; FIGS. 6c, 6d, 13b). In CML, STAT5 activation by phosphorylation is driven by Bcr-Abl (Ren, 2005). Bruton agammaglobulinemia tyrosine kinase (BTK), constitutively phosphorylated and activated by Bcr-Abl in pre-B lymphoblastic leukemia cell (Hendriks & Kersseboom, 2006), can also signal through STAT5 (Mahajan et al., 2001). BTK is another Hsp90-regulated protein that we identified in CML (Tables 5a, 5d; FIGS. 6c, 6d, 13b). In addition to phosphorylation, STATs can be activated in myeloid cells by calpain (CAPN1)-mediated proteolytic cleavage, leading to truncated STAT species (Oda et al., 2002). CAPN1 is also found in the PU-bound Hsp90 pulldowns, as is activated Ca(2+)/calmodulin-dependent protein kinase IIgamma (CaMKIIgamma), which is also activated by Bcr-Abl (Si & Collins, 2008) (Tables 5a, 5d). CaMKIIgamma activity in CML is associated with the activation of multiple critical signal transduction networks involving the MAPK and STAT pathways. Specifically, in myeloid leukemia cells, CaMKIIgamma also directly phosphorylates STAT3 and enhances its transcriptional activity (Si & Collins, 2008).

### the Focal Adhesion Pathway

Retention and homing of progenitor blood cells to the marrow microenvironment are regulated by receptors and agonists of survival and proliferation. Bcr-Abl induces adhesion independence resulting in aberrant release of hematopoietic stem cells from the bone marrow, and leading to activation of adhesion receptor signaling pathways in the absence of ligand binding. The focal adhesion pathway was well represented in PU-H71 pulldowns (Network 12, 16 focus molecules, score=13, Table 5f, FIG. 15d). The focal adhesion-associated proteins paxillin, FAK, vinculin, talin, and tensin are constitutively phosphorylated in Bcr-Abl-transfected cell lines (Salgia et al., 1995), and these too were isolated in PU-Hsp90 complexes (Tables 5a, 5d and FIG. 6c). In CML cells, FAK can activate STAT5 (Le et al., 2009).

Other important transforming pathways in CML, those driven by MYC (Sawyers, 1993) (Network 7, 15 focus molecules, score=22, FIGS. 6a and 15e, Table 5f) and TGF-β (Naka et al., 2010) (Network 10, 13 focus molecules, score=18, FIGS. 6a and 15f, Table 5f), were identified here as well. Among the identified networks were also those important for disease progression and aberrant cell cycle and proliferation of CML (Network 3, 20 focus molecules, score=33, Network 4, 20 focus molecules, score=33, Network 5, 20 focus molecules, score=32, Network 6, 19 focus molecules, score=30, Network 9, 14 focus molecules, score=20, Network 11, 12 focus molecules, score=17 and Network 13, 10 focus molecules, score=12, FIG. 6a and Table 50.

In summary, PU-H71 enriches a broad cross-section of proteins that participate in signaling pathways vital to the malignant phenotype in CML (FIG. 6). The interaction of PU-bound Hsp90 with the aberrant CML signalosome was retained in primary CML samples (FIGS. 6d, 13b).

### PU-H71 Identified Proteins and Networks are Those Important for the Malignant Phenotype

We demonstrate that the presence of these proteins in the PU-bead pull-downs is functionally significant and suggests a role for Hsp90 in broadly supporting the malignant signalosome in CML cells.

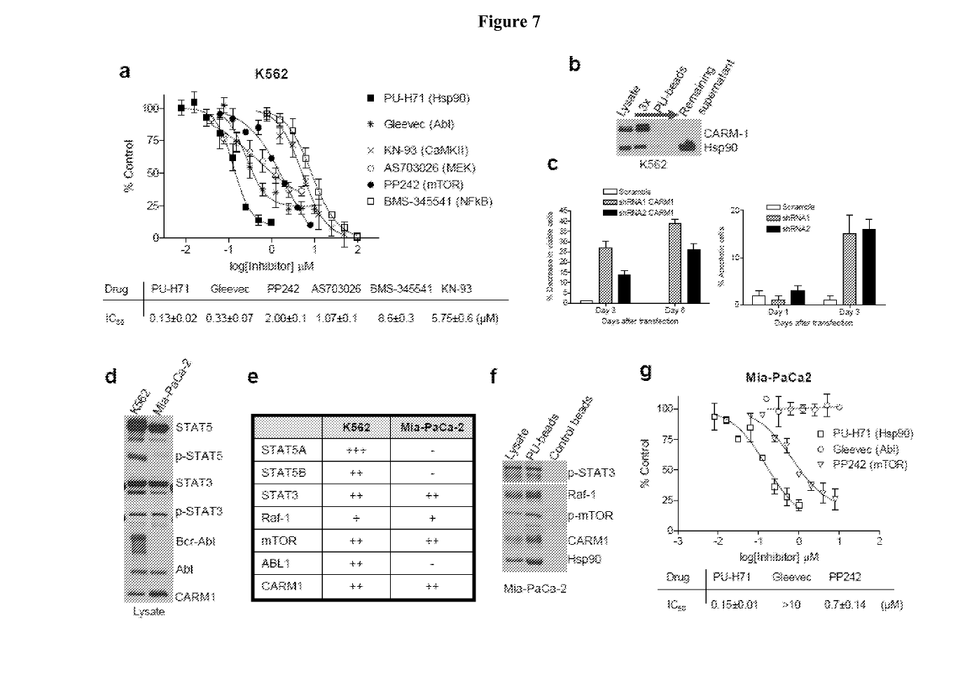

To demonstrate that the networks identified by PU-beads are important for transformation in K562, we next showed that inhibitors of key nodal proteins from individual networks (FIG. 6b, yellow boxes—Bcr-Abl, NFκB, mTOR, MEK and CAMIIK) diminish the growth and proliferation potential of K562 cells (FIG. 7a).

Next we demonstrated that PU-beads identified Hsp90 interactors with yet no assigned role in CML, also contribute to the transformed phenotype. The histone-arginine methyltransferase CARM1, a transcriptional co-activator of many genes (Bedford & Clarke, 2009), was validated in the PU-bead pull-downs from CML cell lines and primary CML cells (FIGS. 6c, 6d, 13). This is the first reported link between Hsp90 and CARM1, although other arginine methyltransferases, such as PRMT5, have been shown to be Hsp90 clients in ovarian cancer cells (Maloney et al., 2007). While elevated CARM1 levels are implicated in the development of prostate and breast cancers, little is known on the importance of CARM1 in CML leukomogenesis (Bedford & Clarke, 2009). We found CARM1 essentially entirely captured by the Hsp90 species recognized by PU-beads (FIG. 7b) and also sensitive to degradation by PU-H71 (FIG. 6c, right). CARM1 therefore, may be a novel Hsp90 onco-protein in CML. Indeed, knock-down experiments with CARM1 but not control shRNAs (FIG. 7c), demonstrate reduced viability and induction of apoptosis in K562 cells, supporting this hypothesis.

To demonstrate that the presence of proteins in the PU-pulldowns is due to their participation in aberrantly activated signaling and not merely their abundant expression, we compared PU-bead pulldowns from K562 and Mia-PaCa-2, a pancreatic cancer cell line (Table 5a). While both cells express high levels of STAT5 protein (FIG. 7d), activation of the STAT5 pathway, as demonstrated by STAT5 phosphorylation (FIG. 7d) and DNA-binding (Jaganathan et al., 2010), was noted only in the K562 cells. In accordance, this protein was identified only in the K562 PU-bead pulldowns (Table 5a and FIG. 7e). In contrast, activated STAT3 was identified in PU-Hsp90 complexes from both K562 (FIGS. 6c, 7e) and Mia-PaCa-2 cells extracts (FIGS. 7e, 7f).

The mTOR pathway was identified by the PU-beads in both K562 and Mia-PaCa-2 cells (FIGS. 7e, 7f), and indeed, its pharmacologic inhibition by PP242, a selective inhibitor that targets the ATP domain of mTOR (Apsel et al., 2008), is toxic to both cells (FIGS. 7a, 7g). On the other hand, the Abl inhibitor Gleevec (Deininger & Druker, 2003) was toxic only to K562 cells (FIGS. 7a, 7g). Both cells express Abl but only K562 has the oncogenic Bcr-Abl (FIG. 7d) and PU-beads identify Abl, as Bcr-Abl, in K562 but not in Mia-PaCa-2 cells (FIG. 7e).

### PU-H71 Identifies a Novel Mechanism of Oncogenic STAT-Activation

PU-bead pull-downs contain several proteins, including Bcr-Abl (Ren, 2005), CAMKIIγ (Si & Collins, 2008), FAK (Salgia et al., 1995), vav-1 (Katzav, 2007) and PRKD2 (Mihailovic et al., 2004) that are constitutively activated in CML leukemogenesis. These are classical Hsp90-regulated clients that depend on Hsp90 for their stability because their steady-state levels decrease upon Hsp90 inhibition (FIG. 6c) (Zuehlke & Johnson, 2010; Workman et al., 2007). Constitutive activation of STAT3 and STAT5 is also reported in CML (Ren, 2005; McCubrey et al., 2008). These proteins, however, do not fit the criteria of classical client proteins because STAT5 and STAT3 levels remain essentially unmodified upon Hsp90 inhibition (FIG. 6c). The PU-pull-downs also contain proteins isolated potentially as part of an active signaling mega-complex, such as mTOR, VSP32, VSP15 and RAPTOR (Carayol et al., 2010). mTOR activity, as measured by cellular levels of p-mTOR, also appears to be more sensitive to Hsp90 inhibition than are the complex components (i.e. compare the relative decrease in p-mTOR and RAPTOR in PU-H71 treated cells, FIG. 6c). Further, PU-Hsp90 complexes contain adapter proteins such as GRB2, DOCK, CRKL and EPS15, which link Bcr-Abl to key effectors of multiple aberrantly activated signaling pathways in K562 (Brehme et al., 2009; Ren, 2005) (FIG. 6b). Their expression also remains unchanged upon Hsp90 inhibition (FIG. 6c). We therefore wondered whether the contribution of Hsp90 to certain oncogenic pathways extends beyond its classical folding actions. Specifically, we hypothesized that Hsp90 might also act as a scaffolding molecule that maintains signaling complexes in their active configuration, as has been previously postulated (Dezwaan & Freeman, 2008; Pratt et al., 2008).

### Hsp90 Binds to and Influences the Conformation of STAT5

To investigate this hypothesis further we focused on STAT5, which is constitutively phosphorylated in CML (de Groot et al., 1999). The overall level of p-STAT5 is determined by the balance of phosphorylation and dephosphorylation events. Thus, the high levels of p-STAT5 in K562 cells may reflect either an increase in upstream kinase activity or a decrease in protein tyrosine phosphatase (PTPase) activity. A direct interaction between Hsp90 and p-STAT5 could also modulate the cellular levels of p-STAT5.

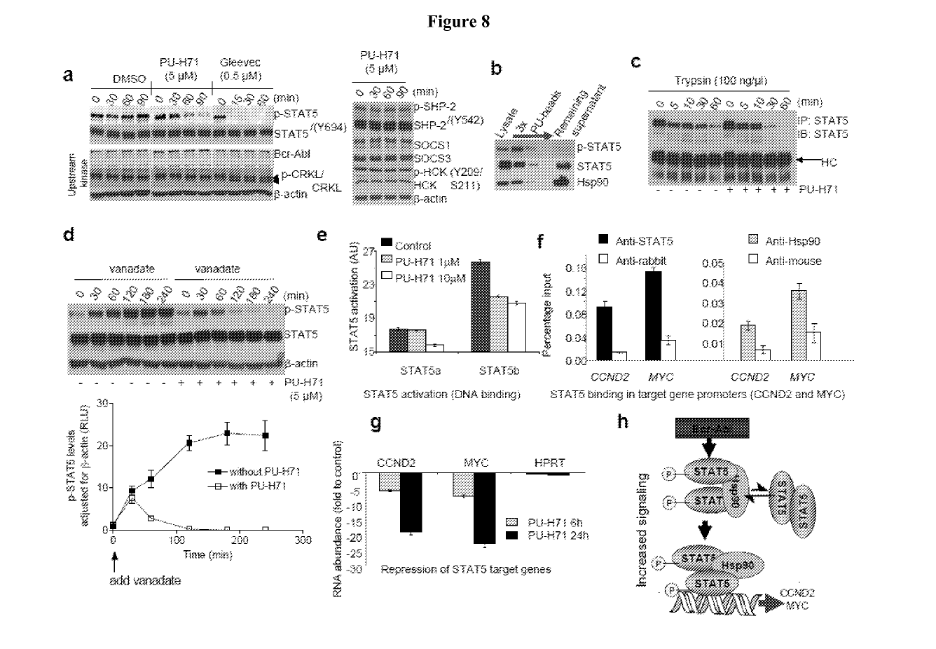

To dissect the relative contribution of these potential mechanisms, we first investigated the effect of PU-H71 on the main kinases and PTPases that regulate STAT5 phosphorylation in K562 cells. Bcr-Abl directly activates STAT5 without the need for JAK phosphorylation (de Groot et al., 1999). Concordantly, STAT5-phosphorylation rapidly decreased in the presence of the Bcr-Abl inhibitor Gleevec (FIG. 8a, left, Gleevec). While Hsp90 regulates Bcr-Abl stability, the reduction in steady-state Bcr-Abl levels following Hsp90 inhibition requires more than 3 h (An et al., 2000). Indeed no change in Bcr-Abl expression (FIG. 8a, left, PU-H71, Bcr-Abl) or function, as evidenced by no decrease in CRKL phosphorylation (FIG. 8a, left, PU-H71, p-CRKL/CRKL), was observed with PU-H71 in the time interval it reduced p-STAT5 levels (FIG. 8a, left, PU-H71, p-STAT5). Also, no change in the activity and expression of HCK, a kinase activator of STAT5 in 32Dcl3 cells transfected with Bcr-Abl Klejman et al., 2002), was noted (FIG. 8a, right, HCK/p-HCK).

Thus reduction of p-STAT5 phosphorylation by PU-H71 in the 0 to 90 min interval (FIG. 8c, left, PU-H71) is unlikely to be explained by destabilization of Bcr-Abl or other kinases.

We therefore examined whether the rapid decrease in p-STAT5 levels in the presence of PU-H71 may be accounted for by an increase in PTPase activity. The expression and activity of SHP2, the major cytosolic STAT5 phosphatase (Xu & Qu, 2008), were also not altered within this time interval (FIG. 8a, right, SHP2/p-SHP2). Similarly, the levels of SOCS1 and SOCS3, which form a negative feedback loop that switches off STAT-signaling Deininger & Druker, 2003) were unaffected by PU-H71 (FIG. 8a, right, SOCS1/3).

Thus no effect on STAT5 in the interval 0-90 min can likely be attributed to a change in kinase or phosphatase activity towards STAT5. As an alternative mechanism, and because the majority of p-STAT5 but not STAT5 is Hsp90 bound in CML cells (FIG. 8b), we hypothesized that the cellular levels of activated STAT5 are fine-tuned by direct binding to Hsp90.

The activation/inactivation cycle of STATs entails their transition between different dimer conformations. Phosphorylation of STATs occurs in an anti-parallel dimer conformation that upon phosphorylation triggers a parallel dimer conformation. Dephosphorylation of STATs on the other hand require extensive spatial reorientation, in that the tyrosine phosphorylated STAT dimers must shift from parallel to anti-parallel configuration to expose the phosphotyrosine as a better target for phosphatases (Lim & Cao, 2006). We find that STAT5 is more susceptible to trypsin cleavage when bound to Hsp90 (FIG. 8c), indicating that binding of Hsp90 directly modulates the conformational state of STAT5, potentially to keep STAT5 in a conformation unfavorable for dephosphorylation and/or favorable for phosphorylation.

To investigate this possibility we used a pulse-chase strategy in which orthovanadate (Na₃VO₄), a non-specific PTPase inhibitor, was added to cells to block the dephosphorylation of STAT5. The residual level of p-STAT5 was then determined at several later time points (FIG. 8d). In the absence of PU-H71, p-STAT5 accumulated rapidly, whereas in its presence, cellular p-STAT5 levels were diminished. The kinetics of this process (FIG. 8d) were similar to the rate of p-STAT5 steady-state reduction (FIG. 8a, left, PU-H71).

Hsp90 Maintains STAT5 in an Active Conformation Directly within STAT5-Containing Transcriptional Complexes

In addition to STAT5 phosphorylation and dimerization, the biological activity of STAT5 requires its nuclear translocation and direct binding to its various target genes (de Groot et al., 1999; Lim & Cao, 2006). We wondered therefore, whether Hsp90 might also facilitate the transcriptional activation of STAT5 genes, and thus participate in promoter-associated STAT5 transcription complexes. Using an ELISA-based assay, we found that STAT5 (FIG. 8e) is constitutively active in K562 cells and binds to a STAT5 binding consensus sequence (5′-TTCCCGGAA-3′). STAT5 activation and DNA binding is partially abrogated, in a dose-dependent manner, upon Hsp90 inhibition with PU-H71 (FIG. 8e). Furthermore, quantitative ChIP assays in K562 cells revealed the presence of both Hsp90 and STAT5 at the critical STAT5 targets MYC and CCND2 (FIG. 8f). Neither protein was present at intergenic control regions (not shown). Accordingly, PU-H71 (1 μM) decreased the mRNA abundance of the STAT5 target genes CCND2, MYC, CCND1, BCL-XL and MCL1 (Katzav, 2007), but not of the control genes HPRT and GAPDH (FIG. 8g and not shown).

Collectively, these data show that STAT5 activity is positively regulated by Hsp90 in CML cells (FIG. 8h). Our findings are consistent with a scenario whereby Hsp90 binding to STAT5 modulates the conformation of the protein and by this mechanism it alters STAT5 phosphorylation/dephosphorylation kinetics, shifting the balance towards increased levels of p-STAT5. In addition, Hsp90 maintains STAT5 in an active conformation directly within STAT5-containing transcriptional complexes. Considering the complexity of the STAT-pathway, other potential mechanisms however, cannot be excluded. Therefore, in addition to its role in promoting protein stability, Hsp90 promotes oncogenesis by maintaining client proteins in an active configuration.

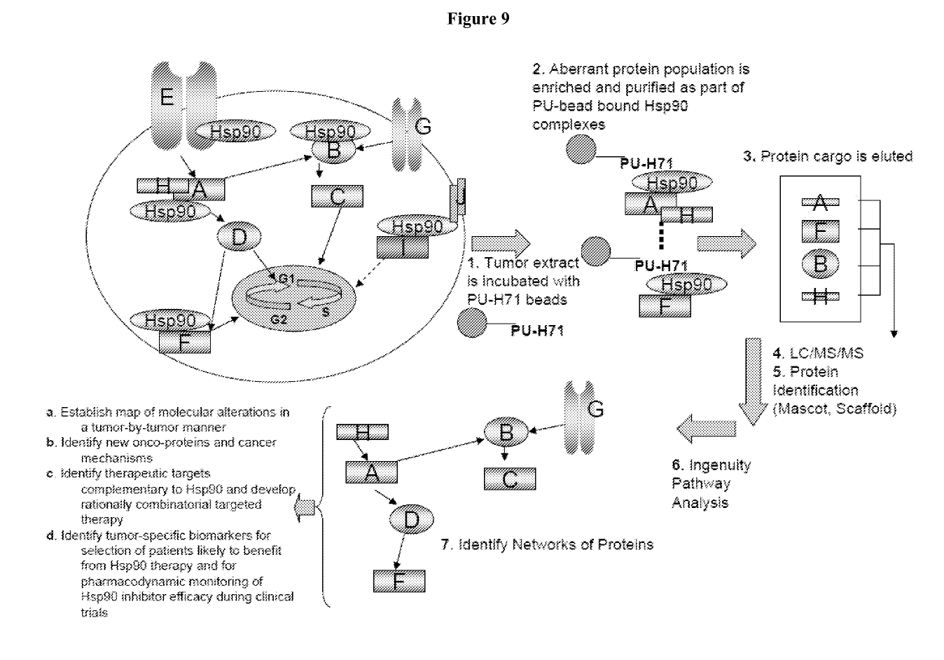

More broadly, the data suggest that it is the PU-H71-Hsp90 fraction of cellular Hsp90 that is most closely involved in supporting oncogenic protein functions in tumor cells, and PU-H71-Hsp90 proteomics can be used to identify a broad cross-section of the protein pathways required to maintain the malignant phenotype in specific tumor cells (FIG. 9).

### Discussion

It is now appreciated that many proteins that are required to maintain tumor cell survival may not present mutations in their coding sequence, and yet identifying these proteins is of extreme importance to understand how individual tumors work. Genome wide mutational studies may not identify these oncoproteins since mutations are not required for many genes to support tumor cell survival (e.g. IRF4 in multiple myeloma and BCL6 in B-cell lymphomas) (Cerchietti et al., 2009). Highly complex, expensive and large-scale methods such as RNAi screens have been the major means for identifying the complement of oncogenic proteins in various tumors (Horn et al., 2010). We present herein a rapid and simple chemical-proteomics method for surveying tumor oncoproteins regardless of whether they are mutated (FIG. 9). The method takes advantage of several properties of PU-H71 which i) binds preferentially to the fraction of Hsp90 that is associated with oncogenic client proteins, and ii) locks Hsp90 in an onco-client bound configuration. Together these features greatly facilitate the chemical affinity-purification of tumor-associated protein clients by mass spectrometry (FIG. 9). We propose that this approach provides a powerful tool in dissecting, tumor-by-tumor, lesions characteristic of distinct cancers. Because of the initial chemical precipitation step, which purifies and enriches the aberrant protein population as part of PU-bead bound Hsp90 complexes, the method does not require expensive SILAC labeling or 2-D gel separations of samples. Instead, protein cargo from PU-bead pull-downs is simply eluted in SDS buffer, submitted to standard SDS-PAGE, and then the separated proteins are extracted and trypsinized for LC/MS/MS analyses.

While this method presents a unique approach to identify the oncoproteins that maintain the malignant phenotype of tumor cells, one needs to be aware that, similarly to other chemical or antibody-based proteomics techniques, it also has potential limitations (Rix & Superti-Furga, 2009). For example, “sticky” or abundant proteins may also bind in a nondiscriminatory fashion to proteins isolated by the PU-H71 beads. Such proteins were catalogued by several investigators (Trinkle-Mulcahy et al., 2008), and we have used these lists to eliminate them from the pull-downs with the clear understanding that some of these proteins may actually be genuine Hsp90 clients. Second, while we have presented several lines of evidence that PU-H71 is specific for Hsp90 (FIG. 11; Taldone & Chiosis, 2009), one must also consider that at the high concentration of PU-H71 present on the beads, unspecific and direct binding of the drug to a small number of proteins is unavoidable.

In spite of the potential limitations described in the preceeding paragraph, we have, using this method, performed the first global evaluation of Hsp90-facilitated aberrant signaling pathways in CML. The Hsp90 interactome identified by PU-H71 affinity purification significantly overlaps with the well-characterized CML signalosome (FIG. 6a), indicating that this method is able to identify a large part of the complex web of pathways and proteins that define the molecular basis of this form of leukemia. We suggest that PU-H71 chemical-proteomics assays may be extended to other forms of cancer in order to identify aberrant signaling networks that drive the malignant phenotype in individual tumors (FIG. 9). For example, we show further here how the method is used to identify the aberrant protein networks in the MDA-MB-468 triple-negative breast cancer cells, the MiaPaCa2 pancreatic cancer cells and the OCI-LY1 diffuse large B-cell lymphoma cells.

Since single agent therapy is not likely to be curative in cancer, it is necessary to design rational combinatorial therapy approaches. Proteomic identification of oncogenic Hsp90-scaffolded signaling networks may identify additional oncoproteins that could be further targeted using specific small molecule inhibitors. Indeed, inhibitors of mTOR and CAMKII, which are identified by our method to contribute to the transformation of K562 CML cells and be key nodal proteins on individual networks (FIG. 6b, yellow boxes), are active as single agents (FIG. 7a) and synergize with Hsp90 inhibition in affecting the growth of these leukemia cells (FIG. 21).

When applied to less well-characterized tumor types, PU-H71 chemical proteomics might provide less obvious and more impactful candidate targets for combinatorial therapy. We exemplify this concept in the MDA-MB-468 triple-negative breast cancer cells, the MiaPaCa2 pancreatic cancer cells and the OCI-LY1 diffuse large B-cell lymphoma cells.

In the triple negative breast cancer cell line MDA-MB-468 major signaling networks identified by the method were the PI3K/AKT, IGF-IR, NRF2-mediated oxidative stress response, MYC, PKA and the IL-6 signaling pathways (FIG. 22). Pathway components as identified by the method are listed in Table 3.

TABLE 3
© 2000-2012 Ingenuity Systems, Inc. All rights reserved.

| ID | Notes | Symbol | Entrez Gene Name | Location | Type(s) | Drug(s) |
|---|---|---|---|---|---|---|
| AAGAB | | AAGAB | alpha- and | Cytoplasm | other | |
| | | | gamma-adaptin | | | |
| | | | binding protein | | | |
| ABHD10 | | ABHD10 | abhydrolase | Cytoplasm | other | |
| | | | domain | | | |
| | | | containing 10 | | | |
| ACAP2 | | ACAP2 | ArfGAP with | Nucleus | other | |
| | | | coiled-coil, | | | |
| | | | ankyrin repeat | | | |
| | | | and PH domains 2 | | | |
| AHSA1 | | AHSA1 | AHA1, activator | Cytoplasm | other | |
| | | | of heat shock | | | |
| | | | 90 kDa protein | | | |
| | | | ATPase | | | |
| | | | homolog 1 | | | |
| | | | (yeast) | | | |
| AKAP8 | | AKAP8 | A kinase | Nucleus | other | |
| | | | (PRKA) anchor | | | |
| | | | protein 8 | | | |
| AKAP8L | | AKAP8L | A kinase | Nucleus | other | |
| | | | (PRKA) anchor | | | |
| | | | protein 8-like | | | |
| ALYREF | | ALYREF | Aly/REF export | Nucleus | transcription | |
| | | | factor | | regulator | |
| ANKRD17 | | ANKRD17 | ankyrin repeat | unknown | other | |
| | | | domain 17 | | | |
| ANKRD50 | | ANKRD50 | ankyrin repeat | unknown | other | |
| | | | domain 50 | | | |
| ANP32A | | ANP32A | acidic (leucine- | Nucleus | other | |
| | | | rich) nuclear | | | |
| | | | phosphoprotein | | | |
| | | | 32 family, | | | |
| | | | member A | | | |
| ANXA11 | | ANXA11 | annexin A11 | Nucleus | other | |
| ANXA2 | | ANXA2 | annexin A2 | Plasma | other | |
| | | | | Membrane | | |
| ANXA7 | | ANXA7 | annexin A7 | Plasma | ion channel | |
| | | | | Membrane | | |
| ARFGAP1 | | ARFGAP1 | ADP-ribosylation | Cytoplasm | transporter | |
| | | | factor GTPase | | | |
| | | | activating | | | |
| | | | protein 1 | | | |
| ARFGEF2 | | ARFGEF2 | ADP-ribosylation | Cytoplasm | other | |
| | | | factor guanine | | | |
| | | | nucleotide- | | | |
| | | | exchange factor | | | |
| | | | 2 (brefeldin A- | | | |
| | | | inhibited) | | | |
| ARFIP2 | | ARFIP2 | ADP-ribosylation | Cytoplasm | other | |
| | | | factor interacting | | | |
| | | | protein 2 | | | |
| ARHGAP29 | | ARHGAP29 | Rho GTPase | Cytoplasm | other | |
| | | | activating | | | |
| | | | protein 29 | | | |
| ARHGEF40 | | ARHGEF40 | Rho guanine | unknown | other | |
| | | | nucleotide | | | |
| | | | exchange factor | | | |
| | | | (GEF) 40 | | | |
| ASAH1 | | ASAH1 | N- | Cytoplasm | enzyme | |
| | | | acylsphingosine | | | |
| | | | amidohydrolase | | | |
| | | | (acid | | | |
| | | | ceramidase) 1 | | | |
| ATL3 | | ATL3 | atlastin GTPase 3 | Cytoplasm | other | |
| BAG4 | | BAG4 | BCL2- | Cytoplasm | other | |
| | | | associated | | | |
| | | | athanogene 4 | | | |
| BAG6 | | BAG6 | BCL2- | Nucleus | enzyme | |
| | | | associated | | | |
| | | | athanogene 6 | | | |
| BECN1 | | BECN1 | beclin 1, | Cytoplasm | other | |
| | | | autophagy | | | |
| | | | related | | | |
| BIRC6 | | BIRC6 | baculoviral IAP | Cytoplasm | enzyme | |
| | | | repeat | | | |
| | | | containing 6 | | | |
| BLMH | | BLMH | bleomycin | Cytoplasm | peptidase | |
| | | | hydrolase | | | |
| BRAT1 | | BRAT1 | BRCA1- | Cytoplasm | other | |
| | | | associated ATM | | | |
| | | | activator 1 | | | |
| BRCC3 | | BRCC3 | BRCA1/BRCA2- | Nucleus | enzyme | |
| | | | containing | | | |
| | | | complex, | | | |
| | | | subunit 3 | | | |
| BRD4 | | BRD4 | bromodomain | Nucleus | kinase | |
| | | | containing 4 | | | |
| BTAF1 | | BTAF1 | BTAF1 RNA | Nucleus | transcription | |
| | | | polymerase II, | | regulator | |
| | | | B-TFIID | | | |
| | | | transcription | | | |
| | | | factor- | | | |
| | | | associated, | | | |
| | | | 170 kDa (Mot1 | | | |
| | | | homolog, | | | |
| | | | S. cerevisiae) | | | |
| BUB1B | | BUB1B | budding | Nucleus | kinase | |
| | | | uninhibited by | | | |
| | | | benzimidazoles | | | |
| | | | 1 homolog beta | | | |
| | | | (yeast) | | | |
| BUB3 | | BUB3 | budding | Nucleus | other | |
| | | (includes | uninhibited by | | | |
| | | EG: 12237) | benzimidazoles | | | |
| | | | 3 homolog | | | |
| | | | (yeast) | | | |
| BYSL | | BYSL | bystin-like | Cytoplasm | other | |
| BZW1 | | BZW1 | basic leucine | Cytoplasm | translation | |
| | | | zipper and W2 | | regulator | |
| | | | domains 1 | | | |
| CACYBP | | CACYBP | calcyclin binding | Nucleus | other | |
| | | | protein | | | |
| CALU | | CALU | calumenin | Cytoplasm | other | |
| CAMK2G | | CAMK2G | calcium/calmodulin- | Cytoplasm | kinase | |
| | | | dependent | | | |
| | | | protein kinase II | | | |
| | | | gamma | | | |
| CAND1 | | CAND1 | cullin-associated | Cytoplasm | transcription | |
| | | | and neddylation- | | regulator | |
| | | | dissociated 1 | | | |
| CANX | | CANX | calnexin | Cytoplasm | other | |
| CAP1 | | CAP1 | CAP, adenylate | Plasma | other | |
| | | | cyclase- | Membrane | | |
| | | | associated | | | |
| | | | protein 1 (yeast) | | | |
| CAPRIN1 | | CAPRIN1 | cell cycle | Plasma | other | |
| | | | associated | Membrane | | |
| | | | protein 1 | | | |
| CAPZA1 | | CAPZA1 | capping protein | Cytoplasm | other | |
| | | | (actin filament) | | | |
| | | | muscle Z-line, | | | |
| | | | alpha 1 | | | |
| CAPZB | | CAPZB | capping protein | Cytoplasm | other | |
| | | | (actin filament) | | | |
| | | | muscle Z-line, | | | |
| | | | beta | | | |
| CARM1 | | CARM1 | coactivator- | Nucleus | transcription | |
| | | | associated | | regulator | |
| | | | arginine | | | |
| | | | methyltransferase 1 | | | |
| CASKIN1 | | CASKIN1 | CASK | Nucleus | transcription | |
| | | | interacting | | regulator | |
| | | | protein 1 | | | |
| CAT | | CAT | catalase | Cytoplasm | enzyme | |
| CBR1 | | CBR1 | carbonyl | Cytoplasm | enzyme | |
| | | | reductase 1 | | | |
| CCDC124 | | CCDC124 | coiled-coil | unknown | other | |
| | | | domain | | | |
| | | | containing 124 | | | |
| CCDC99 | | CCDC99 | coiled-coil | Nucleus | other | |
| | | | domain | | | |
| | | | containing 99 | | | |
| CDC37 | | CDC37 | cell division | Cytoplasm | other | |
| | | | cycle 37 | | | |
| | | | homolog | | | |
| | | | (S. cerevisiae) | | | |
| CDC37L1 | | CDC37L1 | cell division | Cytoplasm | other | |
| | | | cycle 37 | | | |
| | | | homolog | | | |
| | | | (S. cerevisiae)- | | | |
| | | | like 1 | | | |
| CDC42BPG | | CDC42BPG | CDC42 binding | Cytoplasm | kinase | |
| | | | protein kinase | | | |
| | | | gamma (DMPK- | | | |
| | | | like) | | | |
| CDH1 | | CDH1 | cadherin 1, type | Plasma | other | |
| | | | 1, E-cadherin | Membrane | | |
| | | | (epithelial) | | | |
| CDK1 | | CDK1 | cyclin- | Nucleus | kinase | flavopiridol |
| | | | dependent | | | |
| | | | kinase 1 | | | |
| CDK13 | | CDK13 | cyclin- | Nucleus | kinase | |
| | | | dependent | | | |
| | | | kinase 13 | | | |
| CDK4 | | CDK4 | cyclin- | Nucleus | kinase | PD-0332991, |
| | | | dependent | | | flavopiridol |
| | | | kinase 4 | | | |
| CDK7 | | CDK7 | cyclin- | Nucleus | kinase | BMS-387032, |
| | | | dependent | | | flavopiridol |
| | | | kinase 7 | | | |
| CHTF18 | | CHTF18 | CTF18, | unknown | other | |
| | | | chromosome | | | |
| | | | transmission | | | |
| | | | fidelity factor 18 | | | |
| | | | homolog | | | |
| | | | (S. cerevisiae) | | | |
| CNDP2 | | CNDP2 | CNDP | Cytoplasm | peptidase | |
| | | | dipeptidase 2 | | | |
| | | | (metallopeptidase | | | |
| | | | M20 family) | | | |
| CNN3 | | CNN3 | calponin 3, | Cytoplasm | other | |
| | | | acidic | | | |
| CNOT1 | | CNOT1 | CCR4-NOT | Cytoplasm | other | |
| | | | transcription | | | |
| | | | complex, | | | |
| | | | subunit 1 | | | |
| CNOT2 | | CNOT2 | CCR4-NOT | Nucleus | transcription | |
| | | | transcription | | regulator | |
| | | | complex, | | | |
| | | | subunit 2 | | | |
| CNOT7 | | CNOT7 | CCR4-NOT | Nucleus | transcription | |
| | | | transcription | | | |
| | | | complex, | | | |
| | | | subunit 7 | | | |
| CPOX | | CPOX | coproporphyrinogen | Cytoplasm | enzyme | |
| | | | oxidase | | | |
| CSDA | | CSDA | cold shock | Nucleus | transcription | |
| | | | domain protein A | | regulator | |
| CSNK1A1 | | CSNK1A1 | casein kinase 1, | Cytoplasm | kinase | |
| | | | alpha 1 | | | |
| CSNK2A1 | | CSNK2A1 | casein kinase 2, | Cytoplasm | kinase | |
| | | | alpha 1 | | | |
| | | | polypeptide | | | |
| CSNK2A2 | | CSNK2A2 | casein kinase 2, | Cytoplasm | kinase | |
| | | | alpha prime | | | |
| | | | polypeptide | | | |
| CTNNB1 | | CTNNB1 | catenin | Nucleus | transcription | |
| | | | (cadherin- | | regulator | |
| | | | associated | | | |
| | | | protein), beta 1, | | | |
| | | | 88 kDa | | | |
| CTNND1 | | CTNND1 | catenin | Nucleus | other | |
| | | | (cadherin- | | | |
| | | | associated | | | |
| | | | protein), delta 1 | | | |
| CTSB | | CTSB | cathepsin B | Cytoplasm | peptidase | |
| CTTN | | CTTN | cortactin | Plasma | other | |
| | | | | Membrane | | |
| CTU1 | | CTU1 | cytosolic | Cytoplasm | other | |
| | | | thiouridylase | | | |
| | | | subunit 1 | | | |
| | | | homolog | | | |
| | | | (S. pombe) | | | |
| CYFIP1 | | CYFIP1 | cytoplasmic | Cytoplasm | other | |
| | | | FMR1 | | | |
| | | | interacting | | | |
| | | | protein 1 | | | |
| DCP1A | | DCP1A | DCP1 | Nucleus | other | |
| | | | decapping | | | |
| | | | enzyme | | | |
| | | | homolog A | | | |
| | | | (S. cerevisiae) | | | |
| DICER1 | | DICER1 | dicer 1, | Cytoplasm | enzyme | |
| | | | ribonuclease | | | |
| | | | type III | | | |
| DNAJA1 | | DNAJA1 | DnaJ (Hsp40) | Nucleus | other | |
| | | | homolog, | | | |
| | | | subfamily A, | | | |
| | | | member 1 | | | |
| DNAJA2 | | DNAJA2 | DnaJ (Hsp40) | Nucleus | enzyme | |
| | | | homolog, | | | |
| | | | subfamily A, | | | |
| | | | member 2 | | | |
| DNAJB1 | | DNAJB1 | DnaJ (Hsp40) | Nucleus | other | |
| | | | homolog, | | | |
| | | | subfamily B, | | | |
| | | | member 1 | | | |
| DNAJB11 | | DNAJB11 | DnaJ (Hsp40) | Cytoplasm | other | |
| | | | homolog, | | | |
| | | | subfamily B, | | | |
| | | | member 11 | | | |
| DNAJB6 | | DNAJB6 | DnaJ (Hsp40) | Nucleus | transcription | |
| | | | homolog, | | regulator | |
| | | | subfamily B, | | | |
| | | | member 6 | | | |
| DNAJC7 | | DNAJC7 | DnaJ (Hsp40) | Cytoplasm | other | |
| | | | homolog, | | | |
| | | | subfamily C, | | | |
| | | | member 7 | | | |
| DSP | | DSP | desmoplakin | Plasma | other | |
| | | | | Membrane | | |
| DTX3L | | DTX3L | deltex 3-like | Cytoplasm | enzyme | |
| | | | (Drosophila) | | | |
| EBNA1BP2 | | EBNA1BP2 | EBNA1 binding | Nucleus | other | |
| | | | protein 2 | | | |
| EDC3 | | EDC3 | enhancer of mRNA | Cytoplasm | other | |
| | | (includes | decapping 3 | | | |
| | | EG: 315708) | homolog | | | |
| | | | (S. cerevisiae) | | | |
| EDC4 | | EDC4 | enhancer of | Cytoplasm | other | |
| | | | mRNA | | | |
| | | | decapping 4 | | | |
| EEF1B2 | | EEF1B2 | eukaryotic | Cytoplasm | translation | |
| | | | translation | | regulator | |
| | | | elongation factor | | | |
| | | | 1 beta 2 | | | |
| EEF2 | | EEF2 | eukaryotic | Cytoplasm | translation | |
| | | | translation | | regulator | |
| | | | elongation factor 2 | | | |
| EFTUD2 | | EFTUD2 | elongation factor | Nucleus | enzyme | |
| | | | Tu GTP binding | | | |
| | | | domain | | | |
| | | | containing 2 | | | |
| EIF2B2 | | EIF2B2 | eukaryotic | Cytoplasm | translation | |
| | | | translation | | regulator | |
| | | | initiation factor | | | |
| | | | 2B, subunit 2 | | | |
| | | | beta, 39 kDa | | | |
| EIF3A | | EIF3A | eukaryotic | Cytoplasm | translation | |
| | | | translation | | regulator | |
| | | | initiation factor | | | |
| | | | 3, subunit A | | | |
| EIF4A1 | | EIF4A1 | eukaryotic | Cytoplasm | translation | |
| | | | translation | | regulator | |
| | | | initiation factor | | | |
| | | | 4A1 | | | |
| EIF6 | | EIF6 | eukaryotic | Cytoplasm | translation | |
| | | | translation | | regulator | |
| | | | initiation factor 6 | | | |
| ELAVL1 | | ELAVL1 | ELAV | Cytoplasm | other | |
| | | | (embryonic | | | |
| | | | lethal, abnormal | | | |
| | | | vision, | | | |
| | | | Drosophila)-like | | | |
| | | | 1 (Hu antigen R) | | | |
| ELP3 | | ELP3 | elongation | Nucleus | enzyme | |
| | | | protein 3 | | | |
| | | | homolog | | | |
| | | | (S. cerevisiae) | | | |
| EMD | | EMD | emerin | Nucleus | other | |
| EPCAM | | EPCAM | epithelial cell | Plasma | other | tucotuzumab |
| | | | adhesion | Membrane | | celmoleukin, |
| | | | molecule | | | catumaxomab, |
| | | | | | | adecatumumab |
| EPPK1 | | EPPK1 | epiplakin 1 | Cytoplasm | other | |
| EPS15 | | EPS15 | epidermal | Plasma | other | |
| | | | growth factor | Membrane | | |
| | | | receptor | | | |
| | | | pathway | | | |
| | | | substrate 15 | | | |
| EPS15L1 | | EPS15L1 | epidermal | Plasma | other | |
| | | | growth factor | Membrane | | |
| | | | receptor | | | |
| | | | pathway | | | |
| | | | substrate 15-like 1 | | | |
| ESRP1 | | ESRP1 | epithelial | Nucleus | other | |
| | | | splicing | | | |
| | | | regulatory | | | |
| | | | protein 1 | | | |
| ESYT1 | | ESYT1 | extended | unknown | other | |
| | | | synaptotagmin- | | | |
| | | | like protein 1 | | | |
| ETF1 | | ETF1 | eukaryotic | Cytoplasm | translation | |
| | | | translation | | regulator | |
| | | | termination | | | |
| | | | factor 1 | | | |
| ETFA | | ETFA | electron- | Cytoplasm | transporter | |
| | | | transfer- | | | |
| | | | flavoprotein, | | | |
| | | | alpha | | | |
| | | | polypeptide | | | |
| ETV3 | | ETV3 | ets variant 3 | Nucleus | transcription | |
| | | | | | regulator | |
| FANCD2 | | FANCD2 | Fanconi anemia, | Nucleus | other | |
| | | | complementation | | | |
| | | | group D2 | | | |
| FASN | | FASN | fatty acid | Cytoplasm | enzyme | |
| | | | synthase | | | |
| FDFT1 | | FDFT1 | farnesyl- | Cytoplasm | enzyme | TAK-475, |
| | | | diphosphate | | | zoledronic |
| | | | farnesyltransferase 1 | | | acid |
| FHL3 | | FHL3 | four and a half | Plasma | other | |
| | | | LIM domains 3 | Membrane | | |
| FKBP4 | | FKBP4 | FK506 binding | Nucleus | enzyme | |
| | | | protein 4, 59 kDa | | | |
| FKBP9 | | FKBP9 | FK506 binding | Cytoplasm | enzyme | |
| | | | protein 9, 63 kDa | | | |
| FLAD1 | | FLAD1 | FAD1 flavin | Cytoplasm | enzyme | |
| | | | adenine | | | |
| | | | dinucleotide | | | |
| | | | synthetase | | | |
| | | | homolog | | | |
| | | | (S. cerevisiae) | | | |
| FLNA | | FLNA | filamin A, alpha | Cytoplasm | other | |
| FLNB | | FLNB | filamin B, beta | Cytoplasm | other | |
| FUBP1 | | FUBP1 | far upstream | Nucleus | transcription | |
| | | | element (FUSE) | | regulator | |
| | | | binding protein 1 | | | |
| FUBP3 | | FUBP3 | far upstream | Nucleus | transcription | |
| | | | element (FUSE) | | regulator | |
| | | | binding protein 3 | | | |
| GAN | | GAN | gigaxonin | Cytoplasm | other | |
| GANAB | | GANAB | glucosidase, | Cytoplasm | enzyme | |
| | | | alpha; neutral AB | | | |
| GAPDH | | GAPDH | glyceraldehyde- | Cytoplasm | enzyme | |
| | | | 3-phosphate | | | |
| | | | dehydrogenase | | | |
| GART | | GART | phosphoribosyl- | Cytoplasm | enzyme | LY231514 |
| | | | glycinamide | | | |
| | | | formyltransferase, | | | |
| | | | phosphoribosyl- | | | |
| | | | glycinamide | | | |
| | | | synthetase, | | | |
| | | | phosphoribosyl- | | | |
| | | | aminoimidazole | | | |
| | | | synthetase | | | |
| GBA | | GBA | glucosidase, | Cytoplasm | enzyme | |
| | | | beta, acid | | | |
| GCA | | GCA | grancalcin, EF- | Cytoplasm | other | |
| | | | hand calcium | | | |
| | | | binding protein | | | |
| GIGYF2 | | GIGYF2 | GRB10 | unknown | other | |
| | | | interacting GYF | | | |
| | | | protein 2 | | | |
| GINS4 | | GINS4 | GINS complex | Nucleus | other | |
| | | | subunit 4 (Sld5 | | | |
| | | | homolog) | | | |
| GLA | | GLA | galactosidase, | Cytoplasm | enzyme | |
| | | | alpha | | | |
| GLB1 | | GLB1 | galactosidase, | Cytoplasm | enzyme | |
| | | | beta 1 | | | |
| GLMN | | GLMN | glomulin, FKBP | Cytoplasm | other | |
| | | | associated | | | |
| | | | protein | | | |
| GPHN | | GPHN | gephyrin | Plasma | enzyme | |
| | | | | Membrane | | |
| GPI | | GPI | glucose-6- | Extracellular | enzyme | |
| | | | phosphate | Space | | |
| | | | isomerase | | | |
| GPS1 | | GPS1 | G protein | Nucleus | other | |
| | | | pathway | | | |
| | | | suppressor 1 | | | |
| GRB2 | | GRB2 | growth factor | Cytoplasm | other | |
| | | | receptor-bound | | | |
| | | | protein 2 | | | |
| GTF2F1 | | GTF2F1 | general | Nucleus | transcription | |
| | | | transcription | | regulator | |
| | | | factor IIF, | | | |
| | | | polypeptide 1, | | | |
| | | | 74 kDa | | | |
| GTF2F2 | | GTF2F2 | general | Nucleus | transcription | |
| | | | transcription | | regulator | |
| | | | factor IIF, | | | |
| | | | polypeptide 2, | | | |
| | | | 30 kDa | | | |
| GTF2I | | GTF2I | general | Nucleus | transcription | |
| | | | transcription | | regulator | |
| | | | factor IIi | | | |
| H1F0 | | H1F0 | H1 histone | Nucleus | other | |
| | | | family, member 0 | | | |
| H1FX | | H1FX | H1 histone | Nucleus | other | |
| | | | family, member X | | | |
| HDAC2 | | HDAC2 | histone | Nucleus | transcription | tributyrin, |
| | | | deacetylase 2 | | regulator | belinostat, |
| | | | | | | pyroxamide, |
| | | | | | | vorinostat, |
| | | | | | | romidepsin |
| HDAC3 | | HDAC3 | histone | Nucleus | transcription | tributyrin, |
| | | | deacetylase 3 | | regulator | belinostat, |
| | | | | | | pyroxamide, |
| | | | | | | MGCD0103, |
| | | | | | | vorinostat, |
| | | | | | | romidepsin |
| HDAC6 | | HDAC6 | histone | Nucleus | transcription | tributyrin, |
| | | | deacetylase 6 | | regulator | belinostat, |
| | | | | | | pyroxamide, |
| | | | | | | vorinostat, |
| | | | | | | romidepsin |
| HIF1AN | | HIF1AN | hypoxia | Nucleus | enzyme | |
| | | | inducible factor 1, | | | |
| | | | alpha subunit | | | |
| | | | inhibitor | | | |
| HIST1H1B | | HIST1H1B | histone cluster 1, | Nucleus | other | |
| | | | H1b | | | |
| HIST1H1D | | HIST1H1D | histone cluster 1, | Nucleus | other | |
| | | | H1d | | | |
| HNRNPA0 | | HNRNPA0 | heterogeneous | Nucleus | other | |
| | | | nuclear | | | |
| | | | ribonucleoprotein | | | |
| | | | A0 | | | |
| HSP90AA1 | | HSP90AA1 | heat shock | Cytoplasm | enzyme | 17-dimethylamino- |
| | | | protein 90 kDa | | | ethylamino- |
| | | | alpha | | | 17-demethoxy- |
| | | | (cytosolic), class | | | geldanamycin, |
| | | | A member 1 | | | IPI-504, |
| | | | | | | cisplatin |
| HSP90AA4P | | HSP90AA4P | heat shock | unknown | other | |
| | | | protein 90 kDa | | | |
| | | | alpha | | | |
| | | | (cytosolic), class | | | |
| | | | A member 4, | | | |
| | | | pseudogene | | | |
| HSP90AB1 | | HSP90AB1 | heat shock | Cytoplasm | enzyme | 17-dimethylamino- |
| | | | protein 90 kDa | | | ethylamino- |
| | | | alpha | | | 17-demethoxy- |
| | | | (cytosolic), class | | | geldanamycin, |
| | | | B member 1 | | | IPI-504, |
| | | | | | | cisplatin |
| HSP90B1 | | HSP90B1 | heat shock | Cytoplasm | other | 17-dimethylamino- |
| | | | protein 90 kDa | | | ethylamino- |
| | | | beta (Grp94), | | | 17-demethoxy- |
| | | | member 1 | | | geldanamycin, |
| | | | | | | IPI-504, |
| | | | | | | cisplatin |
| HSPA4 | | HSPA4 | heat shock | Cytoplasm | other | |
| | | | 70 kDa protein 4 | | | |
| HSPA5 | | HSPA5 | heat shock | Cytoplasm | enzyme | |
| | | | 70 kDa protein 5 | | | |
| | | | (glucose- | | | |
| | | | regulated | | | |
| | | | protein, 78 kDa) | | | |
| HSPA8 | | HSPA8 | heat shock | Cytoplasm | enzyme | |
| | | | 70 kDa protein 8 | | | |
| HSPB1 | | HSPB1 | heat shock | Cytoplasm | other | |
| | | | 27 kDa protein 1 | | | |
| HSPD1 | | HSPD1 | heat shock | Cytoplasm | enzyme | |
| | | | 60 kDa protein 1 | | | |
| | | | (chaperonin) | | | |
| HSPH1 | | HSPH1 | heat shock | Cytoplasm | other | |
| | | | 105 kDa/110 kDa | | | |
| | | | protein 1 | | | |
| IDH2 | | IDH2 | isocitrate | Cytoplasm | enzyme | |
| | | | dehydrogenase | | | |
| | | | 2 (NADP+), | | | |
| | | | mitochondrial | | | |
| IGBP1 | | IGBP1 | immunoglobulin | Cytoplasm | phosphatase | |
| | | | (CD79A) binding | | | |
| | | | protein 1 | | | |
| IGF2BP3 | | IGF2BP3 | insulin-like | Cytoplasm | translation | |
| | | | growth factor 2 | | regulator | |
| | | | mRNA binding | | | |
| | | | protein 3 | | | |
| IKBKAP | | IKBKAP | inhibitor of | Cytoplasm | other | |
| | | | kappa light | | | |
| | | | polypeptide | | | |
| | | | gene enhancer | | | |
| | | | in B-cells, | | | |
| | | | kinase complex- | | | |
| | | | associated | | | |
| | | | protein | | | |
| ILF2 | | ILF2 | interleukin | Nucleus | transcription | |
| | | | enhancer | | regulator | |
| | | | binding factor 2, | | | |
| | | | 45 kDa | | | |
| ILF3 | | ILF3 | interleukin | Nucleus | transcription | |
| | | | enhancer | | | |
| | | | binding factor 3, | | | |
| | | | 90 kDa | | | |
| IMPDH1 | | IMPDH1 | IMP (inosine 5′- | Cytoplasm | enzyme | thioguanine, |
| | | | monophosphate) | | | VX-944, |
| | | | dehydrogenase 1 | | | interferon |
| | | | | | | alfa- |
| | | | | | | 2b/ribavirin, |
| | | | | | | mycophenolic |
| | | | | | | acid, ribavirin |
| IMPDH2 | | IMPDH2 | IMP (inosine 5′- | Cytoplasm | enzyme | thioguanine, |
| | | | monophosphate) | | | VX-944, |
| | | | dehydrogenase 2 | | | interferon |
| | | | | | | alfa- |
| | | | | | | 2b/ribavirin, |
| | | | | | | mycophenolic |
| | | | | | | acid, ribavirin |
| INF2 | | INF2 | inverted formin, | Cytoplasm | other | |
| | | | FH2 and WH2 | | | |
| | | | domain | | | |
| | | | containing | | | |
| INTS3 | | INTS3 | integrator | Nucleus | other | |
| | | | complex subunit 3 | | | |
| IRAKI | | IRAKI | interleukin-1 | Plasma | kinase | |
| | | | receptor- | Membrane | | |
| | | | associated | | | |
| | | | kinase 1 | | | |
| ISYNA1 | | ISYNA1 | inositol-3- | unknown | enzyme | |
| | | | phosphate | | | |
| | | | synthase 1 | | | |
| ITCH | | ITCH | itchy E3 | Nucleus | enzyme | |
| | | | ubiquitin protein | | | |
| | | | ligase homolog | | | |
| | | | (mouse) | | | |
| KHDRBS1 | | KHDRBS1 | KH domain | Nucleus | transcription | |
| | | | containing, RNA | | regulator | |
| | | | binding, signal | | | |
| | | | transduction | | | |
| | | | associated 1 | | | |
| KHSRP | | KHSRP | KH-type splicing | Nucleus | enzyme | |
| | | | regulatory | | | |
| | | | protein | | | |
| LGALS3 | | LGALS3 | lectin, | Extracellular | other | |
| | | | galactoside- | Space | | |
| | | | binding, | | | |
| | | | soluble, 3 | | | |
| LGALS3BP | | LGALS3BP | lectin, | Plasma | transmembrane | |
| | | | galactoside- | Membrane | receptor | |
| | | | binding, soluble, | | | |
| | | | 3 binding protein | | | |
| LIPA | | LIPA | lipase A, | Cytoplasm | enzyme | |
| | | | lysosomal acid, | | | |
| | | | cholesterol | | | |
| | | | esterase | | | |
| LMAN2 | | LMAN2 | lectin, mannose- | Cytoplasm | transporter | |
| | | | binding 2 | | | |
| LMNA | | LMNA | lamin A/C | Nucleus | other | |
| LRBA | | LRBA | LPS-responsive | Cytoplasm | other | |
| | | | vesicle | | | |
| | | | trafficking, | | | |
| | | | beach and | | | |
| | | | anchor | | | |
| | | | containing | | | |
| LRPPRC | | LRPPRC | leucine-rich | Cytoplasm | other | |
| | | | PPR-motif | | | |
| | | | containing | | | |
| LSM14A | | LSM14A | LSM14A, SCD6 | Cytoplasm | other | |
| | | | homolog A | | | |
| | | | (S. cerevisiae) | | | |
| MAGI3 | | MAGI3 | membrane | Cytoplasm | kinase | |
| | | | associated | | | |
| | | | guanylate | | | |
| | | | kinase, WW and | | | |
| | | | PDZ domain | | | |
| | | | containing 3 | | | |
| MAP3K7 | | MAP3K7 | mitogen- | Cytoplasm | kinase | |
| | | (includes | activated protein | | | |
| | | EG: 172842) | kinase kinase | | | |
| | | | kinase 7 | | | |
| MAPK1 | | MAPK1 | mitogen- | Cytoplasm | kinase | |
| | | | activated protein | | | |
| | | | kinase 1 | | | |
| MAPK3 | | MAPK3 | mitogen- | Cytoplasm | kinase | |
| | | | activated protein | | | |
| | | | kinase 3 | | | |
| MAPK9 | | MAPK9 | mitogen- | Cytoplasm | kinase | |
| | | | activated protein | | | |
| | | | kinase 9 | | | |
| MCM2 | | MCM2 | minichromosome | Nucleus | enzyme | |
| | | | maintenance | | | |
| | | | complex | | | |
| | | | component 2 | | | |
| MEMO1 | | MEMO1 | mediator of cell | Cytoplasm | other | |
| | | (includes | motility 1 | | | |
| | | EG: 298787) | | | | |
| MKI67 | | MKI67 | antigen | Nucleus | other | |
| | | | identified by | | | |
| | | | monoclonal | | | |
| | | | antibody Ki-67 | | | |
| MLF2 | | MLF2 | myeloid | Nucleus | other | |
| | | | leukemia factor 2 | | | |
| MSH6 | | MSH6 | mutS homolog 6 | Nucleus | enzyme | |
| | | | (E. coli) | | | |
| MSI1 | | MSI1 | musashi | Cytoplasm | other | |
| | | (includes | homolog 1 | | | |
| | | EG: 17690) | (Drosophila) | | | |
| MSI2 | | MSI2 | musashi | Cytoplasm | other | |
| | | | homolog 2 | | | |
| | | | (Drosophila) | | | |
| MTA2 | | MTA2 | metastasis | Nucleus | transcription | |
| | | | associated 1 | | regulator | |
| | | | family, member 2 | | | |
| MTOR | | MTOR | mechanistic | Nucleus | kinase | deforolimus, |
| | | | target of | | | OSI-027, |
| | | | rapamycin | | | NVP-BEZ235, |
| | | | (serine/threonine | | | temsirolimus, |
| | | | kinase) | | | tacrolimus, |
| | | | | | | everolimus |
| MTX1 | | MTX1 | metaxin 1 | Cytoplasm | transporter | |
| MYBBP1A | | MYBBP1A | MYB binding | Nucleus | transcription | |
| | | | protein (P160) 1a | | regulator | |
| MYCBP2 | | MYCBP2 | MYC binding | Nucleus | enzyme | |
| | | | protein 2 | | | |
| NACC1 | | NACC1 | nucleus | Nucleus | transcription | |
| | | | accumbens | | regulator | |
| | | | associated 1, | | | |
| | | | BEN and BTB | | | |
| | | | (POZ) domain | | | |
| | | | containing | | | |
| NAT10 | | NAT10 | N- | Nucleus | enzyme | |
| | | | acetyltransferase | | | |
| | | | 10 (GCN5- | | | |
| | | | related) | | | |
| NCBP1 | | NCBP1 | nuclear cap | Nucleus | other | |
| | | | binding protein | | | |
| | | | subunit 1, | | | |
| | | | 80 kDa | | | |
| NCKAP1 | | NCKAP1 | NCK-associated | Plasma | other | |
| | | | protein 1 | Membrane | | |
| NCKIPSD | | NCKIPSD | NCK interacting | Nucleus | other | |
| | | | protein with SH3 | | | |
| | | | domain | | | |
| NCL | | NCL | nucleolin | Nucleus | other | |
| NCOR1 | | NCOR1 | nuclear receptor | Nucleus | transcription | |
| | | | corepressor 1 | | regulator | |
| NCOR2 | | NCOR2 | nuclear receptor | Nucleus | transcription | |
| | | | corepressor 2 | | regulator | |
| NFKB2 | | NFKB2 | nuclear factor of | Nucleus | transcription | |
| | | | kappa light | | regulator | |
| | | | polypeptide | | | |
| | | | gene enhancer | | | |
| | | | in B-cells 2 | | | |
| | | | (p49/p100) | | | |
| NKRF | | NKRF | NFKB | Nucleus | transcription | |
| | | | repressing factor | | regulator | |
| NME7 | | NME7 | non-metastatic | Cytoplasm | kinase | |
| | | | cells 7, protein | | | |
| | | | expressed in | | | |
| | | | (nucleoside- | | | |
| | | | diphosphate | | | |
| | | | kinase) | | | |
| NNMT | | NNMT | nicotinamide N- | Cytoplasm | enzyme | |
| | | | methyltransferase | | | |
| NOL6 | | NOL6 | nucleolar protein | Nucleus | other | |
| | | | family 6 (RNA- | | | |
| | | | associated) | | | |
| NPM1 | | NPM1 | nucleophosmin | Nucleus | transcription | |
| | | | (nucleolar | | regulator | |
| | | | phosphoprotein | | | |
| | | | B23, numatrin) | | | |
| NQO1 | | NQO1 | NAD(P)H | Cytoplasm | enzyme | |
| | | | dehydrogenase, | | | |
| | | | quinone 1 | | | |
| NQO2 | | NQO2 | NAD(P)H | Cytoplasm | enzyme | |
| | | | dehydrogenase, | | | |
| | | | quinone 2 | | | |
| NUCB1 | | NUCB1 | nucleobindin 1 | Cytoplasm | other | |
| NUDCD1 | | NUDCD1 | NudC domain | unknown | other | |
| | | | containing 1 | | | |
| NUDCD3 | | NUDCD3 | NudC domain | unknown | other | |
| | | | containing 3 | | | |
| NUDT5 | | NUDT5 | nudix | Cytoplasm | phosphatase | |
| | | | (nucleoside | | | |
| | | | diphosphate | | | |
| | | | linked moiety X)- | | | |
| | | | type motif 5 | | | |
| NUF2 | | NUF2 | NUF2, NDC80 | Nucleus | other | |
| | | | kinetochore | | | |
| | | | complex | | | |
| | | | component, | | | |
| | | | homolog | | | |
| | | | (S. cerevisiae) | | | |
| OTUB1 | | OTUB1 | OTU domain, | unknown | enzyme | |
| | | | ubiquitin | | | |
| | | | aldehyde | | | |
| | | | binding 1 | | | |
| OTUD4 | | OTUD4 | OTU domain | unknown | other | |
| | | | containing 4 | | | |
| PA2G4 | | PA2G4 | proliferation- | Nucleus | transcription | |
| | | | associated 2G4, | | regulator | |
| | | | 38 kDa | | | |
| PCNA | | PCNA | proliferating cell | Nucleus | enzyme | |
| | | | nuclear antigen | | | |
| PDAP1 | | PDAP1 | PDGFA | Cytoplasm | other | |
| | | | associated | | | |
| | | | protein 1 | | | |
| PDCD2L | | PDCD2L | programmed cell | unknown | other | |
| | | | death 2-like | | | |
| PDCD6IP | | PDCD6IP | programmed cell | Cytoplasm | other | |
| | | | death 6 | | | |
| | | | interacting | | | |
| | | | protein | | | |
| PDIA6 | | PDIA6 | protein disulfide | Cytoplasm | enzyme | |
| | | | isomerase | | | |
| | | | family A, | | | |
| | | | member 6 | | | |
| PDK3 | | PDK3 | pyruvate | Cytoplasm | kinase | |
| | | | dehydrogenase | | | |
| | | | kinase, isozyme 3 | | | |
| PDLIM1 | | PDLIM1 | PDZ and LIM | Cytoplasm | transcription | |
| | | | domain 1 | | regulator | |
| PDLIM5 | | PDLIM5 | PDZ and LIM | Cytoplasm | other | |
| | | | domain 5 | | | |
| PIK3C2B | | PIK3C2B | phosphoinositide- | Cytoplasm | kinase | |
| | | | 3-kinase, | | | |
| | | | class 2, beta | | | |
| | | | polypeptide | | | |
| PIK3C3 | | PIK3C3 | phosphoinositide- | Cytoplasm | kinase | |
| | | | 3-kinase, | | | |
| | | | class 3 | | | |
| PIK3R4 | | PIK3R4 | phosphoinositide- | Cytoplasm | other | |
| | | | 3-kinase, | | | |
| | | | regulatory | | | |
| | | | subunit 4 | | | |
| PLAA | | PLAA | phospholipase | Cytoplasm | other | |
| | | | A2-activating | | | |
| | | | protein | | | |
| PLBD2 | | PLBD2 | phospholipase B | Extracellular | other | |
| | | | domain | Space | | |
| | | | containing 2 | | | |
| POLD1 | | POLD1 | polymerase | Nucleus | enzyme | nelarabine, |
| | | | (DNA directed), | | | MB07133, |
| | | | delta 1, catalytic | | | clofarabine, |
| | | | subunit 125 kDa | | | cytarabine, |
| | | | | | | trifluridine, |
| | | | | | | vidarabine, |
| | | | | | | entecavir |
| POLR2A | | POLR2A | polymerase | Nucleus | enzyme | |
| | | | (RNA) II (DNA | | | |
| | | | directed) | | | |
| | | | polypeptide A, | | | |
| | | | 220 kDa | | | |
| PPIE | | PPIE | peptidylprolyl | Nucleus | enzyme | |
| | | | isomerase E | | | |
| | | | (cyclophilin E) | | | |
| PPP1CB | | PPP1CB | protein | Cytoplasm | phosphatase | |
| | | | phosphatase 1, | | | |
| | | | catalytic subunit, | | | |
| | | | beta isozyme | | | |
| PPP2CA | | PPP2CA | protein | Cytoplasm | phosphatase | |
| | | | phosphatase 2, | | | |
| | | | catalytic subunit, | | | |
| | | | alpha isozyme | | | |
| PPP3CA | | PPP3CA | protein | Cytoplasm | phosphatase | ISAtx-247, |
| | | | phosphatase 3, | | | tacrolimus, |
| | | | catalytic subunit, | | | pimecrolimus, |
| | | | alpha isozyme | | | cyclosporin A |
| PPP4C | | PPP4C | protein | Cytoplasm | phosphatase | |
| | | | phosphatase 4, | | | |
| | | | catalytic subunit | | | |
| PPP5C | | PPP5C | protein | Nucleus | phosphatase | |
| | | | phosphatase 5, | | | |
| | | | catalytic subunit | | | |
| PPP6C | | PPP6C | protein | Nucleus | phosphatase | |
| | | | phosphatase 6, | | | |
| | | | catalytic subunit | | | |
| PRIM2 | | PRIM2 | primase, DNA, | Nucleus | enzyme | fludarabine |
| | | | polypeptide 2 | | | phosphate |
| | | | (58 kDa) | | | |
| PRKAA1 | | PRKAA1 | protein kinase, | Cytoplasm | kinase | |
| | | | AMP-activated, | | | |
| | | | alpha 1 catalytic | | | |
| | | | subunit | | | |
| PRKAB1 | | PRKAB1 | protein kinase, | Nucleus | kinase | |
| | | | AMP-activated, | | | |
| | | | beta 1 non- | | | |
| | | | catalytic subunit | | | |
| PRKAB2 | | PRKAB2 | protein kinase, | Cytoplasm | kinase | |
| | | | AMP-activated, | | | |
| | | | beta 2 non- | | | |
| | | | catalytic subunit | | | |
| PRKAG1 | | PRKAG1 | protein kinase, | Nucleus | kinase | |
| | | | AMP-activated, | | | |
| | | | gamma 1 non- | | | |
| | | | catalytic subunit | | | |
| PRKCSH | | PRKCSH | protein kinase C | Cytoplasm | enzyme | |
| | | | substrate 80K-H | | | |
| PRKDC | | PRKDC | protein kinase, | Nucleus | kinase | |
| | | | DNA-activated, | | | |
| | | | catalytic | | | |
| | | | polypeptide | | | |
| PRMT1 | | PRMT1 | protein arginine | Nucleus | enzyme | |
| | | | methyltransferase 1 | | | |
| PRMT5 | | PRMT5 | protein arginine | Cytoplasm | enzyme | |
| | | | methyltransferase 5 | | | |
| PSMA1 | | PSMA1 | proteasome | Cytoplasm | peptidase | |
| | | | (prosome, | | | |
| | | | macropain) | | | |
| | | | subunit, alpha | | | |
| | | | type, 1 | | | |
| PSMC1 | | PSMC1 | proteasome | Nucleus | peptidase | |
| | | | (prosome, | | | |
| | | | macropain) 26S | | | |
| | | | subunit, | | | |
| | | | ATPase, 1 | | | |
| PSMD1 | | PSMD1 | proteasome | Cytoplasm | other | |
| | | | (prosome, | | | |
| | | | macropain) 26S | | | |
| | | | subunit, non- | | | |
| | | | ATPase, 1 | | | |
| PSME1 | | PSME1 | proteasome | Cytoplasm | other | |
| | | | (prosome, | | | |
| | | | macropain) | | | |
| | | | activator subunit | | | |
| | | | 1 (PA28 alpha) | | | |
| PSPC1 | | PSPC1 | paraspeckle | Nucleus | other | |
| | | | component 1 | | | |
| PTCD3 | | PTCD3 | Pentatricopeptide | Cytoplasm | other | |
| | | | repeat domain 3 | | | |
| PTGES2 | | PTGES2 | prostaglandin E | Cytoplasm | transcription | |
| | | | synthase 2 | | regulator | |
| PTK2 | | PTK2 | PTK2 protein | Cytoplasm | kinase | |
| | | (includes | tyrosine kinase 2 | | | |
| | | EG: 14083) | | | | |
| PUM1 | | PUM1 | pumilio homolog | Cytoplasm | other | |
| | | | 1 (Drosophila) | | | |
| RAB3D | | RAB3D | RAB3D, | Cytoplasm | enzyme | |
| | | | member RAS | | | |
| | | | oncogene family | | | |
| RAB3GAP1 | | RAB3GAP1 | RAB3 GTPase | Cytoplasm | other | |
| | | | activating | | | |
| | | | protein subunit 1 | | | |
| | | | (catalytic) | | | |
| RAB3GAP2 | | RAB3GAP2 | RAB3 GTPase | Cytoplasm | enzyme | |
| | | | activating | | | |
| | | | protein subunit 2 | | | |
| | | | (non-catalytic) | | | |
| RAB5C | | RAB5C | RAB5C, | Cytoplasm | enzyme | |
| | | | member RAS | | | |
| | | | oncogene family | | | |
| RABGGTB | | RABGGTB | Rab | Cytoplasm | enzyme | |
| | | | geranylgeranyl- | | | |
| | | | transferase, beta | | | |
| | | | subunit | | | |
| RAD23B | | RAD23B | RAD23 homolog | Nucleus | other | |
| | | | B (S. cerevisiae) | | | |
| RAE1 | | RAE1 | RAE1 RNA | Nucleus | other | |
| | | | export 1 | | | |
| | | | homolog | | | |
| | | | (S. pombe) | | | |
| RANBP2 | | RANBP2 | RAN binding | Nucleus | enzyme | |
| | | | protein 2 | | | |
| RANGAP1 | | RANGAP1 | Ran GTPase | Cytoplasm | other | |
| | | | activating | | | |
| | | | protein 1 | | | |
| RBCK1 | | RBCK1 | RanBP-type and | Cytoplasm | transcription | |
| | | | C3HC4-type | | regulator | |
| | | | zinc finger | | | |
| | | | containing 1 | | | |
| RBM10 | | RBM10 | RNA binding | Nucleus | other | |
| | | | motif protein 10 | | | |
| RELA | | RELA | v-rel | Nucleus | transcription | NF-kappaB |
| | | | reticuloendotheliosis | | regulator | decoy |
| | | | viral | | | |
| | | | oncogene | | | |
| | | | homolog A | | | |
| | | | (avian) | | | |
| RFC2 | | RFC2 | replication factor | Nucleus | other | |
| | | | C (activator 1) 2, | | | |
| | | | 40 kDa | | | |
| RPA2 | | RPA2 | replication | Nucleus | other | |
| | | | protein A2, | | | |
| | | | 32 kDa | | | |
| RPS6 | | RPS6 | ribosomal | Cytoplasm | other | |
| | | | protein S6 | | | |
| RPS6KA3 | | RPS6KA3 | ribosomal | Cytoplasm | kinase | |
| | | | protein S6 | | | |
| | | | kinase, 90 kDa, | | | |
| | | | polypeptide 3 | | | |
| RPSA | | RPSA | ribosomal | Cytoplasm | translation | |
| | | | protein SA | | regulator | |
| RUVBL1 | | RUVBL1 | RuvB-like 1 | Nucleus | transcription | |
| | | | (E. coli) | | regulator | |
| RUVBL2 | | RUVBL2 | RuvB-like 2 | Nucleus | transcription | |
| | | | (E. coli) | | regulator | |
| S100A8 | | S100A8 | S100 calcium | Cytoplasm | other | |
| | | | binding protein | | | |
| | | | A8 | | | |
| S100A9 | | S100A9 | S100 calcium | Cytoplasm | other | |
| | | | binding protein | | | |
| | | | A9 | | | |
| SAMHD1 | | SAMHD1 | SAM domain | Nucleus | enzyme | |
| | | | and HD domain 1 | | | |
| SELO | | SELO | selenoprotein O | Extracellular | enzyme | |
| | | | | Space | | |
| SETD2 | | SETD2 | SET domain | Cytoplasm | enzyme | |
| | | | containing 2 | | | |
| SF1 | | SF1 | splicing factor 1 | Nucleus | transcription | |
| | | | | | regulator | |
| SHARPIN | | SHARPIN | SHANK- | Plasma | other | |
| | | | associated RH | Membrane | | |
| | | | domain | | | |
| | | | interactor | | | |
| SIRT1 | | SIRT1 | sirtuin 1 | Nucleus | transcription | |
| | | | | | regulator | |
| SIRT3 | | SIRT3 | sirtuin 3 | Cytoplasm | enzyme | |
| SMARCA2 | | SMARCA2 | SWI/SNF | Nucleus | transcription | |
| | | | related, matrix | | regulator | |
| | | | associated, actin | | | |
| | | | dependent | | | |
| | | | regulator of | | | |
| | | | chromatin, | | | |
| | | | subfamily a, | | | |
| | | | member 2 | | | |
| SMARCA4 | | SMARCA4 | SWI/SNF | Nucleus | transcription | |
| | | | related, matrix | | regulator | |
| | | | associated, actin | | | |
| | | | dependent | | | |
| | | | regulator of | | | |
| | | | chromatin, | | | |
| | | | subfamily a, | | | |
| | | | member 4 | | | |
| SNRNP200 | | SNRNP200 | small nuclear | Nucleus | enzyme | |
| | | | ribonucleoprotein | | | |
| | | | 200 kDa (U5) | | | |
| SNX9 | | SNX9 | sorting nexin 9 | Cytoplasm | transporter | |
| SON | | SON | SON DNA | Nucleus | other | |
| | | | binding protein | | | |
| SPC24 | | SPC24 | SPC24, NDC80 | Cytoplasm | other | |
| | | (includes | kinetochore | | | |
| | | EG: 147841) | complex | | | |
| | | | component, | | | |
| | | | homolog | | | |
| | | | (S. cerevisiae) | | | |
| SQSTM1 | | SQSTM1 | sequestosome 1 | Cytoplasm | transcription | |
| | | | | | regulator | |
| SRPK2 | | SRPK2 | SRSF protein | Nucleus | kinase | |
| | | | kinase 2 | | | |
| ST13 | | ST13 | suppression of | Cytoplasm | other | |
| | | | tumorigenicity | | | |
| | | | 13 (colon | | | |
| | | | carcinoma) | | | |
| | | | (Hsp70 | | | |
| | | | interacting | | | |
| | | | protein) | | | |
| STAM | | STAM | signal | Cytoplasm | other | |
| | | | transducing | | | |
| | | | adaptor | | | |
| | | | molecule (SH3 | | | |
| | | | domain and | | | |
| | | | ITAM motif) 1 | | | |
| STAT3 | | STAT3 | signal | Nucleus | transcription | |
| | | | transducer and | | regulator | |
| | | | activator of | | | |
| | | | transcription 3 | | | |
| | | | (acute-phase | | | |
| | | | response factor) | | | |
| STAT5B | | STAT5B | signal | Nucleus | transcription | |
| | | | transducer and | | regulator | |
| | | | activator of | | | |
| | | | transcription 5B | | | |
| STIP1 | | STIP1 | stress-induced- | Cytoplasm | other | |
| | | | phosphoprotein 1 | | | |
| STK3 | | STK3 | serine/threonine | Cytoplasm | kinase | |
| | | | kinase 3 | | | |
| STRAP | | STRAP | serine/threonine | Plasma | other | |
| | | | kinase receptor | Membrane | | |
| | | | associated | | | |
| | | | protein | | | |
| STUB1 | | STUB1 | STIP1 homology | Cytoplasm | enzyme | |
| | | | and U-box | | | |
| | | | containing | | | |
| | | | protein 1, E3 | | | |
| | | | ubiquitin protein | | | |
| | | | ligase | | | |
| SULT1A1 | | SULT1A1 | sulfotransferase | Cytoplasm | enzyme | |
| | | | family, cytosolic, | | | |
| | | | 1A, phenol- | | | |
| | | | preferring, | | | |
| | | | member 1 | | | |
| SULT2B1 | | SULT2B1 | sulfotransferase | Cytoplasm | enzyme | |
| | | | family, cytosolic, | | | |
| | | | 2B, member 1 | | | |
| SURF4 | | SURF4 | surfeit 4 | Cytoplasm | other | |
| TAB1 | | TAB1 | TGF-beta | Cytoplasm | enzyme | |
| | | | activated kinase | | | |
| | | | 1/MAP3K7 | | | |
| | | | binding protein 1 | | | |
| TBC1D15 | | TBC1D15 | TBC1 domain | Cytoplasm | other | |
| | | | family, member 15 | | | |
| TBC1D9B | | TBC1D9B | TBC1 domain | unknown | other | |
| | | | family, member | | | |
| | | | 9B (with GRAM | | | |
| | | | domain) | | | |
| TBK1 | | TBK1 | TANK-binding | Cytoplasm | kinase | |
| | | | kinase 1 | | | |
| TBRG4 | | TBRG4 | transforming | Cytoplasm | other | |
| | | | growth factor | | | |
| | | | beta regulator 4 | | | |
| TCEAL4 | | TCEAL4 | transcription | unknown | other | |
| | | | elongation factor | | | |
| | | | A (SII)-like 4 | | | |
| TFRC | | TFRC | transferrin | Plasma | transporter | |
| | | | receptor (p90, | Membrane | | |
| | | | CD71) | | | |
| TIPRL | | TIPRL | TIP41, TOR | unknown | other | |
| | | | signaling | | | |
| | | | pathway | | | |
| | | | regulator-like | | | |
| | | | (S. cerevisiae) | | | |
| TJP2 | | TJP2 | tight junction | Plasma | kinase | |
| | | | protein 2 (zona | Membrane | | |
| | | | occludens 2) | | | |
| TLN1 | | TLN1 | talin 1 | Plasma | other | |
| | | | | Membrane | | |
| TMCO6 | | TMCO6 | transmembrane | unknown | other | |
| | | | and coiled-coil | | | |
| | | | domains 6 | | | |
| TNRC6B | | TNRC6B | trinucleotide | unknown | other | |
| | | | repeat | | | |
| | | | containing 6B | | | |
| TOMM34 | | TOMM34 | translocase of | Cytoplasm | other | |
| | | | outer | | | |
| | | | mitochondrial | | | |
| | | | membrane 34 | | | |
| TP53 | | TP53 | tumor protein | Nucleus | transcription | |
| | | (includes | p53 | | regulator | |
| | | EG: 22059) | | | | |
| TP53I3 | | TP53I3 | tumor protein | unknown | enzyme | |
| | | | p53 inducible | | | |
| | | | protein 3 | | | |
| TP53RK | | TP53RK | TP53 regulating | Nucleus | kinase | |
| | | | kinase | | | |
| TPD52L2 | | TPD52L2 | tumor protein | Cytoplasm | other | |
| | | | D52-like 2 | | | |
| TPM3 | | TPM3 | tropomyosin 3 | Cytoplasm | other | |
| TPP1 | | TPP1 | tripeptidyl | Cytoplasm | peptidase | |
| | | (includes | peptidase I | | | |
| | | EG: 1200) | | | | |
| TPP2 | | TPP2 | tripeptidyl | Cytoplasm | peptidase | |
| | | | peptidase II | | | |
| TRA2A | | TRA2A | transformer 2 | Nucleus | other | |
| | | | alpha homolog | | | |
| | | | (Drosophila) | | | |
| TRA2B | | TRA2B | transformer 2 | Nucleus | other | |
| | | | beta homolog | | | |
| | | | (Drosophila) | | | |
| TRAP1 | | TRAP1 | TNF receptor- | Cytoplasm | enzyme | |
| | | | associated | | | |
| | | | protein 1 | | | |
| TRIM28 | | TRIM28 | tripartite motif | Nucleus | transcription | |
| | | | containing 28 | | regulator | |
| TRIO | | TRIO | triple functional | Plasma | kinase | |
| | | | domain (PTPRF | Membrane | | |
| | | | interacting) | | | |
| TTC1 | | TTC1 | tetratricopeptide | unknown | other | |
| | | | repeat domain 1 | | | |
| TTC19 | | TTC19 | tetratricopeptide | Cytoplasm | other | |
| | | | repeat domain 19 | | | |
| TTC35 | | TTC35 | tetratricopeptide | Nucleus | other | |
| | | | repeat domain 35 | | | |
| TTC5 | | TTC5 | tetratricopeptide | unknown | other | |
| | | | repeat domain 5 | | | |
| TYMS | | TYMS | thymidylate | Nucleus | enzyme | flucytosine, |
| | | | synthetase | | | 5-fluorouracil, |
| | | | | | | plevitrexed, |
| | | | | | | nolatrexed, |
| | | | | | | capecitabine, |
| | | | | | | trifluridine, |
| | | | | | | floxuridine, |
| | | | | | | LY231514 |
| UBA1 | | UBA1 | ubiquitin-like | Cytoplasm | enzyme | |
| | | | modifier | | | |
| | | | activating | | | |
| | | | enzyme 1 | | | |
| UBA7 | | UBA7 | ubiquitin-like | Cytoplasm | enzyme | |
| | | | modifier | | | |
| | | | activating | | | |
| | | | enzyme 7 | | | |
| UBAC1 | | UBAC1 | UBA domain | Nucleus | other | |
| | | | containing 1 | | | |
| UBAP2 | | UBAP2 | ubiquitin | Cytoplasm | other | |
| | | | associated | | | |
| | | | protein 2 | | | |
| UBAP2L | | UBAP2L | ubiquitin | unknown | other | |
| | | | associated | | | |
| | | | protein 2-like | | | |
| UBASH3B | | UBASH3B | ubiquitin | unknown | enzyme | |
| | | | associated and | | | |
| | | | SH3 domain | | | |
| | | | containing B | | | |
| UBE3A | | UBE3A | ubiquitin protein | Nucleus | enzyme | |
| | | | ligase E3A | | | |
| UBE4B | | UBE4B | ubiquitination | Cytoplasm | enzyme | |
| | | | factor E4B | | | |
| UBQLN1 | | UBQLN1 | ubiquilin 1 | Cytoplasm | other | |
| UBQLN2 | | UBQLN2 | ubiquilin 2 | Nucleus | other | |
| UBQLN4 | | UBQLN4 | ubiquilin 4 | Cytoplasm | other | |
| UBR1 | | UBR1 | ubiquitin protein | Cytoplasm | enzyme | |
| | | (includes | ligase E3 | | | |
| | | EG: 197131) | component n- | | | |
| | | | recognin 1 | | | |
| UBR4 | | UBR4 | ubiquitin protein | Nucleus | other | |
| | | | ligase E3 | | | |
| | | | component n- | | | |
| | | | recognin 4 | | | |
| UCHL5 | | UCHL5 | ubiquitin | Cytoplasm | peptidase | |
| | | | carboxyl- | | | |
| | | | terminal | | | |
| | | | hydrolase L5 | | | |
| UFD1L | | UFD1L | ubiquitin fusion | Cytoplasm | peptidase | |
| | | | degradation 1 | | | |
| | | | like (yeast) | | | |
| UNC45A | | UNC45A | unc-45 homolog | Plasma | other | |
| | | | A (C. elegans) | Membrane | | |
| USP10 | | USP10 | ubiquitin specific | Cytoplasm | peptidase | |
| | | | peptidase 10 | | | |
| USP11 | | USP11 | ubiquitin specific | Nucleus | peptidase | |
| | | | peptidase 11 | | | |
| USP13 | | USP13 | ubiquitin specific | unknown | peptidase | |
| | | | peptidase 13 | | | |
| | | | (isopeptidase T-3) | | | |
| USP14 | | USP14 | ubiquitin specific | Cytoplasm | peptidase | |
| | | | peptidase 14 | | | |
| | | | (tRNA-guanine | | | |
| | | | transglycosylase) | | | |
| USP15 | | USP15 | ubiquitin specific | Cytoplasm | peptidase | |
| | | | peptidase 15 | | | |
| USP24 | | USP24 | ubiquitin specific | unknown | peptidase | |
| | | | peptidase 24 | | | |
| USP28 | | USP28 | ubiquitin specific | Nucleus | peptidase | |
| | | | peptidase 28 | | | |
| USP32 | | USP32 | ubiquitin specific | Cytoplasm | enzyme | |
| | | | peptidase 32 | | | |
| USP34 | | USP34 | ubiquitin specific | unknown | peptidase | |
| | | | peptidase 34 | | | |
| USP47 | | USP47 | ubiquitin specific | Cytoplasm | peptidase | |
| | | | peptidase 47 | | | |
| USP5 | | USP5 | ubiquitin specific | Cytoplasm | peptidase | |
| | | | peptidase 5 | | | |
| | | | (isopeptidase T) | | | |
| USP7 | | USP7 | ubiquitin specific | Nucleus | peptidase | |
| | | | peptidase 7 | | | |
| | | | (herpes virus- | | | |
| | | | associated) | | | |
| USP9X | | USP9X | ubiquitin specific | Plasma | peptidase | |
| | | | peptidase 9, X- | Membrane | | |
| | | | linked | | | |
| VGLL1 | | VGLL1 | vestigial like 1 | Nucleus | transcription | |
| | | | (Drosophila) | | regulator | |
| VPS11 | | VPS11 | vacuolar protein | Cytoplasm | transporter | |
| | | | sorting 11 | | | |
| | | | homolog | | | |
| | | | (S. cerevisiae) | | | |
| WBP2 | | WBP2 | WW domain | Cytoplasm | other | |
| | | | binding protein 2 | | | |
| WBP4 | | WBP4 | WW domain | Cytoplasm | other | |
| | | | binding protein 4 | | | |
| | | | (formin binding | | | |
| | | | protein 21) | | | |
| WDR11 | | WDR11 | WD repeat | unknown | other | |
| | | | domain 11 | | | |
| WDR18 | | WDR18 | WD repeat | Nucleus | other | |
| | | | domain 18 | | | |
| WDR5 | | WDR5 | WD repeat | Nucleus | other | |
| | | | domain 5 | | | |
| WDR6 | | WDR6 | WD repeat | Cytoplasm | other | |
| | | | domain 6 | | | |
| WDR61 | | WDR61 | WD repeat | unknown | other | |
| | | | domain 61 | | | |
| WDR77 | | WDR77 | WD repeat | Nucleus | transcription | |
| | | | domain 77 | | regulator | |
| WDR82 | | WDR82 | WD repeat | Nucleus | other | |
| | | | domain 82 | | | |
| XAB2 | | XAB2 | XPA binding | Nucleus | other | |
| | | | protein 2 | | | |
| XIAP | | XIAP | X-linked inhibitor | Cytoplasm | other | |
| | | | of apoptosis | | | |
| YWHAB | | YWHAB | tyrosine 3- | Cytoplasm | transcription | |
| | | | monooxygenase/ | | regulator | |
| | | | tryptophan 5- | | | |
| | | | monooxygenase | | | |
| | | | activation | | | |
| | | | protein, beta | | | |
| | | | polypeptide | | | |
| YWHAE | | YWHAE | tyrosine 3- | Cytoplasm | other | |
| | | | monooxygenase/ | | | |
| | | | tryptophan 5- | | | |
| | | | monooxygenase | | | |
| | | | activation | | | |
| | | | protein, epsilon | | | |
| | | | polypeptide | | | |
| YWHAG | | YWHAG | tyrosine 3- | Cytoplasm | other | |
| | | | monooxygenase/ | | | |
| | | | tryptophan 5- | | | |
| | | | monooxygenase | | | |
| | | | activation | | | |
| | | | protein, gamma | | | |
| | | | polypeptide | | | |
| YWHAH | | YWHAH | tyrosine 3- | Cytoplasm | transcription | |
| | | | monooxygenase/ | | regulator | |
| | | | tryptophan 5- | | | |
| | | | monooxygenase | | | |
| | | | activation | | | |
| | | | protein, eta | | | |
| | | | polypeptide | | | |
| YWHAQ | | YWHAQ | tyrosine 3- | Cytoplasm | other | |
| | | | monooxygenase/ | | | |
| | | | tryptophan 5- | | | |
| | | | monooxygenase | | | |
| | | | activation | | | |
| | | | protein, theta | | | |
| | | | polypeptide | | | |
| YWHA | | YWHA | tyrosine 3- | Cytoplasm | enzyme | |
| | | | monooxygenase/ | | | |
| | | | tryptophan 5- | | | |
| | | | monooxygenase | | | |
| | | | activation | | | |
| | | | protein, zeta | | | |
| | | | polypeptide | | | |
| ZBED1 | | ZBED1 | zinc finger, | Nucleus | enzyme | |
| | | | BED-type | | | |
| | | | containing 1 | | | |
| ZC3H13 | | ZC3H13 | zinc finger | unknown | other | |
| | | | CCCH-type | | | |
| | | | containing 13 | | | |
| ZC3H4 | | ZC3H4 | zinc finger | unknown | other | |
| | | | CCCH-type | | | |
| | | | containing 4 | | | |
| ZC3HAV1 | | ZC3HAV1 | zinc finger | Plasma | other | |
| | | | CCCH-type, | Membrane | | |
| | | | antiviral 1 | | | |
| ZFR | | ZFR | zinc finger RNA | Nucleus | other | |
| | | | binding protein | | | |
| ZNF511 | | ZNF511 | zinc finger | Nucleus | other | |
| | | | protein 511 | | | |
| ZW10 | | ZW10 | ZW10, | Nucleus | other | |
| | | | kinetochore | | | |
| | | | associated, | | | |
| | | | homolog | | | |
| | | | (Drosophila) | | | |
| ZWILCH | | ZWILCH | Zwilch, | Nucleus | other | |
| | | | kinetochore | | | |
| | | | associated, | | | |
| | | | homolog | | | |
| | | | (Drosophila) | | | |

### PI3K-AKT-mTOR Pathway

Phosphatidylinositol 3 kinases (PI3K) are a family of lipid kinases whose inositol lipid products play a central role in signal transduction pathways of cytokines, growth factors and other extracellular matrix proteins. PI3Ks are divided into three classes: Class I, II and III with Class I being the best studied one. It is a heterodimer consisting of a catalytic and regulatory subunit. These are most commonly found to be p110 and p85. Phosphorylation of phosphoinositide(4,5)bisphosphate (PIP2) by Class I PI3K generates PtdIns(3,4,5)P3. The different PI3ks are involved in a variety of signaling pathways. This is mediated through their interaction with molecules like the receptor tyrosine kinases (RTKs), the adapter molecules GAB1-GRB2, and the kinase JAK. These converge to activate PDK1 which then phosphorylates AKT. AKT follows two distinct paths: 1) Inhibitory role—for example, AKT inhibits apoptosis by phosphorylating the Bad component of the Bad/Bcl-XL complex, allowing for cell survival. 2) Activating role—AKT activates IKK leading to NF-κB activation and cell survival. By its inhibitory as well as activating role, AKT is involved in numerous cellular processes like energy storage, cell cycle progression, protein synthesis and angiogenesis.

This pathway is composed of, but not restricted to 1-phosphatidyl-D-myo-inositol 4,5-bisphosphate, 14-3-3, 14-3-3-Cdkn1b, Akt, BAD, BCL2, BCL2L1, CCND1, CDC37, CDKN1A, CDKN1B, citrulline, CTNNB1, EIF4E, EIF4EBP1, ERK1/2, FKHR, GAB1/2, GDF15, Glycogen synthase, GRB2, Gsk3, Ikb, IkB-NfkB, IKK (complex), ILK, Integrin, JAK, L-arginine, LIMS1, MAP2K1/2, MAP3K5, MAP3K8, MAPK8IP1, MCL1, MDM2, MTOR, NANOG, NFkB (complex), nitric oxide, NOS3, P110, p70 S6k, PDPK1, phosphatidylinositol-3,4,5-triphosphate, PI3K p85, PP2A, PTEN, PTGS2, RAF1, Ras, RHEB, SFN, SHC1 (includes EG:20416), SHIP, Sos, THEM4, TP53 (includes EG:22059), TSC1, Tsc1-Tsc2, TSC2, YWHAE

### IGF-IR Signaling Network

Insulin-like growth factor-1 (IGF-1) is a peptide hormone under control of the growth hormone. IGF-1 promotes cell proliferation, growth and survival. Six specific binding proteins, IGFBP 1-6, allow for a more nuanced control of IGF activity. The IGF-1 receptor (IGF-1R) is a transmembrane tyrosine kinase protein. IGF-1-induced receptor activation results in autophosphorylation followed by an enhanced capability to activate downstream pathways. Activated IGF-1R phosphorylates SHC and IRS-1. SHC along with adapter molecules GRB2 and SOS forms a signaling complex that activates the Ras/Raf/MEK/ERK pathway. ERK translocation to the nucleus results in the activation of transcriptional regulators ELK-1, c-Jun and c-Fos which induce genes that promote cell growth and differentiation. IRS-1 activates pathways for cell survival via the PI3K/PDK1/AKT/BAD pathway. IRS-1 also activates pathways for cell growth via the PI3K/PDK1/p70RSK pathway. IGF-1 also signals via the JAK/STAT pathway by inducing tyrosine phosphorylation of JAK-1, JAK-2 and STAT-3. SOCS proteins are able to inhibit the JAKs thereby inhibiting this pathway. The adapter protein GRB10 interacts with IGF-IR. GRB10 also binds the E3 ubiquitin ligase NEDD4 and promotes ligand stimulated ubiquitination, internalization, and degradation of the IGF-IR as a means of long-term attenuation of signaling.

This pathway is composed of, but not restricted to 1-phosphatidyl-D-myo-inositol 4,5-bisphosphate, 14-3-3, 14-3-3-Bad, Akt, atypical protein kinase C, BAD, CASP9 (includes EG:100140945), Ck2, ELK1, ERK1/2, FKHR, FOS, GRB10, GRB2, IGF1, Igf1-Igfbp, IGF1R, Igfbp, IRS1/2, JAK1/2, JUN, MAP2K1/2, MAPK8, NEDD4, p70 S6k, PDPK1, phosphatidylinositol-3,4,5-triphosphate, PI3K (complex), Pka, PTK2 (includes EG:14083), PTPN11, PXN, RAFT, Ras, RASA1, SHC1 (includes EG:20416), SOCS, SOCS3, Sos, SRF, STAT3, Stat3-Stat3

### NRF2-Mediated Oxidative Stress Response

Oxidative stress is caused by an imbalance between the production of reactive oxygen and the detoxification of reactive intermediates. Reactive intermediates such as peroxides and free radicals can be very damaging to many parts of cells such as proteins, lipids and DNA. Severe oxidative stress can trigger apoptosis and necrosis. Oxidative stress is involved in many diseases such as atherosclerosis, Parkinson's disease and Alzheimer's disease. Oxidative stress has also been linked to aging. The cellular defense response to oxidative stress includes induction of detoxifying enzymes and antioxidant enzymes. Nuclear factor-erythroid 2-related factor 2 (Nrf2) binds to the antioxidant response elements (ARE) within the promoter of these enzymes and activates their transcription. Inactive Nrf2 is retained in the cytoplasm by association with an actin-binding protein Keap1. Upon exposure of cells to oxidative stress, Nrf2 is phosphorylated in response to the protein kinase C, phosphatidylinositol 3-kinase and MAP kinase pathways. After phosphorylation, Nrf2 translocates to the nucleus, binds AREs and transactivates detoxifying enzymes and antioxidant enzymes, such as glutathione S-transferase, cytochrome P450, NAD(P)H quinone oxidoreductase, heme oxygenase and superoxide dismutase.

This pathway is composed of, but not restricted to ABCC1, ABCC2, ABCC4 (includes EG:10257), Actin, Actin-Nrf2, Afar, AKR1A1, AKT1, AOX1, ATF4, BACH1, CAT, Cbp/p300, CBR1, CCT7, CDC34, CLPP, CUL3 (includes EG:26554), Cul3-Roc1, Cyp1a/2a/3a/4a/2c, EIF2AK3, ENC1, EPHX1, ERK1/2, ERP29, FKBP5, FMO1 (includes EG:14261), FOS, FOSL1, FTH1 (includes EG:14319), FTL, GCLC, GCLM, GPX2, GSK3B, GSR, GST, HERPUD1, HMOX1, Hsp22/Hsp40/Hsp90, JINK1/2, Jnkk, JUN/JUNB/JUND, KEAP1, Keap1-Nrf2, MAF, MAP2K1/2, MAP2K5, MAP3K1, MAP3K5, MAP3K7 (includes EG:172842), MAPK14, MAPK7, MKK3/6, musculoaponeurotic fibrosarcoma oncogene, NFE2L2, NQO, PI3K (complex), Pkc(s), PMF1, PPIB, PRDX1, Psm, PTPLAD1, RAFT, Ras, RBX1, reactive oxygen species, SCARB1, SLC35A2, Sod, SQSTM1, STIP1, TXN (includes EG:116484), TXNRD1, UBB, UBE2E3, UBE2K, USP14, VCP

### Protein Kinase A Signaling Pathway

Protein kinase A (PKA) regulates processes as diverse as growth, development, memory, and metabolism. It exists as a tetrameric complex of two catalytic subunits (PKA-C) and a regulatory (PKA-R) subunit dimer. Type-II PKA is anchored to specific locations within the cell by AKAPs. Extracellular stimuli such as neurotransmitters, hormones, inflammatory stimuli, stress, epinephrine and norepinephrine activate G-proteins through receptors such as GPCRs and ADR-α/β. These receptors along with others such as CRHR, GcgR and DCC are responsible for cAMP accumulation which leads to activation of PKA. The conversion of ATP to cAMP is mediated by the 9 transmembrane AC enzymes and one soluble AC. The transmembrane AC are regulated by heterotrimeric G-proteins, Gαs, Gαq and Gαi. Gαs and Gαq activate while Gαi inhibits AC. Gβ and Gγ subunits act synergistically with Gαs and Gαq to activate ACII, IV and VII. However the β and γ subunits along with Gαi inhibit the activity of ACI, V and VI.

G-proteins indirectly influence cAMP signaling by activating PLC, which generates DAG and IP3. DAG in turn activates PKC. IP3 modulates proteins upstream to cAMP signaling with the release of Ca2+ from the ER through IP3R. Ca2+ is also released by CaCn and CNG. Ca2+ release activates Calmodulin, CamKKs and CamKs, which take part in cAMP modulation by activating ACI. Gα13 activates MEKK1 and RhoA via two independent pathways which induce phosphorylation and degradation of IκBα and activation of PKA. High levels of cAMP under stress conditions like hypoxia, ischemia and heat shock also directly activate PKA. TGF-β activates PKA independent of cAMP through phosphorylation of SMAD proteins. PKA phosphorylates Phospholamban which regulates the activity of SERCA2 leading to myocardial contraction, whereas phosphorylation of TnnI mediates relaxation. PKA also activates KDELR to promote protein retrieval thereby maintaining steady state of the cell. Increase in concentration of Ca2+ followed by PKA activation enhances eNOS activity which is essential for cardiovascular homeostasis. Activated PKA represses ERK activation by inhibition of Raf1. PKA inhibits the interaction of 14-3-3 proteins with BAD and NFAT to promote cell survival. PKA phosphorylates endothelial MLCK leading to decreased basal MLC phosphorylation. It also phosphorylates filamin, adducin, paxillin and FAK and is involved in the disappearance of stress fibers and F-actin accumulation in membrane ruffles. PKA also controls phosphatase activity by phosphorylation of a specific PPtase1 inhibitor, DARPP32. Other substrates of PKA include histone H1, histone H2B and CREB.

This pathway is composed of, but not restricted to 1-phosphatidyl-D-myo-inositol 4,5-bisphosphate, 14-3-3, ADCY, ADCY1/5/6, ADCY2/4/7, ADCY9, Adducin, AKAP, APC, ATF1 (includes EG:100040260), ATP, BAD, BRAF, Ca2+, Calcineurin protein(s), Calmodulin, CaMKII, CHUK, Cng Channel, Creb, CREBBP, CREM, CTNNB1, cyclic AMP, DCC, diacylglycerol, ELK1, ERK1/2, Filamin, Focal adhesion kinase, G protein alphai, G protein beta gamma, G-protein beta, G-protein gamma, GLI3, glycogen, glycogen phosphorylase, Glycogen synthase, GNA13, GNAQ, GNAS, GRK1/7, Gsk3, Hedgehog, Histone H1, Histone h3, Ikb, IkB-NfkB, inositol triphosphate, ITPR, KDELR, LIPE, MAP2K1/2, MAP3K1, Mlc, myosin-light-chain kinase, Myosin2, Nfat (family), NFkB (complex), NGFR, NOS3, NTN1, Patched, Pde, Phk, Pka, Pka catalytic subunit, PKAr, Pkc(s), PLC, PLN, PP1 protein complex group, PPP1R1B, PTPase, PXN, RAF1, Rap1, RHO, RHOA, Rock, Ryr, SMAD3, Smad3-Smad4, SMAD4, SMO, TCF/LEF, Tgf beta, Tgf beta receptor, TGFBR1, TGFBR2, TH, Tni, VASP

### IL-6 Signaling Pathway

The central role of IL-6 in inflammation makes it an important target for the management of inflammation associated with cancer. IL-6 responses are transmitted through Glycoprotein 130 (GP130), which serves as the universal signal-transducing receptor subunit for all IL-6-related cytokines IL-6-type cytokines utilize tyrosine kinases of the Janus Kinase (JAK) family and signal transducers and activators of transcription (STAT) family as major mediators of signal transduction. Upon receptor stimulation by IL-6, the JAK family of kinases associated with GP130 are activated, resulting in the phosphorylation of GP130. Several phosphotyrosine residues of GP130 serve as docking sites for STAT factors mainly STAT3 and STAT1. Subsequently, STATs are phosphorylated, form dimers and translocate to the nucleus, where they regulate transcription of target genes. In addition to the JAK/STAT pathway of signal transduction, IL-6 also activates the extracellular signal-regulated kinases (ERK1/2) of the mitogen activated protein kinase (MAPK) pathway. The upstream activators of ERK1/2 include RAS and the src homology-2 containing proteins GRB2 and SHC. The SHC protein is activated by JAK2 and thus serves as a link between the IL-6 activated JAK/STAT and RAS-MAPK pathways. The phosphorylation of MAPKs in response to IL-6 activated RAS results in the activation of nuclear factor IL-6 (NF-IL6), which in turn stimulates the transcription of the IL-6 gene. The transcription of the IL-6 gene is also stimulated by tumor necrosis factor (TNF) and Interleukin-1 (IL-1) via the activation of nuclear factor kappa B (NFκB).

Based on the findings by the method described here in MDA-MB-468 cells, combination of an inhibitor of components of these identified pathways, such as those targeting but not limited to AKT, mTOR, PI3K, IGF1R, IKK, Bcl2, PKA complex, phosphodiesterases are proposed to be efficacious when used in combination with an Hsp90 inhibitor.

Example of AKT inhibitors are PF-04691502, Triciribine phosphate (NSC-280594), A-674563, CCT128930, AT7867, PHT-427, GSK690693, MK-2206

Example of PI3K inhibitors are 2-(1H-indazol-4-yl)-6-(4-methanesulfonylpiperazin-1-ylmethyl)-4-morpholin-4-ylthieno(3,2-d)pyrimidine, BKM120, NVP-BEZ235, PX-866, SF 1126, XL147.

Example of mTOR inhibitors are deforolimus, everolimus, NVP-BEZ235, OSI-027, tacrolimus, temsirolimus, Ku-0063794, WYE-354, PP242, OSI-027, GSK2126458, WAY-600, WYE-125132

Examples of Bcl2 inhibitors are ABT-737, Obatoclax (GX15-070), ABT-263, TW-37

Examples of IGF1R inhibitors are NVP-ADW742, BMS-754807, AVE1642, BIIB022, cixutumumab, ganitumab, IGF1, OSI-906

Examples of JAK inhibitors are Tofacitinib citrate (CP-690550), AT9283, AG-490, INCB018424 (Ruxolitinib), AZD1480, LY2784544, NVP-BSK805, TG101209, TG-101348

Examples of IkK inhibitors are SC-514, PF 184

Examples of inhibitors of phosphodiesterases are aminophylline, anagrelide, arofylline, caffeine, cilomilast, dipyridamole, dyphylline, L 869298, L-826,141, milrinone, nitroglycerin, pentoxifylline, roflumilast, rolipram, tetomilast, theophylline, tolbutamide, amrinone, anagrelide, arofylline, caffeine, cilomilast, L 869298, L-826,141, milrinone, pentoxifylline, roflumilast, rolipram, tetomilast

In the Diffuse large B-cell lymphoma (DLBCL) cell line OCI-LY1, major signaling networks identified by the method were the B cell receptor, PKCteta, PI3K/AKT, CD40, CD28 and the ERK/MAPK signaling pathways (FIG. 23). Pathway components as identified by the method are listed in Table 4.

TABLE 4
© 2000-2012 Ingenuity Systems, Inc. All rights reserved.

| ID | Notes | Symbol | Entrez Gene Name | Location | Type(s) | Drug(s) |
|---|---|---|---|---|---|---|
| AAGAB | | AAGAB | alpha- and | Cytoplasm | other | |
| | | | gamma-adaptin | | | |
| | | | binding protein | | | |
| ABI1 | | ABI1 | abl-interactor 1 | Cytoplasm | other | |
| ABR | | ABR | active BCR-related | Cytoplasm | other | |
| | | | gene | | | |
| AHSA1 | | AHSA1 | AHA1, activator of | Cytoplasm | other | |
| | | | heat shock 90 kDa | | | |
| | | | protein ATPase | | | |
| | | | homolog 1 (yeast) | | | |
| AIFM1 | | AIFM1 | apoptosis-inducing | Cytoplasm | enzyme | |
| | | | factor, | | | |
| | | | mitochondrion- | | | |
| | | | associated, 1 | | | |
| AKAP8 | | AKAP8 | A kinase (PRKA) | Nucleus | other | |
| | | | anchor protein 8 | | | |
| AKAP8L | | AKAP8L | A kinase (PRKA) | Nucleus | other | |
| | | | anchor protein 8- | | | |
| | | | like | | | |
| ALKBH8 | | ALKBH8 | alkB, alkylation | Cytoplasm | enzyme | |
| | | | repair homolog 8 | | | |
| | | | (E. coli) | | | |
| ALOX5 | | ALOX5 | arachidonate 5- | Cytoplasm | enzyme | TA 270, |
| | | | lipoxygenase | | | benoxaprofen, |
| | | | | | | meclofenamic |
| | | | | | | acid, zileuton, |
| | | | | | | sulfasalazine, |
| | | | | | | balsalazide, 5- |
| | | | | | | aminosalicylic |
| | | | | | | acid, masoprocol |
| ANAPC7 | | ANAPC7 | anaphase | Nucleus | other | |
| | | | promoting complex | | | |
| | | | subunit 7 | | | |
| ANKFY1 | | ANKFY1 | ankyrin repeat and | Nucleus | transcription | |
| | | | FYVE domain | | regulator | |
| | | | containing 1 | | | |
| ANKRD17 | | ANKRD17 | ankyrin repeat | unknown | other | |
| | | | domain 17 | | | |
| ANP32B | | ANP32B | acidic (leucine- | Nucleus | other | |
| | | | rich) nuclear | | | |
| | | | phosphoprotein 32 | | | |
| | | | family, member B | | | |
| AP1B1 | | AP1B1 | adaptor-related | Cytoplasm | transporter | |
| | | | protein complex 1, | | | |
| | | | beta 1 subunit | | | |
| AP2A1 | | AP2A1 | adaptor-related | Cytoplasm | transporter | |
| | | | protein complex 2, | | | |
| | | | alpha 1 subunit | | | |
| APIP | | APIP | APAF1 interacting | Cytoplasm | enzyme | |
| | | | protein | | | |
| APOBEC3G | | APOBEC3G | apolipoprotein B | Nucleus | enzyme | |
| | | | mRNA editing | | | |
| | | | enzyme, catalytic | | | |
| | | | polypeptide-like 3G | | | |
| ARFGAP1 | | ARFGAP1 | ADP-ribosylation | Cytoplasm | transporter | |
| | | | factor GTPase | | | |
| | | | activating protein 1 | | | |
| ARFGEF2 | | ARFGEF2 | ADP-ribosylation | Cytoplasm | other | |
| | | | factor guanine | | | |
| | | | nucleotide- | | | |
| | | | exchange factor 2 | | | |
| | | | (brefeldin A- | | | |
| | | | inhibited) | | | |
| ARFIP2 | | ARFIP2 | ADP-ribosylation | Cytoplasm | other | |
| | | | factor interacting | | | |
| | | | protein 2 | | | |
| ARHGEF1 | | ARHGEF1 | Rho guanine | Cytoplasm | other | |
| | | | nucleotide | | | |
| | | | exchange factor | | | |
| | | | (GEF) 1 | | | |
| ARID1A | | ARID1A | AT rich interactive | Nucleus | transcription | |
| | | | domain 1A (SWI- | | regulator | |
| | | | like) | | | |
| ASAH1 | | ASAH1 | N-acylsphingosine | Cytoplasm | enzyme | |
| | | | amidohydrolase | | | |
| | | | (acid ceramidase) 1 | | | |
| ASMTL | | ASMTL | acetylserotonin O- | Cytoplasm | enzyme | |
| | | | methyltransferase- | | | |
| | | | like | | | |
| ASNA1 | | ASNA1 | arsA arsenite | Nucleus | transporter | |
| | | | transporter, ATP- | | | |
| | | | binding, homolog 1 | | | |
| | | | (bacterial) | | | |
| ASPSCR1 | | ASPSCR1 | alveolar soft part | Cytoplasm | other | |
| | | | sarcoma | | | |
| | | | chromosome | | | |
| | | | region, candidate 1 | | | |
| ATM | | ATM | ataxia | Nucleus | kinase | |
| | | | telangiectasia | | | |
| | | | mutated | | | |
| ATR | | ATR | ataxia | Nucleus | kinase | |
| | | | telangiectasia and | | | |
| | | | Rad3 related | | | |
| ATXN10 | | ATXN10 | ataxin 10 | Cytoplasm | other | |
| ATXN2L | | ATXN2L | ataxin 2-like | unknown | other | |
| BABAM1 | | BABAM1 | BRISC and | Nucleus | other | |
| | | | BRCA1 A complex | | | |
| | | | member 1 | | | |
| BAG6 | | BAG6 | BCL2-associated | Nucleus | enzyme | |
| | | | athanogene 6 | | | |
| BIRC6 | | BIRC6 | baculoviral IAP | Cytoplasm | enzyme | |
| | | | repeat containing 6 | | | |
| BRAT1 | | BRAT1 | BRCA1-associated | Cytoplasm | other | |
| | | | ATM activator 1 | | | |
| BRCC3 | | BRCC3 | BRCA1/BRCA2- | Nucleus | enzyme | |
| | | | containing | | | |
| | | | complex, subunit 3 | | | |
| BTAF1 | | BTAF1 | BTAF1 RNA | Nucleus | transcription | |
| | | | polymerase II, B- | | regulator | |
| | | | TFIID transcription | | | |
| | | | factor-associated, | | | |
| | | | 170 kDa (Mot1 | | | |
| | | | homolog, | | | |
| | | | S. cerevisiae) | | | |
| BTK | | BTK | Bruton | Cytoplasm | kinase | |
| | | | agammaglobulinemia | | | |
| | | | tyrosine kinase | | | |
| BUB1B | | BUB1B | budding | Nucleus | kinase | |
| | | | uninhibited by | | | |
| | | | benzimidazoles 1 | | | |
| | | | homolog beta | | | |
| | | | (yeast) | | | |
| BUB3 | | BUB3 | budding | Nucleus | other | |
| | | (includes | uninhibited by | | | |
| | | EG: 12237) | benzimidazoles 3 | | | |
| | | | homolog (yeast) | | | |
| BZW1 | | BZW1 | basic leucine | Cytoplasm | translation | |
| | | | zipper and W2 | | regulator | |
| | | | domains 1 | | | |
| CACYBP | | CACYBP | calcyclin binding | Nucleus | other | |
| | | | protein | | | |
| CALU | | CALU | calumenin | Cytoplasm | other | |
| CAMK1D | | CAMK1D | calcium/calmodulin- | Cytoplasm | kinase | |
| | | | dependent protein | | | |
| | | | kinase ID | | | |
| CAMK2D | | CAMK2D | calcium/calmodulin- | Cytoplasm | kinase | |
| | | | dependent protein | | | |
| | | | kinase II delta | | | |
| CAMK2G | | CAMK2G | calcium/calmodulin- | Cytoplasm | kinase | |
| | | | dependent protein | | | |
| | | | kinase II gamma | | | |
| CAMK4 | | CAMK4 | calcium/calmodulin- | Nucleus | kinase | |
| | | | dependent protein | | | |
| | | | kinase IV | | | |
| CAND1 | | CAND1 | cullin-associated | Cytoplasm | transcription | |
| | | | and neddylation- | | regulator | |
| | | | dissociated 1 | | | |
| CANX | | CANX | calnexin | Cytoplasm | other | |
| CAP1 | | CAP1 | CAP, adenylate | Plasma | other | |
| | | | cyclase-associated | Membrane | | |
| | | | protein 1 (yeast) | | | |
| CAPN1 | | CAPN1 | calpain 1, (mu/l) | Cytoplasm | peptidase | |
| | | | large subunit | | | |
| CAPRIN1 | | CAPRIN1 | cell cycle | Plasma | other | |
| | | | associated protein 1 | Membrane | | |
| CARM1 | | CARM1 | coactivator- | Nucleus | transcription | |
| | | | associated | | regulator | |
| | | | arginine | | | |
| | | | methyltransferase 1 | | | |
| CCNY | | CCNY | cyclin Y | Nucleus | other | |
| CD38 | | CD38 | CD38 molecule | Plasma | enzyme | |
| | | | | Membrane | | |
| CD74 | | CD74 | CD74 molecule, | Plasma | transmembrane | |
| | | | major | Membrane | receptor | |
| | | | histocompatibility | | | |
| | | | complex, class II | | | |
| | | | invariant chain | | | |
| CDC37 | | CDC37 | cell division cycle | Cytoplasm | other | |
| | | | 37 homolog | | | |
| | | | (S. cerevisiae) | | | |
| CDC37L1 | | CDC37L1 | cell division cycle | Cytoplasm | other | |
| | | | 37 homolog | | | |
| | | | (S. cerevisiae)-like 1 | | | |
| CDK1 | | CDK1 | cyclin-dependent | Nucleus | kinase | flavopiridol |
| | | | kinase 1 | | | |
| CDK4 | | CDK4 | cyclin-dependent | Nucleus | kinase | PD-0332991, |
| | | | kinase 4 | | | flavopiridol |
| CDK7 | | CDK7 | cyclin-dependent | Nucleus | kinase | BMS-387032, |
| | | | kinase 7 | | | flavopiridol |
| CDK9 | | CDK9 | cyclin-dependent | Nucleus | kinase | BMS-387032, |
| | | | kinase 9 | | | flavopiridol |
| CHAF1B | | CHAF1B | chromatin | Nucleus | other | |
| | | | assembly factor 1, | | | |
| | | | subunit B (p60) | | | |
| CHD8 | | CHD8 | chromodomain | Nucleus | enzyme | |
| | | | helicase DNA | | | |
| | | | binding protein 8 | | | |
| CHTF18 | | CHTF18 | CTF18, | unknown | other | |
| | | | chromosome | | | |
| | | | transmission | | | |
| | | | fidelity factor 18 | | | |
| | | | homolog | | | |
| | | | (S. cerevisiae) | | | |
| CNN2 | | CNN2 | calponin 2 | Cytoplasm | other | |
| CNOT1 | | CNOT1 | CCR4-NOT | Cytoplasm | other | |
| | | | transcription | | | |
| | | | complex, subunit 1 | | | |
| CNP | | CNP | 2′,3′-cyclic | Cytoplasm | enzyme | |
| | | | nucleotide 3′ | | | |
| | | | phosphodiesterase | | | |
| CNTLN | | CNTLN | centlein, | unknown | other | |
| | | | centrosomal | | | |
| | | | protein | | | |
| COBRA1 | | COBRA1 | cofactor of BRCA1 | Nucleus | other | |
| CORO7 | | CORO7 | coronin 7 | Cytoplasm | other | |
| CRKL | | CRKL | v-crk sarcoma | Cytoplasm | kinase | |
| | | | virus CT10 | | | |
| | | | oncogene homolog | | | |
| | | | (avian)-like | | | |
| CSDE1 | | CSDE1 | cold shock domain | Cytoplasm | enzyme | |
| | | | containing E1, | | | |
| | | | RNA-binding | | | |
| CSNK1A1 | | CSNK1A1 | casein kinase 1, | Cytoplasm | kinase | |
| | | | alpha 1 | | | |
| CSNK2A1 | | CSNK2A1 | casein kinase 2, | Cytoplasm | kinase | |
| | | | alpha 1 | | | |
| | | | polypeptide | | | |
| CSNK2A2 | | CSNK2A2 | casein kinase 2, | Cytoplasm | kinase | |
| | | | alpha prime | | | |
| | | | polypeptide | | | |
| CTBP2 | | CTBP2 | C-terminal binding | Nucleus | transcription | |
| | | | protein 2 | | regulator | |
| CTS | | CTS | cathepsin | Cytoplasm | peptidase | |
| CUTC | | CUTC | cutC copper | Cytoplasm | other | |
| | | | transporter | | | |
| | | | homolog (E. coli) | | | |
| CYB5R3 | | CYB5R3 | cytochrome b5 | Cytoplasm | enzyme | |
| | | | reductase 3 | | | |
| CYFIP1 | | CYFIP1 | cytoplasmic FMR1 | Cytoplasm | other | |
| | | | interacting protein 1 | | | |
| CYFIP2 | | CYFIP2 | cytoplasmic FMR1 | Cytoplasm | other | |
| | | | interacting protein 2 | | | |
| DBNL | | DBNL | drebrin-like | Cytoplasm | other | |
| DCAF7 | | DCAF7 | DDB1 and CUL4 | Cytoplasm | other | |
| | | | associated factor 7 | | | |
| DICER1 | | DICER1 | dicer 1, | Cytoplasm | enzyme | |
| | | | ribonuclease type | | | |
| | | | III | | | |
| DIMT1 | | DIMT1 | DIM1 | Cytoplasm | enzyme | |
| | | | dimethyladenosine | | | |
| | | | transferase 1 | | | |
| | | | homolog | | | |
| | | | (S. cerevisiae) | | | |
| DIS3L | | DIS3L | DIS3 mitotic | Cytoplasm | enzyme | |
| | | | control homolog | | | |
| | | | (S. cerevisiae)-like | | | |
| DNAJA1 | | DNAJA1 | DnaJ (Hsp40) | Nucleus | other | |
| | | | homolog, | | | |
| | | | subfamily A, | | | |
| | | | member 1 | | | |
| DNAJA2 | | DNAJA2 | DnaJ (Hsp40) | Nucleus | enzyme | |
| | | | homolog, | | | |
| | | | subfamily A, | | | |
| | | | member 2 | | | |
| DNAJB1 | | DNAJB1 | DnaJ (Hsp40) | Nucleus | other | |
| | | | homolog, | | | |
| | | | subfamily B, | | | |
| | | | member 1 | | | |
| DNAJB11 | | DNAJB11 | DnaJ (Hsp40) | Cytoplasm | other | |
| | | | homolog, | | | |
| | | | subfamily B, | | | |
| | | | member 11 | | | |
| DNAJB2 | | DNAJB2 | DnaJ (Hsp40) | Nucleus | other | |
| | | | homolog, | | | |
| | | | subfamily B, | | | |
| | | | member 2 | | | |
| DNAJC10 | | DNAJC10 | DnaJ (Hsp40) | Cytoplasm | enzyme | |
| | | | homolog, | | | |
| | | | subfamily C, | | | |
| | | | member 10 | | | |
| DNAJC21 | | DNAJC21 | DnaJ (Hsp40) | unknown | other | |
| | | | homolog, | | | |
| | | | subfamily C, | | | |
| | | | member 21 | | | |
| DNAJC7 | | DNAJC7 | DnaJ (Hsp40) | Cytoplasm | other | |
| | | | homolog, | | | |
| | | | subfamily C, | | | |
| | | | member 7 | | | |
| DNMT1 | | DNMT1 | DNA (cytosine-5-)- | Nucleus | enzyme | |
| | | | methyltransferase 1 | | | |
| DOCK2 | | DOCK2 | dedicator of | Cytoplasm | other | |
| | | | cytokinesis 2 | | | |
| DPH5 | | DPH5 | DPH5 homolog | unknown | enzyme | |
| | | | (S. cerevisiae) | | | |
| DPYSL2 | | DPYSL2 | dihydropyrimidinase- | Cytoplasm | enzyme | |
| | | | like 2 | | | |
| DRG1 | | DRG1 | developmentally | Cytoplasm | other | |
| | | | regulated GTP | | | |
| | | | binding protein 1 | | | |
| DTX3L | | DTX3L | deltex 3-like | Cytoplasm | enzyme | |
| | | | (Drosophila) | | | |
| EBNA1BP2 | | EBNA1BP2 | EBNA1 binding | Nucleus | other | |
| | | | protein 2 | | | |
| EEF1A1 | | EEF1A1 | eukaryotic | Cytoplasm | translation | |
| | | | translation | | regulator | |
| | | | elongation factor 1 | | | |
| | | | alpha 1 | | | |
| EHD1 | | EHD1 | EH-domain | Cytoplasm | other | |
| | | | containing 1 | | | |
| EIF2B2 | | EIF2B2 | eukaryotic | Cytoplasm | translation | |
| | | | translation initiation | | regulator | |
| | | | factor 2B, subunit | | | |
| | | | 2 beta, 39 kDa | | | |
| ELMO1 | | ELMO1 | engulfment and | Cytoplasm | other | |
| | | | cell motility 1 | | | |
| EPG5 | | EPG5 | ectopic P-granules | unknown | other | |
| | | | autophagy protein | | | |
| | | | 5 homolog | | | |
| | | | (C. elegans) | | | |
| EPS15 | | EPS15 | epidermal growth | Plasma | other | |
| | | | factor receptor | Membrane | | |
| | | | pathway substrate 15 | | | |
| EPS15L1 | | EPS15L1 | epidermal growth | Plasma | other | |
| | | | factor receptor | Membrane | | |
| | | | pathway substrate | | | |
| | | | 15-like 1 | | | |
| ETF1 | | ETF1 | eukaryotic | Cytoplasm | translation | |
| | | | translation | | regulator | |
| | | | termination factor 1 | | | |
| EXOSC2 | | EXOSC2 | exosome | Nucleus | enzyme | |
| | | | component 2 | | | |
| EXOSC5 | | EXOSC5 | exosome | Nucleus | enzyme | |
| | | | component 5 | | | |
| EXOSC6 | | EXOSC6 | exosome | Nucleus | other | |
| | | | component 6 | | | |
| EXOSC7 | | EXOSC7 | exosome | Nucleus | enzyme | |
| | | | component 7 | | | |
| FANCD2 | | FANCD2 | Fanconi anemia, | Nucleus | other | |
| | | | complementation | | | |
| | | | group D2 | | | |
| FANCI | | FANCI | Fanconi anemia, | Nucleus | other | |
| | | | complementation | | | |
| | | | group I | | | |
| FBXL12 | | FBXL12 | F-box and leucine- | Cytoplasm | other | |
| | | | rich repeat protein 12 | | | |
| FBXO22 | | FBXO22 | F-box protein 22 | unknown | enzyme | |
| FBXO3 | | FBXO3 | F-box protein 3 | unknown | enzyme | |
| FCHSD2 | | FCHSD2 | FCH and double | unknown | other | |
| | | | SH3 domains 2 | | | |
| FCRLA | | FCRLA | Fc receptor-like A | Plasma | other | |
| | | | | Membrane | | |
| FDFT1 | | FDFT1 | farnesyl- | Cytoplasm | enzyme | TAK-475, |
| | | | diphosphate | | | zoledronic |
| | | | farnesyltransferase 1 | | | acid |
| FKBP4 | | FKBP4 | FK506 binding | Nucleus | enzyme | |
| | | | protein 4, 59 kDa | | | |
| FKBP5 | | FKBP5 | FK506 binding | Nucleus | enzyme | |
| | | | protein 5 | | | |
| FLI1 | | FLI1 | Friend leukemia | Nucleus | transcription | |
| | | | virus integration 1 | | regulator | |
| FLII | | FLII | flightless I homolog | Nucleus | other | |
| | | | (Drosophila) | | | |
| FLNA | | FLNA | filamin A, alpha | Cytoplasm | other | |
| FN3KRP | | FN3KRP | fructosamine 3 | unknown | kinase | |
| | | | kinase related | | | |
| | | | protein | | | |
| FNBP1 | | FNBP1 | formin binding | Nucleus | enzyme | |
| | | | protein 1 | | | |
| G3BP1 | | G3BP1 | GTPase activating | Nucleus | enzyme | |
| | | | protein (SH3 | | | |
| | | | domain) binding | | | |
| | | | protein 1 | | | |
| G3BP2 | | G3BP2 | GTPase activating | Nucleus | enzyme | |
| | | | protein (SH3 | | | |
| | | | domain) binding | | | |
| | | | protein 2 | | | |
| GAPVD1 | | GAPVD1 | GTPase activating | Cytoplasm | other | |
| | | | protein and VPS9 | | | |
| | | | domains 1 | | | |
| GARS | | GARS | glycyl-tRNA | Cytoplasm | enzyme | |
| | | | synthetase | | | |
| GART | | GART | phosphoribosyl- | Cytoplasm | enzyme | LY231514 |
| | | | glycinamide | | | |
| | | | formyltransferase, | | | |
| | | | phosphoribosyl- | | | |
| | | | glycinamide | | | |
| | | | synthetase, | | | |
| | | | phosphoribosylamino- | | | |
| | | | imidazole | | | |
| | | | synthetase | | | |
| GIGYF2 | | GIGYF2 | GRB10 interacting | unknown | other | |
| | | | GYF protein 2 | | | |
| GLMN | | GLMN | glomulin, FKBP | Cytoplasm | other | |
| | | | associated protein | | | |
| GLRX3 | | GLRX3 | glutaredoxin 3 | Cytoplasm | enzyme | |
| GOLPH3L | | GOLPH3L | golgi | Cytoplasm | other | |
| | | | phosphoprotein 3- | | | |
| | | | like | | | |
| GPATCH8 | | GPATCH8 | G patch domain | unknown | other | |
| | | | containing 8 | | | |
| GTF2B | | GTF2B | general | Nucleus | transcription | |
| | | | transcription factor | | regulator | |
| | | | IIB | | | |
| GTF2F1 | | GTF2F1 | general | Nucleus | transcription | |
| | | | transcription factor | | regulator | |
| | | | IIF, polypeptide 1, | | | |
| | | | 74 kDa | | | |
| GTF2F2 | | GTF2F2 | general | Nucleus | transcription | |
| | | | transcription factor | | regulator | |
| | | | IIF, polypeptide 2, | | | |
| | | | 30 kDa | | | |
| GTF2I | | GTF2I | general | Nucleus | transcription | |
| | | | transcription factor | | regulator | |
| | | | IIi | | | |
| GTF3C1 | | GTF3C1 | general | Nucleus | transcription | |
| | | | transcription factor | | regulator | |
| | | | IIIC, polypeptide 1, | | | |
| | | | alpha 220 kDa | | | |
| GTPBP4 | | GTPBP4 | GTP binding | Nucleus | enzyme | |
| | | | protein 4 | | | |
| HAT1 | | HAT1 | histone | Nucleus | enzyme | |
| | | | acetyltransferase 1 | | | |
| HCLS1 | | HCLS1 | hematopoietic cell- | Nucleus | transcription | |
| | | | specific Lyn | | regulator | |
| | | | substrate 1 | | | |
| HDAC1 | | HDAC1 | histone | Nucleus | transcription | tributyrin, |
| | | | deacetylase 1 | | regulator | belinostat, |
| | | | | | | pyroxamide, |
| | | | | | | MGCD0103, |
| | | | | | | vorinostat, |
| | | | | | | romidepsin |
| HDAC2 | | HDAC2 | histone | Nucleus | transcription | tributyrin, |
| | | | deacetylase 2 | | regulator | belinostat, |
| | | | | | | pyroxamide, |
| | | | | | | vorinostat, |
| | | | | | | romidepsin |
| HDAC3 | | HDAC3 | histone | Nucleus | transcription | tributyrin, |
| | | | deacetylase 3 | | regulator | belinostat, |
| | | | | | | pyroxamide, |
| | | | | | | MGCD0103, |
| | | | | | | vorinostat, |
| | | | | | | romidepsin |
| HDAC6 | | HDAC6 | histone | Nucleus | transcription | tributyrin, |
| | | | deacetylase 6 | | regulator | belinostat, |
| | | | | | | pyroxamide, |
| | | | | | | vorinostat, |
| | | | | | | romidepsin |
| HDLBP | | HDLBP | high density | Nucleus | transporter | |
| | | | lipoprotein binding | | | |
| | | | protein | | | |
| HECTD1 | | HECTD1 | HECT domain | unknown | enzyme | |
| | | | containing 1 | | | |
| HERC1 | | HERC1 | hect (homologous | Cytoplasm | other | |
| | | | to the E6-AP | | | |
| | | | (UBE3A) carboxyl | | | |
| | | | terminus) domain | | | |
| | | | and RCC1 | | | |
| | | | (CHC1)-like | | | |
| | | | domain (RLD) 1 | | | |
| HIF1AN | | HIF1AN | hypoxia inducible | Nucleus | enzyme | |
| | | | factor 1, alpha | | | |
| | | | subunit inhibitor | | | |
| HIRIP3 | | HIRIP3 | HIRA interacting | Nucleus | other | |
| | | | protein 3 | | | |
| HIST1H1B | | HIST1H1B | histone cluster 1, | Nucleus | other | |
| | | | H1b | | | |
| HIST1H1D | | HIST1H1D | histone cluster 1, | Nucleus | other | |
| | | | H1d | | | |
| HK2 | | HK2 | hexokinase 2 | Cytoplasm | kinase | |
| HLA-DQB1 | | HLA-DQB1 | major | Plasma | other | |
| | | | histocompatibility | Membrane | | |
| | | | complex, class II, | | | |
| | | | DQ beta 1 | | | |
| HLA-DRA | | HLA-DRA | major | Plasma | transmembrane | |
| | | | histocompatibility | Membrane | receptor | |
| | | | complex, class II, | | | |
| | | | DR alpha | | | |
| HLA-DRB1 | | HLA-DRB1 | major | Plasma | transmembrane | apolizumab |
| | | | histocompatibility | Membrane | receptor | |
| | | | complex, class II, | | | |
| | | | DR beta 1 | | | |
| HNRNPAB | | HNRNPAB | heterogeneous | Nucleus | enzyme | |
| | | | nuclear | | | |
| | | | ribonucleoprotein | | | |
| | | | A/B | | | |
| HNRNPD | | HNRNPD | heterogeneous | Nucleus | transcription | |
| | | | nuclear | | regulator | |
| | | | ribonucleoprotein | | | |
| | | | D (AU-rich element | | | |
| | | | RNA binding | | | |
| | | | protein 1, 37 kDa) | | | |
| HNRNPU | | HNRNPU | heterogeneous | Nucleus | transporter | |
| | | | nuclear | | | |
| | | | ribonucleoprotein | | | |
| | | | U (scaffold | | | |
| | | | attachment factor | | | |
| | | | A) | | | |
| HSP90AA1 | | HSP90AA1 | heat shock protein | Cytoplasm | enzyme | 17- |
| | | | 90 kDa alpha | | | dimethylamino- |
| | | | (cytosolic), class A | | | ethylamino-17- |
| | | | member 1 | | | demethoxy- |
| | | | | | | geldanamycin, |
| | | | | | | IPI-504, |
| | | | | | | cisplatin |
| HSP90AB1 | | HSP90AB1 | heat shock protein | Cytoplasm | enzyme | 17- |
| | | | 90 kDa alpha | | | dimethylamino- |
| | | | (cytosolic), class B | | | ethylamino-17- |
| | | | member 1 | | | demethoxy- |
| | | | | | | geldanamycin, |
| | | | | | | IPI-504, |
| | | | | | | cisplatin |
| HSP90B1 | | HSP90B1 | heat shock protein | Cytoplasm | other | 17- |
| | | | 90 kDa beta | | | dimethylamino- |
| | | | (Grp94), member 1 | | | ethylamino-17- |
| | | | | | | demethoxy- |
| | | | | | | geldanamycin, |
| | | | | | | IPI-504, |
| | | | | | | cisplatin |
| HSPA4 | | HSPA4 | heat shock 70 kDa | Cytoplasm | other | |
| | | | protein 4 | | | |
| HSPA5 | | HSPA5 | heat shock 70 kDa | Cytoplasm | enzyme | |
| | | | protein 5 (glucose- | | | |
| | | | regulated protein, | | | |
| | | | 78 kDa) | | | |
| HSPA8 | | HSPA8 | heat shock 70 kDa | Cytoplasm | enzyme | |
| | | | protein 8 | | | |
| HSPA9 | | HSPA9 | heat shock 70 kDa | Cytoplasm | other | |
| | | | protein 9 (mortalin) | | | |
| HSPD1 | | HSPD1 | heat shock 60 kDa | Cytoplasm | enzyme | |
| | | | protein 1 | | | |
| | | | (chaperonin) | | | |
| HSPH1 | | HSPH1 | heat shock | Cytoplasm | other | |
| | | | 105 kDa/110 kDa | | | |
| | | | protein 1 | | | |
| HTRA2 | | HTRA2 | HtrA serine | Cytoplasm | peptidase | |
| | | | peptidase 2 | | | |
| IFIH1 | | IFIH1 | interferon induced | Nucleus | enzyme | |
| | | | with helicase C | | | |
| | | | domain 1 | | | |
| IFIT1 | | IFIT1 | interferon-induced | Cytoplasm | other | |
| | | | protein with | | | |
| | | | tetratricopeptide | | | |
| | | | repeats 1 | | | |
| IFIT3 | | IFIT3 | interferon-induced | Cytoplasm | other | |
| | | | protein with | | | |
| | | | tetratricopeptide | | | |
| | | | repeats 3 | | | |
| IGBP1 | | IGBP1 | immunoglobulin | Cytoplasm | phosphatase | |
| | | | (CD79A) binding | | | |
| | | | protein 1 | | | |
| IGF2BP3 | | IGF2BP3 | insulin-like growth | Cytoplasm | translation | |
| | | | factor 2 mRNA | | regulator | |
| | | | binding protein 3 | | | |
| IKBKAP | | IKBKAP | inhibitor of kappa | Cytoplasm | other | |
| | | | light polypeptide | | | |
| | | | gene enhancer in | | | |
| | | | B-cells, kinase | | | |
| | | | complex- | | | |
| | | | associated protein | | | |
| ILF2 | | ILF2 | interleukin | Nucleus | transcription | |
| | | | enhancer binding | | regulator | |
| | | | factor 2, 45 kDa | | | |
| INPP5B | | INPP5B | inositol | Plasma | phosphatase | |
| | | | polyphosphate-5- | Membrane | | |
| | | | phosphatase, | | | |
| | | | 75 kDa | | | |
| INPP5D | | INPP5D | inositol | Cytoplasm | phosphatase | |
| | | | polyphosphate-5- | | | |
| | | | phosphatase, | | | |
| | | | 145 kDa | | | |
| ISY1 | | ISY1 | ISY1 splicing factor | Nucleus | other | |
| | | (includes | homolog | | | |
| | | EG: 362394) | (S. cerevisiae) | | | |
| ITCH | | ITCH | itchy E3 ubiquitin | Nucleus | enzyme | |
| | | | protein ligase | | | |
| | | | homolog (mouse) | | | |
| ITFG2 | | ITFG2 | integrin alpha FG- | unknown | other | |
| | | | GAP repeat | | | |
| | | | containing 2 | | | |
| ITIH3 | | ITIH3 | inter-alpha-trypsin | Extracellular | other | |
| | | | inhibitor heavy | Space | | |
| | | | chain 3 | | | |
| ITSN2 | | ITSN2 | intersectin 2 | Cytoplasm | other | |
| KARS | | KARS | lysyl-tRNA | Cytoplasm | enzyme | |
| | | | synthetase | | | |
| KCNAB2 | | KCNAB2 | potassium voltage- | Plasma | ion channel | |
| | | | gated channel, | Membrane | | |
| | | | shaker-related | | | |
| | | | subfamily, beta | | | |
| | | | member 2 | | | |
| KIAA0368 | | KIAA0368 | KIAA0368 | Cytoplasm | other | |
| KIAA0564 | | KIAA0564 | KIAA0564 | Cytoplasm | other | |
| KIAA0664 | | KIAA0664 | KIAA0664 | Cytoplasm | translation | |
| | | | | | regulator | |
| KIAA1524 | | KIAA1524 | KIAA1524 | Cytoplasm | other | |
| KIAA1797 | | KIAA1797 | KIAA1797 | unknown | other | |
| KIAA1967 | | KIAA1967 | KIAA1967 | Cytoplasm | peptidase | |
| LARS | | LARS | leucyl-tRNA | Cytoplasm | enzyme | |
| | | | synthetase | | | |
| LPXN | | LPXN | leupaxin | Cytoplasm | other | |
| LTN1 | | LTN1 | listerin E3 ubiquitin | Nucleus | enzyme | |
| | | | protein ligase 1 | | | |
| LYAR | | LYAR | Ly1 antibody | Plasma | other | |
| | | | reactive homolog | Membrane | | |
| | | | (mouse) | | | |
| MAGI1 | | MAGI1 | membrane | Plasma | kinase | |
| | | (includes | associated | Membrane | | |
| | | EG: 14924) | guanylate kinase, | | | |
| | | | WW and PD | | | |
| | | | domain containing 1 | | | |
| MAP3K1 | | MAP3K1 | mitogen-activated | Cytoplasm | kinase | |
| | | | protein kinase | | | |
| | | | kinase kinase 1 | | | |
| MAPK1 | | MAPK1 | mitogen-activated | Cytoplasm | kinase | |
| | | | protein kinase 1 | | | |
| MAPK14 | | MAPK14 | mitogen-activated | Cytoplasm | kinase | SCIO-469, |
| | | | protein kinase 14 | | | RO-3201195 |
| MAPK3 | | MAPK3 | mitogen-activated | Cytoplasm | kinase | |
| | | | protein kinase 3 | | | |
| MAPK9 | | MAPK9 | mitogen-activated | Cytoplasm | kinase | |
| | | | protein kinase 9 | | | |
| MCM2 | | MCM2 | minichromosome | Nucleus | enzyme | |
| | | | maintenance | | | |
| | | | complex | | | |
| | | | component 2 | | | |
| MCMBP | | MCMBP | minichromosome | Nucleus | other | |
| | | | maintenance | | | |
| | | | complex binding | | | |
| | | | protein | | | |
| MED1 | | MED1 | mediator complex | Nucleus | transcription | |
| | | (includes | subunit 1 | | regulator | |
| | | EG: 19014) | | | | |
| MEMO1 | | MEMO1 | mediator of cell | Cytoplasm | other | |
| | | (includes | motility 1 | | | |
| | | EG: 298787) | | | | |
| MEPCE | | MEPCE | methylphosphate | unknown | enzyme | |
| | | | capping enzyme | | | |
| METTL15 | | METTL15 | methyltransferase | unknown | other | |
| | | | like 15 | | | |
| MLH1 | | MLH1 | mutL homolog 1, | Nucleus | enzyme | |
| | | | colon cancer, | | | |
| | | | nonpolyposis type | | | |
| | | | 2 (E. coli) | | | |
| MLST8 | | MLST8 | MTOR associated | Cytoplasm | other | |
| | | | protein, LST8 | | | |
| | | | homolog | | | |
| | | | (S. cerevisiae) | | | |
| MMS19 | | MMS19 | MMS19 nucleotide | Nucleus | transcription | |
| | | | excision repair | | regulator | |
| | | | homolog | | | |
| | | | (S. cerevisiae) | | | |
| MS4A1 | | MS4A1 | membrane- | Plasma | other | tositumomab, |
| | | | spanning 4- | Membrane | | rituximab, |
| | | | domains, subfamily | | | ofatumumab, |
| | | | A, member 1 | | | veltuzumab, |
| | | | | | | afutuzumab, |
| | | | | | | ibritumomab |
| | | | | | | tiuxetan |
| MSH2 | | MSH2 | mutS homolog 2, | Nucleus | enzyme | |
| | | | colon cancer, | | | |
| | | | nonpolyposis type | | | |
| | | | 1 (E. coli) | | | |
| MSH6 | | MSH6 | mutS homolog 6 | Nucleus | enzyme | |
| | | | (E. coli) | | | |
| MSI2 | | MSI2 | musashi homolog | Cytoplasm | other | |
| | | | 2 (Drosophila) | | | |
| MSTO1 | | MSTO1 | misato homolog 1 | Cytoplasm | other | |
| | | | (Drosophila) | | | |
| MTHFD1 | | MTHFD1 | methylenetetra- | Cytoplasm | enzyme | |
| | | | hydrofolate | | | |
| | | | dehydrogenase | | | |
| | | | (NADP+ | | | |
| | | | dependent) 1, | | | |
| | | | methenyltetra- | | | |
| | | | hydrofolate | | | |
| | | | cyclohydrolase, | | | |
| | | | formyltetra- | | | |
| | | | hydrofolate | | | |
| | | | synthetase | | | |
| MTOR | | MTOR | mechanistic target | Nucleus | kinase | deforolimus, |
| | | | of rapamycin | | | OSI-027, |
| | | | (serine/threonine | | | NVP-BEZ235, |
| | | | kinase) | | | temsirolimus, |
| | | | | | | tacrolimus, |
| | | | | | | everolimus |
| MX1 | | MX1 | myxovirus | Nucleus | enzyme | |
| | | | (influenza virus) | | | |
| | | | resistance 1, | | | |
| | | | interferon-inducible | | | |
| | | | protein p78 | | | |
| | | | (mouse) | | | |
| MYBBP1A | | MYBBP1A | MYB binding | Nucleus | transcription | |
| | | | protein (P160) 1a | | regulator | |
| MYCBP2 | | MYCBP2 | MYC binding | Nucleus | enzyme | |
| | | | protein 2 | | | |
| MYH9 | | MYH9 | myosin, heavy | Cytoplasm | enzyme | |
| | | | chain 9, non- | | | |
| | | | muscle | | | |
| MYO9A | | MYO9A | myosin IXA | Cytoplasm | enzyme | |
| NADKD1 | | NADKD1 | NAD kinase | Cytoplasm | other | |
| | | | domain containing 1 | | | |
| NASP | | NASP | nuclear | Nucleus | other | |
| | | | autoantigenic | | | |
| | | | sperm protein | | | |
| | | | (histone-binding) | | | |
| NAT10 | | NAT10 | N- | Nucleus | enzyme | |
| | | | acetyltransferase | | | |
| | | | 10 (GCN5-related) | | | |
| NCAPD2 | | NCAPD2 | non-SMC | Nucleus | other | |
| | | | condensin I | | | |
| | | | complex, subunit D2 | | | |
| NCAPG2 | | NCAPG2 | non-SMC | Nucleus | other | |
| | | | condensin II | | | |
| | | | complex, subunit G2 | | | |
| NCBP1 | | NCBP1 | nuclear cap | Nucleus | other | |
| | | | binding protein | | | |
| | | | subunit 1, 80 kDa | | | |
| NCKAP1L | | NCKAP1L | NCK-associated | Plasma | other | |
| | | | protein 1-like | Membrane | | |
| NCKIPSD | | NCKIPSD | NCK interacting | Nucleus | other | |
| | | | protein with SH3 | | | |
| | | | domain | | | |
| NCL | | NCL | nucleolin | Nucleus | other | |
| NCOR1 | | NCOR1 | nuclear receptor | Nucleus | transcription | |
| | | | corepressor 1 | | regulator | |
| NCOR2 | | NCOR2 | nuclear receptor | Nucleus | transcription | |
| | | | corepressor 2 | | regulator | |
| NDE1 | | NDE1 | nudE nuclear | Nucleus | other | |
| | | (includes | distribution gene E | | | |
| | | EG: 54820) | homolog 1 | | | |
| | | | (A. nidulans) | | | |
| NEDD4L | | NEDD4L | neural precursor | Cytoplasm | enzyme | |
| | | | cell expressed, | | | |
| | | | developmentally | | | |
| | | | down-regulated 4- | | | |
| | | | like | | | |
| NEK9 | | NEK9 | NIMA (never in | Nucleus | kinase | |
| | | | mitosis gene a)- | | | |
| | | | related kinase 9 | | | |
| NFKB1 | | NFKB1 | nuclear factor of | Nucleus | transcription | |
| | | | kappa light | | regulator | |
| | | | polypeptide gene | | | |
| | | | enhancer in B-cells 1 | | | |
| NFKB2 | | NFKB2 | nuclear factor of | Nucleus | transcription | |
| | | | kappa light | | regulator | |
| | | | polypeptide gene | | | |
| | | | enhancer in B-cells | | | |
| | | | 2 (p49/p100) | | | |
| NFKBIB | | NFKBIB | nuclear factor of | Nucleus | transcription | |
| | | | kappa light | | regulator | |
| | | | polypeptide gene | | | |
| | | | enhancer in B-cells | | | |
| | | | inhibitor, beta | | | |
| NFKBIE | | NFKBIE | nuclear factor of | Nucleus | transcription | |
| | | | kappa light | | regulator | |
| | | | polypeptide gene | | | |
| | | | enhancer in B-cells | | | |
| | | | inhibitor, epsilon | | | |
| NISCH | | NISCH | nischarin | Plasma | transmembrane | |
| | | | | Membrane | receptor | |
| NOSIP | | NOSIP | nitric oxide | Cytoplasm | other | |
| | | | synthase | | | |
| | | | interacting protein | | | |
| NPM1 | | NPM1 | nucleophosmin | Nucleus | transcription | |
| | | | (nucleolar | | regulator | |
| | | | phosphoprotein | | | |
| | | | B23, numatrin) | | | |
| NSDHL | | NSDHL | NAD(P) dependent | Cytoplasm | enzyme | |
| | | | steroid | | | |
| | | | dehydrogenase- | | | |
| | | | like | | | |
| NSFL1C | | NSFL1C | NSFL1 (p97) | Cytoplasm | other | |
| | | | cofactor (p47) | | | |
| NSUN2 | | NSUN2 | NOP2/Sun domain | Nucleus | enzyme | |
| | | | family, member 2 | | | |
| NUDT5 | | NUDT5 | nudix (nucleoside | Cytoplasm | phosphatase | |
| | | | diphosphate linked | | | |
| | | | moiety X)-type | | | |
| | | | motif 5 | | | |
| OAS2 | | OAS2 | 2′-5′- | Cytoplasm | enzyme | |
| | | | oligoadenylate | | | |
| | | | synthetase 2, | | | |
| | | | 69/71 kDa | | | |
| OGDH | | OGDH | oxoglutarate | Cytoplasm | enzyme | |
| | | | (alpha- | | | |
| | | | ketoglutarate) | | | |
| | | | dehydrogenase | | | |
| | | | (lipoamide) | | | |
| OPA1 | | OPA1 | optic atrophy 1 | Cytoplasm | enzyme | |
| | | | (autosomal | | | |
| | | | dominant) | | | |
| OTUB1 | | OTUB1 | OTU domain, | unknown | enzyme | |
| | | | ubiquitin aldehyde | | | |
| | | | binding 1 | | | |
| PA2G4 | | PA2G4 | proliferation- | Nucleus | transcription | |
| | | | associated 2G4, | | regulator | |
| | | | 38 kDa | | | |
| PABPC1 | | PABPC1 | poly(A) binding | Cytoplasm | translation | |
| | | | protein, | | regulator | |
| | | | cytoplasmic 1 | | | |
| PARN | | PARN | poly(A)-specific | Nucleus | enzyme | |
| | | | ribonuclease | | | |
| PARP9 | | PARP9 | poly (ADP-ribose) | Nucleus | other | |
| | | | polymerase family, | | | |
| | | | member 9 | | | |
| PARVG | | PARVG | parvin, gamma | Cytoplasm | other | |
| PCBP1 | | PCBP1 | poly(rC) binding | Nucleus | translation | |
| | | | protein 1 | | regulator | |
| PCBP2 | | PCBP2 | poly(rC) binding | Nucleus | other | |
| | | | protein 2 | | | |
| PCDHGB6 | | PCDHGB6 | protocadherin | unknown | other | |
| | | | gamma subfamily | | | |
| | | | B, 6 | | | |
| PCID2 | | PCID2 | PCI domain | Nucleus | transcription | |
| | | | containing 2 | | regulator | |
| PCNA | | PCNA | proliferating cell | Nucleus | enzyme | |
| | | | nuclear antigen | | | |
| PDCD2L | | PDCD2L | programmed cell | unknown | other | |
| | | | death 2-like | | | |
| PDCD6IP | | PDCD6IP | programmed cell | Cytoplasm | other | |
| | | | death 6 interacting | | | |
| | | | protein | | | |
| PDE4DIP | | PDE4DIP | phosphodiesterase | Cytoplasm | enzyme | |
| | | | 4D interacting | | | |
| | | | protein | | | |
| PDHB | | PDHB | pyruvate | Cytoplasm | enzyme | |
| | | | dehydrogenase | | | |
| | | | (lipoamide) beta | | | |
| PDIA6 | | PDIA6 | protein disulfide | Cytoplasm | enzyme | |
| | | | isomerase family | | | |
| | | | A, member 6 | | | |
| PDK1 | | PDK1 | pyruvate | Cytoplasm | kinase | |
| | | | dehydrogenase | | | |
| | | | kinase, isozyme 1 | | | |
| PDP1 | | PDP1 | pyruvate | Cytoplasm | phosphatase | |
| | | | dehyrogenase | | | |
| | | | phosphatase | | | |
| | | | catalytic subunit 1 | | | |
| PDPR | | PDPR | pyruvate | Cytoplasm | enzyme | |
| | | | dehydrogenase | | | |
| | | | phosphatase | | | |
| | | | regulatory subunit | | | |
| PHKB | | PHKB | phosphorylase | Cytoplasm | kinase | |
| | | | kinase, beta | | | |
| PI4KA | | PI4KA | phosphatidylinositol | Cytoplasm | kinase | |
| | | | 4-kinase, | | | |
| | | | catalytic, alpha | | | |
| PIK3AP1 | | PIK3AP1 | phosphoinositide- | Cytoplasm | other | |
| | | | 3-kinase adaptor | | | |
| | | | protein 1 | | | |
| PIK3C2B | | PIK3C2B | phosphoinositide- | Cytoplasm | kinase | |
| | | | 3-kinase, class 2, | | | |
| | | | beta polypeptide | | | |
| PIK3C3 | | PIK3C3 | phosphoinositide- | Cytoplasm | kinase | |
| | | | 3-kinase, class 3 | | | |
| PIK3R4 | | PIK3R4 | phosphoinositide- | Cytoplasm | other | |
| | | | 3-kinase, | | | |
| | | | regulatory subunit 4 | | | |
| PLAA | | PLAA | phospholipase A2- | Cytoplasm | other | |
| | | | activating protein | | | |
| PLBD2 | | PLBD2 | phospholipase B | Extracellular | other | |
| | | | domain containing 2 | Space | | |
| PLCG2 | | PLCG2 | phospholipase C, | Cytoplasm | enzyme | |
| | | | gamma 2 | | | |
| | | | (phosphatidyl- | | | |
| | | | inositol-specific) | | | |
| PM20D2 | | PM20D2 | peptidase M20 | unknown | other | |
| | | | domain containing 2 | | | |
| PMS1 | | PMS1 | PMS1 postmeiotic | Nucleus | enzyme | |
| | | | segregation | | | |
| | | | increased 1 | | | |
| | | | (S. cerevisiae) | | | |
| PMS2 | | PMS2 | PMS2 postmeiotic | Nucleus | other | |
| | | | segregation | | | |
| | | | increased 2 | | | |
| | | | (S. cerevisiae) | | | |
| PNP | | PNP | purine nucleoside | Nucleus | enzyme | forodesine, |
| | | | phosphorylase | | | 9-deaza-9- |
| | | | | | | (3-thienyl- |
| | | | | | | methyl)guanine |
| POLD1 | | POLD1 | polymerase (DNA | Nucleus | enzyme | nelarabine, |
| | | | directed), delta 1, | | | MB07133, |
| | | | catalytic subunit | | | clofarabine, |
| | | | 125 kDa | | | cytarabine, |
| | | | | | | trifluridine, |
| | | | | | | vidarabine, |
| | | | | | | entecavir |
| POLR1C | | POLR1C | polymerase (RNA) | Nucleus | enzyme | |
| | | | I polypeptide C, | | | |
| | | | 30 kDa | | | |
| POLR2A | | POLR2A | polymerase (RNA) | Nucleus | enzyme | |
| | | | II (DNA directed) | | | |
| | | | polypeptide A, | | | |
| | | | 220 kDa | | | |
| PPAT | | PPAT | phosphoribosyl | Cytoplasm | enzyme | 6-mercaptopurine, |
| | | | pyrophosphate | | | thioguanine, |
| | | | amidotransferase | | | azathioprine |
| PPM1A | | PPM1A | protein | Cytoplasm | phosphatase | |
| | | | phosphatase, | | | |
| | | | Mg2+/Mn2+ | | | |
| | | | dependent, 1A | | | |
| PPP1CC | | PPP1CC | protein | Cytoplasm | phosphatase | |
| | | | phosphatase 1, | | | |
| | | | catalytic subunit, | | | |
| | | | gamma isozyme | | | |
| PPP2R1A | | PPP2R1A | protein | Cytoplasm | phosphatase | |
| | | | phosphatase 2, | | | |
| | | | regulatory subunit | | | |
| | | | A, alpha | | | |
| PPP3CA | | PPP3CA | phosphatase 3, | Cytoplasm | phosphatase | ISAtx-247, |
| | | | catalytic subunit, | | | tacrolimus, |
| | | | alpha isozyme | | | pimecrolimus, |
| | | | | | | cyclosporin A |
| PPP4C | | PPP4C | protein | Cytoplasm | phosphatase | |
| | | | phosphatase 4, | | | |
| | | | catalytic subunit | | | |
| PPP5C | | PPP5C | protein | Nucleus | phosphatase | |
| | | | phosphatase 5, | | | |
| | | | catalytic subunit | | | |
| PPP6C | | PPP6C | protein | Nucleus | phosphatase | |
| | | | phosphatase 6, | | | |
| | | | catalytic subunit | | | |
| PRKAA1 | | PRKAA1 | protein kinase, | Cytoplasm | kinase | |
| | | | AMP-activated, | | | |
| | | | alpha 1 catalytic | | | |
| | | | subunit | | | |
| PRKAB1 | | PRKAB1 | protein kinase, | Nucleus | kinase | |
| | | | AMP-activated, | | | |
| | | | beta 1 non- | | | |
| | | | catalytic subunit | | | |
| PRKAB2 | | PRKAB2 | protein kinase, | Cytoplasm | kinase | |
| | | | AMP-activated, | | | |
| | | | beta 2 non- | | | |
| | | | catalytic subunit | | | |
| PRKAG1 | | PRKAG1 | protein kinase, | Nucleus | kinase | |
| | | | AMP-activated, | | | |
| | | | gamma 1 non- | | | |
| | | | catalytic subunit | | | |
| PRKCSH | | PRKCSH | protein kinase C | Cytoplasm | enzyme | |
| | | | substrate 80K-H | | | |
| PRKD2 | | PRKD2 | protein kinase D2 | Cytoplasm | kinase | |
| PRKDC | | PRKDC | protein kinase, | Nucleus | kinase | |
| | | | DNA-activated, | | | |
| | | | catalytic | | | |
| | | | polypeptide | | | |
| PRMT1 | | PRMT1 | protein arginine | Nucleus | enzyme | |
| | | | methyltransferase 1 | | | |
| PRMT10 | | PRMT10 | protein arginine | unknown | other | |
| | | | methyltransferase | | | |
| | | | 10 (putative) | | | |
| PRMT3 | | PRMT3 | protein arginine | Nucleus | enzyme | |
| | | | methyltransferase 3 | | | |
| PRMT5 | | PRMT5 | protein arginine | Cytoplasm | enzyme | |
| | | | methyltransferase 5 | | | |
| PSD4 | | PSD4 | pleckstrin and | Cytoplasm | other | |
| | | | Sec7 domain | | | |
| | | | containing 4 | | | |
| PSMA1 | | PSMA1 | proteasome | Cytoplasm | peptidase | |
| | | | (prosome, | | | |
| | | | macropain) | | | |
| | | | subunit, alpha | | | |
| | | | type, 1 | | | |
| PSMC1 | | PSMC1 | proteasome | Nucleus | peptidase | |
| | | | (prosome, | | | |
| | | | macropain) 26S | | | |
| | | | subunit, ATPase, 1 | | | |
| PSME1 | | PSME1 | proteasome | Cytoplasm | other | |
| | | | (prosome, | | | |
| | | | macropain) | | | |
| | | | activator subunit 1 | | | |
| | | | (PA28 alpha) | | | |
| PTCD3 | | PTCD3 | Pentatricopeptide | Cytoplasm | other | |
| | | | repeat domain 3 | | | |
| PTGES2 | | PTGES2 | prostaglandin E | Cytoplasm | transcription | |
| | | | synthase 2 | | regulator | |
| PTK2 | | PTK2 | PTK2 protein | Cytoplasm | kinase | |
| | | (includes | tyrosine kinase 2 | | | |
| | | EG: 14083) | | | | |
| PTK2B | | PTK2B | PTK2B protein | Cytoplasm | kinase | |
| | | (includes | tyrosine kinase 2 | | | |
| | | EG: 19229) | beta | | | |
| PTPN1 | | PTPN1 | protein tyrosine | Cytoplasm | phosphatase | |
| | | | phosphatase, non- | | | |
| | | | receptor type 1 | | | |
| PTPN6 | | PTPN6 | protein tyrosine | Cytoplasm | phosphatase | |
| | | | phosphatase, non- | | | |
| | | | receptor type 6 | | | |
| PTPRJ | | PTPRJ | protein tyrosine | Plasma | phosphatase | |
| | | | phosphatase, | Membrane | | |
| | | | receptor type, J | | | |
| PUF60 | | PUF60 | poly-U binding | Nucleus | other | |
| | | | splicing factor | | | |
| | | | 60 KDa | | | |
| RAB3GAP1 | | RAB3GAP1 | RAB3 GTPase | Cytoplasm | other | |
| | | | activating protein | | | |
| | | | subunit 1 | | | |
| | | | (catalytic) | | | |
| RAB3GAP2 | | RAB3GAP2 | RAB3 GTPase | Cytoplasm | enzyme | |
| | | | activating protein | | | |
| | | | subunit 2 (non- | | | |
| | | | catalytic) | | | |
| RABGGTB | | RABGGTB | Rab | Cytoplasm | enzyme | |
| | | | geranylgeranyl- | | | |
| | | | transferase, beta | | | |
| | | | subunit | | | |
| RAD23B | | RAD23B | RAD23 homolog B | Nucleus | other | |
| | | | (S. cerevisiae) | | | |
| RAD51 | | RAD51 | RAD51 homolog | Nucleus | enzyme | |
| | | | (S. cerevisiae) | | | |
| RAE1 | | RAE1 | RAE1 RNA export | Nucleus | other | |
| | | | 1 homolog | | | |
| | | | (S. pombe) | | | |
| RANBP2 | | RANBP2 | RAN binding | Nucleus | enzyme | |
| | | | protein 2 | | | |
| RAPGEF6 | | RAPGEF6 | Rap guanine | Plasma | other | |
| | | | nucleotide | Membrane | | |
| | | | exchange factor | | | |
| | | | (GEF) 6 | | | |
| RARS | | RARS | arginyl-tRNA | Cytoplasm | enzyme | |
| | | | synthetase | | | |
| RASSF2 | | RASSF2 | Ras association | Nucleus | other | |
| | | | (RalGDS/AF-6) | | | |
| | | | domain family | | | |
| | | | member 2 | | | |
| RBCK1 | | RBCK1 | RanBP-type and | Cytoplasm | transcription | |
| | | | C3HC4-type zinc | | regulator | |
| | | | finger containing 1 | | | |
| RCOR1 | | RCOR1 | REST corepressor 1 | Nucleus | transcription | |
| | | | | | regulator | |
| REL | | REL | v-rel | Nucleus | transcription | |
| | | | reticuloendotheliosis | | regulator | |
| | | | viral oncogene | | | |
| | | | homolog (avian) | | | |
| RELA | | RELA | v-rel | Nucleus | transcription | NF-kappaB |
| | | | reticuloendotheliosis | | regulator | decoy |
| | | | viral oncogene | | | |
| | | | homolog A (avian) | | | |
| REM1 | | REM1 | RAS (RAD and | unknown | enzyme | |
| | | | GEM)-like GTP- | | | |
| | | | binding 1 | | | |
| RG9MTD1 | | RG9MTD1 | RNA (guanine-9-) | Cytoplasm | other | |
| | | | methyltransferase | | | |
| | | | domain containing 1 | | | |
| RNF138 | | RNF138 | ring finger protein 138 | unknown | other | |
| RNF20 | | RNF20 | ring finger protein 20 | Nucleus | enzyme | |
| RNF213 | | RNF213 | ring finger protein 213 | Plasma | other | |
| | | | | Membrane | | |
| RNF31 | | RNF31 | ring finger protein 31 | Cytoplasm | enzyme | |
| RNMT | | RNMT | RNA (guanine-7-) | Nucleus | enzyme | |
| | | | methyltransferase | | | |
| RPA1 | | RPA1 | replication protein | Nucleus | other | |
| | | | A1, 70 kDa | | | |
| RPA2 | | RPA2 | replication protein | Nucleus | other | |
| | | | A2, 32 kDa | | | |
| RPS6 | | RPS6 | ribosomal protein | Cytoplasm | other | |
| | | | S6 | | | |
| RPS6KA3 | | RPS6KA3 | ribosomal protein | Cytoplasm | kinase | |
| | | | S6 kinase, 90 kDa, | | | |
| | | | polypeptide 3 | | | |
| RTN4IP1 | | RTN4IP1 | reticulon 4 | Cytoplasm | enzyme | |
| | | | interacting protein 1 | | | |
| RUVBL1 | | RUVBL1 | RuvB-like 1 | Nucleus | transcription | |
| | | | (E. coli) | | regulator | |
| RUVBL2 | | RUVBL2 | RuvB-like 2 | Nucleus | transcription | |
| | | | (E. coli) | | regulator | |
| SAMHD1 | | SAMHD1 | SAM domain and | Nucleus | enzyme | |
| | | | HD domain 1 | | | |
| SCAF8 | | SCAF8 | SR-related CTD- | Nucleus | other | |
| | | | associated factor 8 | | | |
| SCFD1 | | SCFD1 | sec1 family domain | Cytoplasm | transporter | |
| | | | containing 1 | | | |
| SCPEP1 | | SCPEP1 | serine | Cytoplasm | peptidase | |
| | | | carboxypeptidase 1 | | | |
| SCYL1 | | SCYL1 | SCY1-like 1 | Cytoplasm | kinase | |
| | | | (S. cerevisiae) | | | |
| SEC23B | | SEC23B | Sec23 homolog B | Cytoplasm | transporter | |
| | | | (S. cerevisiae) | | | |
| SEC23IP | | SEC23IP | SEC23 interacting | Cytoplasm | other | |
| | | | protein | | | |
| SEPHS1 | | SEPHS1 | selenophosphate | unknown | enzyme | |
| | | | synthetase 1 | | | |
| SEPSECS | | SEPSECS | Sep (O- | Cytoplasm | other | |
| | | | phosphoserine) | | | |
| | | | tRNA: Sec | | | |
| | | | (selenocysteine) | | | |
| | | | tRNA synthase | | | |
| SEPT2 | | SEPT2 | septin 2 | Cytoplasm | enzyme | |
| SEPT9 | | SEPT9 | septin 9 | Cytoplasm | enzyme | |
| SERBP1 | | SERBP1 | SERPINE1 mRNA | Nucleus | other | |
| | | | binding protein 1 | | | |
| SERPINB9 | | SERPINB9 | serpin peptidase | Cytoplasm | other | |
| | | | inhibitor, clade B | | | |
| | | | (ovalbumin), | | | |
| | | | member 9 | | | |
| SET | | SET | SET nuclear | Nucleus | phosphatase | |
| | | | oncogene | | | |
| SETD2 | | SETD2 | SET domain | Cytoplasm | enzyme | |
| | | | containing 2 | | | |
| SF3A1 | | SF3A1 | splicing factor 3a, | Nucleus | other | |
| | | | subunit 1, 120 kDa | | | |
| SFPQ | | SFPQ | splicing factor | Nucleus | other | |
| | | | proline/glutamine- | | | |
| | | | rich | | | |
| SHARPIN | | SHARPIN | SHANK-associated | Plasma | other | |
| | | | RH domain | Membrane | | |
| | | | interactor | | | |
| SIRT3 | | SIRT3 | sirtuin 3 | Cytoplasm | enzyme | |
| SIRT5 | | SIRT5 | sirtuin 5 | Cytoplasm | enzyme | |
| SLBP | | SLBP | stem-loop binding | Nucleus | other | |
| | | | protein | | | |
| SLC1A5 | | SLC1A5 | solute carrier | Plasma | transporter | |
| | | | family 1 (neutral | Membrane | | |
| | | | amino acid | | | |
| | | | transporter), | | | |
| | | | member 5 | | | |
| SLC25A3 | | SLC25A3 | solute carrier | Cytoplasm | transporter | |
| | | | family 25 | | | |
| | | | (mitochondrial | | | |
| | | | carrier; phosphate | | | |
| | | | carrier), member 3 | | | |
| SLC25A5 | | SLC25A5 | solute carrier | Cytoplasm | transporter | |
| | | | family 25 | | | |
| | | | (mitochondrial | | | |
| | | | carrier; adenine | | | |
| | | | nucleotide | | | |
| | | | translocator), | | | |
| | | | member 5 | | | |
| SLC3A2 | | SLC3A2 | solute carrier | Plasma | transporter | |
| | | | family 3 (activators | Membrane | | |
| | | | of dibasic and | | | |
| | | | neutral amino acid | | | |
| | | | transport), member 2 | | | |
| SMAD2 | | SMAD2 | SMAD family | Nucleus | transcription | |
| | | | member 2 | | regulator | |
| SMARCA4 | | SMARCA4 | SWI/SNF related, | Nucleus | transcription | |
| | | | matrix associated, | | regulator | |
| | | | actin dependent | | | |
| | | | regulator of | | | |
| | | | chromatin, | | | |
| | | | subfamily a, | | | |
| | | | member 4 | | | |
| SMARCC2 | | SMARCC2 | SWI/SNF related, | Nucleus | transcription | |
| | | | matrix associated, | | regulator | |
| | | | actin dependent | | | |
| | | | regulator of | | | |
| | | | chromatin, | | | |
| | | | subfamily c, | | | |
| | | | member 2 | | | |
| SMARCD2 | | SMARCD2 | SWI/SNF related, | Nucleus | transcription | |
| | | | matrix associated, | | regulator | |
| | | | actin dependent | | | |
| | | | regulator of | | | |
| | | | chromatin, | | | |
| | | | subfamily d, | | | |
| | | | member 2 | | | |
| SMC1A | | SMC1A | structural | Nucleus | transporter | |
| | | | maintenance of | | | |
| | | | chromosomes 1A | | | |
| SMC2 | | SMC2 | structural | Nucleus | transporter | |
| | | | maintenance of | | | |
| | | | chromosomes 2 | | | |
| SMC3 | | SMC3 | structural | Nucleus | other | |
| | | | maintenance of | | | |
| | | | chromosomes 3 | | | |
| SMC4 | | SMC4 | structural | Nucleus | transporter | |
| | | | maintenance of | | | |
| | | | chromosomes 4 | | | |
| SMG1 | | SMG1 | smg-1 homolog, | Cytoplasm | kinase | |
| | | | phosphatidylinositol | | | |
| | | | 3-kinase-related | | | |
| | | | kinase | | | |
| | | | (C. elegans) | | | |
| SMNDC1 | | SMNDC1 | survival motor | Nucleus | other | |
| | | | neuron domain | | | |
| | | | containing 1 | | | |
| SNRNP200 | | SNRNP200 | small nuclear | Nucleus | enzyme | |
| | | | ribonucleoprotein | | | |
| | | | 200 kDa (U5) | | | |
| SPG21 | | SPG21 | spastic paraplegia | Plasma | enzyme | |
| | | | 21 (autosomal | Membrane | | |
| | | | recessive, Mast | | | |
| | | | syndrome) | | | |
| SRPK1 | | SRPK1 | SRSF protein | Nucleus | kinase | |
| | | | kinase 1 | | | |
| SRR | | SRR | serine racemase | Cytoplasm | enzyme | |
| SRSF7 | | SRSF7 | serine/arginine-rich | Nucleus | other | |
| | | | splicing factor 7 | | | |
| SSBP2 | | SSBP2 | single-stranded | Nucleus | transcription | |
| | | | DNA binding | | regulator | |
| | | | protein 2 | | | |
| ST13 | | ST13 | suppression of | Cytoplasm | other | |
| | | | tumorigenicity 13 | | | |
| | | | (colon carcinoma) | | | |
| | | | (Hsp70 interacting | | | |
| | | | protein) | | | |
| STAT1 | | STAT1 | signal transducer | Nucleus | transcription | |
| | | | and activator of | | regulator | |
| | | | transcription 1, | | | |
| | | | 91 kDa | | | |
| STAT3 | | STAT3 | signal transducer | Nucleus | transcription | |
| | | | and activator of | | regulator | |
| | | | transcription 3 | | | |
| | | | (acute-phase | | | |
| | | | response factor) | | | |
| STAT5B | | STAT5B | signal transducer | Nucleus | transcription | |
| | | | and activator of | | regulator | |
| | | | transcription 5B | | | |
| STIP1 | | STIP1 | stress-induced- | Cytoplasm | other | |
| | | | phosphoprotein 1 | | | |
| STK4 | | STK4 | serine/threonine | Cytoplasm | kinase | |
| | | | kinase 4 | | | |
| STRAP | | STRAP | serine/threonine | Plasma | other | |
| | | | kinase receptor | Membrane | | |
| | | | associated protein | | | |
| STUB1 | | STUB1 | STIP1 homology | Cytoplasm | enzyme | |
| | | | and U-box | | | |
| | | | containing protein | | | |
| | | | 1, E3 ubiquitin | | | |
| | | | protein ligase | | | |
| STX12 | | STX12 | syntaxin 12 | Plasma | other | |
| | | | | Membrane | | |
| SYK | | SYK | spleen tyrosine | Cytoplasm | kinase | |
| | | | kinase | | | |
| SYMPK | | SYMPK | symplekin | Cytoplasm | other | |
| SYNE1 | | SYNE1 | spectrin repeat | Nucleus | other | |
| | | | containing, nuclear | | | |
| | | | envelope 1 | | | |
| SYNE2 | | SYNE2 | spectrin repeat | Nucleus | other | |
| | | | containing, nuclear | | | |
| | | | envelope 2 | | | |
| TAB1 | | TAB1 | TGF-beta activated | Cytoplasm | enzyme | |
| | | | kinase 1/MAP3K7 | | | |
| | | | binding protein 1 | | | |
| TACC3 | | TACC3 | transforming, | Nucleus | other | |
| | | | acidic coiled-coil | | | |
| | | | containing protein 3 | | | |
| TARBP1 | | TARBP1 | TAR (HIV-1) RNA | Nucleus | transcription | |
| | | | binding protein 1 | | regulator | |
| TARDBP | | TARDBP | TAR DNA binding | Nucleus | transcription | |
| | | | protein | | regulator | |
| TBCD | | TBCD | tubulin folding | Cytoplasm | other | |
| | | | cofactor D | | | |
| TBK1 | | TBK1 | TANK-binding | Cytoplasm | kinase | |
| | | | kinase 1 | | | |
| TBL1XR1 | | TBL1XR1 | transducin (beta)- | Nucleus | transcription | |
| | | | like 1 X-linked | | regulator | |
| | | | receptor 1 | | | |
| TBL3 | | TBL3 | transducin (beta)- | Cytoplasm | peptidase | |
| | | | like 3 | | | |
| TBRG4 | | TBRG4 | transforming | Cytoplasm | other | |
| | | | growth factor beta | | | |
| | | | regulator 4 | | | |
| TFIP11 | | TFIP11 | tuftelin interacting | Extracellular | other | |
| | | | protein 11 | Space | | |
| TH1L | | TH1L | TH1-like | Nucleus | other | |
| | | | (Drosophila) | | | |
| THG1L | | THG1L | tRNA-histidine | Cytoplasm | enzyme | |
| | | | guanylyltransferase | | | |
| | | | 1-like | | | |
| | | | (S. cerevisiae) | | | |
| THOC2 | | THOC2 | THO complex 2 | Nucleus | other | |
| THUMPD1 | | THUMPD1 | THUMP domain | unknown | other | |
| | | | containing 1 | | | |
| THUMPD3 | | THUMPD3 | THUMP domain | unknown | other | |
| | | | containing 3 | | | |
| TIMM50 | | TIMM50 | translocase of | Cytoplasm | phosphatase | |
| | | | inner mitochondrial | | | |
| | | | membrane 50 | | | |
| | | | homolog | | | |
| | | | (S. cerevisiae) | | | |
| TIPRL | | TIPRL | TIP41, TOR | unknown | other | |
| | | | signaling pathway | | | |
| | | | regulator-like | | | |
| | | | (S. cerevisiae) | | | |
| TKT | | TKT | transketolase | Cytoplasm | enzyme | |
| TLE3 | | TLE3 | transducin-like | Nucleus | other | |
| | | | enhancer of split 3 | | | |
| | | | (E(sp1) homolog, | | | |
| | | | Drosophila) | | | |
| TLN1 | | TLN1 | talin 1 | Plasma | other | |
| | | | | Membrane | | |
| TOE1 | | TOE1 | target of EGR1, | Nucleus | other | |
| | | | member 1 | | | |
| | | | (nuclear) | | | |
| TOMM34 | | TOMM34 | translocase of | Cytoplasm | other | |
| | | | outer mitochondrial | | | |
| | | | membrane 34 | | | |
| TP53RK | | TP53RK | TP53 regulating | Nucleus | kinase | |
| | | | kinase | | | |
| TPP1 | | TPP1 | tripeptidyl | Cytoplasm | peptidase | |
| | | (includes | peptidase I | | | |
| | | EG: 1200) | | | | |
| TPP2 | | TPP2 | tripeptidyl | Cytoplasm | peptidase | |
| | | | peptidase II | | | |
| TRAP1 | | TRAP1 | TNF receptor- | Cytoplasm | enzyme | |
| | | | associated protein 1 | | | |
| TRIM25 | | TRIM25 | tripartite motif | Cytoplasm | transcription | |
| | | | containing 25 | | regulator | |
| TRIM28 | | TRIM28 | tripartite motif | Nucleus | transcription | |
| | | | containing 28 | | regulator | |
| TRIO | | TRIO | triple functional | Plasma | kinase | |
| | | | domain (PTPRF | Membrane | | |
| | | | interacting) | | | |
| TROVE2 | | TROVE2 | TROVE domain | Nucleus | other | |
| | | | family, member 2 | | | |
| TTC1 | | TTC1 | tetratricopeptide | unknown | other | |
| | | | repeat domain 1 | | | |
| TTC19 | | TTC19 | tetratricopeptide | Cytoplasm | other | |
| | | | repeat domain 19 | | | |
| TTC37 | | TTC37 | tetratricopeptide | unknown | other | |
| | | | repeat domain 37 | | | |
| TTC5 | | TTC5 | tetratricopeptide | unknown | other | |
| | | | repeat domain 5 | | | |
| TTN | | TTN | titin | Cytoplasm | kinase | |
| | | (includes | | | | |
| | | EG: 22138) | | | | |
| TUT1 | | TUT1 | terminal uridylyl | Nucleus | enzyme | |
| | | | transferase 1, U6 | | | |
| | | | snRNA-specific | | | |
| UBA1 | | UBA1 | ubiquitin-like | Cytoplasm | enzyme | |
| | | | modifier activating | | | |
| | | | enzyme 1 | | | |
| UBAC1 | | UBAC1 | UBA domain | Nucleus | other | |
| | | | containing 1 | | | |
| UBAP2 | | UBAP2 | ubiquitin | Cytoplasm | other | |
| | | | associated protein 2 | | | |
| UBAP2L | | UBAP2L | ubiquitin | unknown | other | |
| | | | associated protein | | | |
| | | | 2-like | | | |
| UBE2O | | UBE2O | ubiquitin- | unknown | enzyme | |
| | | | conjugating | | | |
| | | | enzyme E2O | | | |
| UBE3A | | UBE3A | ubiquitin protein | Nucleus | enzyme | |
| | | | ligase E3A | | | |
| UBQLN1 | | UBQLN1 | ubiquilin 1 | Cytoplasm | other | |
| UBR1 | | UBR1 | ubiquitin protein | Cytoplasm | enzyme | |
| | | (includes | ligase E3 | | | |
| | | EG: 197131) | component n- | | | |
| | | | recognin 1 | | | |
| UBR4 | | UBR4 | ubiquitin protein | Nucleus | other | |
| | | | ligase E3 | | | |
| | | | component n- | | | |
| | | | recognin 4 | | | |
| UBR5 | | UBR5 | ubiquitin protein | Nucleus | enzyme | |
| | | | ligase E3 | | | |
| | | | component n- | | | |
| | | | recognin 5 | | | |
| UBXN1 | | UBXN1 | UBX domain | Cytoplasm | other | |
| | | | protein 1 | | | |
| UCHL5 | | UCHL5 | ubiquitin carboxyl- | Cytoplasm | peptidase | |
| | | | terminal hydrolase | | | |
| | | | L5 | | | |
| UCK2 | | UCK2 | uridine-cytidine | Cytoplasm | kinase | |
| | | | kinase 2 | | | |
| UFD1L | | UFD1L | ubiquitin fusion | Cytoplasm | peptidase | |
| | | | degradation 1 like | | | |
| | | | (yeast) | | | |
| UHRF1BP1 | | UHRF1BP1 | UHRF1 binding | unknown | other | |
| | | | protein 1 | | | |
| UPF1 | | UPF1 | UPF1 regulator of | Nucleus | enzyme | |
| | | | nonsense | | | |
| | | | transcripts | | | |
| | | | homolog (yeast) | | | |
| USO1 | | USO1 | USO1 vesicle | Cytoplasm | transporter | |
| | | | docking protein | | | |
| | | | homolog (yeast) | | | |
| USP11 | | USP11 | ubiquitin specific | Nucleus | peptidase | |
| | | | peptidase 11 | | | |
| USP13 | | USP13 | ubiquitin specific | unknown | peptidase | |
| | | | peptidase 13 | | | |
| | | | (isopeptidase T-3) | | | |
| USP15 | | USP15 | ubiquitin specific | Cytoplasm | peptidase | |
| | | | peptidase 15 | | | |
| USP24 | | USP24 | ubiquitin specific | unknown | peptidase | |
| | | | peptidase 24 | | | |
| USP25 | | USP25 | ubiquitin specific | unknown | peptidase | |
| | | | peptidase 25 | | | |
| USP28 | | USP28 | ubiquitin specific | Nucleus | peptidase | |
| | | | peptidase 28 | | | |
| USP34 | | USP34 | ubiquitin specific | unknown | peptidase | |
| | | | peptidase 34 | | | |
| USP47 | | USP47 | ubiquitin specific | Cytoplasm | peptidase | |
| | | | peptidase 47 | | | |
| USP5 | | USP5 | ubiquitin specific | Cytoplasm | peptidase | |
| | | | peptidase 5 | | | |
| | | | (isopeptidase T) | | | |
| USP7 | | USP7 | ubiquitin specific | Nucleus | peptidase | |
| | | | peptidase 7 | | | |
| | | | (herpes virus- | | | |
| | | | associated) | | | |
| USP9X | | USP9X | ubiquitin specific | Plasma | peptidase | |
| | | | peptidase 9, X- | Membrane | | |
| | | | linked | | | |
| VAV1 | | VAV1 | vav 1 guanine | Nucleus | transcription | |
| | | | nucleotide | | regulator | |
| | | | exchange factor | | | |
| VCP | | VCP | valosin containing | Cytoplasm | enzyme | |
| | | | protein | | | |
| VDAC1 | | VDAC1 | voltage-dependent | Cytoplasm | ion channel | |
| | | | anion channel 1 | | | |
| VPRBP | | VPRBP | Vpr (HIV-1) binding | Nucleus | other | |
| | | | protein | | | |
| WBP2 | | WBP2 | WW domain | Cytoplasm | other | |
| | | | binding protein 2 | | | |
| WDFY4 | | WDFY4 | WDFY family | unknown | other | |
| | | | member 4 | | | |
| WDR11 | | WDR11 | WD repeat domain 11 | unknown | other | |
| WDR5 | | WDR5 | WD repeat domain 5 | Nucleus | other | |
| WDR6 | | WDR6 | WD repeat domain 6 | Cytoplasm | other | |
| WDR61 | | WDR61 | WD repeat domain 61 | unknown | other | |
| WDR82 | | WDR82 | WD repeat domain 82 | Nucleus | other | |
| WDR92 | | WDR92 | WD repeat domain 92 | unknown | other | |
| YWHAB | | YWHAB | tyrosine 3- | Cytoplasm | transcription | |
| | | | monooxygenase/ | | regulator | |
| | | | tryptophan 5- | | | |
| | | | monooxygenase | | | |
| | | | activation protein, | | | |
| | | | beta polypeptide | | | |
| YWHAE | | YWHAE | tyrosine 3- | Cytoplasm | other | |
| | | | monooxygenase/ | | | |
| | | | tryptophan 5- | | | |
| | | | monooxygenase | | | |
| | | | activation protein, | | | |
| | | | epsilon polypeptide | | | |
| YWHAG | | YWHAG | tyrosine 3- | Cytoplasm | other | |
| | | | monooxygenase/ | | | |
| | | | tryptophan 5- | | | |
| | | | monooxygenase | | | |
| | | | activation protein, | | | |
| | | | gamma | | | |
| | | | polypeptide | | | |
| YWHAH | | YWHAH | tyrosine 3- | Cytoplasm | transcription | |
| | | | monooxygenase/ | | regulator | |
| | | | tryptophan 5- | | | |
| | | | monooxygenase | | | |
| | | | activation protein, | | | |
| | | | eta polypeptide | | | |
| YWHAQ | | YWHAQ | tyrosine 3- | Cytoplasm | other | |
| | | | monooxygenase/ | | | |
| | | | tryptophan 5- | | | |
| | | | monooxygenase | | | |
| | | | activation protein, | | | |
| | | | theta polypeptide | | | |
| YWHA | | YWHA | tyrosine 3- | Cytoplasm | enzyme | |
| | | | monooxygenase/ | | | |
| | | | tryptophan 5- | | | |
| | | | monooxygenase | | | |
| | | | activation protein, | | | |
| | | | zeta polypeptide | | | |
| ZC3H11A | | ZC3H11A | zinc finger CCCH- | unknown | other | |
| | | | type containing | | | |
| | | | 11A | | | |
| ZC3H18 | | ZC3H18 | zinc finger CCCH- | Nucleus | other | |
| | | | type containing 18 | | | |
| ZC3H4 | | ZC3H4 | zinc finger CCCH- | unknown | other | |
| | | | type containing 4 | | | |
| ZFR | | ZFR | zinc finger RNA | Nucleus | other | |
| | | | binding protein | | | |
| ZFYVE26 | | ZFYVE26 | zinc finger, FYVE | Cytoplasm | other | |
| | | | domain containing | | | |
| | | | 26 | | | |
| ZNF259 | | ZNF259 | zinc finger protein | Nucleus | other | |
| | | | 259 | | | |

### B Cell Receptor Signaling

Signals propagated through the B cell antigen receptor (BCR) are crucial to the development, survival and activation of B lymphocytes. These signals also play a central role in the removal of potentially self-reactive B lymphocytes. The BCR is composed of surface-bound antigen recognizing membrane antibody and associated Ig-α and Ig-β heterodimers which are capable of signal transduction via cytosolic motifs called immunoreceptor tyrosine based activation motifs (ITAM). The recognition of polyvalent antigens by the B cell antigen receptor (BCR) initiates a series of interlinked signaling events that culminate in cellular responses. The engagement of the BCR induces the phosphorylation of tyrosine residues in the ITAM. The phosphorylation of ITAM is mediated by SYK kinase and the SRC family of kinases which include LYN, FYN and BLK. These kinases which are reciprocally activated by phosphorylated ITAMs in turn trigger a cascade of interlinked signaling pathways. Activation of the BCR leads to the stimulation of nuclear factor kappa B (NFκB). Central to BCR signaling via NF-kB is the complex formed by the Bruton's tyrosine kinase (BTK), the adaptor B-cell linker (BLNK) and phospholipase C gamma 2 (PLCγ2). Tyrosine phosphorylated adaptor proteins act as bridges between BCR associated tyrosine kinases and downstream effector molecules. BLNK is phosphorylated on BCR activation and serves to couple the tyrosine kinase SYK to the activation of PLCγ2. The complete stimulation of PLCγ2 is facilitated by BTK. Stimulated PLCγ2 triggers the DAG and Ca2+ mediated activation of Protein kinase (PKC) which in turn activates IkB kinase (IKK) and thereafter NFκB. In addition to the activation of NFκB, BLNK also interacts with other proteins like VAV and GRB2 resulting in the activation of the mitogen activated protein kinase (MAPK) pathway. This results in the transactivation of several factors like c-JUN, activation of transcription factor (ATF) and ELK6. Another adaptor protein, B cell adaptor for phosphoinositide 3-kinase (PI3K), termed BCAP once activated by SYK, goes on to trigger a PI3K/AKT signaling pathway. This pathway inhibits Glycogen synthase kinase 3 (GSK3), resulting in the nuclear accumulation of transcription factors like nuclear factor of activated T cells (NFAT) and enhancement of protein synthesis. Activation of PI3K/AKT pathway also leads to the inhibition of apoptosis in B cells. This pathway highlights the important components of B cell receptor antigen signaling.

This pathway is composed of, but not restricted to 1-phosphatidyl-D-myo-inositol 4,5-bisphosphate, ABL1, Akt, ATF2, BAD, BCL10, Bcl10-Card10-Malt1, BCL2A1, BCL2L1, BCL6, BLNK, BTK, Calmodulin, CaMKII, CARD10, CD19, CD22, CD79A, CD79B, Creb, CSK, DAPP1, EGR1, ELK1, ERK1/2, ETS1, Fcgr2, GAB1/2, GRB2, Gsk3, Ikb, IkB-NfkB, IKK (complex), JINK1/2, Jnkk, JUN, LYN, MALT1, MAP2K1/2, MAP3K, MKK3/4/6, MTOR, NFAT (complex), NFkB (complex), P38 MAPK, p70 S6k, PAG1, phosphatidylinositol-3,4,5-triphosphate, PI3K (complex), PIK3AP1, PKC(β,θ), PLCG2, POU2F2, Pp2b, PTEN, PTPN11, PTPN6, PTPRC, Rac/Cdc42, RAF1, Ras, SHC1 (includes EG:20416), SHIP, Sos, SYK, VAV

### PKCteta Pathway

An effective immune response depends on the ability of specialized leukocytes to identify foreign molecules and respond by differentiation into mature effector cells. A cell surface antigen recognition apparatus and a complex intracellular receptor-coupled signal transducing machinery mediate this tightly regulated process which operates at high fidelity to discriminate self antigens from non-self antigens. Activation of T cells requires sustained physical interaction of the TCR with an MHC-presented peptide antigen that results in a temporal and spatial reorganization of multiple cellular elements at the T-Cell-APC contact region, a specialized region referred to as the immunological synapse or supramolecular activation cluster. Recent studies have identified PKCθ, a member of the Ca-independent PKC family, as an essential component of the T-Cell supramolecular activation cluster that mediates several crucial functions in TCR signaling leading to cell activation, differentiation, and survival through IL-2 gene induction. High levels of PKCθ are expressed in skeletal muscle and lymphoid tissues, predominantly in the thymus and lymph nodes, with lower levels in spleen. T cells constitute the primary location for PKCθ expression. Among T cells, CD4+/CD8+ single positive peripheral blood T cells and CD4+/CD8+ double positive thymocytes are found to express high levels of PKCθ. On the surface of T cells, TCR/CD3 engagement induces activation of Src, Syk, ZAP70 and Tec-family PTKs leading to stimulation and membrane recruitment of PLCγ1, PI3K and Vav. A Vav mediated pathway, which depends on Rac and actin cytoskeleton reorganization as well as on PI3K, is responsible for the selective recruitment of PKCθ to the supramolecular activation cluster. PLCγ1-generated DAG also plays a role in the initial recruitment of PKCθ. The transcription factors NF-κB and AP-1 are the primary physiological targets of PKCθ. Efficient activation of these transcription factors by PKCθ requires integration of TCR and CD28 co-stimulatory signals. CD28 with its CD80/CD86 (B7-1/B7-2) ligands on APCs is required for the recruitment of PKCθ specifically to the supramolecular activation cluster. The transcriptional element which serves as a target for TCR/CD28 costimulation is CD28RE in the IL-2 promoter. CD28RE is a combinatorial binding site for NF-κB and AP-1. Recent studies suggest that regulation of TCR coupling to NF-κB by PKCθ is affected through a variety of distinct mechanisms. PKCθ may directly associate with and regulate the IKK complex; PKCθ may regulate the IKK complex indirectly though CaMKII; It may act upstream of a newly described pathway involving BCL10 and MALT1, which together regulate NF-κB and IκB via the IKK complex. PKCθ has been found to promote Activation-induced T cell death (AICD), an important process that limits the expansion of activated antigen-specific T cells and ensures termination of an immune response once the specific pathogen has been cleared. Enzymatically active PKCθ selectively synergizes with calcineurin to activate a caspase 8-mediated Fas/FasL-dependent AICD. CD28 co-stimulation plays an essential role in TCR-mediated IL-2 production, and in its absence the T cell enters a stable state of unresponsiveness termed anergy. PKCθ-mediated CREB phosphorylation and its subsequent binding to a cAMP-response element in the IL-2 promoter negatively regulates IL-2 transcription thereby driving the responding T cells into an anergic state. The selective expression of PKCθ in T-Cells and its essential role in mature T cell activation establish it as an attractive drug target for immunosuppression in transplantation and autoimmune diseases.

This pathway is composed of, but not restricted to Apt, BCL10, Bcl10-Card11-Malt1, Calcineurin protein(s), CaMKII, CARD11, CD28, CD3, CD3-TCR, CD4, CD80 (includes EG:12519), CD86, diacylglycerol, ERK1/2, FOS, FYN, GRAP2, GRB2, Ikb, IkB-NfkB, Ikk (family), IL2, inositol triphosphate, JUN, LAT, LCK, LCP2, MALT1, MAP2K4, MAP3K, MAPK8, MHC Class II (complex), Nfat (family), NFkB (complex), phorbol myristate acetate, PI3K (complex), PLC gamma, POU2F1, PRKCQ, Rac, Ras, Sos, TCR, VAV, voltage-gated calcium channel, ZAP70

### CD40 Signaling

CD40 is a member of the tumor necrosis factor superfamily of cell surface receptors that transmits survival signals to B cells. Upon ligand binding, canonical signaling evoked by cell-surface CD40 follows a multistep cascade requiring cytoplasmic adaptors (called TNF-receptor-associated factors [TRAFs], which are recruited by CD40 in the lipid rafts) and the IKK complex. Through NF-κB activation, the CD40 signalosome activates transcription of multiple genes involved in B-cell growth and survival. Because the CD40 signalosome is active in aggressive lymphoma and contributes to tumor growth, immunotherapeutie strategies directed against CD40 are being designed and currently tested in clinical trials [Bayes 2007 and Fanale 2007).

CD40-mediated signal transduction induces the transcription of a large number of genes implicated in host defense against pathogens. This is accomplished by the activation of multiple pathways including NF-κB, MAPK and STAT3 which regulate gene expression through activation of c-Jun, ATF2 and Rel transcription factors. Receptor clustering of CD40L is mediated by an association of the ligand with p53, a translocation of ASM to the plasma membrane, activation of ASM, and formation of ceramide. Ceramide serves to cluster CD40L and several TRAF proteins (including TRAF1, TRAF2, TRAF3, TRAF5, and TRAF6) with CD40. TRAF2, TRAF3 and TRAF6 bind to CD40 directly. TRAF1 does not directly bind CD40 but is recruited to membrane micro domains through heterodimerization with TRAF2. Analogous to the recruitment of TRAF1,TRAF5 is also indirectly recruited to CD40 in a TRAF3-dependent manner. Act1 links TRAF proteins to TAK1/IKK to activate NF-κB/I-κB, and MKK complex to activate JNK, p38 MAPK and ERK1/2. NIK also plays a leading role in activating IKK. Act1-dependent CD40-mediated NF-κB activation protects cells from CD40L-induced apoptosis. On stimulation with CD40L or other inflammatory mediators, I-κB proteins are phosphorylated by IKK and NF-κB is activated through the Act1-TAK1 pathway. Phosphorylated I-κB is then rapidly ubiquitinated and degraded. The liberated NF-κB translocates to the nucleus and activates transcription. A20, which is induced by TNF inhibits NF-κB activation as well as TNF-mediated apoptosis. TRAF3 initiates signaling pathways that lead to the activation of p38 and JNK but inhibits Act1-dependent CD40-mediated NF-κB activation and initiates CD40L-induced apoptosis. TRAF2 is required for activation of SAPK pathways and also plays a role in CD40-mediated surface upregulation, IgM secretion in B-Cells and up-regulation of ICAM1. CD40 ligation by CD40L stimulates MCP1 and IL-8 production in primary cultures of human proximal tubule cells, and this occurs primarily via recruitment of TRAF6 and activation of the ERK1/2, SAPK/JNK and p38 MAPK pathways. Activation of SAPK/JNK and p38 MAPK pathways is mediated via TRAF6 whereas ERK1/2 activity is potentially mediated via other TRAF members. However, stimulation of all three MAPK pathways is required for MCP1 and IL-8 production. Other pathways activated by CD40 stimulation include the JAK3-STAT3 and PI3K-Akt pathways, which contribute to the anti-apoptotic properties conferred by CD40L to B-Cells. CD40 directly binds to JAK3 and mediates STAT3 activation followed by up-regulation of ICAM1, CD23, and LT-α.

This pathway is composed of, but not restricted to Act1, Apt, ATF1 (includes EG:100040260), CD40, CD40LG, ERK1/2, FCER2, I kappa b kinase, ICAM1, Ikb, IkB-NfkB, JAK3, Jnk, LTA, MAP3K14, MAP3K7 (includes EG:172842), MAPKAPK2, Mek, NFkB (complex), P38 MAPK, PI3K (complex), STAT3, Stat3-Stat3, TANK, TNFAIP3, TRAF1, TRAF2, TRAF3, TRAF5, TRAF6

### CD28 Signaling Pathway

CD28 is a co-receptor for the TCR/CD3 and is a major positive co-stimulatory molecule. Upon ligation with CD80 and CD86, CTLA4 provides a negative co-stimulatory signal for the termination of activation. Further binding of CD28 to Class-I regulatory PI3K recruits PI3K to the membrane, resulting in generation of PIP3 and recruitment of proteins that contain a pleckstrin-homology domain to the plasma membrane, such as PIK3C3. PI3K is required for activation of Akt, which in turn regulates many downstream targets that to promote cell survival. In addition to NFAT, NF-κB has a crucial role in the regulation of transcription of the IL-2 promoter and anti-apoptotic factors. For this, PLC-γ utilizes PIP2 as a substrate to generate IP3 and DAG. IP3 elicits release of Ca2+ via IP3R, and DAG activates PKC-θ. Under the influence of RLK, PLC-γ, and Ca2+; PKC-θ regulates the phosphorylation state of IKK complex through direct as well as indirect interactions. Moreover, activation of CARMA1 phosphorylates BCL10 and dimerizes MALT1, an event that is sufficient for the activation of IKKs. The two CD28-responsive elements in the IL-2 promoter have NF-κB binding sites. NF-κB dimers are normally retained in cytoplasm by binding to inhibitory I-κBs. Phosphorylation of I-κBs initiates its ubiquitination and degradation, thereby freeing NF-κB to translocate to the nucleus. Likewise, translocation of NFAT to the nucleus as a result of calmodulin-calcineurin interaction effectively promotes IL-2 expression. Activation of Vav1 by TCR-CD28-PI3K signaling connects CD28 with the activation of Rac and CDC42, and this enhances TCR-CD3-CD28 mediated cytoskeletal re-organization. Rac regulates actin polymerization to drive lamellipodial protrusion and membrane ruffling, whereas CDC42 generates polarity and induces formation of filopodia and microspikes. CDC42 and Rac GTPases function sequentially to activate downstream effectors like WASP and PAK1 to induce activation of ARPs resulting in cytoskeletal rearrangements. CD28 impinges on the Rac/PAK1-mediated IL-2 transcription through subsequent activation of MEKK1, MKKs and JNKs. JNKs phosphorylate and activate c-Jun and c-Fos, which is essential for transcription of IL-2. Signaling through CD28 promotes cytokine IL-2 mRNA production and entry into the cell cycle, T-cell survival, T-Helper cell differentiation and Immunoglobulin isotype switching.

This pathway is composed of, but not restricted to 1,4,5-IP3, 1-phosphatidyl-D-myo-inositol 4,5-bisphosphate, Akt, Ap1, Arp2/3, BCL10, Ca2+, Calcineurin protein(s), Calmodulin, CARD11, CD28, CD3, CD3-TCR, CD4, CD80 (includes EG:12519), CD86, CDC42, CSK, CTLA4, diacylglycerol, FOS, FYN, GRAP2, GRB2, Ikb, IkB-NfkB, IKK (complex), IL2, ITK, ITPR, Jnk, JUN, LAT, LCK, LCP2, MALT1, MAP2K1/2, MAP3K1, MHC Class II (complex), Nfat (family), NFkB (complex), PAK1, PDPK1, phosphatidylinositol-3,4,5-triphosphate, PI3K (complex), PLCG1, PRKCQ, PTPRC, RAC1, SHP, SYK, TCR, VAV1, WAS, ZAP70

### ERK-MAPK Pathway

The ERK (extracellular-regulated kinase)/MAPK (mitogen activated protein kinase) pathway is a key pathway that transduces cellular information on meiosis/mitosis, growth, differentiation and carcinogenesis within a cell. Membrane bound receptor tyrosine kinases (RTK), which are often growth factor receptors, are the starting point for this pathway. Binding of ligand to RTK activates the intrinsic tyrosine kinase activity of RTK. Adaptor molecules like growth factor receptor bound protein 2 (GRB2), son of sevenless (SOS) and Shc form a signaling complex on tyrosine phosphorylated RTK and activate Ras. Activated Ras initiates a kinase cascade, beginning with Raf (a MAPK kinase kinase) which activates and phosphorylates MEK (a MAPK kinase); MEK activates and phosphorylates ERK (a MAPK). ERK in the cytoplasm can phosphorylate a variety of targets which include cytoskeleton proteins, ion channels/receptors and translation regulators. ERK is also translocated across into the nucleus where it induces gene transcription by interacting with transcriptional regulators like ELK-1, STAT-1 and -3, ETS and MYC. ERK activation of p90RSK in the cytoplasm leads to its nuclear translocation where it indirectly induces gene transcription through interaction with transcriptional regulators, CREB, c-Fos and SRF. RTK activation of Ras and Raf sometimes takes alternate pathways. For example, integrins activate ERK via a FAK mediated pathway. ERK can also be activated by a CAS-CRK-Rap 1 mediated activation of B-Raf and a PLCγ-PKC-Ras-Raf activation of ERK.

This pathway is be composed of, but not restricted to 1,4,5-IP3, 1-phosphatidyl-D-myo-inositol 4,5-bisphosphate, 14-3-3(β,γ,θ,η,ζ), 14-3-3(η,θ,ζ), ARAF, ATF1 (includes EG:100040260), BAD, BCAR1, BRAF, c-Myc/N-Myc, cAMP-Gef, CAS-Crk-DOCK 180, Cpla2, Creb, CRK/CRKL, cyclic AMP, diacylglycerol, DOCK1, DUSP2, EIF4E, EIF4EBP1, ELK1, ERK1/2, Erk1/2 dimer, ESR1, ETS, FOS, FYN, GRB2, Histone h3, Hsp27, Integrin, KSR1, LAMTOR3, MAP2K1/2, MAPKAPK5, MKP1/2/3/4, MNK1/2, MOS, MSK1/2, NFATC1, Pak, PI3K (complex), Pka, PKC (α,β,γ,δ,ε,t), PLC gamma, PP1/PP2A, PPARG, PTK2 (includes EG:14083), PTK2B (includes EG:19229), PXN, Rac, RAFT, Rap1, RAPGEF1, Ras, RPS6KA1 (includes EG:20111), SHC1 (includes EG:20416), Sos, SRC, SRF, Stat1/3, Talin, VRK2

Based on the findings by the method described here in the DLBCL OCI-LY1, combination of an inhibitor of components of these pathways, such as those targeting but not limited to SYK, BTK, mTOR, PI3K, Ikk, CD40, MEK, Raf, JAK, the MHC complex components, CD80, CD3 are proposed to be efficacious when used in combination with an Hsp90 inhibitor.

Examples of BTK inhibitors are PCI-32765

Examples of SYK inhibitors are R-406, R406, R935788 (Fostamatinib disodium)

Examples of CD40 inhibitors are SGN-40 (anti-huCD40 mAb)

Examples of inhibitors of the CD28 pathway are abatacept, belatacept, blinatumomab, muromonab-CD3, visilizumab.

Example of inhibitors of major histocompatibility complex, class II are apolizumab

Example of PI3K inhibitors are 2-(1H-indazol-4-yl)-6-(4-methanesulfonylpiperazin-1-ylmethyl)-4-morpholin-4-ylthieno(3,2-d)pyrimidine, BKM120, NVP-BEZ235, PX-866, SF 1126, XL147.

Example of mTOR inhibitors are deforolimus, everolimus, NVP-BEZ235, OSI-027, tacrolimus, temsirolimus, Ku-0063794, WYE-354, PP242, OSI-027, GSK2126458, WAY-600, WYE-125132

Examples of JAK inhibitors are Tofacitinib citrate (CP-690550), AT9283, AG-490, INCB018424 (Ruxolitinib), AZD1480, LY2784544, NVP-BSK805, TG101209, TG-101348

Examples of IkK inhibitors are SC-514, PF 184

Example of inhibitors of Raf are sorafenib, vemurafenib, GDC-0879, PLX-4720, PLX4032 (Vemurafenib), NVP-BHG712, SB590885, AZ628, ZM 336372

Example of inhibitors of SRC are AZM-475271, dasatinib, saracatinib

In the MiaPaCa2 pancreatic cancer cell line major signaling networks identified by the method were the PI3K/AKT, IGF1, cell cycle-G2/M DNA damage checkpoint regulation, ERK/MAPK and the PKA signaling pathways (FIG. 24).

Interactions between the several network component proteins are exemplified in FIG. 16.

Pancreatic adenocarcinoma continues to be one of the most lethal cancers, representing the fourth leading cause of cancer deaths in the United States. More than 80% of patients present with advanced disease at diagnosis and therefore, are not candidates for potentially curative surgical resection. Gemcitabine-based chemotherapy remains the main treatment of locally advanced or metastatic pancreatic adenocarcinoma since a pivotal Phase III trial in 1997. Although treatment with gemcitabine does achieve significant symptom control in patients with advanced pancreatic cancer, its response rates still remain low and is associated with a median survival of approximately 6 months. These results reflect the inadequacy of existing treatment strategies for this tumor type, and a concerted effort is required to develop new and more effective therapies for patients with a pancreatic cancer.

A current review of Pub Med. literature, clinical trial database (clinicaltrials.gov), American Society of Clinical Oncology (ASCO) and American Association of Cancer Research (AACR) websites, concluded that the molecular pathogenesis of a pancreatic cancer involves multiple pathways and defined mutations, suggesting this complexity as a major reason for failure of targeted therapy in this disease. Faced with a complex mechanism of activating oncogenic pathways that regulate cellular proliferation, survival and metastasis, therapies that target a single activating molecule cannot thus, overpower the multitude of aberrant cellular processes, and may be of limited therapeutic benefit in advanced disease.

Based on the findings by the method described here in MiaPaCa2 cells, combination of an inhibitor of components of these identified pathways, such as those targeting but not limited to AKT, mTOR, PI3K, JAK, STAT3, IKK, Bcl2, PKA complex, phosphodiesterases, ERK, Raf, JNK are proposed to be efficacious when used in combination with an Hsp90 inhibitor.

Example of AKT inhibitors are PF-04691502, Triciribine phosphate (NSC-280594), A-674563, CCT128930, AT7867, PHT-427, GSK690693, MK-2206 dihydrochloride

Example of PI3K inhibitors are 2-(1H-indazol-4-yl)-6-(4-methanesulfonylpiperazin-1-ylmethyl)-4-morpholin-4-ylthieno(3,2-d)pyrimidine, BKM120, NVP-BEZ235, PX-866, SF 1126, XL147.

Example of mTOR inhibitors are deforolimus, everolimus, NVP-BEZ235, OSI-027, tacrolimus, temsirolimus, Ku-0063794, WYE-354, PP242, OSI-027, GSK2126458, WAY-600, WYE-125132

Examples of Bcl2 inhibitors are ABT-737, Obatoclax (GX15-070), ABT-263, TW-37

Examples of JAK inhibitors are Tofacitinib citrate (CP-690550), AT9283, AG-490, INCB018424 (Ruxolitinib), AZD1480, LY2784544, NVP-BSK805, TG101209, TG-101348

Examples of IkK inhibitors are SC-514, PF 184

Examples of inhibitors of phosphodiesterases are aminophylline, anagrelide, arofylline, caffeine, cilomilast, dipyridamole, dyphylline, L 869298, L-826,141, milrinone, nitroglycerin, pentoxifylline, roflumilast, rolipram, tetomilast, theophylline, tolbutamide, amrinone, anagrelide, arofylline, caffeine, cilomilast, L 869298, L-826,141, milrinone, pentoxifylline, roflumilast, rolipram, tetomilast

Indeed, inhibitors of mTOR, which is identified by our method to potentially contribute to the transformation of MiaPaCa2 cells (FIG. 7e), are active as single agents (FIG. 7f) and synergize with Hsp90 inhibition in affecting the growth of these pancreatic cancer cells (FIG. 17).

Quantitative analysis of synergy between mTOR and Hsp90 inhibitors: To determine the drug interaction between pp242 (mTOR inhibitor) and PU-H71 (Hsp90 inhibitor), the combination index (CI) isobologram method of Chou-Talalay was used as previously described. This method, based on the median-effect principle of the law of mass action, quantifies synergism or antagonism for two or more drug combinations, regardless of the mechanisms of each drug, by computerized simulation. Based on algorithms, the computer software displays median-effect plots, combination index plots and normalized isobolograms (where non constant ratio combinations of 2 drugs are used). PU-H71 (0.5, 0.25, 0.125, 0.0625, 0.03125, 0.0125 μM) and pp242 (0.5, 0.125, 0.03125, 0.0008, 0.002, 0.001 μM) were used as single agents in the concentrations mentioned or combined in a non constant ratio (PU-H71: pp242; 1:1, 1:2, 1:4, 1:7.8, 1:15.6, 1:12.5). The Fa (fraction killed cells) was calculated using the formulae Fa=1−Fu; Fu is the fraction of unaffected cells and was used for a dose effect analysis using the computer software (CompuSyn, Paramus, N.J., USA).

In a similar fashion, inhibitors of the PI3K-AKT-mTOR pathway which is identified by our method to contribute to the transformation of MDA-MB-468 cells, are more efficacious in the MDA-MB-468 breast cancer cells when combined with the Hsp90 inhibitor.

### Cell Cycle: G2/M DNA Damage Checkpoint Regulation

G2/M checkpoint is the second checkpoint within the cell cycle. This checkpoint prevents cells with damaged DNA from entering the M phase, while also pausing so that DNA repair can occur. This regulation is important to maintain genomic stability and prevent cells from undergoing malignant transformation. Ataxia telangiectasia mutated (ATM) and ataxia telangiectasia mutated and rad3 related (ATR) are key kinases that respond to DNA damage. ATR responds to UV damage, while ATM responds to DNA double-strand breaks (DSB). ATM and ATR activate kinases Chk1 and Chk2 which in turn inhibit Cdc25, the phosphatase that normally activates Cdc2. Cdc2, a cyclin-dependent kinase, is a key molecule that is required for entry into M phase. It requires binding to cyclin B1 for its activity. The tumor suppressor gene p53 is an important molecule in G2/M checkpoint regulation. ATM, ATR and Chk2 contribute to the activation of p53. Further, p19Arf functions mechanistically to prevent MDM2's neutralization of p53. Mdm4 is a transcriptional inhibitor of p53. DNA damage-induced phosphorylation of Mdm4 activates p53 by targeting Mdm4 for degradation. Well known p53 target genes like Gadd45 and p21 are involved in inhibiting Cdc2. Another p53 target gene, 14-3-3σ, binds to the Cdc2-cyclin B complex rendering it inactive. Repression of the cyclin B1 gene by p53 also contributes to blocking entry into mitosis. In this way, numerous checks are enforced before a cell is allowed to enter the M phase.

This pathway is composed of, but not limited to 14-3-3, 14-3-3 (β,ε,ζ), 14-3-3-Cdc25, ATM, ATM/ATR, BRCA1, Cdc2-CyclinB, Cdc2-CyclinB-Sfn, Cdc25B/C, CDK1, CDK7, CDKN1A, CDKN2A, Cdkn2a-Mdm2, CHEK1, CHEK2, CKS1B, CKS2, Cyclin B, EP300, Ep300/Pcaf, GADD45A, KAT2B, MDM2, Mdm2-Tp53-Mdm4, MDM4, PKMYT1, PLK1, PRKDC, RPRM, RPS6KA1 (includes EG:20111), Scf, SFN, Top2, TP53 (includes EG:22059), WEE1

Based on the findings by the method described here, combination of an inhibitor of components of this pathway, such as those targeting CDK1, CDK7, CHEK1, PLK1 and TOP2A(B) are proposed to be efficacious when used in combination with an Hsp90 inhibitor.

Examples of inhibitors are AQ4N, becatecarin, BN 80927, CPI-0004Na, daunorubicin, dexrazoxane, doxorubicin, elsamitrucin, epirubicin, etoposide, gatifloxacin, gemifloxacin, mitoxantrone, nalidixic acid, nemorubicin, norfloxacin, novobiocin, pixantrone, tafluposide, TAS-103, tirapazamine, valrubicin, XK469, BI2536

PU-beads also identify proteins of the DNA damage, replication and repair, homologous recombination and cellular response to ionizing radiation as Hsp90-regulated pathways in select CML, pancreatic cancer and breast cancer cells. PU-H71 synergized with agents that act on these pathways.

Specifically, among the Hsp90-regulated pathways identified in the K562 CML cells, MDA-MB-468 breast cancer cells and the Mia-PaCa-2 pancreatic cancer cells are several involved in DNA damage, replication and repair response and/or homologous recombination (Tables 3, 5a-5f). Hsp90 inhibition may synergize or be additive with agents that act on DNA damage and/or homologous recombination (i.e. potentiate DNA damage sustained post treatment with IR/chemotherapy or other agents, such as PARP inhibitors that act on the proteins that are important for the repair of double-strand DNA breaks by the error-free homologous recombinational repair pathway). Indeed, we found that PU-H71 radiosensitized the Mia-PaCa-2 human pancreatic cancer cells. We also found PU-H71 to synergize with the PARP inhibitor olaparib in the MDA-MB-468 and HCC1937 breast cancer cells (FIG. 25).

Identification of Hsp90 clients required for tumor cell survival may also serve as tumor-specific biomarkers for selection of patients likely to benefit from Hsp90 therapy and for pharmacodynamic monitoring of Hsp90 inhibitor efficacy during clinical trials (i.e. clients in FIG. 6, 20 whose expression or phosphorylation changes upon Hsp90 inhibition). Tumor specific Hsp90 client profiling could ultimately yield an approach for personalized therapeutic targeting of tumors (FIG. 9).

This work substantiates and significantly extends the work of Kamal et al, providing a more sophisticated understanding of the original model in which Hsp90 in tumors is described as present entirely in multi-chaperone complexes, whereas Hsp90 from normal tissues exists in a latent, uncomplexed state (Kamal et al., 2003). We propose that Hsp90 forms biochemically distinct complexes in cancer cells (FIG. 11a). In this view, a major fraction of cancer cell Hsp90 retains “house keeping” chaperone functions similar to normal cells, whereas a functionally distinct Hsp90 pool enriched or expanded in cancer cells specifically interacts with oncogenic proteins required to maintain tumor cell survival. Perhaps this Hsp90 fraction represents a cell stress specific form of chaperone complex that is expanded and constitutively maintained in the tumor cell context. Our data suggest that it may execute functions necessary to maintain the malignant phenotype. One such role is to regulate the folding of mutated (i.e. mB-Raf) or chimeric proteins (i.e. Bcr-Abl) (Zuehlke & Johnson, 2010; Workman et al, 2007). We now present experimental evidence for an additional role; that is, to facilitate scaffolding and complex formation of molecules involved in aberrantly activated signaling complexes. Herein we describe such a role for Hsp90 in maintaining constitutive STAT5 signaling in CML (FIG. 8h). These data are consistent with previous work in which we showed that Hsp90 was required to maintain functional transcriptional repression complexes by the BCL6 oncogenic transcriptional repressor in B cell lymphoma cells (Cerchietti et al., 2009).

In sum, our work uses chemical tools to provide new insights into the heterogeneity of tumor associated Hsp90 and harnesses the biochemical features of a particular Hsp90 inhibitor to identify tumor-specific biological pathways and proteins (FIG. 9). We believe the functional proteomics method described here will allow identification of the critical proteome subset that becomes dysregulated in distinct tumors. This will allow for the identification of new cancer mechanisms, as exemplified by the STAT mechanism described herein, the identification of new onco-proteins, as exemplified by CARM1 described herein, and the identification of therapeutic targets for the development of rationally combined targeted therapies complementary to Hsp90.

### Materials and Methods

### Cell Lines and Primary Cells

The CML cell lines K562, Kasumi-4, MEG-01 and KU182, triple-negative breast cancer cell line MDA-MB-468, HER2+ breast cancer cell line SKBr3, melanoma cell line SK-Mel-28, prostate cancer cell lines LNCaP and DU145, pancreatic cancer cell line Mia-PaCa-2, colon fibroblast, CCCD18Co cell lines were obtained from the American Type Culture Collection. The CML cell line KCL-22 was obtained from the Japanese Collection of Research Bioresources. The NIH-3T3 fibroblast cells were transfected as previously described (An et al., 2000). Cells were cultured in DMEM/F12 (MDA-MB-468, SKBr3 and Mia-PaCa-2), RPMI (K562, SK-Mel-28, LNCaP, DU145 and NIH-3T3) or MEM (CCD18Co) supplemented with 10% FBS, 1% L-glutamine, 1% penicillin and streptomycin. Kasumi-4 cells were maintained in IMDM supplemented with 20% FBS, 10 ng/ml Granulocyte macrophage colony-stimulating factor (GM-CSF) and 1× Pen/Strep. PBL (human peripheral blood leukocytes) and cord blood were obtained from patient blood purchased from the New York Blood Center. Thirty five ml of the cell suspension was layered over 15 ml of Ficoll-Paque plus (GE Healthcare). Samples were centrifuged at 2,000 rpm for 40 min at 4° C., and the leukocyte interface was collected. Cells were plated in RPMI medium with 10% FBS and used as indicated. Primary human blast crisis CML and AML cells were obtained with informed consent. The manipulation and analysis of specimens was approved by the University of Rochester, Weill Cornell Medical College and University of Pennsylvania Institutional Review Boards. Mononuclear cells were isolated using Ficoll-Plaque (Pharmacia Biotech, Piscataway, N.Y.) density gradient separation. Cells were cryopreserved in freezing medium consisting of Iscove's modified Dulbecco medium (IMDM), 40% fetal bovine serum (FBS), and 10% dimethylsulfoxide (DMSO) or in CryoStor™ CS-10 (Biolife). When cultured, cells were kept in a humidified atmosphere of 5% CO₂ at 37° C.

### Cell Lysis for Chemical and Immuno Precipitation

Cells were lysed by collecting them in Felts Buffer (HEPES 20 mM, KCl 50 mM, MgCl₂ 5 mM, NP40 0.01%, freshly prepared Na₂MoO₄ 20 mM, pH 7.2-7.3) with added 1 μg/μL of protease inhibitors (leupeptin and aprotinin), followed by three successive freeze (in dry ice) and thaw steps. Total protein concentration was determined using the BCA kit (Pierce) according to the manufacturer's instructions.

### Immunoprecipitation

The Hsp90 antibody (H9010) or normal IgG (Santa Cruz Biotechnology) was added at a volume of 10 μL to the indicated amount of cell lysate together with 40 μL of protein G agarose beads (Upstate), and the mixture incubated at 4° C. overnight. The beads were washed five times with Felts lysis buffer and separated by SDS-PAGE, followed by a standard western blotting procedure.

### Chemical Precipitation

Hsp90 inhibitors beads or Control beads, containing an Hsp90 inactive chemical (ethanolamine) conjugated to agarose beads, were washed three times in lysis buffer. Unless otherwise indicated, the bead conjugates (80 μL) were then incubated at 4° C. with the indicated amounts of cell lysates (120-500 μg), and the volume was adjusted to 200 μL with lysis buffer. Following incubation, bead conjugates were washed 5 times with the lysis buffer and proteins in the pull-down analyzed by Western blot. For depletion studies, 2-4 successive chemical precipitations were performed, followed by immunoprecipitation steps, where indicated.

Additional methods are also described herein at pages 173-183.

### Supplementary Materials

### Table 5 Legend

Table 5. (a-d) List of proteins isolated in the PU-beads pull-downs and identified as indicated in Supplementary Materials and Methods. (e) Dataset of mapped proteins used for analysis in the Ingenuity Pathway. (f) Protein regulatory networks generated by bioinformatic pathways analysis through the use of the Ingenuity Pathways Analysis (IPA) software. Proteins listed in Table 5e were analyzed by IPA.

TABLE 5a
Putative Hsp90 interacting proteins identified using the QSTAR-Elite hybrid
quadrupole time-of-flight mass spectrometer (QT of MS) (AB/MDS Sciex)
#GChiosis_K562 and MiPaca2_All, Samples Report created on Aug. 05, 2010
GChiosis_K562 and MiPaca2_All
Displaying: Number of Assigned Spectra

| Entrez- | UniProt- | | Accession | Molecular | K562 | K562 | Mia- |
|---|---|---|---|---|---|---|---|
| Gene | KB | | Number | Weight | Prep 1 | Prep 2 | Paca 2 |
| HSP90AA1 | P07900 | heat shock 90 kDa protein | IPI00382470 | 98 kDa | 563 | 2018 | 1514 |
| | | 1, alpha isoform 1 | (+1) | | | | |
| HSP90AB1 | P08238 | Heat shock protein HSP 90- | IPI00414676 | 83 kDa | 300 | 1208 | 578 |
| | | beta | | | | | |
| ABL1 | P00519 | Isoform IA of Proto- | IPI00216969 | 123 kDa | 3 | 4 | 0 |
| | | oncogene tyrosine-protein | (+1) | | | | |
| | | kinase ABL1 | | | | | |
| BCR | P11274 | Isoform 1 of Breakpoint | IPI00004497 | 143 kDa | 1 | 4 | 0 |
| | | cluster region protein | (+1) | | | | |
| RPS6KA3 | P51812 | Ribosomal protein S6 | IPI00020898 | 84 kDa | 13 | 10 | 3 |
| | | kinase alpha-3 | | | | | |
| RPS6KA1 | Q15418 | Ribosomal protein S6 | IPI00017305 | 83 kDa | 6 | 1 | 0 |
| | | kinase alpha-1 | (+1) | | | | |
| MTOR; | P42345 | FKBP12-rapamycin | IPI00031410 | 289 kDa | 43 | 14 | 13 |
| FRAP | | complex-associated protein | | | | | |
| RPTOR | Q8N122 | Isoform 1 of Regulatory- | IPI00166044 | 149 kDa | 7 | 3 | 2 |
| | | associated protein of | | | | | |
| | | mTOR | | | | | |
| PIK3R4; | Q99570 | Phosphoinositide 3-kinase | IPI00024006 | 153 kDa | 8 | 9 | 4 |
| VPS15 | | regulatory subunit 4 | | | | | |
| hVps34; | Q8NEB9 | Phosphatidylinositol 3- | IPI00299755 | 102 kDa | 5 | 1 | 1 |
| PIK3C3 | | kinase catalytic subunit | (+1) | | | | |
| | | type 3 | | | | | |
| Sin1; | Q9BPZ7 | Isoform 1 of Target of | IPI00028195 | 59 kDa | 2 | 0 | 0 |
| MAPKAP1 | | rapamycin complex 2 | (+4) | | | | |
| | | subunit MAPKAP1 | | | | | |
| STAT5A | P42229 | Signal transducer and | IPI00030783 | 91 kDa | 48 | 25 | 0 |
| | | activator of transcription 5A | | | | | |
| STAT5B | P51692 | Signal transducer and | IPI00103415 | 90 kDa | 10 | 5 | 0 |
| | | activator of transcription 5B | | | | | |
| RAF1 | P04049 | Isoform 1 of RAF proto- | IPI00021786 | 73 kDa | 5 | 1 | 1 |
| | | oncogene serine/threonine- | | | | | |
| | | protein kinase | | | | | |
| ARAF | P10398 | A-Raf proto-oncogene | IPI00020578 | 68 kDa | 2 | 0 | 1 |
| | | serine/threonine-protein | (+1) | | | | |
| | | kinase | | | | | |
| VAV1 | P15498 | Proto-oncogene vav | IPI00011696 | 98 kDa | 3 | 1 | 0 |
| BTK | Q06187 | Tyrosine-protein kinase | IPI00029132 | 76 kDa | 11 | 8 | 0 |
| | | BTK | | | | | |
| PTK2; | Q05397 | Isoform 1 of Focal adhesion | IPI00012885 | 119 kDa | 4 | 5 | 4 |
| FAK1 | | kinase 1 | (+1) | | | | |
| PTPN23 | Q9H3S7 | Tyrosine-protein | IPI00034006 | 179 kDa | 8 | 8 | 2 |
| | | phosphatase non-receptor | | | | | |
| | | type 23 | | | | | |
| STAT3 | P40763 | Isoform Del-701 of Signal | IPI00306436 | 88 kDa | 15 | 4 | 6 |
| | | transducer and activator of | (+2) | | | | |
| | | transcription 3 | | | | | |
| IRAK1 | P51617 | interleukin-1 receptor- | IPI00060149 | 68 kDa | 7 | 2 | 1 |
| | | associated kinase 1 isoform 3 | (+3) | | | | |
| MAPK1; | P28482 | Mitogen-activated protein | IPI00003479 | 41 kDa | 23 | 5 | 14 |
| ERK2 | | kinase 1, ERK2 | | | | | |
| MAP3K4; | Q9Y6R4 | Isoform A of Mitogen- | IPI00186536 | 182 kDa | 3 | 7 | 0 |
| MEKK4 | | activated protein kinase | (+2) | | | | |
| | | kinase kinase 4 | | | | | |
| TAB1 | Q15750 | Mitogen-activated protein | IPI00019459 | 55 kDa | 1 | 3 | 2 |
| | | kinase kinase kinase 7- | (+1) | | | | |
| | | interacting protein 1 | | | | | |
| MAPK14; | Q16539 | Isoform CSBP2 of Mitogen- | IPI00002857 | 41 kDa | 1 | 0 | 0 |
| p38 | | activated protein kinase 14 | (+1) | | | | |
| MAP2K3; | P46734 | Isoform 3 of Dual specificity | IPI00220438 | 39 kDa | 0 | 0 | 2 |
| MEK3 | | mitogen-activated protein | | | | | |
| | | kinase kinase 3 | | | | | |
| CAPN1 | P07384 | Calpain-1 catalytic subunit | IPI00011285 | 82 kDa | 10 | 11 | 0 |
| IGF2BP2 | O00425 | Isoform 1 of Insulin-like | IPI00658000 | 64 kDa | 18 | 14 | 20 |
| | | growth factor 2 mRNA- | | | | | |
| | | binding protein 3 | | | | | |
| IGF2BP1 | O88477 | Insulin-like growth factor 2 | IPI00008557 | 63 kDa | 11 | 19 | 0 |
| | | mRNA-binding protein 1 | | | | | |
| CAPNS1 | P04632 | Calpain small subunit 1 | IPI00025084 | 28 kDa | 0 | 0 | 3 |
| RUVBL1 | Q9Y265 | Isoform 1 of RuvB-like 1 | IPI00021187 | 50 kDa | 10 | 17 | 30 |
| RUVBL2 | Q9Y230 | RuvB-like 2 | IPI00009104 | 51 kDa | 20 | 30 | 26 |
| MYCBP | Q99417 | MYCBP protein | IPI00871174 | 14 kDa | 2 | 0 | 3 |
| AKAP8 | O43823 | A-kinase anchor protein 8 | IPI00014474 | 76 kDa | 4 | 0 | 0 |
| AKAP8L | Q9ULX6 | A-kinase anchor protein 8- | IPI00297455 | 72 kDa | 3 | 3 | 2 |
| | | like | | | | | |
| NPM1 | P06748 | Isoform 2 of | IPI00220740 | 29 kDa | 8 | 4 | 49 |
| | | Nucleophosmin | (+1) | | | | |
| CARM1 | Q86X55 | Isoform 1 of Histone- | IPI00412880 | 63 kDa | 12 | 16 | 9 |
| | | arginine methyltransferase | (+1) | | | | |
| | | CARM1 | | | | | |
| CALM | P62158 | Calmodulin | IPI00075248 | 17 kDa | 0 | 0 | 34 |
| CAMK1 | Q14012 | Calcium/calmodulin- | IPI00028296 | 41 kDa | 0 | 0 | 3 |
| | | dependent protein kinase | | | | | |
| | | type 1 | | | | | |
| CAMK2G | Q13555 | Isoform 4 of | IPI00172450 | 60 kDa | 2 | 3 | 0 |
| | | Calcium/calmodulin- | (+11) | | | | |
| | | dependent protein kinase | | | | | |
| | | type II gamma chain | | | | | |
| TYK2 | P29597 | Non-receptor tyrosine- | IPI00022353 | 134 kDa | 2 | 0 | 0 |
| | | protein kinase TYK2 | | | | | |
| TBK1 | Q9UHD2 | Serine/threonine-protein | IPI00293613 | 84 kDa | 10 | 0 | 0 |
| | | kinase TBK1 | | | | | |
| PI4KA | P42356 | Isoform 1 of | IPI00070943 | 231 kDa | 15 | 4 | 0 |
| | | Phosphatidylinositol 4- | | | | | |
| | | kinase alpha | | | | | |
| SMG1 | Q96Q15 | Isoform 3 of | IPI00183368 | 341 kDa | 1 | 9 | 0 |
| | | Serine/threonine-protein | (+5) | | | | |
| | | kinase SMG1 | | | | | |
| PHKB | Q93100 | Isoform 4 of Phosphorylase | IPI00181893 | 124 kDa | 10 | 3 | 9 |
| | | b kinase regulatory subunit | (+1) | | | | |
| | | beta | | | | | |
| PANK4 | Q9NVE7 | cDNA FLJ56439, highly | IPI00018946 | 87 kDa | 7 | 7 | 0 |
| | | similar to Pantothenate | | | | | |
| | | kinase 4 | | | | | |
| PRKACA | P17612 | Isoform 2 of cAMP- | IPI00217960 | 40 kDa | 0 | 0 | 4 |
| | | dependent protein kinase | (+1) | | | | |
| | | catalytic subunit alpha, PKA | | | | | |
| PRKAA1 | Q13131 | protein kinase, AMP- | IPI00410287 | 66 kDa | 11 | 6 | 1 |
| | | activated, alpha 1 catalytic | (+3) | | | | |
| | | subunit isoform 2 | | | | | |
| PRKAG1 | Q8N7V9 | cDNA FLJ40287 fis, clone | IPI00473047 | 39 kDa | 10 | 0 | 1 |
| | | TESTI2027909, highly | (+1) | | | | |
| | | similar to 5′-AMP- | | | | | |
| | | ACTIVATED PROTEIN | | | | | |
| | | KINASE, GAMMA-1 | | | | | |
| | | SUBUNIT | | | | | |
| SCYL1 | Q96KG9 | Isoform 4 of N-terminal | IPI00062264 | 86 kDa | 8 | 2 | 0 |
| | | kinase-like protein | (+5) | | | | |
| ATM | Q13315 | Serine-protein kinase ATM | IPI00298306 | 351 kDa | 2 | 4 | 1 |
| ATR | Q13535 | Isoform 1 of | IPI00412298 | 301 kDa | 5 | 0 | 3 |
| | | Serine/threonine-protein | (+1) | | | | |
| | | kinase ATR | | | | | |
| STRAP | Q9Y3F4 | cDNA FLJ51909, highly | IPI00294536 | 40 kDa | 13 | 0 | 4 |
| | | similar to Serine-threonine | | | | | |
| | | kinase receptor-associated | | | | | |
| | | protein | | | | | |
| RIOK2 | Q9BVS4 | Serine/threonine-protein | IPI00306406 | 63 kDa | 7 | 6 | 1 |
| | | kinase RIO2 | | | | | |
| PRKD2 | Q9BZL6 | cDNA FLJ60070, highly | IPI00009334 | 98 kDa | 4 | 0 | 0 |
| | | similar to Serine/threonine- | (+1) | | | | |
| | | protein kinase D2 | | | | | |
| CSNK1A1 | P48729 | Isoform 2 of Casein kinase I | IPI00448798 | 42 kDa | 5 | 0 | 1 |
| | | isoform alpha | | | | | |
| CSNK2B | P67870 | Casein kinase II subunit | IPI00010865 | 25 kDa | 1 | 0 | 1 |
| | | beta | (+1) | | | | |
| KSR1 | Q8IVT5 | Isoform 2 of Kinase | IPI00013384 | 97 kDa | 3 | 0 | 0 |
| | | suppressor of Ras 1 | (+1) | | | | |
| BMP2K | Q9NSY1 | Isoform 1 of BMP-2- | IPI00337426 | 129 kDa | 4 | 3 | 0 |
| | | inducible protein kinase | | | | | |
| SRPK1 | Q96SB4 | Isoform 2 of | IPI00290439 | 74 kDa | 11 | 2 | 7 |
| | | Serine/threonine-protein | (+1) | | | | |
| | | kinase SRPK1 | | | | | |
| SRPK2 | P78362 | Serine/threonine-protein | IPI00333420 | 78 kDa | 1 | 1 | 0 |
| | | kinase SRPK2 | (+3) | | | | |
| PLK1 | P53350 | Serine/threonine-protein | IPI00021248 | 68 kDa | 3 | 0 | 0 |
| | | kinase PLK1 | (+1) | | | | |
| CDK7 | P50613 | Cell division protein kinase 7 | IPI00000685 | 39 kDa | 2 | 0 | 1 |
| CDK12 | Q9NYV4 | Isoform 1 of Cell division | IPI00021175 | 164 kDa | 0 | 0 | 3 |
| | | cycle 2-related protein | (+1) | | | | |
| | | kinase 7 | | | | | |
| CCAR1 | Q8IX12 | Cell division cycle and | IPI00217357 | 133 kDa | 3 | 0 | 0 |
| | | apoptosis regulator protein 1 | | | | | |
| CDC27 | P30260 | Cell division cycle protein | IPI00294575 | 92 kDa | 7 | 2 | 1 |
| | | 27 homolog | (+1) | | | | |
| CDC23 | Q9UJX2 | cell division cycle protein 23 | IPI00005822 | 69 kDa | 1 | 4 | 4 |
| CDK9 | P50750 | Isoform 1 of Cell division | IPI00301923 | 43 kDa | 3 | 0 | 1 |
| | | protein kinase 9 | (+1) | | | | |
| BUB1B | O60566 | Isoform 1 of Mitotic | IPI00141933 | 120 kDa | 3 | 1 | 0 |
| | | checkpoint | | | | | |
| | | serine/threonine-protein | | | | | |
| | | kinase BUB1 beta | | | | | |
| BUB1 | O43683 | Mitotic checkpoint | IPI00783305 | 122 kDa | 1 | 0 | 0 |
| | | serine/threonine-protein | | | | | |
| | | kinase BUB1 | | | | | |
| ANAPC1 | Q9H1A4 | Anaphase-promoting | IPI00033907 | 217 kDa | 12 | 6 | 7 |
| | | complex subunit 1 | | | | | |
| ANAPC7 | Q9UJX3 | anaphase-promoting | IPI00008248 | 67 kDa | 3 | 8 | 0 |
| | | complex subunit 7 isoform a | (+1) | | | | |
| ANAPC5 | Q9UJX4 | Isoform 1 of Anaphase- | IPI00008247 | 85 kDa | 9 | 3 | 0 |
| | | promoting complex subunit 5 | | | | | |
| ANAPC4 | Q9UJX5 | Isoform 1 of Anaphase- | IPI00002551 | 92 kDa | 3 | 0 | 0 |
| | | promoting complex subunit 4 | | | | | |
| NEK9 | Q8TD19 | Serine/threonine-protein | IPI00301609 | 107 kDa | 3 | 3 | 5 |
| | | kinase Nek9 | | | | | |
| CDC45 | O75419 | CDC45-related protein | IPI00025695 | 66 kDa | 7 | 7 | 0 |
| | | | (+2) | | | | |
| CRKL | P46109 | Crk-like protein | IPI00004839 | 34 kDa | 5 | 0 | 0 |
| DOCK2 | Q92608 | Isoform 1 of Dedicator of | IPI00022449 | 212 kDa | 2 | 3 | 1 |
| | | cytokinesis protein 2 | | | | | |
| DOCK7 | Q96N67 | Isoform 2 of Dedicator of | IPI00183572 | 241 kDa | 2 | 0 | 0 |
| | | cytokinesis protein 7 | (+5) | | | | |
| DOCK11 | Q5JSL3 | Putative uncharacterized | IPI00411452 | 238 kDa | 0 | 0 | 1 |
| | | protein DOCK11 | (+1) | | | | |
| EPS15 | P42566 | Isoform 1 of Epidermal | IPI00292134 | 99 kDa | 23 | 26 | 3 |
| | | growth factor receptor | | | | | |
| | | substrate 15 | | | | | |
| GRB2 | P62993 | Isoform 1 of Growth factor | IPI00021327 | 25 kDa | 5 | 1 | 2 |
| | | receptor-bound protein 2 | (+1) | | | | |
| BTF3 | P20290 | Isoform 1 of Transcription | IPI00221035 | 22 kDa | 0 | 0 | 3 |
| | | factor BTF3 | (+1) | | | | |
| LGALS3 | P17931 | Galectin-3 | IPI00465431 | 26 kDa | 0 | 0 | 9 |
| NONO | Q15233 | Non-POU domain- | IPI00304596 | 54 kDa | 0 | 0 | 4 |
| | | containing octamer-binding | | | | | |
| | | protein | | | | | |
| ITPA | Q9BY32 | Inosine triphosphate | IPI00018783 | 21 kDa | 0 | 0 | 5 |
| | | pyrophosphatase | | | | | |
| RBX1 | P62877 | RING-box protein 1 | IPI00003386 | 12 kDa | 0 | 0 | 5 |
| RIPK1 | Q13546 | Receptor-interacting | IPI00013773 | 76 kDa | 2 | 0 | 0 |
| | | serine/threonine-protein | | | | | |
| | | kinase 1 | | | | | |
| HINT1 | P49773 | Histidine triad nucleotide- | IPI00239077 | 14 kDa | 0 | 0 | 9 |
| | | binding protein 1 | | | | | |
| GSE1 | Q14687 | Isoform 1 of Genetic | IPI00215963 | 136 kDa | 11 | 2 | 0 |
| KIAA0182 | | suppressor element 1 | (+1) | | | | |
| PDAP1 | Q13442 | 28 kDa heat- and acid- | IPI00013297 | 21 kDa | 0 | 0 | 5 |
| | | stable phosphoprotein | | | | | |
| SQSTM1 | Q13501 | Isoform 1 of | IPI00179473 | 48 kDa | 3 | 5 | 1 |
| | | Sequestosome-1 | (+1) | | | | |
| TBL1XR1 | Q9BZK7 | F-box-like/WD repeat- | IPI00002922 | 56 kDa | 3 | 12 | 3 |
| | | containing protein | | | | | |
| | | TBL1XR1 | | | | | |
| PRMT5 | O14744 | Protein arginine N- | IPI00441473 | 73 kDa | 12 | 11 | 3 |
| | | methyltransferase 5 | | | | | |
| PRMT6 | Q96LA8 | Protein arginine N- | IPI00102128 | 42 kDa | 2 | 0 | 0 |
| | | methyltransferase 6 | (+1) | | | | |
| PRMT3 | Q8WUV3 | PRMT3 protein (Fragment) | IPI00103026 | 62 kDa | 6 | 1 | 1 |
| | | | (+2) | | | | |
| ATG2A | Q2TAZ0 | Isoform 1 of Autophagy- | IPI00304926 | 213 kDa | 2 | 3 | 0 |
| | | related protein 2 homolog A | (+1) | | | | |
| AMBRA1 | Q9C0C7 | Isoform 2 of Activating | IPI00106552 | 136 kDa | 2 | 2 | 1 |
| | | molecule in BECN1- | (+3) | | | | |
| | | regulated autophagy | | | | | |
| | | protein 1 | | | | | |
| ATG5 | Q9H1Y0 | Isoform Long of Autophagy | IPI00006800 | 32 kDa | 2 | 1 | 0 |
| | | protein 5 | | | | | |
| YWHAE | P62258 | 14-3-3 protein epsilon | IPI00000816 | 29 kDa | 13 | 1 | 13 |
| MYBBP1A | Q9BQG0 | Isoform 1 of Myb-binding | IPI00005024 | 149 kDa | 4 | 4 | 29 |
| | | protein 1A | (+1) | | | | |
| RQCD1 | Q92600 | Cell differentiation protein | IPI00023101 | 34 kDa | 5 | 1 | 8 |
| | | RCD1 homolog | | | | | |
| YWHAQ | P27348 | 14-3-3 protein theta | IPI00018146 | 28 kDa | 0 | 0 | 4 |
| DDB1 | Q16531 | DNA damage-binding | IPI00293464 | 127 kDa | 25 | 15 | 2 |
| | | protein 1 | | | | | |
| YBX1 | P67809 | Nuclease-sensitive | IPI00031812 | 36 kDa | 6 | 13 | 40 |
| | | element-binding protein 1 | | | | | |
| RCOR1 | Q9UKL0 | REST corepressor 1 | IPI00008531 | 53 kDa | 9 | 5 | 0 |
| HDAC1 | Q13547 | Histone deacetylase 1 | IPI00013774 | 55 kDa | 10 | 11 | 1 |
| KDM1A | O60341 | Isoform 2 of Lysine-specific | IPI00217540 | 95 kDa | 13 | 4 | 0 |
| | | histone demethylase 1 | (+1) | | | | |
| HDAC6 | Q9UBN7 | cDNA FLJ56474, highly | IPI00005711 | 133 kDa | 4 | 6 | 2 |
| | | similar to Histone | | | | | |
| | | deacetylase 6 | | | | | |
| RBBP7 | Q16576 | Histone-binding protein | IPI00395865 | 48 kDa | 5 | 4 | 3 |
| | | RBBP7 | (+2) | | | | |
| HIST1H1C | P16403 | Histone H1.2 | IPI00217465 | 21 kDa | 1 | 0 | 7 |
| HDAC2 | Q92769 | histone deacetylase 2 | IPI00289601 | 66 kDa | 2 | 3 | 1 |
| HIST1H1B | P16401 | Histone H1.5 | IPI00217468 | 23 kDa | 0 | 0 | 5 |
| H1FX | Q92522 | Histone H1x | IPI00021924 | 22 kDa | 0 | 0 | 3 |
| SMARCC1 | Q92922 | SWI/SNF complex subunit | IPI00234252 | 123 kDa | 15 | 17 | 0 |
| | | SMARCC1 | | | | | |
| SMARCC2 | Q8TAQ2 | Isoform 2 of SWI/SNF | IPI00150057 | 125 kDa | 6 | 7 | 0 |
| | | complex subunit SMARCC2 | (+1) | | | | |
| TNFAIP2 | Q03169 | Tumor necrosis factor, | IPI00304866 | 73 kDa | 2 | 1 | 0 |
| | | alpha-induced protein 2 | | | | | |
| PICALM | Q13492 | Isoform 2 of | IPI00216184 | 69 kDa | 1 | 7 | 0 |
| | | Phosphatidylinositol-binding | (+5) | | | | |
| | | clathrin assembly protein | | | | | |
| KIAA1967 | Q8N163 | Isoform 1 of Protein | IPI00182757 | 103 kDa | 17 | 23 | 3 |
| | | KIAA1967 | | | | | |
| MCM5 | P33992 | DNA replication licensing | IPI00018350 | 82 kDa | 24 | 18 | 2 |
| | | factor MCM5 | (+2) | | | | |
| TFRC | P02786 | Transferrin receptor protein 1 | IPI00022462 | 85 kDa | 25 | 7 | 0 |
| TRIM28 | Q13263 | Isoform 1 of Transcription | IPI00438229 | 89 kDa | 16 | 14 | 4 |
| | | intermediary factor 1-beta | | | | | |
| TLN1 | Q9Y490 | Talin-1 | IPI00298994 | 270 kDa | 12 | 12 | 0 |
| NDC80 | O14777 | Kinetochore protein NDC80 | IPI00005791 | 74 kDa | 13 | 4 | 0 |
| | | homolog | | | | | |
| IQGAP2 | Q13576 | Isoform 1 of Ras GTPase- | IPI00299048 | 181 kDa | 18 | 21 | 1 |
| | | activating-like protein | | | | | |
| | | IQGAP2 | | | | | |
| MIF | P14174 | Macrophage migration | IPI00293276 | 12 kDa | 3 | 0 | 25 |
| | | inhibitory factor | | | | | |
| PA2G4 | Q9UQ80 | Proliferation-associated | IPI00299000 | 44 kDa | 3 | 8 | 14 |
| | | protein 2G4 | | | | | |
| CYFIP1 | Q7L576 | Isoform 1 of Cytoplasmic | IPI00644231 | 145 kDa | 8 | 4 | 4 |
| | | FMR1-interacting protein 1 | (+1) | | | | |
| PCNA | P12004 | Proliferating cell nuclear | IPI00021700 | 29 kDa | 9 | 3 | 10 |
| | | antigen | | | | | |
| NSUN2 | Q08J23 | tRNA (cytosine-5-)- | IPI00306369 | 86 kDa | 11 | 8 | 5 |
| | | methyltransferase NSUN2 | | | | | |
| NCOR1 | O75376 | Isoform 1 of Nuclear | IPI00289344 | 270 kDa | 11 | 13 | 1 |
| | | receptor corepressor 1 | (+1) | | | | |
| NCOR2 | Q9Y618 | Isoform 1 of Nuclear | IPI00001735 | 275 kDa | 8 | 5 | 2 |
| | | receptor corepressor 2 | | | | | |
| ILF3 | Q12906 | Isoform 1 of Interleukin | IPI00298788 | 95 kDa | 25 | 16 | 20 |
| | | enhancer-binding factor 3 | | | | | |
| ILF2 | Q12905 | Interleukin enhancer- | IPI00005198 | 43 kDa | 8 | 11 | 18 |
| | | binding factor 2 | | | | | |
| KHDRBS1 | Q07666 | Isoform 1 of KH domain- | IPI00008575 | 48 kDa | 8 | 15 | 2 |
| | | containing, RNA-binding, | | | | | |
| | | signal transduction- | | | | | |
| | | associated protein 1 | | | | | |
| RNF213 | Q9HCF4 | Isoform 1 of Protein ALO17 | IPI00642126 | 576 kDa | 12 | 49 | 16 |
| MTA2 | O94776 | Metastasis-associated | IPI00171798 | 75 kDa | 14 | 12 | 3 |
| | | protein MTA2 | | | | | |
| TRMT112 | Q9UI30 | TRM112-like protein | IPI00009010 | 14 kDa | 0 | 0 | 3 |
| ERH | P84090 | Enhancer of rudimentary | IPI00029631 | 12 kDa | 0 | 0 | 3 |
| | | homolog | | | | | |
| FBXO22 | Q8NEZ5 | Isoform 1 of F-box only | IPI00183208 | 45 kDa | 0 | 0 | 3 |
| | | protein 22 | | | | | |
| TP63 | Q9H3D4 | Isoform 1 of Tumor protein | IPI00301360 | 77 kDa | 0 | 0 | 3 |
| | | 63 | (+5) | | | | |
| PPP5C | P53041 | Serine/threonine-protein | IPI00019812 | 57 kDa | 3 | 1 | 0 |
| | | phosphatase 5 | | | | | |
| DIAPH1 | O60610 | Isoform 1 of Protein | IPI00852685 | 141 kDa | 6 | 7 | 0 |
| | | diaphanous homolog 1 | (+1) | | | | |
| RPA1 | P27694 | Replication protein A 70 kDa | IPI00020127 | 68 kDa | 22 | 8 | 0 |
| | | DNA-binding subunit | | | | | |
| SERBP1 | Q8NC51 | Isoform 3 of Plasminogen | IPI00470498 | 43 kDa | 0 | 6 | 16 |
| | | activator inhibitor 1 RNA- | | | | | |
| | | binding protein | | | | | |
| PPP2R5E | Q16537 | Serine/threonine-protein | IPI00002853 | 55 kDa | 0 | 0 | 2 |
| | | phosphatase 2A 56 kDa | (+1) | | | | |
| | | regulatory subunit epsilon | | | | | |
| | | isoform | | | | | |
| PPP2R1B | P30154 | Isoform 1 of | IPI00294178 | 66 kDa | 3 | 2 | 0 |
| | | Serine/threonine-protein | (+3) | | | | |
| | | phosphatase 2A 65 kDa | | | | | |
| | | regulatory subunit A beta | | | | | |
| | | isoform | | | | | |
| PPP2R2A | P63151 | Serine/threonine-protein | IPI00332511 | 52 kDa | 9 | 1 | 5 |
| | | phosphatase 2A 55 kDa | | | | | |
| | | regulatory subunit B alpha | | | | | |
| | | isoform | | | | | |
| PPP6R1 | Q9UPN7 | Isoform 1 of | IPI00402008 | 103 kDa | 5 | 2 | 5 |
| | | Serine/threonine-protein | (+1) | | | | |
| | | phosphatase 6 regulatory | | | | | |
| | | subunit 1 | | | | | |
| TGFBRAP1 | Q8WUH2 | Transforming growth factor- | IPI00550891 | 97 kDa | 1 | 0 | 0 |
| | | beta receptor-associated | | | | | |
| | | protein 1 | | | | | |
| OLA1 | Q9NTK5 | Isoform 1 of Obg-like | IPI00290416 | 45 kDa | 8 | 4 | 3 |
| | | ATPase 1 | | | | | |
| CTSB | P07858 | Cathepsin B | IPI00295741 | 38 kDa | 0 | 0 | 2 |
| | | | (+2) | | | | |
| CTSZ | Q9UBR2 | Cathepsin Z | IPI00002745 | 34 kDa | 1 | 0 | 0 |
| | | | (+1) | | | | |
| ACAP2 | Q15057 | ARFGAP with coiled-coil, | IPI00014264 | 88 kDa | 3 | 2 | 1 |
| | | ANK repeat and PH | | | | | |
| | | domain-containing protein 2 | | | | | |
| GIT1 | Q9Y2X7 | Isoform 1 of ARF GTPase- | IPI00384861 | 84 kDa | 2 | 0 | 0 |
| | | activating protein GIT1 | (+2) | | | | |
| ARHGEF1 | Q92888 | Isoform 2 of Rho guanine | IPI00339379 | 99 kDa | 4 | 3 | 0 |
| | | nucleotide exchange factor 1 | (+2) | | | | |
| ARHGEF2 | Q92974 | Isoform 1 of Rho guanine | IPI00291316 | 112 kDa | 14 | 7 | 2 |
| | | nucleotide exchange factor 2 | | | | | |
| RANGAP1 | P46060 | Ran GTPase-activating | IPI00294879 | 64 kDa | 13 | 4 | 1 |
| | | protein 1 | | | | | |
| GAPVD1 | Q14C86 | Isoform 6 of GTPase- | IPI00292753 | 166 kDa | 4 | 6 | 6 |
| | | activating protein and VPS9 | (+4) | | | | |
| | | domain-containing protein 1 | | | | | |
| RAB3GAP1 | Q15042 | Isoform 1 of Rab3 GTPase- | IPI00014235 | 111 kDa | 9 | 6 | 3 |
| | | activating protein catalytic | | | | | |
| | | subunit | | | | | |
| RAN | P62826 | GTP-binding nuclear | IPI00643041 | 24 kDa | 7 | 2 | 6 |
| | | protein Ran | (+1) | | | | |
| SAR1A | Q9NR31 | GTP-binding protein SAR1a | IPI00015954 | 22 kDa | 3 | 1 | 1 |
| RAB11B | Q15907 | Ras-related protein Rab- | IPI00020436 | 24 kDa | 6 | 1 | 0 |
| | | 11B | (+1) | | | | |
| TBC1D15 | Q8TC07 | TBC1 domain family, | IPI00794613 | 80 kDa | 6 | 4 | 4 |
| | | member 15 isoform 3 | | | | | |
| TELO2 | Q9Y4R8 | Telomere length regulation | IPI00016868 | 92 kDa | 11 | 1 | 1 |
| | | protein TEL2 homolog | | | | | |
| RIF1 | Q5UIP0 | Isoform 1 of Telomere- | IPI00293845 | 274 kDa | 2 | 0 | 2 |
| | | associated protein RIF1 | (+1) | | | | |
| WRAP53 | Q9BUR4 | Telomerase Cajal body | IPI00306087 | 59 kDa | 3 | 0 | 0 |
| | | protein 1 | | | | | |
| TNKS1BP1 | Q9C0C2 | Isoform 1 of 182 kDa | IPI00304589 | 182 kDa | 23 | 79 | 12 |
| | | tankyrase-1-binding protein | (+1) | | | | |
| PDCD4 | Q53EL6 | programmed cell death 4 | IPI00240675 | 51 kDa | 2 | 5 | 3 |
| | | isoform 2 | (+1) | | | | |
| FERMT3 | Q86UX7 | Isoform 2 of Fermitin family | IPI00216699 | 75 kDa | 8 | 0 | 0 |
| | | homolog 3 | (+1) | | | | |
| PTK2B | Q14289 | Isoform 1 of Protein | IPI00029702 | 116 kDa | 2 | 0 | 0 |
| | | tyrosine kinase 2 beta; | (+1) | | | | |
| | | PYK2; FAK2 | | | | | |
| MLLT4 | P55196 | Isoform 4 of Afadin | IPI00023461 | 207 kDa | 1 | 2 | 0 |
| | | | (+1) | | | | |
| TRIM56 | Q9BRZ2 | Isoform 1 of Tripartite motif- | IPI00514832 | 81 kDa | 0 | 0 | 3 |
| | | containing protein 56 | (+1) | | | | |
| HYOU1 | Q9Y4L1 | Hypoxia up-regulated | IPI00000877 | 111 kDa | 0 | 3 | 0 |
| | | protein 1 | (+1) | | | | |
| ZG16B | Q96DA0 | Zymogen granule protein | IPI00060800 | 23 kDa | 0 | 3 | 0 |
| | | 16 homolog B | | | | | |
| INPP4A | Q96PE3 | Isoform 3 of Type I inositol- | IPI00044388 | 109 kDa | 3 | 0 | 0 |
| | | 3,4-bisphosphate 4- | (+3) | | | | |
| | | phosphatase | | | | | |
| INF2 | Q27J81 | Putative uncharacterized | IPI00872508 | 55 kDa | 0 | 0 | 3 |
| | | protein INF2 | (+3) | | | | |
| GNL1 | P36915 | HSR1 protein | IPI00384745 | 62 kDa | 2 | 1 | 0 |
| | | | (+1) | | | | |
| SAMHD1 | Q9Y3Z3 | SAM domain and HD | IPI00294739 | 72 kDa | 11 | 2 | 6 |
| | | domain-containing protein 1 | | | | | |
| TJP1 | Q07157 | Isoform Long of Tight | IPI00216219 | 195 kDa | 6 | 3 | 0 |
| | | junction protein ZO-1 | (+2) | | | | |
| BAT3 | P46379 | Isoform 1 of Large proline- | IPI00465128 | 119 kDa | 4 | 5 | 3 |
| | | rich protein BAT3 | (+4) | | | | |
| SPTA1 | D3DVD8 | spectrin, alpha, erythrocytic 1 | IPI00220741 | 280 kDa | 43 | 62 | 0 |
| FLNA | P21333 | Isoform 2 of Filamin-A | IPI00302592 | 280 kDa | 26 | 91 | 0 |
| | | | (+2) | | | | |
| FLNC | Q14315 | Isoform 1 of Filamin-C | IPI00178352 | 291 kDa | 55 | 183 | 0 |
| | | | (+1) | | | | |
| KIAA1468 | Q9P260 | Isoform 2 of LisH domain | IPI00023330 | 139 kDa | 0 | 0 | 3 |
| | | and HEAT repeat- | | | | | |
| | | containing protein | | | | | |
| | | KIAA1468 | | | | | |
| HEATR2 | Q86Y56 | Isoform 1 of HEAT repeat- | IPI00242630 | 94 kDa | 5 | 2 | 11 |
| | | containing protein 2 | | | | | |
| HEATR6 | Q6AI08 | HEAT repeat-containing | IPI00464999 | 129 kDa | 2 | 1 | 0 |
| | | protein 6 | | | | | |
| HSPG2 | P98160 | Basement membrane- | IPI00024284 | 469 kDa | 4 | 9 | 0 |
| | | specific heparan sulfate | | | | | |
| | | proteoglycan core protein | | | | | |
| CTTN | Q14247 | Src substrate cortactin | IPI00029601 | 62 kDa | 6 | 6 | 2 |
| | | | (+1) | | | | |
| AIP | O00170 | AH receptor-interacting | IPI00010460 | 38 kDa | 10 | 0 | 0 |
| | | protein | | | | | |
| NAT10 | Q9H0A0 | N-acetyltransferase 10 | IPI00300127 | 116 kDa | 8 | 3 | 1 |
| DICER1 | Q9UPY3 | dicer1 | IPI00219036 | 219 kDa | 8 | 3 | 1 |
| FAM120A | Q9NZB2 | Isoform A of Constitutive | IPI00472054 | 122 kDa | 1 | 1 | 12 |
| | | coactivator of PPAR- | (+1) | | | | |
| | | gamma-like protein 1 | | | | | |
| NUMA1 | Q14980 | Isoform 2 of Nuclear mitotic | IPI00006196 | 237 kDa | 4 | 4 | 4 |
| | | apparatus protein 1 | (+2) | | | | |
| TRIPI3 | Q15645 | Isoform 1 of Thyroid | IPI00003505 | 49 kDa | 3 | 3 | 8 |
| | | receptor-interacting protein | | | | | |
| | | 13 | | | | | |
| FAM115A | Q9Y4C2 | Isoform 1 of Protein | IPI00006050 | 102 kDa | 9 | 1 | 0 |
| | | FAM115A | (+3) | | | | |
| SUPV3L1 | Q8IYB8 | ATP-dependent RNA | IPI00412404 | 88 kDa | 8 | 3 | 0 |
| | | helicase SUPV3L1, | | | | | |
| | | mitochondrial | | | | | |
| LTV1 | Q96GA3 | Protein LTV1 homolog | IPI00153032 | 55 kDa | 5 | 6 | 0 |
| LYAR | Q9NX58 | Cell growth-regulating | IPI00015838 | 44 kDa | 1 | 2 | 6 |
| | | nucleolar protein | | | | | |
| ASAH1 | Q13510 | Acid ceramidase | IPI00013698 | 45 kDa | 8 | 1 | 0 |
| FIP1L1 | Q6UN15 | Isoform 3 of Pre-mRNA 3′- | IPI00008449 | 58 kDa | 6 | 3 | 0 |
| | | end-processing factor FIP1 | (+3) | | | | |
| TP53BP1 | Q12888 | Isoform 1 of Tumor | IPI00029778 | 214 kDa | 0 | 6 | 3 |
| | | suppressor p53-binding | (+3) | | | | |
| | | protein 1 | | | | | |
| BAX | Q07812 | Isoform Epsilon of | IPI00071059 | 18 kDa | 3 | 0 | 6 |
| | | Apoptosis regulator BAX | (+3) | | | | |
| APRT | P07741 | Adenine | IPI00218693 | 20 kDa | 0 | 0 | 6 |
| | | phosphoribosyltransferase | | | | | |
| FHOD1 | Q9Y613 | FH1/FH2 domain- | IPI00001730 | 127 kDa | 5 | 2 | 0 |
| | | containing protein 1 | | | | | |
| CPNE3 | O75131 | Copine-3 | IPI00024403 | 60 kDa | 4 | 5 | 0 |
| TLE1 | Q04724 | Isoform 2 of Transducin-like | IPI00177938 | 82 kDa | 5 | 2 | 1 |
| | | enhancer protein 3 | (+4) | | | | |
| TPP1 | O14773 | Putative uncharacterized | IPI00554538 | 60 kDa | 4 | 1 | 1 |
| | | protein TPP1 | (+2) | | | | |
| SDCCAG1 | O60524 | Isoform 1 of Serologically | IPI00301618 | 123 kDa | 2 | 2 | 3 |
| | | defined colon cancer | | | | | |
| | | antigen 1 | | | | | |
| NCKAP1 | Q9Y2A7 | Isoform 1 of Nck-associated | IPI00031982 | 129 kDa | 5 | 1 | 2 |
| | | protein 1 | (+1) | | | | |
| NUP54 | Q7Z3B4 | Nucleoporin 54 kDa variant | IPI00172580 | 56 kDa | 1 | 7 | 0 |
| | | (Fragment) | | | | | |
| NUP85 | Q9BW27 | Nucleoporin NUP85 | IPI00790530 | 75 kDa | 14 | 2 | 0 |
| NUP160 | Q12769 | nucleoporin 160 kDa | IPI00221235 | 162 kDa | 13 | 1 | 0 |
| NOP14 | P78316 | Isoform 1 of Nucleolar | IPI00022613 | 98 kDa | 9 | 2 | 0 |
| | | protein 14 | | | | | |
| PRPF31 | Q8WWY3 | Isoform 1 of U4/U6 small | IPI00292000 | 55 kDa | 3 | 2 | 0 |
| | | nuclear ribonucleoprotein | (+1) | | | | |
| | | Prp31 | | | | | |
| PRPF3 | O43395 | Isoform 1 of U4/U6 small | IPI00005861 | 78 kDa | 3 | 0 | 0 |
| | | nuclear ribonucleoprotein | (+1) | | | | |
| | | Prp3 | | | | | |
| CNOT1 | A5YKK6 | Isoform 1 of CCR4-NOT | IPI00166010 | 267 kDa | 53 | 73 | 23 |
| | | transcription complex | | | | | |
| | | subunit 1 | | | | | |
| LRRC40 | Q9H9A6 | Leucine-rich repeat- | IPI00152998 | 68 kDa | 4 | 3 | 0 |
| | | containing protein 40 | | | | | |
| PHB2 | Q99623 | Prohibitin-2 | IPI00027252 | 33 kDa | 8 | 0 | 0 |
| VAC14 | Q08AM6 | Protein VAC14 homolog | IPI00025160 | 88 kDa | 5 | 2 | 0 |
| NOP2 | P46087 | Putative uncharacterized | IPI00294891 | 94 kDa | 0 | 0 | 7 |
| | | protein NOP2 | (+2) | | | | |
| NOB1 | Q9ULX3 | RNA-binding protein NOB1 | IPI00022373 | 48 kDa | 5 | 0 | 0 |
| SARM1 | Q6SZW1 | Isoform 1 of Sterile alpha | IPI00448630 | 79 kDa | 0 | 0 | 5 |
| | | and TIR motif-containing | | | | | |
| | | protein 1 | | | | | |
| FTSJD2 | Q8N1G2 | FtsJ methyltransferase | IPI00166153 | 95 kDa | 3 | 1 | 0 |
| | | domain-containing protein 2 | | | | | |
| NFKB1 | P19838 | Isoform 2 of Nuclear factor | IPI00292537 | 105 kDa | 1 | 0 | 2 |
| | | NF-kappa-B p105 subunit | (+1) | | | | |
| SLC3A2 | P08195 | 4F2 cell-surface antigen | IPI00027493 | 58 kDa | 3 | 0 | 0 |
| | | heavy chain | (+5) | | | | |
| WIGB | Q9BRP8 | Putative uncharacterized | IPI00914992 | 23 kDa | 0 | 0 | 4 |
| | | protein WIBG (Fragment) | (+2) | | | | |
| DIABLO | Q9NR28 | Diablo homolog, | IPI00008418 | 36 kDa | 1 | 0 | 2 |
| | | mitochondrial precursor | (+4) | | | | |
| AIFM1 | O95831 | Isoform 1 of Apoptosis- | IPI00000690 | 67 kDa | 2 | 0 | 0 |
| | | inducing factor 1, | (+1) | | | | |
| | | mitochondrial | | | | | |
| ZC3HAV1 | Q7Z2W4 | Isoform 1 of Zinc finger | IPI00410067 | 101 kDa | 7 | 0 | 0 |
| | | CCCH-type antiviral protein 1 | | | | | |
| PSPC1 | Q8WXF1 | Isoform 1 of Paraspeckle | IPI00103525 | 59 kDa | 5 | 2 | 0 |
| | | component 1 | (+1) | | | | |
| STRN | O43815 | Isoform 1 of Striatin | IPI00014456 | 86 kDa | 5 | 1 | 0 |
| PHB | P35232 | Prohibitin | IPI00017334 | 30 kDa | 5 | 0 | 0 |
| | | | (+1) | | | | |
| SDPR | O95810 | Serum deprivation- | IPI00005809 | 47 kDa | 0 | 0 | 4 |
| | | response protein | | | | | |
| GPS2 | Q13227 | G protein pathway | IPI00012301 | 37 kDa | 5 | 0 | 0 |
| | | suppressor 2 | (+1) | | | | |
| CSDE1 | O75534 | Isoform Long of Cold shock | IPI00470891 | 89 kDa | 4 | 0 | 0 |
| | | domain-containing protein | (+2) | | | | |
| | | E1 | | | | | |
| CHD4 | Q14839 | Isoform 1 of | IPI00000846 | 218 kDa | 12 | 45 | 2 |
| | | Chromodomain-helicase- | (+1) | | | | |
| | | DNA-binding protein 4 | | | | | |
| RID1A | O14497 | Isoform 1 of AT-rich | IPI00643722 | 242 kDa | 20 | 37 | 0 |
| | | interactive domain- | | | | | |
| | | containing protein 1A | | | | | |
| PTPLAD1 | Q9P035 | Protein tyrosine | IPI00008998 | 43 kDa | 2 | 0 | 0 |
| | | phosphatase-like protein | (+1) | | | | |
| | | PTPLAD1 | | | | | |
| PLBD1 | Q6P4A8 | hypothetical protein | IPI00016255 | 63 kDa | 0 | 0 | 2 |
| | | LOC79887 | | | | | |
| MALT1 | Q9UDY8 | Isoform 1 of Mucosa- | IPI00009540 | 92 kDa | 0 | 0 | 2 |
| | | associated lymphoid tissue | (+2) | | | | |
| | | lymphoma translocation | | | | | |
| | | protein 1 | | | | | |
| BCL7C | Q8WUZ0 | Isoform 1 of B-cell | IPI00006266 | 23 kDa | 2 | 0 | 0 |
| | | CLL/lymphoma 7 protein | (+2) | | | | |
| | | family member C | | | | | |
| PRCC | Q92733 | Proline-rich protein PRCC | IPI00294618 | 52 kDa | 2 | 0 | 0 |
| | | | (+2) | | | | |
| WASF2 | Q9Y6W5 | Wiskott-Aldrich syndrome | IPI00472164 | 54 kDa | 2 | 0 | 0 |
| | | protein family member 2 | | | | | |
| PSD4 | Q8NDX1 | Isoform 1 of PH and SEC7 | IPI00304670 | 116 kDa | 2 | 0 | 0 |
| | | domain-containing protein 4 | (+2) | | | | |
| ZBED1 | O96006 | Zinc finger BED domain- | IPI00006203 | 78 kDa | 2 | 0 | 0 |
| | | containing protein 1 | | | | | |
| NCSTN | Q92542 | Isoform 1 of Nicastrin | IPI00021983 | 78 kDa | 2 | 0 | 0 |
| | | | (+3) | | | | |
| CT45A5 | Q6NSH3 | Cancer/testis antigen 45-5 | IPI00431697 | 21 kDa | 2 | 0 | 0 |
| | | | (+4) | | | | |
| MOBKL3 | Q9Y3A3 | Isoform 1 of Mps one | IPI00386122 | 26 kDa | 0 | 0 | 1 |
| | | binder kinase activator-like 3 | (+2) | | | | |
| SKP1 | P63208 | Isoform 2 of S-phase | IPI00172421 | 18 kDa | 0 | 0 | 4 |
| | | kinase-associated protein 1 | (+1) | | | | |
| KIF14 | Q15058 | Kinesin-like protein KIF14 | IPI00299554 | 186 kDa | 1 | 1 | 0 |
| ASCC2 | Q9H1I8 | Isoform 1 of Activating | IPI00549736 | 86 kDa | 0 | 0 | 1 |
| | | signal cointegrator 1 | | | | | |
| | | complex subunit 2 | | | | | |
| ZZEF1 | O43149 | Isoform 1 of Zinc finger ZZ- | IPI00385631 | 331 kDa | 0 | 0 | 1 |
| | | type and EF-hand domain- | (+1) | | | | |
| | | containing protein 1 | | | | | |
| MLF2 | Q15773 | Myeloid leukemia factor 2 | IPI00023095 | 28 kDa | 2 | 0 | 1 |
| PRAME | P78395 | preferentially expressed | IPI00893980 | 21 kDa | 4 | 0 | 0 |
| | | antigen in melanoma | (+3) | | | | |
| | O60613 | 15 kDa selenoprotein | IPI00030877 | 18 kDa | 0 | 0 | 2 |
| | | isoform 1 precursor | | | | | |

TABLE 5b
Putative Hsp90 interacting co-chaperones identified using the QSTAR-Elite
hybrid quadrupole time-of-flight mass spectrometer (QT of MS) (AB/MDS Sciex)

| | UniProt- | Identified Proteins | Accession | Molecular | K562 | K562 | Mia- | |
|---|---|---|---|---|---|---|---|---|
| EntrezGene | KB | (1559) | Number | Weight | Prep1 | Prep2 | Paca2 | |
| HSP90AA1 | P07900 | heat shock 90 kDa | IPI00382470 | 98 kDa | 563 | 2018 | 1514 | Hsp90 |
| | | protein 1, alpha | (+1) | | | | | alpha |
| | | isoform 1 | | | | | | |
| HSP90AB1 | P08238 | Heat shock protein | IPI00414676 | 83 kDa | 300 | 1208 | 578 | Hsp90 |
| | | HSP 90-beta | | | | | | beta |
| | | Putative heat shock | IPI00555565 | 58 kDa | 2 | 12 | 4 | |
| | | protein HSP 90-beta 4 | | | | | | |
| | | Putative heat shock | IPI00555957 | 48 kDa | 6 | 1 | 1 | |
| | | protein HSP 90- | | | | | | |
| | | alpha A4 | | | | | | |
| TRAP1 | Q12931 | Heat shock protein | IPI00030275 | 80 kDa | 65 | 411 | 21 | Trap- |
| | | 75 kDa, | | | | | | 1* |
| | | mitochondrial | | | | | | |
| HSP90B1 | P14625 | Endoplasmin; | IPI00027230 | 92 kDa | 55 | 194 | 1 | Grp94* |
| | | GRP94 | | | | | | |
| HSPA8 | P11142 | Isoform 1 of Heat | IPI00003865 | 71 kDa | 78 | 217 | 25 | Hsc70 |
| | | shock cognate 71 kDa | | | | | | |
| | | protein, Hsc70 | | | | | | |
| HSPA1B; | P08107 | Heat shock 70 kDa | IPI00304925 | 70 kDa | 47 | 61 | 3 | Hsp70 |
| HSPA1A | | protein 1 | (+1) | | | | | |
| | | Heat shock 70 kDa | IPI00002966 | 94 kDa | 6 | 1 | 0 | |
| | | protein 4 | | | | | | |
| STIP1 | P31948 | Stress-induced- | IPI00013894 | 63 kDa | 40 | 45 | 5 | HOP |
| | | phosphoprotein 1; | | | | | | |
| | | HOP | | | | | | |
| ST13 | P50502 | Hsc70-interacting | IPI00032826 | 41 kDa | 8 | 5 | 4 | HIP |
| | | protein | | | | | | |
| CDC37 | Q16543 | Hsp90 co- | IPI00013122 | 44 kDa | 1 | 1 | 3 | Cdc37 |
| | | chaperone Cdc37 | | | | | | |
| AHSA1 | O95433 | Activator of 90 kDa | IPI00030706 | 38 kDa | 1 | 0 | 3 | AHA-1 |
| | | heat shock protein | | | | | | |
| | | ATPase homolog 1 | | | | | | |
| HSPH1 | Q92598 | Isoform Beta of Heat | IPI00218993 | 92 kDa | 2 | 0 | 0 | Hsp110 |
| | | shock protein 105 kDa | (+2) | | | | | |
| DNAJC7 | Q99615 | DnaJ homolog | IPI00329629 | 56 kDa | 4 | 4 | 2 | Hsp40s |
| | | subfamily C member 7 | | | | | | |
| DNAJA2 | O60884 | DnaJ homolog | IPI00032406 | 46 kDa | 5 | 0 | 3 | |
| | | subfamily A member 2 | | | | | | |
| DNAJB6 | O75190 | Isoform A of DnaJ | IPI00024523 | 36 kDa | 5 | 0 | 2 | |
| | | homolog subfamily | (+1) | | | | | |
| | | B member 6 | | | | | | |
| DNAJB1 | P25685 | DnaJ homolog | IPI00012535 | 45 kDa | 6 | 0 | 2 | |
| | | subfamily A member 1 | | | | | | |
| DNAJB4 | Q9UDY4 | DnaJ homolog | IPI00008454 | 41 kDa | 4 | 2 | 1 | |
| | | subfamily B member | | | | | | |
| | | 11 | | | | | | |
| DNAJB1 | P25685 | DnaJ homolog | IPI00015947 | 38 kDa | 3 | 0 | 1 | |
| | | subfamily B member 1 | | | | | | |
| DNAJC13 | O75165 | DnaJ homolog | IPI00307259 | 254 kDa | 0 | 0 | 3 | |
| | | subfamily C member | | | | | | |
| | | 13 | | | | | | |
| DNAJC8 | O75937 | DnaJ homolog | IPI00003438 | 30 kDa | 1 | 0 | 0 | |
| | | subfamily C member 8 | | | | | | |
| DNAJC9 | Q8WXX5 | DnaJ homolog | IPI00154975 | 30 kDa | 3 | 0 | 1 | |
| | | subfamily C member 9 | | | | | | |
| SACS | Q9NZJ4 | Isoform 2 of Sacsin | IPI00784002 | 505 kDa | 2 | 1 | 0 | |
| | | | (+1) | | | | | |
| PPIB | P23284 | Peptidyl-prolyl cis- | IPI00646304 | 24 kDa | 4 | 0 | 0 | PPlase |
| | | trans isomerase B | | | | | | |
| PPIL1 | Q9Y3C6 | Isoform 1 of | IPI00003824 | 59 kDa | 13 | 1 | 0 | (peptidylprolylisomerase) |
| | | Peptidyl-prolyl cis- | | | | | | |
| | | trans isomerase-like 2 | | | | | | |
| PPIA | P62937 | Peptidyl-prolyl cis- | IPI00419585 | 18 kDa | 0 | 0 | 6 | |
| | | trans isomerase A | | | | | | |
| PPID | Q08752 | 40 kDa peptidyl- | IPI00003927 | 41 kDa | 3 | 1 | 0 | |
| | | prolyl cis-trans | | | | | | |
| | | isomerase | | | | | | |
| PPIE | Q9UNP9 | Isoform A of | IPI00009316 | 33 kDa | 0 | 0 | 3 | |
| | | Peptidyl-prolyl cis- | (+2) | | | | | |
| | | trans isomerase E | | | | | | |
| P4HB | P07237 | Protein disulfide- | IPI00010796 | 57 kDa | 11 | 36 | 1 | |
| | | isomerase | | | | | | |
| FKBP4 | Q02790 | FK506-binding | IPI00219005 | 52 kDa | 21 | 12 | 8 | |
| | | protein 4 | | | | | | |
| FKBP10 | Q96AY3 | FK506-binding | IPI00303300 | 64 kDa | 0 | 0 | 7 | |
| | | protein 10 | | | | | | |
| FKBP9 | O95302 | FK506-binding | IPI00182126 | 63 kDa | 1 | 0 | 0 | |
| | | protein 9 | (+1) | | | | | |
| BAG4 | O95429 | BAG family | IPI00030695 | 50 kDa | 4 | 0 | 0 | BAG |
| | | molecular | (+1) | | | | | |
| | | chaperone regulator 4 | | | | | | |
| BAG2 | O95816 | BAG family | IPI00000643 | 24 kDa | 1 | 1 | 3 | |
| | | molecular | | | | | | |
| | | chaperone regulator 2 | | | | | | |
| TTC27 | Q6P3X3 | Tetratricopeptide | IPI00183938 | 97 kDa | 13 | 3 | 2 | |
| | | repeat protein 27 | | | | | | |
| TTC4 | O95801 | Tetratricopeptide | IPI00000606 | 45 kDa | 1 | 0 | 0 | |
| | | repeat protein 4 | (+1) | | | | | |
| TTC19 | Q6DKK2 | Tetratricopeptide | IPI00170855 | 56 kDa | 2 | 0 | 0 | |
| | | repeat protein 19 | (+1) | | | | | |
| PTCD1 | O75127 | Pentatricopeptide | IPI00171925 | 79 kDa | 2 | 0 | 0 | |
| | | repeat-containing | | | | | | |
| | | protein 1 | | | | | | |
| | B3KU92 | Isoform 1 of TPR | IPI00395476 | 95 kDa | 3 | 0 | 0 | |
| | | repeat-containing | | | | | | |
| | | protein LOC90826 | | | | | | |
| TOMM40 | O96008 | Isoform 1 of | IPI00014053 | 38 kDa | 3 | 0 | 0 | TOM40 |
| | | Mitochondrial import | | | | | | |
| | | receptor subunit | | | | | | |
| | | TOM40 homolog | | | | | | |
| UNC45B | Q8IWX7 | Isoform 2 of Protein | IPI00735181 | 102 kDa | 33 | 6 | 2 | UNC45 |
| | | unc-45 homolog A | | | | | | |
| HSPA9 | P38646 | Stress-70 protein, | IPI00007765 | 74 kDa | 19 | 25 | 4 | GRP75 |
| | | mitochondrial; | | | | | | |
| | | GRP75 | | | | | | |
| HSPD1 | P10809 | 60 kDa heat shock | IPI00784154 | 61 kDa | 19 | 29 | 1 | HSP60 |
| | | protein, | | | | | | |
| | | mitochondrial; | | | | | | |
| | | HSP60 | | | | | | |

*Grp94 and Trap-1 are Hsp90 isoforms to which PU-H71 binds directly

TABLE 5c
Putative Hsp90 interacting proteins acting in the proteasome pathway identified
using the QSTAR-Elite hybrid quadrupole time-of-flight mass spectrometer (GT of MS)
(AB/MDS Sciex)

| | | | Accession | Molecular | K562 | K562 | Mia- |
|---|---|---|---|---|---|---|---|
| EntrezGene | UniProtKB | | Number | Weight | Prep1 | Prep2 | Paca2 |
| TRIM33 | Q9UPN9 | Isoform Alpha of E3 | IPI00010252 | 123 kDa | 1 | 1 | 0 |
| | | ubiquitin-protein ligase | (+1) | | | | |
| | | TRIM33 | | | | | |
| ITCH | Q96J02 | Isoform 1 of E3 ubiquitin- | IPI00061780 | 103 kDa | 2 | 0 | 0 |
| | | protein ligase Itchy | (+1) | | | | |
| | | homolog | | | | | |
| UBR3 | Q6ZT12 | Isoform 1 of E3 ubiquitin- | IPI00335581 | 212 kDa | 0 | 2 | 1 |
| | | protein ligase UBR3 | (+1) | | | | |
| UBR1 | Q8IWV7 | Isoform 1 of E3 ubiquitin- | IPI00217405 | 200 kDa | 3 | 1 | 1 |
| | | protein ligase UBR1 | | | | | |
| UBR2 | Q8IWV8 | Isoform 4 of E3 ubiquitin- | IPI00217407 | 201 kDa | 1 | 5 | 0 |
| | | protein ligase UBR2 | (+1) | | | | |
| UBR4 | Q5T4S7 | Isoform 3 of E3 ubiquitin- | IPI00646605 | 572 kDa | 40 | 61 | 8 |
| | | protein ligase UBR4 | (+2) | | | | |
| UBR5 | O95071 | E3 ubiquitin-protein ligase | IPI00026320 | 309 kDa | 15 | 34 | 0 |
| | | UBR5 | | | | | |
| UBE3C | Q15386 | Isoform 1 of Ubiquitin- | IPI00604464 | 124 kDa | 12 | 0 | 5 |
| | | protein ligase E3C | | | | | |
| UBE3A | Q05086 | Isoform II of Ubiquitin- | IPI00011609 | 101 kDa | 13 | 0 | 0 |
| | | protein ligase E3A | (+2) | | | | |
| UBE4B | O95155 | Isoform 1 of Ubiquitin | IPI00005715 | 146 kDa | 6 | 2 | 0 |
| | | conjugation factor E4 B | (+1) | | | | |
| HECTD3 | A1A4G1 | Isoform 1 of Probable E3 | IPI00456642 | 97 kDa | 4 | 1 | 2 |
| | | ubiquitin-protein ligase | (+1) | | | | |
| | | HECTD3 | | | | | |
| NEDD4 | P46934 | E3 ubiquitin-protein ligase | IPI00009322 | 115 kDa | 5 | 0 | 1 |
| | | NEDD4 | | | | | |
| RNF123 | Q5XPI4 | Isoform 1 of E3 ubiquitin- | IPI00335085 | 149 kDa | 2 | 0 | 0 |
| | | protein ligase RNF123 | (+2) | | | | |
| HERC4 | Q5GLZ8 | Isoform 1 of Probable E3 | IPI00333067 | 119 kDa | 3 | 0 | 0 |
| | | ubiquitin-protein ligase | (+3) | | | | |
| | | HERC4 | | | | | |
| HERC1 | Q15751 | Probable E3 ubiquitin- | IPI00022479 | 532 kDa | 1 | 2 | 0 |
| | | protein ligase HERC1 | | | | | |
| KCMF1 | Q9P0J7 | E3 ubiquitin-protein ligase | IPI00306661 | 42 kDa | 1 | 0 | 0 |
| | | KCMF1 | | | | | |
| TRIP12 | Q14669 | TRIP12 protein; Probable | IPI00032342 | 226 kDa | 0 | 0 | 6 |
| | | E3 ubiquitin-protein ligase | (+1) | | | | |
| | | TRIP12 | | | | | |
| USP47 | Q96K76 | Isoform 1 of Ubiquitin | IPI00607554 | 157 kDa | 11 | 8 | 2 |
| | | carboxyl-terminal | | | | | |
| | | hydrolase 47 | | | | | |
| USP34 | Q70CQ2 | Isoform 1 of Ubiquitin | IPI00297593 | 404 kDa | 15 | 6 | 3 |
| | | carboxyl-terminal | (+2) | | | | |
| | | hydrolase 34 | | | | | |
| USP15 | Q9Y4E8 | Isoform 1 of Ubiquitin | IPI00000728 | 112 kDa | 12 | 10 | 2 |
| | | carboxyl-terminal | | | | | |
| | | hydrolase 15 | | | | | |
| USP9X | Q93008 | ubiquitin specific protease | IPI00003964 | 290 kDa | 24 | 52 | 9 |
| | | 9, X-linked isoform 4 | (+1) | | | | |
| UBAP2L | Q14157 | Isoform 1 of Ubiquitin- | IPI00514856 | 115 kDa | 9 | 12 | 17 |
| | | associated protein 2-like | | | | | |
| UBA1 | P22314 | Ubiquitin-like modifier- | IPI00645078 | 118 kDa | 6 | 6 | 26 |
| | | activating enzyme 1 | | | | | |
| UCHL5 | Q9Y5K5 | Isoform 2 of Ubiquitin | IPI00219512 | 36 kDa | 12 | 0 | 5 |
| | | carboxyl-terminal | (+6) | | | | |
| | | hydrolase isozyme L5 | | | | | |
| USP7 | Q93009 | Ubiquitin carboxyl-terminal | IPI00003965 | 128 kDa | 8 | 3 | 0 |
| | | hydrolase 7 | (+1) | | | | |
| USP10 | Q14694 | Ubiquitin carboxyl-terminal | IPI00291946 | 87 kDa | 5 | 2 | 2 |
| | | hydrolase 10 | | | | | |
| USP32 | Q8NFA0 | Ubiquitin carboxyl-terminal | IPI00185661 | 182 kDa | 5 | 1 | 2 |
| | | hydrolase 32 | (+1) | | | | |
| USP28 | Q96RU2 | Isoform 1 of Ubiquitin | IPI00045496 | 122 kDa | 1 | 1 | 2 |
| | | carboxyl-terminal | (+1) | | | | |
| | | hydrolase 28 | | | | | |
| USP14 | P54578 | Ubiquitin carboxyl-terminal | IPI00219913 | 56 kDa | 2 | 2 | 0 |
| | | hydrolase 14 | (+2) | | | | |
| CDC16 | Q13042 | Isoform 1 of Cell division | IPI00022091 | 72 kDa | 1 | 3 | 0 |
| | | cycle protein 16 homolog | (+3) | | | | |
| USP11 | P51784 | ubiquitin specific protease | IPI00184533 | 110 kDa | 9 | 2 | 5 |
| | | 11 | | | | | |
| UFD1L | Q92890 | Isoform Short of Ubiquitin | IPI00218292 | 35 kDa | 10 | 0 | 7 |
| | | fusion degradation protein | (+2) | | | | |
| | | 1 homolog | | | | | |
| UBAP2 | Q5T6F2 | Ubiquitin-associated | IPI00171127 | 117 kDa | 6 | 2 | 1 |
| | | protein 2 | | | | | |
| UBAC1 | Q9BSL1 | Ubiquitin-associated | IPI00305442 | 45 kDa | 6 | 0 | 0 |
| | | domain-containing protein 1 | | | | | |
| FAU | P62861 | ubiquitin-like protein fubi | IPI00019770 | 14 kDa | 0 | 0 | 2 |
| | | and ribosomal protein S30 | (+1) | | | | |
| | | precursor | | | | | |
| NUB1 | Q9Y5A7 | NEDD8 ultimate buster 1 | IPI00157365 | 72 kDa | 4 | 1 | 0 |
| | | (Negative regulator of | (+1) | | | | |
| | | ubiquitin-like proteins 1) | | | | | |
| | | (Renal carcinoma antigen | | | | | |
| | | NY-REN-18). Isoform 2 | | | | | |
| VCPIP1 | Q96JH7 | Deubiquitinating protein | IPI00064162 | 134 kDa | 1 | 0 | 0 |
| | | VCIP135 | | | | | |
| GAN | Q9H2C0 | Gigaxonin | IPI00022758 | 68 kDa | 2 | 2 | 1 |
| UBQLN2 | Q9UHD9 | Ubiquilin-2 | IPI00409659 | 66 kDa | 0 | 0 | 3 |
| | | | (+1) | | | | |
| KEAP1 | Q14145 | Kelch-like ECH-associated | IPI00106502 | 70 kDa | 5 | 2 | 0 |
| | | protein 1 | (+1) | | | | |
| CUL2 | B7Z6K8 | cDNA FLJ56037, highly | IPI00014311 | 90 kDa | 10 | 6 | 3 |
| | | similar to Cullin-2 | | | | | |
| CUL1 | Q13616 | Cullin-1 | IPI00014310 | 90 kDa | 11 | 2 | 1 |
| CAND2 | O75155 | Isoform 2 of Cullin- | IPI00374208 | 123 kDa | 5 | 2 | 0 |
| | | associated NEDD8- | | | | | |
| | | dissociated protein 2 | | | | | |
| CUL3 | Q13618 | Isoform 1 of Cullin-3 | IPI00014312 | 89 kDa | 7 | 0 | 1 |
| | | | (+1) | | | | |
| CUL4A | Q13619 | Isoform 1 of Cullin-4A | IPI00419273 | 88 kDa | 4 | 0 | 0 |
| CUL4B | Q13620 | Isoform 1 of Cullin-4B | IPI00179057 | 102 kDa | 2 | 0 | 0 |
| | | | (+2) | | | | |
| CUL5 | Q93034 | Cullin-5 | IPI00216003 | 97 kDa | 1 | 0 | 0 |
| | | | (+1) | | | | |

TABLE 5d
Putative Hsp90 interacting proteins identified using the Waters Xevo QTof MS

| | | | | gel size | Run1 | | | | | | | Run2 | | | | | | | |
|---|---|---|---|---|---|---|---|---|---|---|---|---|---|---|---|---|---|---|---|
| | | | | cut | >200 | 150-200 | 110-150 | 80-110 | 60-80 | 40-60 | <40 | >200 | 150-200 | 110-150 | 80-110 | 60-80 | 40-60 | <40 | |
| | | | Matched Peptides by Fraction | | | | | | | | | | | | | | | | MAXIMUM |
| Protein.Name. | UniProt- | | | Total | | | | | | | | | | | | | | | matched |
| Abbrev | KB | Reference | MW | fmol | JA01 | JA02 | JA03 | JA04 | JA05 | JA06 | JA07 | JA08 | JA09 | JA10 | JA11 | JA12 | JA13 | JA14 | peptides |
| Heat shock | P08238 | | 83264.4 | 2708.8638 | 14 | 5 | 11 | 260 | 54 | 55 | 20 | 25 | 5 | 24 | 242 | 57 | 51 | 19 | 260 |
| protein HSP | | | | | | | | | | | | | | | | | | | |
| 90-beta | | | | | | | | | | | | | | | | | | | |
| Heat shock | P07900 | | 84659.9 | 1351.4965 | 6 | | 7 | 209 | 47 | 38 | 14 | 14 | | 20 | 234 | | | 11 | 234 |
| protein HSP | | | | | | | | | | | | | | | | | | | |
| 90-alpha | | | | | | | | | | | | | | | | | | | |
| Signal | P42229 | | 90647.2 | 33.6765 | | | | 78 | | | | | | | 73 | | | | 78 |
| transducer and | | | | | | | | | | | | | | | | | | | |
| activator of | | | | | | | | | | | | | | | | | | | |
| transcription | | | | | | | | | | | | | | | | | | | |
| 5A | | | | | | | | | | | | | | | | | | | |
| Signal | P51692 | | 89866.1 | 21.2998 | | | | 64 | | | | | | | 62 | | | | 64 |
| transducer and | | | | | | | | | | | | | | | | | | | |
| activator of | | | | | | | | | | | | | | | | | | | |
| transcription | | | | | | | | | | | | | | | | | | | |
| 5B | | | | | | | | | | | | | | | | | | | |
| Mitogen- | P28482 | | 41389.8 | 79.3199 | | | | | | 79 | | | | | | | 65 | | 79 |
| activated | | | | | | | | | | | | | | | | | | | |
| protein kinase | | | | | | | | | | | | | | | | | | | |
| 1; MAPK1; | | | | | | | | | | | | | | | | | | | |
| ERK-2 | | | | | | | | | | | | | | | | | | | |
| Serine/threonine- | P42345 | | 288892.5 | 16.4969 | 22 | 18 | | | | | | 48 | 16 | | | | | | 48 |
| protein | | | | | | | | | | | | | | | | | | | |
| kinase mTOR | | | | | | | | | | | | | | | | | | | |
| Serine/threonine- | Q9UHD2 | | 83642.4 | 5.3258 | | | | 9 | | | | | | | 16 | | | | 16 |
| protein | | | | | | | | | | | | | | | | | | | |
| kinase TBK1 | | | | | | | | | | | | | | | | | | | |
| Phosphoinositide | Q99570 | | 153103.9 | 6.7192 | | | 13 | | | | | | | 14 | | | | | 14 |
| 3-kinase | | | | | | | | | | | | | | | | | | | |
| regulatory | | | | | | | | | | | | | | | | | | | |
| subunit 4 | | | | | | | | | | | | | | | | | | | |
| Cell division | P06493 | | 34095.5 | 33.2760 | | | | | | | 27 | | | | | | | 24 | 27 |
| protein kinase | | | | | | | | | | | | | | | | | | | |
| 1; CDK1 | | | | | | | | | | | | | | | | | | | |
| Calpain-1 | P07384 | | 81890.2 | 18.7642 | | | | | 22 | | | | | | | 27 | | | 27 |
| catalytic | | | | | | | | | | | | | | | | | | | |
| subunit; | | | | | | | | | | | | | | | | | | | |
| CAPN1 | | | | | | | | | | | | | | | | | | | |
| Mitogen- | P27361 | | 43135.7 | 6.6438 | | | | | | 27 | | | | | | | 27 | | 27 |
| activated | | | | | | | | | | | | | | | | | | | |
| protein kinase | | | | | | | | | | | | | | | | | | | |
| 3; ERK-1 | | | | | | | | | | | | | | | | | | | |
| Ribosomal | P51812 | | 83736.2 | 11.9267 | | | | 20 | | | | | | | 15 | | | | 20 |
| protein S6 | | | | | | | | | | | | | | | | | | | |
| kinase alpha-3; | | | | | | | | | | | | | | | | | | | |
| RSK2 | | | | | | | | | | | | | | | | | | | |
| Inosine-5′- | P12268 | PubMed | 55805.1 | 174.2461 | | | | | 66 | 7 | | | | | | 70 | 14 | | 70 |
| monophosphate | | | | | | | | | | | | | | | | | | | |
| dehydrogenase 2 | | | | | | | | | | | | | | | | | | | |
| Signal | P40763 | | 88068.1 | 15.8176 | | | | 22 | | | | | | | 24 | | | | 24 |
| transducer and | | | | | | | | | | | | | | | | | | | |
| activator of | | | | | | | | | | | | | | | | | | | |
| transcription 3 | | | | | | | | | | | | | | | | | | | |
| Tyrosine- | Q06187 | | 76281.5 | 10.8031 | | | | | 11 | | | | | | | 14 | | | 14 |
| protein kinase | | | | | | | | | | | | | | | | | | | |
| BTK | | | | | | | | | | | | | | | | | | | |
| Regulatory- | Q8N122 | | 149038.0 | 4.8217 | | | 13 | | | | | | | 14 | | | | | 14 |
| associated | | | | | | | | | | | | | | | | | | | |
| protein of | | | | | | | | | | | | | | | | | | | |
| mTOR; | | | | | | | | | | | | | | | | | | | |
| RAPTOR | | | | | | | | | | | | | | | | | | | |
| Rapamycin- | Q6R327 | | 192218.0 | 1.0407 | | 7 | | | | | | | | | | | | | 7 |
| insensitive | | | | | | | | | | | | | | | | | | | |
| companion of | | | | | | | | | | | | | | | | | | | |
| mTOR; | | | | | | | | | | | | | | | | | | | |
| RICTOR | | | | | | | | | | | | | | | | | | | |
| Mitogen- | Q9Y6R4 | | 181552.1 | 4.3965 | | 6 | | | | | | | 11 | | | | | | 11 |
| activated | | | | | | | | | | | | | | | | | | | |
| protein kinase | | | | | | | | | | | | | | | | | | | |
| kinase kinase | | | | | | | | | | | | | | | | | | | |
| 4; MEKK4 | | | | | | | | | | | | | | | | | | | |
| Dedicator of | Q92608 | | 211949.0 | 4.2624 | | 5 | | | | | | | 16 | | | | | | 16 |
| cytokinesis | | | | | | | | | | | | | | | | | | | |
| protein 2; | | | | | | | | | | | | | | | | | | | |
| DOCK2 | | | | | | | | | | | | | | | | | | | |
| Growth factor | P62993 | | 25206.4 | 20.7753 | | | | | | | 15 | | | | | | | 16 | 16 |
| receptor- | | | | | | | | | | | | | | | | | | | |
| bound protein | | | | | | | | | | | | | | | | | | | |
| 2; Grb2 | | | | | | | | | | | | | | | | | | | |
| Epidermal | P42566 | PubMed | 98655.9 | 20.4881 | | | 24 | | | | | | | 33 | | | | | 33 |
| growth factor | | | | | | | | | | | | | | | | | | | |
| receptor | | | | | | | | | | | | | | | | | | | |
| substrate 15 | | | | | | | | | | | | | | | | | | | |
| Phosphatidylinositol | P42356 | | 231319.9 | 5.5247 | | 12 | | | | | | | 18 | | | | | | 18 |
| 4-kinase | | | | | | | | | | | | | | | | | | | |
| alpha | | | | | | | | | | | | | | | | | | | |
| Serine/threonine- | Q9UBE8 | http://www.ncbi.nim.nih.gov/ | 57048.5 | 7.0941 | | | | | | 7 | | | | | | | 14 | | 14 |
| protein | | pubmed/15764709 | | | | | | | | | | | | | | | | | |
| kinase NLK | | | | | | | | | | | | | | | | | | | |
| Histone- | Q86X55 | | 63460.1 | 50.3460 | 5 | | | | 22 | | | 7 | | | | 25 | | | 25 |
| arginine | | | | | | | | | | | | | | | | | | | |
| methyltransferase | | | | | | | | | | | | | | | | | | | |
| CARM1 | | | | | | | | | | | | | | | | | | | |
| Protein | Q14744 | | 72684.1 | 17.3556 | | | | | 27 | | | | | | | 31 | | | 31 |
| arginine N- | | | | | | | | | | | | | | | | | | | |
| methyltransferase 5 | | | | | | | | | | | | | | | | | | | |
| Crk-like | P46109 | | 33777.1 | 4.4171 | | | | | | 11 | | | | | | | | | 11 |
| protein; CRKL | | | | | | | | | | | | | | | | | | | |
| Proliferation- | Q9UQ80 | | 43787.0 | 28.0444 | | | | | | 18 | | | | | | | 27 | | 27 |
| associated | | | | | | | | | | | | | | | | | | | |
| protein 2G4 | | | | | | | | | | | | | | | | | | | |
| Serine/threonine- | P30153 | | 65308.8 | 125.6820 | | | | | 78 | | | | | | | 76 | 11 | | 78 |
| protein | | | | | | | | | | | | | | | | | | | |
| phosphatase | | | | | | | | | | | | | | | | | | | |
| 2A 65 kDa | | | | | | | | | | | | | | | | | | | |
| regulatory | | | | | | | | | | | | | | | | | | | |
| subunit A | | | | | | | | | | | | | | | | | | | |
| alpha isoform | | | | | | | | | | | | | | | | | | | |
| Serine/threonine- | P30154 | | 66213.7 | 5.3180 | | | | | 34 | | | | | | | 37 | | | 37 |
| protein | | | | | | | | | | | | | | | | | | | |
| phosphatase | | | | | | | | | | | | | | | | | | | |
| 2A 65 kDa | | | | | | | | | | | | | | | | | | | |
| regulatory | | | | | | | | | | | | | | | | | | | |
| subunit A beta | | | | | | | | | | | | | | | | | | | |
| isoform | | | | | | | | | | | | | | | | | | | |
| Mitogen- | Q16539 | | 41293.4 | 2.1763 | | | | | | 9 | | | | | | | 11 | | 11 |
| activated | | | | | | | | | | | | | | | | | | | |
| protein kinase | | | | | | | | | | | | | | | | | | | |
| 14; p38 | | | | | | | | | | | | | | | | | | | |
| Protein ALO17 | Q9HCF4 | | 174897.6 | 9.9440 | 22 | | | | | | | 34 | | | | | | | 34 |
| Vascular | P17948 | PubMed | 150769.1 | 2.0434 | | | | | | 23 | | | | | | | 14 | | 23 |
| endothelial | | | | | | | | | | | | | | | | | | | |
| growth factor | | | | | | | | | | | | | | | | | | | |
| receptor 1; | | | | | | | | | | | | | | | | | | | |
| VEGFR-1 | | | | | | | | | | | | | | | | | | | |
| Beta-type | P09619 | | 122828.1 | 2.0664 | | | | | | 13 | | | | | | | 16 | | 16 |
| platelet- | | | | | | | | | | | | | | | | | | | |
| derived growth | | | | | | | | | | | | | | | | | | | |
| factor | | | | | | | | | | | | | | | | | | | |
| receptor; | | | | | | | | | | | | | | | | | | | |
| PDGFRB | | | | | | | | | | | | | | | | | | | |
| Protein- | Q14289 | | 115875.0 | 1.3365 | | | 4 | | | | | | | | | | | | 4 |
| tyrosine kinase | | | | | | | | | | | | | | | | | | | |
| 2-beta; FAK-2 | | | | | | | | | | | | | | | | | | | |
| Talin-1; TLN-1 | Q9Y490 | | 269767.8 | 3.1856 | 19 | | | | | | | 25 | | | | | | | 25 |
| Vinculin | P18206 | | 123799.6 | 17.7700 | | | 35 | | | | | | | 46 | | | | | 46 |
| Filamin-A | P21333 | | 280739.6 | 8.4872 | 42 | | | | | | | 46 | | | | | | | 46 |
| Transforming | Q8WUH2 | | 97158.1 | 1.7989 | | | | 15 | | | | | | | | | | | 15 |
| growth factor- | | | | | | | | | | | | | | | | | | | |
| beta receptor- | | | | | | | | | | | | | | | | | | | |
| associated | | | | | | | | | | | | | | | | | | | |
| protein 1 | | | | | | | | | | | | | | | | | | | |
| DNA- | P78527 | | 469090.2 | 71.4210 | 236 | 30 | | | | | | 251 | 41 | | | | | | 251 |
| dependent | | | | | | | | | | | | | | | | | | | |
| protein kinase | | | | | | | | | | | | | | | | | | | |
| catalytic | | | | | | | | | | | | | | | | | | | |
| subunit | | | | | | | | | | | | | | | | | | | |
| Plasminogen | Q8NC51 | | 44965.4 | 19.2385 | | | | | 17 | | | | | | | 20 | | | 20 |
| activator | | | | | | | | | | | | | | | | | | | |
| inhibitor 1 | | | | | | | | | | | | | | | | | | | |
| RNA-binding | | | | | | | | | | | | | | | | | | | |
| protein; | | | | | | | | | | | | | | | | | | | |
| SERBP1 | | | | | | | | | | | | | | | | | | | |
| Metastasis- | Q94776 | PubMed | 75023.3 | 17.8585 | | | | | 26 | | | | | | | 24 | | | 26 |
| associated | | | | | | | | | | | | | | | | | | | |
| protein MTA2 | | | | | | | | | | | | | | | | | | | |
| Serine/threonine- | Q98ZL6 | | 96722.5 | 3.5358 | | | 6 | | | | | | | 9 | | | | | 9 |
| protein | | | | | | | | | | | | | | | | | | | |
| kinase D2; | | | | | | | | | | | | | | | | | | | |
| PRKD2 | | | | | | | | | | | | | | | | | | | |
| RuvB-like 2; | Q9Y230 | | 51156.7 | 96.1562 | | | | | | 51 | | | | | | | 59 | | 59 |
| TIP48 | | | | | | | | | | | | | | | | | | | |
| RuvB-like 1; | Q9Y265 | | 50228.1 | 111.9313 | | | | | 10 | 53 | | | | | | | 56 | | 56 |
| TIP49 | | | | | | | | | | | | | | | | | | | |
| Casein kinase | P19784 | | 41213.3 | 1.6994 | | | | | | 9 | | | | | | | 11 | | 11 |
| II subunit | | | | | | | | | | | | | | | | | | | |
| alpha′ | | | | | | | | | | | | | | | | | | | |
| Casein kinase | P67870 | | 24942.5 | 9.0324 | | | | | | | 3 | | | | | | | 5 | 5 |
| II subunit beta | | | | | | | | | | | | | | | | | | | |
| Casein kinase I | P48729 | | 38915.0 | 7.8446 | | | | | | | 5 | | | | | | | 7 | 7 |
| isoform alpha | | | | | | | | | | | | | | | | | | | |
| N-terminal | Q96KG9 | | 89631.5 | 14.6654 | | | 11 | | | | | | | 21 | | | | | 21 |
| kinase-like | | | | | | | | | | | | | | | | | | | |
| protein; | | | | | | | | | | | | | | | | | | | |
| SCYL1, | | | | | | | | | | | | | | | | | | | |
| telomerase i | | | | | | | | | | | | | | | | | | | |
| Telomere | Q9Y4R8 | PubMed: | 91747.2 | 7.6607 | | | | 25 | | | | | | | 20 | | | | 25 |
| length | | 12670948 | | | | | | | | | | | | | | | | | |
| regulation | | | | | | | | | | | | | | | | | | | |
| protein TEL2 | | | | | | | | | | | | | | | | | | | |
| homolog | | | | | | | | | | | | | | | | | | | |
| 182 kDa | Q9C0C2 | | 181781.8 | 7.9788 | 12 | | | | | | | 22 | | | | | | | 22 |
| tankyrase-1- | | | | | | | | | | | | | | | | | | | |
| binding protein | | | | | | | | | | | | | | | | | | | |
| Serine/threonine- | Q5H9R7 | | 97669.4 | 10.1079 | | | 16 | | | | | | | 24 | | | | | 24 |
| protein | | | | | | | | | | | | | | | | | | | |
| phosphatase 6 | | | | | | | | | | | | | | | | | | | |
| regulatory | | | | | | | | | | | | | | | | | | | |
| subunit 3; | | | | | | | | | | | | | | | | | | | |
| SAPS3 | | | | | | | | | | | | | | | | | | | |
| CDC27; | P30260 | | 91867.6 | 4.4289 | | | | 17 | | | | | | | 20 | | | | 20 |
| Anaphase- | | | | | | | | | | | | | | | | | | | |
| promoting | | | | | | | | | | | | | | | | | | | |
| complex | | | | | | | | | | | | | | | | | | | |
| subunit 3 | | | | | | | | | | | | | | | | | | | |
| Inhibitor of | Q15111 | | 84729.2 | 2.1707 | | | | 16 | | | | | | | | | | | 16 |
| nuclear factor | | | | | | | | | | | | | | | | | | | |
| kappa-B kinase | | | | | | | | | | | | | | | | | | | |
| subunit alpha | | | | | | | | | | | | | | | | | | | |
| Serine/threonine- | P67775 | | 35594.3 | 63.3310 | | | | | | | 20 | | | | | | | 16 | 20 |
| protein | | | | | | | | | | | | | | | | | | | |
| phosphatase | | | | | | | | | | | | | | | | | | | |
| 2A catalytic | | | | | | | | | | | | | | | | | | | |
| subunit alpha | | | | | | | | | | | | | | | | | | | |
| isoform | | | | | | | | | | | | | | | | | | | |
| Arf-GAP with | Q15057 | | 88028.9 | 4.8244 | | | | 18 | | | | | | | 22 | | | | 22 |
| coiled-coil, | | | | | | | | | | | | | | | | | | | |
| ANK repeat | | | | | | | | | | | | | | | | | | | |
| and PH | | | | | | | | | | | | | | | | | | | |
| domain- | | | | | | | | | | | | | | | | | | | |
| containing | | | | | | | | | | | | | | | | | | | |
| protein 2 | | | | | | | | | | | | | | | | | | | |
| Interleukin | Q12905 | | 43062.2 | 48.8644 | | | | | | 25 | | | | | | | 20 | | 25 |
| enhancer- | | | | | | | | | | | | | | | | | | | |
| binding factor | | | | | | | | | | | | | | | | | | | |
| 2; ILF2 | | | | | | | | | | | | | | | | | | | |
| Interleukin | Q12906 | | 95338.6 | 16.2442 | | | 9 | 20 | | | | | | 9 | 21 | | | | 21 |
| enhancer- | | | | | | | | | | | | | | | | | | | |
| binding factor | | | | | | | | | | | | | | | | | | | |
| 3; ILF3 | | | | | | | | | | | | | | | | | | | |
| 14-3-3 protein | P62258 | | 29174.0 | 20.1372 | | | | | | | 15 | | | | | | | 17 | 17 |
| epsilon; | | | | | | | | | | | | | | | | | | | |
| YWHAE | | | | | | | | | | | | | | | | | | | |
| 14-3-3 protein | P61981 | | 28302.7 | 25.6664 | | | | | | | 12 | | | | | | | 12 | 12 |
| gamma; | | | | | | | | | | | | | | | | | | | |
| YWHAG | | | | | | | | | | | | | | | | | | | |
| Serine/threonine- | Q8TD19 | | 107168.8 | 5.5558 | | | 5 | | | | | | | 11 | | | | | 11 |
| protein | | | | | | | | | | | | | | | | | | | |
| kinase Nek9 | | | | | | | | | | | | | | | | | | | |
| Serine- | Q9Y3F4 | | 38438.4 | 9.5433 | | | | | | 16 | | | | | | | 10 | | 16 |
| threonine | | | | | | | | | | | | | | | | | | | |
| kinase | | | | | | | | | | | | | | | | | | | |
| receptor- | | | | | | | | | | | | | | | | | | | |
| associated | | | | | | | | | | | | | | | | | | | |
| protein; | | | | | | | | | | | | | | | | | | | |
| STRAP | | | | | | | | | | | | | | | | | | | |
| Transforming | Q969Z0 | | 70738.2 | 7.4653 | | | | | 14 | | | | | | | 14 | | | 14 |
| growth factor | | | | | | | | | | | | | | | | | | | |
| beta regulator 4 | | | | | | | | | | | | | | | | | | | |
| Insulin-like | Q00425 | | 63720.1 | 14.2841 | | | | | 18 | | | | | | | 16 | | | 18 |
| growth factor 2 | | | | | | | | | | | | | | | | | | | |
| mRNA-binding | | | | | | | | | | | | | | | | | | | |
| protein 3 | | | | | | | | | | | | | | | | | | | |
| Insulin-like | Q9NZI8 | | 63456.6 | 26.2110 | | | | | 32 | | | | | | | 22 | | | 32 |
| growth factor 2 | | | | | | | | | | | | | | | | | | | |
| mRNA-binding | | | | | | | | | | | | | | | | | | | |
| protein 1; | | | | | | | | | | | | | | | | | | | |
| IGF2BP1 | | | | | | | | | | | | | | | | | | | |
| Cell | Q92600 | | 33631.3 | 16.2644 | | | | | | | 9 | | | | | | | 10 | 10 |
| differentiation | | | | | | | | | | | | | | | | | | | |
| protein RCD1 | | | | | | | | | | | | | | | | | | | |
| homolog | | | | | | | | | | | | | | | | | | | |
| 5′-AMP- | Q13131 | | 62807.9 | 11.2910 | | | | | 12 | | | | | | | 9 | | | 12 |
| activated | | | | | | | | | | | | | | | | | | | |
| protein kinase | | | | | | | | | | | | | | | | | | | |
| catalytic | | | | | | | | | | | | | | | | | | | |
| subunit alpha- | | | | | | | | | | | | | | | | | | | |
| 1; PRKAA1 | | | | | | | | | | | | | | | | | | | |
| 5′-AMP- | P54619 | | 37579.5 | 25.9468 | | | | | | | 19 | | | | | | | 19 | 19 |
| activated | | | | | | | | | | | | | | | | | | | |
| protein kinase | | | | | | | | | | | | | | | | | | | |
| subunit | | | | | | | | | | | | | | | | | | | |
| gamma-1; | | | | | | | | | | | | | | | | | | | |
| PRKAG1 | | | | | | | | | | | | | | | | | | | |
| Calpain small | P04632 | | 28315.8 | 10.0635 | | | | | | | 9 | | | | | | | 6 | 9 |
| subunit 1; | | | | | | | | | | | | | | | | | | | |
| CAPNS1 | | | | | | | | | | | | | | | | | | | |
| Cell growth- | Q9NX58 | | 43614.9 | 4.7794 | | | | | | 4 | | | | | | | 7 | | 7 |
| regulating | | | | | | | | | | | | | | | | | | | |
| nucleolar | | | | | | | | | | | | | | | | | | | |
| protein; LYAR | | | | | | | | | | | | | | | | | | | |
| Serine | Q43464 | | 48840.9 | 8.0093 | | | | | | | 6 | | | | | | | 6 | 6 |
| protease | | | | | | | | | | | | | | | | | | | |
| HTRA2 | | | | | | | | | | | | | | | | | | | |
| Kelch-like | Q14145 | | 69666.5 | 12.8272 | | | | | 21 | | | | | | | 20 | | | 21 |
| ECH- | | | | | | | | | | | | | | | | | | | |
| associated | | | | | | | | | | | | | | | | | | | |
| protein 1 | | | | | | | | | | | | | | | | | | | |
| THUMP | Q9BV44 | | 57002.9 | 15.3092 | | | | | 18 | | | | | | | 19 | | | 19 |
| domain- | | | | | | | | | | | | | | | | | | | |
| containing | | | | | | | | | | | | | | | | | | | |
| protein 3 | | | | | | | | | | | | | | | | | | | |
| Histone | Q14929 | | 49512.7 | 10.9424 | | | | | | 4 | | | | | | | 18 | | 18 |
| acetyltransferase | | | | | | | | | | | | | | | | | | | |
| type B | | | | | | | | | | | | | | | | | | | |
| catalytic | | | | | | | | | | | | | | | | | | | |
| subunit; HAT1 | | | | | | | | | | | | | | | | | | | |
| Proliferating | P12004 | | 28768.9 | 38.3707 | | | | | | | 18 | | | | | | | 16 | 18 |
| cell nuclear | | | | | | | | | | | | | | | | | | | |
| antigen | | | | | | | | | | | | | | | | | | | |
| Mitotic | Q43684 | | 37154.9 | 12.0013 | | | | | | 8 | | | | | | | 10 | | 10 |
| checkpoint | | | | | | | | | | | | | | | | | | | |
| protein BUB3 | | | | | | | | | | | | | | | | | | | |
| Histone | Q13547 | | 55103.1 | 19.2088 | | | | | 11 | | | | | | | 16 | | | 16 |
| deacetylase 1; | | | | | | | | | | | | | | | | | | | |
| HDAC1 | | | | | | | | | | | | | | | | | | | |
| Histone | Q13547 | | 48847.9 | 9.1175 | | | | | | 9 | | | | | | | 13 | | 13 |
| deacetylase 3; | | | | | | | | | | | | | | | | | | | |
| HDAC3 | | | | | | | | | | | | | | | | | | | |
| Histone | Q92769 | | 55364.4 | 15.8525 | | | | | 7 | | | | | | | 11 | | | 11 |
| deacetylase 2; | | | | | | | | | | | | | | | | | | | |
| HDAC2 | | | | | | | | | | | | | | | | | | | |
| Histone | Q9UBN7 | | 131419.6 | 8.6654 | | 11 | | | | | | | 9 | | | | | | 11 |
| deacetylase 6; | | | | | | | | | | | | | | | | | | | |
| HDAC6 | | | | | | | | | | | | | | | | | | | |
| N- | Q9H0A0 | | 115704.1 | 3.0039 | | | 4 | | | | | | | 14 | | | | | 14 |
| acetyltransferase | | | | | | | | | | | | | | | | | | | |
| 10; NAT10 | | | | | | | | | | | | | | | | | | | |
| Histone H1.2 | P16403 | | 21364.8 | 7.5569 | | | | | | | 7 | | | | | | | 6 | 7 |
| BRCA1-A | Q9NXR7 | | 46974.6 | 11.1230 | | | | | | 8 | | | | | | | 12 | | 12 |
| complex | | | | | | | | | | | | | | | | | | | |
| subunit BRE | | | | | | | | | | | | | | | | | | | |
| S-adenosyl-L- | Q8N1G2 | | 95321.1 | 3.4876 | | | 9 | | | | | | | 10 | | | | | 10 |
| methionine- | | | | | | | | | | | | | | | | | | | |
| dependent | | | | | | | | | | | | | | | | | | | |
| methyltransferase | | | | | | | | | | | | | | | | | | | |
| FTSJD2 | | | | | | | | | | | | | | | | | | | |
| Cell division | Q75419 | | 65568.8 | 13.0274 | | | | | 14 | | | | | | | 14 | | | 14 |
| control protein | | | | | | | | | | | | | | | | | | | |
| 45 homolog | | | | | | | | | | | | | | | | | | | |
| Probable | Q76071 | | 37840.1 | 15.5890 | | | | | | 8 | | | | | | | 13 | | 13 |
| cytosolic iron- | | | | | | | | | | | | | | | | | | | |
| sulfur protein | | | | | | | | | | | | | | | | | | | |
| assembly | | | | | | | | | | | | | | | | | | | |
| protein CIAO1 | | | | | | | | | | | | | | | | | | | |
| Serine/threonine- | Q96SB34 | | 74325.0 | 7.2125 | | | 6 | | | | | | | 10 | | | | | 10 |
| protein | | | | | | | | | | | | | | | | | | | |
| kinase SRPK1 | | | | | | | | | | | | | | | | | | | |
| Regulator of | Q95758 | | 59689.7 | 0.5622 | | | | | | | | | | | | 13 | | | 13 |
| differentiation | | | | | | | | | | | | | | | | | | | |
| 1′ ROD1 | | | | | | | | | | | | | | | | | | | |
| Mitogen- | P45983 | | 48295.7 | 6.6247 | | | | | | 13 | | | | | | | 6 | | 13 |
| activated | | | | | | | | | | | | | | | | | | | |
| protein kinase | | | | | | | | | | | | | | | | | | | |
| 8; JNK1; | | | | | | | | | | | | | | | | | | | |
| SAPK1 | | | | | | | | | | | | | | | | | | | |
| Transducin- | Q04726 | | 83416.9 | 3.7256 | | | | | | | | | | | 13 | | | | 13 |
| like enhancer | | | | | | | | | | | | | | | | | | | |
| protein 3; TLE3 | | | | | | | | | | | | | | | | | | | |
| Mitogen- | P45984 | | 48139.2 | 3.5130 | | | | | 7 | 12 | | | | | | | | | 12 |
| activated | | | | | | | | | | | | | | | | | | | |
| protein kinase | | | | | | | | | | | | | | | | | | | |
| 9; JNK2 | | | | | | | | | | | | | | | | | | | |
| Serine/threonine- | Q66LE6 | | 52042.6 | 5.9742 | | | | | | 13 | | | | | | | 10 | | 13 |
| protein | | | | | | | | | | | | | | | | | | | |
| phosphatase | | | | | | | | | | | | | | | | | | | |
| 2A 55 kDa | | | | | | | | | | | | | | | | | | | |
| regulatory | | | | | | | | | | | | | | | | | | | |
| subunit B delta | | | | | | | | | | | | | | | | | | | |
| isoform | | | | | | | | | | | | | | | | | | | |
| Serine/threonine- | Q8TF05 | | 107004.4 | 9.6747 | | | 13 | | | | | | | 15 | | | | | 15 |
| protein | | | | | | | | | | | | | | | | | | | |
| phosphatase 4 | | | | | | | | | | | | | | | | | | | |
| regulatory | | | | | | | | | | | | | | | | | | | |
| subunit 1 | | | | | | | | | | | | | | | | | | | |
| Mitogen- | P31152 | | 65921.9 | 1.9160 | | | | | | 7 | | | | | | | 6 | | 7 |
| activated | | | | | | | | | | | | | | | | | | | |
| protein kinase | | | | | | | | | | | | | | | | | | | |
| 4; ERK4 | | | | | | | | | | | | | | | | | | | |
| Mitogen- | Q16659 | | 82681.0 | 3.0471 | | | | | | 9 | | | | | | | 11 | | 11 |
| activated | | | | | | | | | | | | | | | | | | | |
| protein kinase | | | | | | | | | | | | | | | | | | | |
| 6; ERK3 | | | | | | | | | | | | | | | | | | | |
| Cell division | P50613 | | 39038.5 | 3.8042 | | | | | | 6 | | | | | | | 9 | | 9 |
| protein kinase 7 | | | | | | | | | | | | | | | | | | | |
| Cell division | P24941 | | 33929.6 | 3.8552 | | | | | | | 9 | | | | | | | 8 | 9 |
| protein kinase 2 | | | | | | | | | | | | | | | | | | | |
| Tyrosine- | Q9H3S7 | | 178974.0 | 5.6692 | | 10 | | | | | | | 13 | | | | | | 13 |
| protein | | | | | | | | | | | | | | | | | | | |
| phosphatase | | | | | | | | | | | | | | | | | | | |
| non-receptor | | | | | | | | | | | | | | | | | | | |
| type 23; | | | | | | | | | | | | | | | | | | | |
| PTPN23 | | | | | | | | | | | | | | | | | | | |
| Tyrosine- | P18031 | | 49967.0 | 3.5169 | | | | | | 9 | | | | | | | | | 9 |
| protein | | | | | | | | | | | | | | | | | | | |
| phosphatase | | | | | | | | | | | | | | | | | | | |
| non-receptor | | | | | | | | | | | | | | | | | | | |
| type 1; PTPN1 | | | | | | | | | | | | | | | | | | | |
| Probable E3 | Q9H000 | | 46940.5 | 7.3243 | | | | | | 11 | | | | | | | 12 | | 12 |
| ubiquitin- | | | | | | | | | | | | | | | | | | | |
| protein ligase | | | | | | | | | | | | | | | | | | | |
| makorin-2 | | | | | | | | | | | | | | | | | | | |
| E3 ubiquitin- | Q9UNE7 | | 34856.3 | 30.9572 | | | | | | | 14 | | | | | | | 12 | 14 |
| protein ligase | | | | | | | | | | | | | | | | | | | |
| CHIP | | | | | | | | | | | | | | | | | | | |
| Protein SET | Q01105 | | 33488.9 | 21.0046 | | | | | | 7 | | | | | | | 9 | | 9 |
| E3 ubiquitin- | Q5T4S7 | | 573842.7 | 20.1396 | 112 | | | | | | | 128 | | | | | | | 128 |
| protein ligase | | | | | | | | | | | | | | | | | | | |
| UBR4 | | | | | | | | | | | | | | | | | | | |
| ELAV-like | Q15717 | | 36092.0 | 55.2953 | | | | | | | 20 | | | | | | | 21 | 21 |
| protein 1 | | | | | | | | | | | | | | | | | | | |
| 28 kDa heat- | Q13442 | | 20630.0 | 3.7688 | | | | | | | 2 | | | | | | | | 2 |
| and acid-stable | | | | | | | | | | | | | | | | | | | |
| phosphoprotein | | | | | | | | | | | | | | | | | | | |
| Autophagy | Q9H1Y0 | | 32447.3 | 2.0138 | | | | | 9 | | | | | | | | | | 9 |
| protein 5 | | | | | | | | | | | | | | | | | | | |
| Serine/threonine- | Q13535 | | 301367.6 | 1.0124 | 10 | | | | | | | | | | | | | | 10 |
| protein | | | | | | | | | | | | | | | | | | | |
| kinase ATR | | | | | | | | | | | | | | | | | | | |
| Protein | Q8N163 | | 102901.7 | 22.1394 | | | 19 | | | | | | | 26 | | | | | 26 |
| KIAA1967 p30 | | | | | | | | | | | | | | | | | | | |
| DBC | | | | | | | | | | | | | | | | | | | |
| Transcriptional | Q8WXI9 | | 65260.9 | 1.5826 | | | | | 13 | | | | | | | | | | 13 |
| repressor p66- | | | | | | | | | | | | | | | | | | | |
| beta | | | | | | | | | | | | | | | | | | | |
| Transcription | Q00267 | | 120999.8 | 6.9075 | | 18 | | | | | | | 16 | | | | | | 18 |
| elongation | | | | | | | | | | | | | | | | | | | |
| factor SPT5 | | | | | | | | | | | | | | | | | | | |
| Phosducin-like | Q9H2J4 | | 27614.4 | 4.3938 | | | | | | | 4 | | | | | | | 5 | 5 |
| protein 3 | | | | | | | | | | | | | | | | | | | |
| Nuclease- | P67809 | | 35924.2 | 45.8457 | | | | | | 26 | | | | | | | 24 | | 26 |
| sensitive | | | | | | | | | | | | | | | | | | | |
| element- | | | | | | | | | | | | | | | | | | | |
| binding protein 1 | | | | | | | | | | | | | | | | | | | |
| Protein CREG1 | Q75629 | | 24074.6 | 8.0371 | | | | | | | 2 | | | | | | | 3 | 3 |
| Ras | Q15404 | | 31540.3 | 3.2914 | | | | | | | 5 | | | | | | | 4 | 5 |
| suppressor | | | | | | | | | | | | | | | | | | | |
| protein 1 | | | | | | | | | | | | | | | | | | | |
| Large proline- | P46379 | | 119409.0 | 5.9599 | | 5 | | | | | | | 6 | | | | | | 6 |
| rich protein | | | | | | | | | | | | | | | | | | | |
| BAT3 | | | | | | | | | | | | | | | | | | | |
| Serine/threonine- | Q9BVS4 | | 63283.2 | 3.6676 | | | | 6 | | | | | | | | | | | 6 |
| protein | | | | | | | | | | | | | | | | | | | |
| kinase RIO2 | | | | | | | | | | | | | | | | | | | |
| Serine/threonine- | P36873 | | 36983.9 | 4.9265 | | | | | | | 8 | | | | | | | 7 | 8 |
| protein | | | | | | | | | | | | | | | | | | | |
| phosphatase | | | | | | | | | | | | | | | | | | | |
| PP1-gamma | | | | | | | | | | | | | | | | | | | |
| catalytic | | | | | | | | | | | | | | | | | | | |
| subunit | | | | | | | | | | | | | | | | | | | |
| Integrin-linked | Q13418 | | 51419.4 | 1.6140 | | | | | | 4 | | | | | | | | | 4 |
| protein kinase; | | | | | | | | | | | | | | | | | | | |
| ILK | | | | | | | | | | | | | | | | | | | |
| Proto- | P11309 | | 45412.5 | 0.6796 | | | | | | | | | | | | | | 4 | 4 |
| oncogene | | | | | | | | | | | | | | | | | | | |
| serine/threonine- | | | | | | | | | | | | | | | | | | | |
| protein | | | | | | | | | | | | | | | | | | | |
| kinase pim-1 | | | | | | | | | | | | | | | | | | | |
| Endoplasmin; | P14625 | | 92469.0 | 127.8154 | | | 21 | 79 | 22 | 14 | 4 | | | 48 | 71 | | 20 | 7 | 79 |
| GRP94 | | | | | | | | | | | | | | | | | | | |
| Heat shock | Q12931 | | 80110.2 | 209.2569 | | | | | 80 | | | | | | | 90 | | | 90 |
| protein 75 kDa, | | | | | | | | | | | | | | | | | | | |
| mitochondrial, | | | | | | | | | | | | | | | | | | | |
| TRAP1 | | | | | | | | | | | | | | | | | | | |
| Hsc70- | P50502 | | 41331.8 | 96.9194 | | | | | | 23 | | | | | | | 19 | | 23 |
| interacting | | | | | | | | | | | | | | | | | | | |
| protein; HIP | | | | | | | | | | | | | | | | | | | |
| Stress- | P31948 | | 62639.5 | 129.2074 | | | | | 68 | | | | | | | 72 | | | 72 |
| induced- | | | | | | | | | | | | | | | | | | | |
| phosphoprotein | | | | | | | | | | | | | | | | | | | |
| 1; HOP | | | | | | | | | | | | | | | | | | | |
| Heat shock | P11142 | | 70898.2 | 211.9690 | | | | | 73 | | | | | | | 105 | | | 105 |
| cognate 71 kDa | | | | | | | | | | | | | | | | | | | |
| protein | | | | | | | | | | | | | | | | | | | |
| Heat shock 70 kDa | P08107 | | 70052.3 | 115.7597 | | | | | 65 | | | | | | | 82 | | | 82 |
| protein | | | | | | | | | | | | | | | | | | | |
| 1A/1B | | | | | | | | | | | | | | | | | | | |
| Heat shock- | P54652 | | 70021.1 | 7.7656 | | | | | 37 | | | | | | | 45 | | | 45 |
| related 70 kDa | | | | | | | | | | | | | | | | | | | |
| protein 2 | | | | | | | | | | | | | | | | | | | |
| Heat shock 70 kDa | P34932 | | 94331.2 | 5.9277 | | | 9 | | | | | | | 17 | | | | | 17 |
| protein 4 | | | | | | | | | | | | | | | | | | | |
| Heat shock 70 kDa | P17066 | | 71028.3 | 1.6158 | | | | | 39 | | | | | | | 44 | | | 44 |
| protein 6 | | | | | | | | | | | | | | | | | | | |
| Hsp90 co- | Q16543 | | 44468.5 | 45.9047 | | | | | | 17 | | | | | | | 16 | | 17 |
| chaperone | | | | | | | | | | | | | | | | | | | |
| Cdc37 | | | | | | | | | | | | | | | | | | | |
| Activator of 90 kDa | Q95433 | | 38274.4 | 19.5699 | | | | | | 12 | | | | | | | 12 | | 12 |
| heat shock | | | | | | | | | | | | | | | | | | | |
| protein ATPase | | | | | | | | | | | | | | | | | | | |
| homolog 1; | | | | | | | | | | | | | | | | | | | |
| AHSA1 | | | | | | | | | | | | | | | | | | | |
| DnaJ homolog | Q75165 | | 29841.7 | 6.8808 | | | | | | | 5 | | | | | | | 6 | 6 |
| subfamily C | | | | | | | | | | | | | | | | | | | |
| member 8 | | | | | | | | | | | | | | | | | | | |
| DnaJ homolog | Q9UBS4 | | 40514.0 | 14.4606 | | | | | | 5 | | | | | | | 6 | | 6 |
| subfamily B | | | | | | | | | | | | | | | | | | | |
| member 11 | | | | | | | | | | | | | | | | | | | |
| DnaJ homolog | Q99615 | | 56441.0 | 19.0068 | | | | | 14 | | | | | | | 24 | | | 24 |
| subfamily C | | | | | | | | | | | | | | | | | | | |
| member 7 | | | | | | | | | | | | | | | | | | | |
| DnaJ homolog | Q60884 | | 45745.8 | 31.2111 | | | | | | 23 | | | | | | | 22 | | 23 |
| subfamily A | | | | | | | | | | | | | | | | | | | |
| member 2 | | | | | | | | | | | | | | | | | | | |
| DnaJ homolog | Q8WXX5 | | 29909.8 | 4.9413 | | | | | | | 3 | | | | | | | 4 | 4 |
| subfamily C | | | | | | | | | | | | | | | | | | | |
| member 9 | | | | | | | | | | | | | | | | | | | |
| DnaJ homolog | P31689 | | 44868.4 | 49.8849 | | | | | | 26 | | | | | | | 26 | | 26 |
| subfamily A | | | | | | | | | | | | | | | | | | | |
| member 1 | | | | | | | | | | | | | | | | | | | |
| DnaJ homolog | Q96EY1 | | 52537.9 | 7.9449 | | | | | | 12 | | | | | | | 11 | | 12 |
| subfamily A | | | | | | | | | | | | | | | | | | | |
| member 3 | | | | | | | | | | | | | | | | | | | |
| Peptidyl-prolyl | Q02790 | | 51804.7 | 58.4334 | | | | | 37 | | | | | | | 50 | | | 50 |
| cis-trans | | | | | | | | | | | | | | | | | | | |
| isomerase | | | | | | | | | | | | | | | | | | | |
| FKBP4 | | | | | | | | | | | | | | | | | | | |
| Peptidyl-prolyl | Q14318 | | 44561.8 | 1.5935 | | | | | 5 | | | | | | | | | | 5 |
| cis-trans | | | | | | | | | | | | | | | | | | | |
| isomerase | | | | | | | | | | | | | | | | | | | |
| FKBP8 | | | | | | | | | | | | | | | | | | | |
| Peptidyl-prolyl | Q13356 | | 58823.6 | 6.0454 | | | | | 11 | | | | | | | 21 | | | 21 |
| cis-trans | | | | | | | | | | | | | | | | | | | |
| isomerase-like 2 | | | | | | | | | | | | | | | | | | | |
| AH receptor- | Q00170 | | 37664.2 | 32.7606 | | | | | | | 20 | | | | | | | 20 | 20 |
| interacting | | | | | | | | | | | | | | | | | | | |
| protein; | | | | | | | | | | | | | | | | | | | |
| Immunophilin | | | | | | | | | | | | | | | | | | | |
| homolog ARA9 | | | | | | | | | | | | | | | | | | | |
| Heat shock | Q92598 | | 96865.2 | 0.8860 | | | | | | | | | | 9 | | | | | 9 |
| protein 105 kDa; | | | | | | | | | | | | | | | | | | | |
| Hsp110 | | | | | | | | | | | | | | | | | | | |
| BAG family | Q95816 | | 23772.0 | 4.0787 | | | | | | | 4 | | | | | | | 2 | 4 |
| molecular | | | | | | | | | | | | | | | | | | | |
| chaperone | | | | | | | | | | | | | | | | | | | |
| regulator 2 | | | | | | | | | | | | | | | | | | | |
| Protein unc-45 | Q9H3U1 | | 103077.2 | 16.4590 | | | | 28 | | | | | | | 45 | | | | 45 |
| homolog A | | | | | | | | | | | | | | | | | | | |
| Mitochondrial | Q94826 | | 67455.0 | 3.4547 | | | | | 14 | | | | | | | 10 | | | 14 |
| import | | | | | | | | | | | | | | | | | | | |
| receptor | | | | | | | | | | | | | | | | | | | |
| subunit TOM70 | | | | | | | | | | | | | | | | | | | |
| Stress-70 | P38646 | | 73680.7 | 31.2908 | | | | | 41 | | | | | | | 38 | | | 41 |
| protein; GRP75 | | | | | | | | | | | | | | | | | | | |
| 78 kDa | P11021 | | 72333.1 | 12.7943 | | | | | 32 | | | | | | | 36 | | | 36 |
| glucose- | | | | | | | | | | | | | | | | | | | |
| regulated | | | | | | | | | | | | | | | | | | | |
| protein; GRP78 | | | | | | | | | | | | | | | | | | | |
| 60 kDa heat | P10809 | | 61054.8 | 27.0126 | | | | | 32 | | | | | | | 28 | | | 32 |
| shock protein; | | | | | | | | | | | | | | | | | | | |
| Hsp60 | | | | | | | | | | | | | | | | | | | |
| Heat shock | P04792 | | 22782.6 | 162.0092 | | | | | | | 24 | | | | | | | 21 | 24 |
| protein beta-1; | | | | | | | | | | | | | | | | | | | |
| Hsp27 | | | | | | | | | | | | | | | | | | | |

*in gray are proteins for which the excized gel size fails to mach the reported MW

TABLE 5e
Function, pathway and network analysis eligible proteins selected
for processing by Ingenuity Pathway from the input list
©2000-2010 Ingenuity Systems, Inc. All rights reserved.

| ID | Gene | Description | Location | Family | Drugs |
|---|---|---|---|---|---|
| P07900 | HSP90AA1 | heat shock protein 90 kDa | Cytoplasm | other | 17- |
| | | alpha (cytosolic), class A | | | dimethylaminoethylamino- |
| | | member 1 | | | 17- |
| | | | | | demethoxygeldanamycin, |
| | | | | | IPI-504 |
| P08238 | HSP90AB1 | heat shock protein 90 kDa | Cytoplasm | other | 17- |
| | | alpha (cytosolic), class B | | | dimethylaminoethylamino- |
| | | member 1 | | | 17- |
| | | | | | demethoxygeldanamycin, |
| | | | | | IPI-504 |
| P00519 | ABL1 | c-abl oncogene 1, receptor | Nucleus | kinase | saracatinib, imatinib, |
| | | tyrosine kinase | | | temozolomide |
| P11274 | BCR | breakpoint cluster region | Cytoplasm | kinase | imatinib |
| P51812 | RPS6KA3 | ribosomal protein S6 | Cytoplasm | kinase | |
| | | kinase, 90 kDa, polypeptide 3 | | | |
| Q15418 | RPS6KA1 | ribosomal protein S6 | Cytoplasm | kinase | |
| | | kinase, 90 kDa, polypeptide 1 | | | |
| P42345 | MTOR | mechanistic target of | Nucleus | kinase | deforolimus, OSI-027, |
| | | rapamycin | | | temsirolimus, tacrolimus, |
| | | (serine/threonine kinase) | | | everolimus |
| Q8N122 | RPTOR | regulatory associated | Cytoplasm | other | |
| | | protein of MTOR, complex 1 | | | |
| Q99570 | PIK3R4 | phosphoinositide-3-kinase, | Cytoplasm | kinase | |
| | | regulatory subunit 4 | | | |
| Q8NEB9 | PIK3C3 | phosphoinositide-3-kinase, | Cytoplasm | kinase | |
| | | class 3 | | | |
| Q9BPZ7 | MAPKAP1 | mitogen-activated protein | unknown | other | |
| | | kinase associated protein 1 | | | |
| P42229 | STAT5A | signal transducer and | Nucleus | transcription | |
| | | activator of transcription 5A | | regulator | |
| P51692 | STAT5B | signal transducer and | Nucleus | transcription | |
| | | activator of transcription 5B | | regulator | |
| P04049 | RAF1 | v-raf-1 murine leukemia | Cytoplasm | kinase | sorafenib |
| | | viral oncogene homolog 1 | | | |
| P10398 | ARAF | v-raf murine sarcoma 3611 | Cytoplasm | kinase | |
| | | viral oncogene homolog | | | |
| P15498 | VAV1 | vav 1 guanine nucleotide | Nucleus | transcription | |
| | | exchange factor | | regulator | |
| Q06187 | BTK | Bruton | Cytoplasm | kinase | |
| | | agammaglobulinemia | | | |
| | | tyrosine kinase | | | |
| Q05397 | PTK2 | PTK2 protein tyrosine | Cytoplasm | kinase | |
| | | kinase 2 | | | |
| Q9H3S7 | PTPN23 | protein tyrosine | Cytoplasm | phosphatase | |
| | | phosphatase, non-receptor | | | |
| | | type 23 | | | |
| P40763 | STAT3 | signal transducer and | Nucleus | transcription | |
| | | activator of transcription 3 | | regulator | |
| | | (acute-phase response factor) | | | |
| P51617 | IRAK1 | interleukin-1 receptor- | Plasma | kinase | |
| | | associated kinase 1 | Membrane | | |
| P28482 | MAPK1 | mitogen-activated protein | Cytoplasm | kinase | |
| | | kinase 1 | | | |
| Q9Y6R4 | MAP3K4 | mitogen-activated protein | Cytoplasm | kinase | |
| | | kinase kinase kinase 4 | | | |
| Q15750 | TAB1 | TGF-beta activated kinase 1/ | Cytoplasm | enzyme | |
| | | MAP3K7 binding protein 1 | | | |
| Q16539 | MAPK14 | mitogen-activated protein | Cytoplasm | kinase | SCIO-469, RO-3201195 |
| | | kinase 14 | | | |
| P07384 | CAPN1 | calpain 1, (mu/l) large | Cytoplasm | peptidase | |
| | | subunit | | | |
| O00425 | IGF2BP3 | insulin-like growth factor 2 | Cytoplasm | translation | |
| | | mRNA binding protein 3 | | regulator | |
| O88477 | IGF2BP1 | insulin-like growth factor 2 | Cytoplasm | translation | |
| | | mRNA binding protein 1 | | regulator | |
| Q9Y6M1 | IGF2BP2 | insulin-like growth factor 2 | Cytoplasm | translation | |
| | | mRNA binding protein 2 | | regulator | |
| Q9Y265 | RUVBL1 | RuvB-like 1 (E. coli) | Nucleus | transcription | |
| | | | | regulator | |
| Q9Y230 | RUVBL2 | RuvB-like 2 (E. coli) | Nucleus | transcription | |
| | | | | regulator | |
| Q99417 | MYCBP | c-myc binding protein | Nucleus | transcription | |
| | | | | regulator | |
| O43823 | AKAP8 | A kinase (PRKA) anchor | Nucleus | other | |
| | | protein 8 | | | |
| Q9ULX6 | AKAP8L | A kinase (PRKA) anchor | Nucleus | other | |
| | | protein 8-like | | | |
| P06748 | NPM1 | nucleophosmin (nucleolar | Nucleus | transcription | |
| | (includes | phosphoprotein B23, | | regulator | |
| | EG: 4869) | numatrin) | | | |
| Q86X55 | CARM1 | coactivator-associated | Nucleus | transcription | |
| | | arginine methyltransferase 1 | | regulator | |
| Q13555 | CAMK2G | calcium/calmodulin- | Cytoplasm | kinase | |
| | | dependent protein kinase II | | | |
| | | gamma | | | |
| P29597 | TYK2 | tyrosine kinase 2 | Plasma | kinase | |
| | | | Membrane | | |
| Q9UHD2 | TBK1 | TANK-binding kinase 1 | Cytoplasm | kinase | |
| P42356 | PI4KA | phosphatidylinositol 4- | Cytoplasm | kinase | |
| | | kinase, catalytic, alpha | | | |
| Q96Q15 | SMG1 | SMG1 homolog, | Cytoplasm | kinase | |
| | | phosphatidylinositol 3- | | | |
| | | kinase-related kinase (C. | | | |
| | | elegans) | | | |
| Q93100 | PHKB | phosphorylase kinase, beta | Cytoplasm | kinase | |
| Q9NVE7 | PANK4 | pantothenate kinase 4 | Cytoplasm | kinase | |
| Q13131 | PRKAA1 | protein kinase, AMP- | Cytoplasm | kinase | |
| | | activated, alpha 1 catalytic | | | |
| | | subunit | | | |
| Q8N7V9 | PRKAG1 | protein kinase, AMP- | Nucleus | kinase | |
| | | activated, gamma 1 non- | | | |
| | | catalytic subunit | | | |
| Q96KG9 | SCYL1 | SCY1-like 1 (S. cerevisiae) | Cytoplasm | kinase | |
| Q13315 | ATM | ataxia telangiectasia | Nucleus | kinase | |
| | | mutated | | | |
| Q13535 | ATR | ataxia telangiectasia | Nucleus | kinase | |
| | (includes | and Rad3 related | | | |
| | EG: 545) | | | | |
| Q9Y3F4 | STRAP | serine/threonine kinase | Plasma | other | |
| | | receptor associated protein | Membrane | | |
| Q9BVS4 | RIOK2 | RIO kinase 2 (yeast) | unknown | kinase | |
| Q9BZL6 | PRKD2 | protein kinase D2 | Cytoplasm | kinase | |
| P48729 | CSNK1A1 | casein kinase 1, alpha 1 | Cytoplasm | kinase | |
| P67870 | CSNK2B | casein kinase 2, beta | Cytoplasm | kinase | |
| | | polypeptide | | | |
| Q8IVT5 | KSR1 | kinase suppressor of ras 1 | Cytoplasm | kinase | |
| Q9NSY1 | BMP2K | BMP2 inducible kinase | Nucleus | kinase | |
| | (includes | | | | |
| | EG: 55589) | | | | |
| Q96SB4 | SRPK1 | SFRS protein kinase 1 | Nucleus | kinase | |
| P78362 | SRPK2 | SFRS protein kinase 2 | Nucleus | kinase | |
| P53350 | PLK1 | polo-like kinase 1 | Nucleus | kinase | BI 2536 |
| | | (Drosophila) | | | |
| P06493 | CDK1 | cyclin-dependent kinase 1 | Nucleus | kinase | flavopiridol |
| P50613 | CDK7 | cyclin-dependent kinase 7 | Nucleus | kinase | BMS-387032, flavopiridol |
| Q8IX12 | CCAR1 | cell division cycle and | Nucleus | other | |
| | | apoptosis regulator 1 | | | |
| P30260 | CDC27 | cell division cycle 27 | Nucleus | other | |
| | | homolog (S. cerevisiae) | | | |
| Q9UJX2 | CDC23 | cell division cycle 23 | Nucleus | enzyme | |
| | (includes | homolog (S. cerevisiae) | | | |
| | EG: 8697) | | | | |
| Q13042 | CDC16 | cell division cycle 16 | Nucleus | other | |
| | | homolog (S. cerevisiae) | | | |
| P50750 | CDK9 | cyclin-dependent kinase 9 | Nucleus | kinase | BMS-387032, flavopiridol |
| O60566 | BUB1B | budding uninhibited by | Nucleus | kinase | |
| | | benzimidazoles 1 homolog | | | |
| | | beta (yeast) | | | |
| O43683 | BUB1 | budding uninhibited by | Nucleus | kinase | |
| | | benzimidazoles 1 homolog | | | |
| | | (yeast) | | | |
| Q9H1A4 | ANAPC1 | anaphase promoting | Nucleus | other | |
| | | complex subunit 1 | | | |
| Q9UJX3 | ANAPC7 | anaphase promoting | unknown | other | |
| | | complex subunit 7 | | | |
| Q9UJX4 | ANAPC5 | anaphase promoting | Nucleus | enzyme | |
| | | complex subunit 5 | | | |
| Q9UJX5 | ANAPC4 | anaphase promoting | unknown | enzyme | |
| | | complex subunit 4 | | | |
| Q8TD19 | NEK9 | NIMA (never in mitosis | Nucleus | kinase | |
| | (includes | gene a)- related kinase 9 | | | |
| | EG: 91754) | | | | |
| O75419 | CDC45L | CDC45 cell division cycle | Nucleus | other | |
| | | 45-like (S. cerevisiae) | | | |
| P46109 | CRKL | v-crk sarcoma virus CT10 | Cytoplasm | kinase | |
| | | oncogene homolog (avian)-like | | | |
| Q92608 | DOCK2 | dedicator of cytokinesis 2 | Cytoplasm | other | |
| Q96N67 | DOCK7 | dedicator of cytokinesis 7 | unknown | other | |
| | (includes | | | | |
| | EG: 85440) | | | | |
| Q5JSL3 | DOCK11 | dedicator of cytokinesis 11 | unknown | other | |
| P42566 | EPS15 | epidermal growth factor | Plasma | other | |
| | | receptor pathway substrate 15 | Membrane | | |
| P62993 | GRB2 | growth factor receptor- | Cytoplasm | other | |
| | | bound protein 2 | | | |
| Q13546 | RIPK1 | receptor (TNFRSF)- | Plasma | kinase | |
| | | interacting serine-threonine | Membrane | | |
| | | kinase 1 | | | |
| Q14687 | KIAA0182 | KIAA0182 | unknown | other | |
| Q13501 | SQSTM1 | sequestosome 1 | Cytoplasm | transcription | |
| | | | | regulator | |
| Q9BZK7 | TBL1XR1 | transducin (beta)-like 1 X- | Nucleus | transcription | |
| | | linked receptor 1 | | regulator | |
| O14744 | PRMT5 | protein arginine | Cytoplasm | enzyme | |
| | | methyltransferase 5 | | | |
| Q96LA8 | PRMT6 | protein arginine | Nucleus | enzyme | |
| | | methyltransferase 6 | | | |
| Q8WUV3 | PRMT3 | protein arginine | Nucleus | enzyme | |
| | | methyltransferase 3 | | | |
| Q2TAZ0 | ATG2A | ATG2 autophagy related 2 | unknown | other | |
| | | homolog A (S. cerevisiae) | | | |
| Q9C0C7 | AMBRA1 | autophagy/beclin-1 | unknown | other | |
| | | regulator 1 | | | |
| Q9H1Y0 | ATG5 | ATG5 autophagy related 5 | Cytoplasm | other | |
| | (includes | homolog (S. cerevisiae) | | | |
| | EG: 9474) | | | | |
| P62258 | YWHAE | tyrosine 3- | Cytoplasm | other | |
| | | monooxygenase/tryptophan | | | |
| | | 5-monooxygenase | | | |
| | | activation protein, epsilon | | | |
| | | polypeptide | | | |
| Q9BQG0 | MYBBP1A | MYB binding protein (P160) 1a | Nucleus | transcription | |
| | | | | regulator | |
| Q92600 | RQCD1 | RCD1 required for cell | unknown | other | |
| | | differentiation1 homolog (S. | | | |
| | | pombe) | | | |
| Q16531 | DDB1 | damage-specific DNA | Nucleus | other | |
| | | binding protein 1, 127 kDa | | | |
| P67809 | YBX1 | Y box binding protein 1 | Nucleus | transcription | |
| | | | | regulator | |
| Q9UKL0 | RCOR1 | REST corepressor 1 | Nucleus | transcription | |
| | | | | regulator | |
| Q13547 | HDAC1 | histone deacetylase 1 | Nucleus | transcription | tributyrin, belinostat, |
| | | | | regulator | pyroxamide, MGCD0103, |
| | | | | | vorinostat, romidepsin |
| O60341 | KDM1A | lysine (K)-specific | Nucleus | enzyme | |
| | | demethylase 1A | | | |
| Q9UBN7 | HDAC6 | histone deacetylase 6 | Nucleus | transcription | tributyrin, belinostat, |
| | | | | regulator | pyroxamide, vorinostat, |
| | | | | | romidepsin |
| Q16576 | RBBP7 | retinoblastoma binding | Nucleus | transcription | |
| | | protein 7 | | regulator | |
| Q92769 | HDAC2 | histone deacetylase 2 | Nucleus | transcription | tributyrin, belinostat, |
| | | | | regulator | pyroxamide, vorinostat, |
| | | | | | romidepsin |
| Q92922 | SMARCC1 | SWI/SNF related, matrix | Nucleus | transcription | |
| | | associated, actin | | regulator | |
| | | dependent regulator of | | | |
| | | chromatin, subfamily c, | | | |
| | | member 1 | | | |
| Q8TAQ2 | SMARCC2 | SWI/SNF related, matrix | Nucleus | transcription | |
| | (includes | associated, actin | | regulator | |
| | EG: 6601) | dependent regulator of | | | |
| | | chromatin, subfamily c, | | | |
| | | member 2 | | | |
| Q03169 | TNFAIP2 | tumor necrosis factor, | Extracellular | other | |
| | | alpha-induced protein 2 | Space | | |
| Q13492 | PICALM | phosphatidylinositol binding | Cytoplasm | other | |
| | | clathrin assembly protein | | | |
| Q8N163 | KIAA1967 | KIAA1967 | Cytoplasm | peptidase | |
| P33992 | MCM5 | minichromosome | Nucleus | enzyme | |
| | | maintenance complex | | | |
| | | component 5 | | | |
| P02786 | TFRC | transferrin receptor (p90, | Plasma | transporter | |
| | | CD71) | Membrane | | |
| Q13263 | TRIM28 | tripartite motif-containing 28 | Nucleus | transcription | |
| | | | | regulator | |
| Q9Y490 | TLN1 | talin 1 | Plasma | other | |
| | | | Membrane | | |
| O14777 | NDC80 | NDC80 homolog, | Nucleus | other | |
| | | kinetochore complex | | | |
| | | component (S. cerevisiae) | | | |
| Q13576 | IQGAP2 | IQ motif containing GTPase | Cytoplasm | other | |
| | | activating protein 2 | | | |
| P14174 | MIF | macrophage migration | Extracellular | cytokine | |
| | | inhibitory factor | Space | | |
| | | (glycosylation-inhibiting | | | |
| | | factor) | | | |
| Q9UQ80 | PA2G4 | proliferation-associated | Nucleus | transcription | |
| | | 2G4, 38 kDa | | regulator | |
| Q7L576 | CYFIP1 | cytoplasmic FMR1 | Cytoplasm | other | |
| | | interacting protein 1 | | | |
| P12004 | PCNA | proliferating cell nuclear | Nucleus | other | |
| | | antigen | | | |
| Q08J23 | NSUN2 | NOP2/Sun domain family, | unknown | enzyme | |
| | | member 2 | | | |
| O75376 | NCOR1 | nuclear receptor co- | Nucleus | transcription | |
| | | repressor 1 | | regulator | |
| Q9Y618 | NCOR2 | nuclear receptor co- | Nucleus | transcription | |
| | | repressor 2 | | regulator | |
| Q12906 | ILF3 | interleukin enhancer | Nucleus | transcription | |
| | | binding factor 3, 90 kDa | | regulator | |
| Q12905 | ILF2 | interleukin enhancer | Nucleus | transcription | |
| | (includes | binding factor 2, 45 kDa | | regulator | |
| | EG: 3608) | | | | |
| Q07666 | KHDRBS1 | KH domain containing, | Nucleus | transcription | |
| | | RNA binding, signal | | regulator | |
| | | transduction associated 1 | | | |
| Q9HCF4 | RNF213 | ring finger protein 213 | Plasma | other | |
| | | | Membrane | | |
| O94776 | MTA2 | metastasis associated 1 | Nucleus | transcription | |
| | | family, member 2 | | regulator | |
| P53041 | PPP5C | protein phosphatase 5, | Nucleus | phosphatase | |
| | | catalytic subunit | | | |
| O60610 | DIAPH1 | diaphanous homolog 1 | Cytoplasm | other | |
| | | (Drosophila) | | | |
| P27694 | RPA1 | replication protein A1, | Nucleus | other | |
| | | 70 kDa | | | |
| Q8NC51 | SERBP1 | SERPINE1 mRNA binding | Nucleus | other | |
| | | protein 1 | | | |
| P30154 | PPP2R1B | protein phosphatase 2 | unknown | phosphatase | |
| | | (formerly 2A), regulatory | | | |
| | | subunit A, beta isoform | | | |
| P63151 | PPP2R2A | protein phosphatase 2 | Cytoplasm | phosphatase | |
| | | (formerly 2A), regulatory | | | |
| | | subunit B, alpha isoform | | | |
| Q9UPN7 | SAPS1 | SAPS domain family, | unknown | other | |
| | | member 1 | | | |
| Q8WUH2 | TGFBRAP1 | transforming growth factor, | Cytoplasm | other | |
| | | beta receptor associated | | | |
| | | protein 1 | | | |
| Q9NTK5 | OLA1 | Obg-like ATPase 1 | Cytoplasm | other | |
| Q9UBR2 | CTSZ | cathepsin Z | Cytoplasm | peptidase | |
| | (includes | | | | |
| | EG: 1522) | | | | |
| Q15057 | ACAP2 | ArfGAP with coiled-coil, | Nucleus | other | |
| | | ankyrin repeat and PH | | | |
| | | domains 2 | | | |
| Q9Y2X7 | GIT1 | G protein-coupled receptor | Nucleus | other | |
| | | kinase interacting ArfGAP 1 | | | |
| Q92888 | ARHGEF1 | Rho guanine nucleotide | Cytoplasm | other | |
| | | exchange factor (GEF) 1 | | | |
| Q92974 | ARHGEF2 | Rho/Rac guanine | Cytoplasm | other | |
| | | nucleotide exchange factor | | | |
| | | (GEF) 2 | | | |
| P46060 | RANGAP1 | Ran GTPase activating | Cytoplasm | other | |
| | | protein 1 | | | |
| Q14C86 | GAPVD1 | GTPase activating protein | unknown | other | |
| | | and VPS9 domains 1 | | | |
| Q15042 | RAB3GAP1 | RAB3 GTPase activating | Cytoplasm | other | |
| | | protein subunit 1 (catalytic) | | | |
| P62826 | RAN | RAN, member RAS | Nucleus | enzyme | |
| | | oncogene family | | | |
| Q9NR31 | SAR1A | SAR1 homolog A | Cytoplasm | enzyme | |
| | | (S. cerevisiae) | | | |
| Q15907 | RAB11B | RAB11B, member RAS | Cytoplasm | enzyme | |
| | | oncogene family | | | |
| Q8TC07 | TBC1D15 | TBC1 domain family, | Cytoplasm | other | |
| | | member 15 | | | |
| Q9Y4R8 | TELO2 | TEL2, telomere | unknown | other | |
| | | maintenance 2, homolog | | | |
| | | (S. cerevisiae) | | | |
| Q5UIP0 | RIF1 | RAP1 interacting factor | Nucleus | other | |
| | | homolog (yeast) | | | |
| Q9BUR4 | WRAP53 | WD repeat containing, | unknown | other | |
| | | antisense to TP53 | | | |
| Q9C0C2 | TNKS1BP1 | tankyrase 1 binding protein 1, | Nucleus | other | |
| | | 182 kDa | | | |
| Q53EL6 | PDCD4 | programmed cell death 4 | Nucleus | other | |
| | | (neoplastic transformation | | | |
| | | inhibitor) | | | |
| Q86UX7 | FERMT3 | fermitin family homolog 3 | Cytoplasm | enzyme | |
| | | (Drosophila) | | | |
| Q14289 | PTK2B | PTK2B protein tyrosine | Cytoplasm | kinase | |
| | | kinase 2 beta | | | |
| P55196 | MLLT4 | myeloid/lymphoid or mixed- | Nucleus | other | |
| | | lineage leukemia (trithorax | | | |
| | | homolog, Drosophila); | | | |
| | | translocated to, 4 | | | |
| Q9Y4L1 | HYOU1 | hypoxia up-regulated 1 | Cytoplasm | other | |
| Q96DA0 | ZG16B | zymogen granule protein | unknown | other | |
| | | 16 homolog B (rat) | | | |
| Q96PE3 | INPP4A | inositol polyphosphate-4- | Cytoplasm | phosphatase | |
| | | phosphatase, type I, | | | |
| | | 107 kDa | | | |
| P36915 | GNL1 | guanine nucleotide binding | unknown | other | |
| | | protein-like 1 | | | |
| Q9Y3Z3 | SAMHD1 | SAM domain and HD | Nucleus | enzyme | |
| | | domain 1 | | | |
| Q07157 | TJP1 | tight junction protein 1 | Plasma | other | |
| | | (zona occludens 1) | Membrane | | |
| P46379 | BAT3 | HLA-B associated | Nucleus | enzyme | |
| | | transcript 3 | | | |
| P21333 | FLNA | filamin A, alpha | Cytoplasm | other | |
| Q14315 | FLNC | filamin C, gamma | Cytoplasm | other | |
| Q86Y56 | HEATR2 | HEAT repeat containing 2 | unknown | other | |
| Q6AI08 | HEATR6 | HEAT repeat containing 6 | unknown | other | |
| P98160 | HSPG2 | heparan sulfate | Plasma | other | |
| | (includes | proteoglycan 2 | Membrane | | |
| | EG: 3339) | | | | |
| Q14247 | CTTN | cortactin | Plasma | other | |
| | | | Membrane | | |
| O00170 | AIP | aryl hydrocarbon receptor | Nucleus | transcription | |
| | | interacting protein | | regulator | |
| Q9H0A0 | NAT10 | N-acetyltransferase 10 | Nucleus | enzyme | |
| | | (GCN5-related) | | | |
| Q9UPY3 | DICER1 | dicer 1, ribonuclease type | Cytoplasm | enzyme | |
| | | III | | | |
| Q9NZB2 | FAM120A | family with sequence | Cytoplasm | other | |
| | | similarity 120A | | | |
| Q14980 | NUMA1 | nuclear mitotic apparatus | Nucleus | other | |
| | | protein 1 | | | |
| Q15645 | TRIP13 | thyroid hormone receptor | Cytoplasm | transcription | |
| | | interactor 13 | | regulator | |
| Q9Y4C2 | FAM115A | family with sequence | unknown | other | |
| | | similarity 115, member A | | | |
| Q8IYB8 | SUPV3L1 | suppressor of var1, 3-like 1 | Cytoplasm | enzyme | |
| | | (S. cerevisiae) | | | |
| Q96GA3 | LTV1 | LTV1 homolog (S. cerevisiae) | unknown | other | |
| Q9NX58 | LYAR | Ly1 antibody reactive | Plasma | other | |
| | | homolog (mouse) | Membrane | | |
| Q13510 | ASAH1 | N-acylsphingosine | Cytoplasm | enzyme | |
| | | amidohydrolase (acid | | | |
| | | ceramidase) 1 | | | |
| Q6UN15 | FIP1L1 | FIP1 like 1 (S. cerevisiae) | Nucleus | other | |
| Q14145 | KEAP1 | kelch-like ECH-associated | Cytoplasm | transcription | |
| | | protein 1 | | regulator | |
| Q12888 | TP53BP1 | tumor protein p53 binding | Nucleus | transcription | |
| | | protein 1 | | regulator | |
| Q07812 | BAX | BCL2-associated X protein | Cytoplasm | other | |
| Q9Y613 | FHOD1 | formin homology 2 domain | Nucleus | other | |
| | | containing 1 | | | |
| O75131 | CPNE3 | copine III | Cytoplasm | kinase | |
| Q04724 | TLE1 | transducin-like enhancer of | Nucleus | transcription | |
| | | split 1 (E(sp1) homolog, | | regulator | |
| | | Drosophila) | | | |
| O14773 | TPP1 | tripeptidyl peptidase I | Cytoplasm | peptidase | |
| O60524 | SDCCAG1 | serologically defined colon | Nucleus | other | |
| | | cancer antigen 1 | | | |
| Q9Y2A7 | NCKAP1 | NCK-associated protein 1 | Plasma | other | |
| | | | Membrane | | |
| Q7Z3B4 | NUP54 | nucleoporin 54 kDa | Nucleus | transporter | |
| Q9BW27 | NUP85 | nucleoporin 85 kDa | Cytoplasm | other | |
| Q12769 | NUP160 | nucleoporin 160 kDa | Nucleus | transporter | |
| A5YKK6 | CNOT1 | CCR4-NOT transcription | unknown | other | |
| | | complex, subunit 1 | | | |
| Q9H9A6 | LRRC40 | leucine rich repeat | Nucleus | other | |
| | | containing 40 | | | |
| Q99623 | PHB2 | prohibitin 2 | Cytoplasm | transcription | |
| | | | | regulator | |
| Q08AM6 | VAC14 | Vac14 homolog (S. cerevisiae) | unknown | other | |
| Q9ULX3 | NOB1 | NIN1/RPN12 binding | Nucleus | other | |
| | | protein 1 homolog | | | |
| | | (S. cerevisiae) | | | |
| P78395 | PRAME | preferentially expressed | Nucleus | other | |
| | (includes | antigen in melanoma | | | |
| | EG: 23532) | | | | |
| Q8N1G2 | FTSJD2 | FtsJ methyltransferase | unknown | other | |
| | | domain containing 2 | | | |
| P19838 | NFKB1 | nuclear factor of kappa light | Nucleus | transcription | |
| | | polypeptide gene enhancer | | regulator | |
| | | in B-cells 1 | | | |
| P08195 | SLC3A2 | solute carrier family 3 | Plasma | transporter | |
| | | (activators of dibasic and | Membrane | | |
| | | neutral amino acid | | | |
| | | transport), member 2 | | | |
| Q15773 | MLF2 | myeloid leukemia factor 2 | Nucleus | other | |
| Q9NR28 | DIABLO | diablo homolog | Cytoplasm | other | |
| | | (Drosophila) | | | |
| O95831 | AIFM1 | apoptosis-inducing factor, | Cytoplasm | enzyme | |
| | | mitochondrion-associated, 1 | | | |
| Q7Z2W4 | ZC3HAV1 | zinc finger CCCH-type, | Plasma | other | |
| | | antiviral 1 | Membrane | | |
| Q8WXF1 | PSPC1 | paraspeckle component 1 | Nucleus | other | |
| O43815 | STRN | striatin, calmodulin binding | Cytoplasm | other | |
| | | protein | | | |
| P35232 | PHB | prohibitin | Nucleus | transcription | |
| | (includes | | | regulator | |
| | EG: 5245) | | | | |
| Q15058 | KIF14 | kinesin family member 14 | Cytoplasm | other | |
| Q13227 | GPS2 | G protein pathway | Nucleus | other | |
| | | suppressor 2 | | | |
| O75534 | CSDE1 | cold shock domain | Cytoplasm | enzyme | |
| | | containing E1, RNA-binding | | | |
| Q14839 | CHD4 | chromodomain helicase | Nucleus | enzyme | |
| | | DNA binding protein 4 | | | |
| O14497 | ARID1A | AT rich interactive domain | Nucleus | transcription | |
| | | 1A (SWI-like) | | regulator | |
| Q9P035 | PTPLAD1 | protein tyrosine | Cytoplasm | other | |
| | | phosphatase-like A domain | | | |
| | | containing 1 | | | |
| Q8WUZ0 | BCL7C | B-cell CLL/lymphoma 7C | unknown | other | |
| Q92733 | PRCC | papillary renal cell | Nucleus | other | |
| | | carcinoma (translocation- | | | |
| | | associated) | | | |
| Q9Y6W5 | WASF2 | WAS protein family, | Cytoplasm | other | |
| | | member 2 | | | |
| Q8NDX1 | PSD4 | pleckstrin and Sec7 domain | unknown | other | |
| | | containing 4 | | | |
| O96006 | ZBED1 | zinc finger, BED-type | Nucleus | enzyme | |
| | | containing 1 | | | |
| Q92542 | NCSTN | nicastrin | Plasma | peptidase | |
| | | | Membrane | | |
| Q6NSH3 | CT45A5 | cancer/testis antigen family | unknown | other | |
| | | 45, member A5 | | | |

TABLE 5f
Significant networks and associated biofunctions assigned by Ingenuity
Pathways Core Analysis to proteins isolated by PU-H71 in the K562 cell line
©2000-2010 Ingenuity Systems, Inc. All rights reserved.

| | | Focus | | |
|---|---|---|---|---|
| ID | Score* | Molecules | Top Functions | Molecules in Network |
| 1 | 38 | 22 | Cell Cycle, | 14-3-3, Akt, AMPK, ATM, ATR (includes EG: 545), Fgf, |
| | | | Carbohydrate | HYOU1, INPP4A, Insulin, KHDRBS1, MAP2K1/2, |
| | | | Metabolism, Lipid | MAPKAP1, MTOR, NGF, p70 S6k, p85 (pik3r), PA2G4, |
| | | | Metabolism | Pi3-kinase, PIK3C3, PIK3R4, PRKAC, PRKAG1, Raf, |
| | | | | RAF1, RPA1, RPS6KA1, RPTOR, SMG1, SRPK2, |
| | | | | Stat1/3, STRAP, TELO2, TP53BP1, YWHAE, YWHAQ |
| | | | | (includes EG: 10971) |
| 2 | 36 | 22 | Cell Signaling, | alcohol group acceptor phosphotransferase, ARAF, BCR, |
| | | | Protein Synthesis, | CAMK2G, Casein, CDK7, CK1, CSNK1A1, CSNK2B, Gm- |
| | | | Infection Mechanism | csf, HINT1, Ifn, IFN TYPE 1, Ikb, IKK (complex), Ikk |
| | | | | (family), IRAK, IRAK1, KEAP1, MALT1, MAP2K3, NFkB |
| | | | | (complex), NFkB (family), PRKAA1, PRKD2, PTPLAD1, |
| | | | | RIPK1, RPS6KA3, SARM1, SQSTM1, TAB1, TBK1, |
| | | | | TFRC, Tnf receptor, TNFAIP2 |
| 3 | 33 | 20 | Cell Death, Cell | ABL1, ANAPC1, ANAPC4, ANAPC5, ANAPC7, APC, |
| | | | Cycle, Cell | ARHGEF1, BUB1B, Caspase, Cdc2, CSDE1, CTSB, |
| | | | Morphology | Cyclin A, Cyclin E, Cytochrome c, DIABLO, E2f, E3 RING, |
| | | | | FBXO22, Hsp27, KIAA1967, Laminin, LGALS3, MAP3K4, |
| | | | | MCM5, Mek, NPM1 (includes EG: 4869), NUMA1, P38 |
| | | | | MAPK, PRAME (includes EG: 23532), Ras, Rb, RBX1 |
| | | | | (includes EG: 9978), Sapk, SKP1 |
| 4 | 33 | 20 | Cell Cycle | 26s Proteasome, AKAP8L, Alp, ASAH1, ASCC2, BAT3, |
| | | | | BAX, BMP2K (includes EG: 55589), DDB1, DICER1, ERH, |
| | | | | Fibrinogen, hCG, Hsp70, IFN Beta, IgG, IL1, IL12 |
| | | | | (complex), IL12 (family), Interferon alpha, LDL, NFKB1, |
| | | | | OLA1 , PCNA, Pka, PRKACA, PRMT5, RNA polymerase |
| | | | | II, RUVBL1, RUVBL2, STAT3, TLE1, TP63, Ubiquitin, |
| | | | | ZC3HAV1 |
| 5 | 32 | 20 | Cellular Assembly | Adaptor protein 2, AIP, Ap1, ARHGEF2, BTF3, |
| | | | and Organization, | Calcineurin protein(s), Calmodulin, CaMKII, Ck2, Collagen |
| | | | Cellular Function | type IV, Creb, EPS15, Estrogen Receptor, G protein |
| | | | and Maintenance | alphai, Hsp90, IGF2BP1, LYAR, Mapk, MAPK14, MIF, |
| | | | | MOBKL3, NAT10, NMDA Receptor, NONO, NOP2, |
| | | | | PDAP1, PDCD4, PI4KA, PICALM, PikSr, PP2A, PSPC1, |
| | | | | RIF1, SRPK1, STRN |
| 6 | 30 | 19 | Gene Expression, | ARID1A, atypical protein kinase C, CARM1, Cbp/p300, |
| | | | Cellular Assembly | CHD4, ERK1/2, Esr1-Esr1-estrogen-estrogen, GIT1, |
| | | | and Organization, | GPS2, Hdac1/2, HISTONE, Histone h3, Histone h4, |
| | | | Cellular | KDM1A, Mi2, MTA2, MYBBP1A, N-cor, NCOR1, NCOR2, |
| | | | Compromise | NCoR/SMRT corepressor, NuRD, PHB2, PHB (includes |
| | | | | EG: 5245), Rar, RBBP7, RCOR1 , Rxr, SLC3A2, |
| | | | | SMARCC1, SMARCC2 (includes EG: 6601), Sos, |
| | | | | TBL1XR1, TIP60, TRIM28 |
| 7 | 22 | 15 | Cell Cycle, | AKAP8, AKAP14, ALDH1B1, CDCA7, CEPT1, CIT, |
| | | | Development | CNBP, CPNE3, DISC1, DOCK11, FTSJD2, HIT, IFNA2, |
| | | | | IGF2BP3, IQGAP3, KIF14, LGMN, MIR124, MIR129-2 |
| | | | | (includes EG: 406918), MIRN339, MYC, MYCBP, NEK9 |
| | | | | (includes EG: 91754), NFkB (complex), NUP160, PANK4, |
| | | | | PEA15, PRPF40B, RNF213, SAMHD1, SCAMP5, TPP1, |
| | | | | TRIM56, WRAP53, YME1L1 |
| 8 | 20 | 14 | Cellular | BCR, BTK, Calpain, CAPN1, CAPNS1, Collagen type I, |
| | | | Compromise, | CRKL, DOCK2, Fcer1, GNRH, Ige, JAK, KSR1, MAPK1, |
| | | | Hypersensitivity | NCK, NFAT (complex), Pdgf, PHKB, Pkg, PLC gamma, |
| | | | Response, | Ptk, PTK2B, STAT, STAT1/3/5, STAT1/3/5/6, STAT3/5, |
| | | | Inflammatory | STAT5A, STAT5a/b, STAT5B, SYK/ZAP, Talin, TLN1, |
| | | | Response | TYK2, VAV, VAV1 |
| 9 | 20 | 14 | Cell Morphology, | ABLIM, ACAP2, AKR1C14, ARF6, ARPC1A, ATP9A, |
| | | | Cellular | BUB1, CREBL2, DHRS3, DYRK3, FHOD1, FLNC, FSH, |
| | | | Development and | GK7P, GNL1, GRB2, HEATR2, Lh, LOC81691, NCSTN, |
| | | | Function | NDC80, PDGF BB, PI4K2A, PRMT6, PTP4A1, QRFP, |
| | | | | RAB11B, RQCD1, SCARB2, SLC2A4, THBS1, TP53I11, |
| | | | | TRIP13, Vegf, ZBED1 |
| 10 | 18 | 13 | Cell Morphology | AGT, AGTRAP, ATG5 (includes EG: 9474), Cathepsin, |
| | | | | COL4A6, CORIN, ENPP1, FAM120A, GATM, H1FX, |
| | | | | HSPG2 (includes EG: 3339), IGF2BP2, ITPA, KIAA0182, |
| | | | | LPCAT3, MCPT1, MIR17 (includes EG: 406952), MYL3, |
| | | | | NOS1, NSUN2, PFK, PLA1A, RPS6, SCYL1, SDPR, |
| | | | | SERBP1, SMOC2, SRF, SRFBP1, STOML2, TGFB1, |
| | | | | TGFBRAP1, TMOD3, VAC14, WIBG |
| 11 | 17 | 12 | Gene Expression, | AMBRA1, AR, CDC45L, CDCA7L, CLDND1, CTDSP2, |
| | | | Developmental | FAM115A, HEATR6, HNF4A, HYAL3, KIAA1468, |
| | | | Disorder | LRRC40, MIR124-1 (includes EG: 406907), NUP54, PECI, |
| | | | | PERP, POLR3G, PRCC, PTPN4, PTPN11, RIOK2, RNF6, |
| | | | | RNPEPL1, SF3B4, SLC17A5, SLC25A20, SLC30A7, |
| | | | | SLC39A7, SSFA2, STK19, SUPV3L1, TBC1D15, TCF19, |
| | | | | ZBED3, ZZEF1 |
| 12 | 16 | 13 | Cell Morphology, | Actin, AIFM1, Arp2/3, CDS, CTTN, CYFIP1, DIAPH1, |
| | | | Cellular Assembly | Dynamin, ERK, F Actin, FERMT3, Focal adhesion kinase, |
| | | | and Organization, | Gpcr, Growth hormone, Integrin, IQGAP2, Jnk, Lfa-1, |
| | | | Cellular | MLF2, MLLT4, NCKAP1, Nfat (family), Pak, PI3K, PI3K |
| | | | Development | p85, Pkc(s), PPP5C, PTK2, Rac, Rap1, Ras homolog, |
| | | | | Rsk, TCR, TJP1, WASF2 |
| 13 | 12 | 10 | Cancer, Cell Cycle, | ANKRD2, APRT, ARL6IP1, BANP, C11ORF82, CAMK1, |
| | | | Gene Expression | CKMT1B, CNOT1, CTSZ (includes EG: 1522), DOCK7 |
| | | | | (includes EG: 85440), FIP1L1, GART, GH1, GIP2, GSK3B, |
| | | | | HDAC5, Hla-abc, IFNG, MAN2B1, NAPSA, NTHL1, |
| | | | | NUP85, ORM2, PTPN23, SLC5A8, SLC6A6, TBX3, |
| | | | | TNKS1BP1, TOB1, TP53, TRIM22, UNC5B, VPS33A, |
| | | | | YBX1, YWHAZ |

*IPA computes a score for each possible network according to the fit of that network to the inputted proteins. The score is calculated as the negative base-10 logarithm of the p-value that indicates the likelihood of the inputted proteins in a given network being found together due to random chance. Therefore, scores of 2 or higher have at least a 99% confidence of not being generated by random chance alone.

### Supplementary Materials and Methods

### Reagents

The Hsp90 inhibitors, the solid-support immobilized and the fluorescein-labeled derivatives were synthesized as previously reported (Taldone et al., 2011, Synthesis and Evaluation of Small . . . ; Taldone et al., 2011, Synthesis and Evaluation of Fluorescent . . . ; He et al., 2006). We purchased Gleevec from LC Laboratories, AS703026 from Selleck, KN-93 from Tocris, and PP242, BMS-345541 and sodium vanadate from Sigma. All compounds were used as DMSO stocks.

### Western Blotting

Cells were either treated with PU-H71 or DMSO (vehicle) for 24 h and lysed in 50 mM Tris, pH 7.4, 150 mM NaCl and 1% NP40 lysis buffer supplemented with leupeptin (Sigma Aldrich) and aprotinin (Sigma Aldrich). Protein concentrations were determined using BCA kit (Pierce) according to the manufacturer's instructions. Protein lysates (15-200 μg) were electrophoretically resolved by SDS/PAGE, transferred to nitrocellulose membrane and probed with the following primary antibodies against: Hsp90 (1:2000, SMC-107A/B; StressMarq), Bcr-Abl (1:75, 554148; BD Pharmingen), PI3K (1:1000, 06-195; Upstate), mTOR (1:200, Sc-1549; Santa Cruz), p-mTOR (1:1000, 2971; Cell Signaling), STAT3 (1:1000, 9132; Cell Signaling), p-STAT3 (1:2000, 9145; Cell Signaling), STAT5 (1:500, Sc-835; Santa Cruz), p-STAT5 (1:1000, 9351; Cell Signaling), RICTOR (1:2000, NB100-611; Novus Biologicals), RAPTOR (1:1000, 2280; Cell Signaling), P90RSK (1:1000, 9347; Cell Signaling), Raf-1 (1:300, Sc-133; Santa Cruz), CARM1 (1:1000, 09-818; Millipore), CRKL (1:200, Sc-319; Santa Cruz), GRB2 (1:1000, 3972; Cell Signaling), FAK (1:1000, Sc-1688; Santa Cruz), BTK (1:1000, 3533; Cell Signaling), A-Raf (1:1000, 4432; Cell Signaling), PRKD2 (1:200, sc-100415, Santa Cruz), HCK (1:500, 06-833; Milipore), p-HCK (1:500, ab52203; Abcam) and β-actin (1:2000, A1978; Sigma). The membranes were then incubated with a 1:3000 dilution of a corresponding horseradish peroxidase conjugated secondary antibody. Detection was performed using the ECL-Enhanced Chemiluminescence Detection System (Amersham Biosciences) according to manufacturer's instructions.

### Densitometry

Gels were scanned in Adobe Photoshop 7.0.1 and quantitative densitometric analysis was performed using Un-Scan-It 5.1 software (Silk Scientific).

### Nano-LC-MS/MS

Lysates prepared as mentioned above were first pre-cleaned by incubation with control beads overnight at 4° C. Pre-cleaned K562 cell extract (1,000 μg) in 200 μl Felts lysis buffer was incubated with PU-H71 or control-beads (80 μl) for 24 h at 4° C. Beads were washed with lysis buffer, proteins eluted by boiling in 2% SDS, separated on a denaturing gel and Coomassie stained according to manufacturer's procedure (Biorad). Gel-resolved proteins from pull-downs were digested with trypsin, as described (Winkler et al., 2002). In-gel tryptic digests were subjected to a micro-clean-up procedure (Erdjument-Bromage et al., 1998) on 2 μL bed-volume of Poros 50 R2 (Applied Biosystems-‘AB’) reversed-phase beads, packed in an Eppendorf gel-loading tip, and the eluant diluted with 0.1% formic acid (FA). Analyses of the batch purified pools were done using a QSTAR-Elite hybrid quadrupole time-of-flight mass spectrometer (QTof MS) (AB/MDS Sciex), equipped with a nano spray ion source. Peptide mixtures (in 20 μL) are loaded onto a trapping guard column (0.3×5-mm PepMap C18 100 cartridge from LC Packings) using an Eksigent nano MDLC system (Eksigent Technologies, Inc) at a flow rate of 20 μL/min. After washing, the flow was reversed through the guard column and the peptides eluted with a 5-45% MeCN gradient (in 0.1% FA) over 85 min at a flow rate of 200 nL/min, onto and over a 75-micron×15-cm fused silica capillary PepMap C18 column (LC Packings); the eluant is directed to a 75-micron (with 10-micron orifice) fused silica nano-electrospray needle (New Objective). Electrospray ionization (ESI) needle voltage was set at about 1800 V. The mass analyzer is operated in automatic, data-dependent MS/MS acquisition mode, with the threshold set to 10 counts per second of doubly or triply charged precursor ions selected for fragmentation scans. Survey scans of 0.25 sec are recorded from 400 to 1800 amu; up to 3 MS/MS scans are then collected sequentially for the selected precursor ions, recording from 100 to 1800 amu. The collision energy is automatically adjusted in accordance with the m/z value of the precursor ions selected for MS/MS. Selected precursor ions are excluded from repeated selection for 60 sec after the end of the corresponding fragmentation duty cycle. Initial protein identifications from LC-MS/MS data was done using the Mascot search engine (Matrix Science, version 2.2.04; www.matrixscience.com) and the NCBI (National Library of Medicine, NIH—human taxonomy containing, 223,695 protein sequences) and IPI (International Protein Index, EBI, Hinxton, UK—human taxonomy, containing 83,947 protein sequences) databases. One missed tryptic cleavage site was allowed, precursor ion mass tolerance=0.4 Da fragment ion mass tolerance=0.4 Da, protein modifications were allowed for Met-oxide, Cys-acrylamide and N-terminal acetylation. MudPit scoring was typically applied with ‘require bold red’ activated, and using significance threshold score p<0.05. Unique peptide counts (or ‘spectral counts’) and percent sequence coverages for all identified proteins were exported to Scaffold Proteome Software (version 2_{—}06_{—}01, www.proteomesoftware.com) for further bioinformatic analysis (Table 5a). Using output from Mascot, Scaffold validates, organizes, and interprets mass spectrometry data, allowing more easily to manage large amounts of data, to compare samples, and to search for protein modifications. Findings were validated in a second MS system, the Waters Xevo QTof MS instrument (Table 5d). Potential unspecific interactors were identified and removed from further analyses as indicated (Trinkle-Mulcahy et al., 2008).

### Bioinformatic Pathways Analysis

Proteins were analyzed further by bioinformatic pathways analysis (Ingenuity Pathway Analysis 8.7 [IPA]; Ingenuity Systems, Mountain View, Calif., www.ingenuity.com) (Munday et al., 2010; Andersen et al., 2010). IPA constructs hypothetical protein interaction clusters based on a regularly updated “Ingenuity Pathways Knowledge Base”. The Ingenuity Pathways Knowledge Base is a very large curated database consisting of millions of individual relationships between proteins, culled from the biological literature. These relationships involve direct protein interactions including physical binding interactions, enzyme substrate relationships, and cis-trans relationships in translational control. The networks are displayed graphically as nodes (individual proteins) and edges (the biological relationships between the nodes). Lines that connect two molecules represent relationships. Thus any two molecules that bind, act upon one another, or that are involved with each other in any other manner would be considered to possess a relationship between them. Each relationship between molecules is created using scientific information contained in the Ingenuity Knowledge Base. Relationships are shown as lines or arrows between molecules. Arrows indicate the directionality of the relationship, such that an arrow from molecule A to B would indicate that molecule A acts upon B. Direct interactions appear in the network diagram as a solid line, whereas indirect interactions as a dashed line. In some cases a relationship may exist as a circular arrow or line originating from one molecule and pointing back at that same molecule. Such relationships are termed “self-referential” and arise from the ability of a molecule to act upon itself. In practice, the dataset containing the UniProtKB identifiers of differentially expressed proteins is uploaded into IPA. IPA then builds hypothetical networks from these proteins and other proteins from the database that are needed fill out a protein cluster. Network generation is optimized for inclusion of as many proteins from the inputted expression profile as possible, and aims for highly connected networks. Proteins are depicted in networks as two circles when the entity is part of a complex; as a single circle when only one unit is present; a triangle pointing up or down to describe a phosphatase or a kinase, respectively; by a horizontal oval to describe a transcription factor; and by circle to depict “other” functions. IPA computes a score for each possible network according to the fit of that network to the inputted proteins. The score is calculated as the negative base-10 logarithm of the p-value that indicates the likelihood of the inputted proteins in a given network being found together due to random chance. Therefore, scores of 2 or higher have at least a 99% confidence of not being generated by random chance alone. All the networks presented here were assigned a score of 10 or higher (Table 5f).

### Radioisotope Binding Studies and Hsp90 Quantification Studies

Saturation studies were performed with ¹³¹I-PU-H71 and cells (K562, MDA-MB-468, SKBr3, LNCaP, DU-145, MRC-5 and PBL). Briefly, triplicate samples of cells were mixed with increasing amount of ¹³¹I-PU-H71 either with or without 1 μM unlabeled PU-H71. The solutions were shaken in an orbital shaker and after 1 hr the cells were isolated and washed with ice cold Tris-buffered saline using a Brandel cell harvester. All the isolated cell samples were counted and the specific uptake of ¹³¹I-PU-H71 determined. These data were plotted against the concentration of ¹³¹I-PU-H71 to give a saturation binding curve. For the quantification of PU-bound Hsp90, 9.2×10⁷ K562 cells, 6.55×10⁷ KCL-22 cells, 2.55×10⁷ KU182 cells and 7.8×10⁷ MEG-01 cells were lysed to result in 6382, 3225, 1349 and 3414 μg of total protein, respectively. To calculate the percentage of Hsp90, cellular Hsp90 expression was quantified by using standard curves created of recombinant Hsp90 purified from HeLa cells (Stressgen#ADI-SPP-770).

### Pulse-Chase

K562 cells were treated with Na₃VO₄ (1 mM) with or without PU-H71 (5 μM), as indicated. Cells were collected at indicated times and lysed in 50 mM Tris pH 7.4, 150 mM NaCl and 1% NP-40 lysis buffer, and were then subjected to western blotting procedure.

### Tryptic Digestion

K562 cells were treated for 30 min with vehicle or PU-H71 (50 μM). Cells were collected and lysed in 50 mM Tris pH 7.4, 150 mM NaCl, 1% NP-40 lysis buffer. STAT5 protein was immunoprecipitated from 500 μg of total protein lysate with an anti-STAT5 antibody (Santa Cruz, sc-835). Protein precipitates bound to protein G agarose beads were washed with trypsin buffer (50 mM Tris pH 8.0, 20 mM CaCl₂) and 33 ng of trypsin has been added to each sample. The samples were incubated at 37° C. and aliquots were collected at the indicated time points. Protein aliquots were subjected to SDS-PAGE and blotted for STAT5.

### Activated STAT5 DNA Binding Assay

The DNA-binding capacity of STAT5a and STAT5b was assayed by an ELISA-based assay (TransAM, Active Motif, Carlsbad, Calif.) following the manufacturer instructions. Briefly, 5×10⁶ K562 cells were treated with PU-H71 1 and 10 μM or control for 24 h. Ten micrograms of cell lysates were added to wells containing pre-adsorbed STAT consensus oligonucleotides (5′-TTCCCGGAA-3′). For control treated cells the assay was performed in the absence or presence of 20 pmol of competitor oligonucleotides that contains either a wild-type or mutated STAT consensus binding site. Interferon-treated HeLa cells (5 μg per well) were used as positive controls for the assay. After incubation and washing, rabbit polyclonal anti-STAT5a or anti-STAT5b antibodies (1:1000, Active Motif) was added to each well, followed by HPR-anti-rabbit secondary antibody (1:1000, Active Motif). After HRP substrate addition, absorbance was read at 450 nm with a reference wavelength of 655 nm (Synergy4, Biotek, Winooski, Vt.). In this assay the absorbance is directly proportional to the quantity of DNA-bound transcription factor present in the sample. Experiments were carried out in four replicates. Results were expressed as arbitrary units (AU) from the mean absorbance values with SEM.

### Quantitative Chromatin Immunoprecipitation (Q-ChIP)

Q-ChIP was made as previously described with modifications (Cerchietti et al., 2009). Briefly, 10⁸ K562 cells were fixed with 1% formaldehyde, lysed and sonicated (Branson sonicator, Branson). STAT5 N20 (Santa Cruz) and Hsp90 (Zymed) antibodies were added to the pre-cleared sample and incubated overnight at 4° C. Then, protein-A or G beads were added, and the sample was eluted from the beads followed by de-crosslinking. The DNA was purified using PCR purification columns (Qiagen). Quantification of the ChIP products was performed by quantitative PCR (Applied Biosystems 7900HT) using Fast SYBR Green (Applied Biosystems). Target genes containing STAT binding site were detected with the following primers: CCND2 (5-GTTGTTCTGGTCCCTTTAATCG and 5-ACCTCGCATACCCAGAGA), MYC (5-ATGCGTTGCTGGGTTATTTT and 5-CAGAGCGTGGGATGTTAGTG) and for the intergenic control region (5-CCACCTGAGTCTGCAATGAG and 5-CAGTCTCCAGCCTTTGTTCC).

### Real Time QPCR

RNA was extracted from PU-H71-treated and control K562 cells using RNeasy Plus kit (Qiagen) following the manufacturer instructions. cDNA was synthesized using High Capacity RNA-to-cDNA kit (Applied Biosystems). We amplified specific genes with the following primers: MYC (5-AGAAGAGCATCTTCCGCATC and 5-CCTTTAAACAGTGCCCAAGC), CCND2 (5-TGAGCTGCTGGCTAAGATCA and 5-ACGGTACTGCTGCAGGCTAT), BCL-XL (5-CTTTTGTGGAACTCTATGGGAACA and 5-CAGCGGTTGAAGCGTTCCT), MCL1 (5-AGACCTTACGACGGGTTGG and 5-ACATTCCTGATGCCACCTTC), CCND1 (5-CCTGTCCTACTACCGCCTCA and 5-GGCTTCGATCTGCTCCTG), HPRT (5-CGTCTTGCTCGAGATGTGATG and 5-GCACACAGAGGGCTACAATGTG), GAPDH (5-CGACCACTTTGTCAAGCTCA and 5-CCCTGTTGCTGTAGCCAAAT), RPL13A (5-TGAGTGAAAGGGAGCCAGAAG and 5-CAGATGCCCCACTCACAAGA). Transcript abundance was detected using the Fast SYBR Green conditions (initial step of 20 sec at 95° C. followed by 40 cycles of 1 sec at 95° C. and 20 sec at 60° C.). The C_{T} value of the housekeeping gene (RPL13A) was subtracted from the correspondent genes of interest (ΔC_{T}). The standard deviation of the difference was calculated from the standard deviation of the C_{T} values (replicates). Then, the ΔC_{T} values of the PU-H71-treated cells were expressed relative to their respective control-treated cells using the ΔΔC_{T} method. The fold expression for each gene in cells treated with the drug relative to control treated cells is determined by the expression: 2^{-ΔΔCT}. Results were represented as fold expression with the standard error of the mean for replicates.

### Hsp70 Knock-Down

Transfections were carried out by electroporation (Amaxa) and the Nucleofector Solution V (Amaxa), according to manufacturer's instructions. Hsp70 knockdown studies were performed using siRNAs designed as previously reported (Powers et al., 2008) against the open reading frame of Hsp70 (HSPA1A; accession number NM 005345). Negative control cells were transfected with inverted control siRNA sequence (Hsp70C; Dharmacon RNA technologies). The active sequences against Hsp70 used for the study are Hsp70A (5′-GGACGAGUUUGAGCACAAG-3′) and Hsp70B (5′-CCAAGCAGACGCAGAUCUU-3′). Sequence for the control is Hsp70C (5′-GGACGAGUUGUAGCACAAG-3′). Three million cells in 2 mL media (RPMI supplemented with 1% L-glutamine, 1% penicillin and streptomycin) were transfected with 0.5 μM siRNA according to the manufacturer's instructions. Transfected cells were maintained in 6-well plates and at 84 h, lysed followed by standard Western blot procedures.

### Kinase Screen (Fabian et al., 2005)

For most assays, kinase-tagged T7 phage strains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 min). The lysates were centrifuged (6,000×g) and filtered (0.2 μm) to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 40× stocks in 100% DMSO and directly diluted into the assay. All reactions were performed in polypropylene 384-well plates in a final volume of 0.04 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μm non-biotinylated affinity ligand) and incubated at room temperature with shaking for 30 minutes. The kinase concentration in the eluates was measured by qPCR. KINOMEscan's selectivity score (S) is a quantitative measure of compound selectivity. It is calculated by dividing the number of kinases that bind to the compound by the total number of distinct kinases tested, excluding mutant variants. TREEspot™ is a proprietary data visualization software tool developed by KINOMEscan (Fabian et al., 2005). Kinases found to bind are marked with red circles, where larger circles indicate higher-affinity binding. The kinase dendrogram was adapted and is reproduced with permission from Science and Cell Signaling Technology, Inc.

### Lentiviral Vectors, Lentiviral Production and K562 Cells Transduction

Lentiviral constructs of shRNA knock-down of CARM1 were purchased from the TRC lentiviral shRNA libraries of Openbiosystem: pLKO.1-shCARM1-KD1 (catalog No: RHS3979-9576107) and pLKO.1-shCARM1-KD2 (catalog No: RHS3979-9576108). The control shRNA (shRNA scramble) was Addgene plasmid 1864. GFP was cloned in to replace puromycin as the selection marker. Lentiviruses were produced by transient transfection of 293T as in the previously described protocol (Moffat et al., 2006). Viral supernatant was collected, filtered through a 0.45-μm filter and concentrated. K562 cells were infected with high-titer lentiviral concentrated suspensions, in the presence of 8 μg/ml polybrene (Aldrich). Transduced K562 cells were sorted for green fluorescence (GFP) after 72 hours transfection.

RNA Extraction and Quantitative Real-Time PCR (qRT-PCR)

For qRT-PCR, total RNA was isolated from 10⁶ cells using the RNeasy mini kit (QIAGEN, Germany), and then subjected to reverse-transcription with random hexamers (SuperScript III kit, Invitrogen). Real-time PCR reactions were performed using an ABI 7500 sequence detection system. The PCR products were detected using either Sybr green I chemistry or TaqMan methodology (PE Applied Biosystems, Norwalk, Conn.). Details for real-time PCR assays were described elsewhere (Zhao et al., 2009). The primer sequences for CARM1 qPCR are TGATGGCCAAGTCTGTCAAG(forward) and TGAAAGCAACGTCAAACCAG(reverse).

### Cell Viability, Apoptosis, and Proliferation Assay

Viability assessment in K562 cells untransfected or transfected with CARM1 shRNA or scramble was performed using Trypan Blue. This chromophore is negatively charged and does not interact with the cell unless the membrane is damaged. Therefore, all the cells that exclude the dye are viable. Apoptosis analysis was assessed using fluorescence microscopy by mixing 2 μL of acridine orange (100 μg/mL), 2 μL of ethidium bromide (100 μg/mL), and 20 μL of the cell suspension. A minimum of 200 cells was counted in at least five random fields. Live apoptotic cells were differentiated from dead apoptotic, necrotic, and normal cells by examining the changes in cellular morphology on the basis of distinctive nuclear and cytoplasmic fluorescence. Viable cells display intact plasma membrane (green color), whereas dead cells display damaged plasma membrane (orange color). An appearance of ultrastructural changes, including shrinkage, heterochromatin condensation, and nuclear degranulation, are more consistent with apoptosis and disrupted cytoplasmic membrane with necrosis. The percentage of apoptotic cells (apoptotic index) was calculated as: % Apoptotic cells=(total number of cells with apoptotic nuclei/total number of cells counted)×100. For the proliferation assay, 5×10³ K562 cells were plated on a 96-well solid black plate (Corning). The assay was performed according to the manufacturer's indications (CellTiter-Glo Luminescent Cell Viability Assay, Promega). All experiments were repeated three times. Where indicated, growth inhibition studies were performed using the Alamar blue assay. This reagent offers a rapid objective measure of cell viability in cell culture, and it uses the indicator dye resazurin to measure the metabolic capacity of cells, an indicator of cell viability. Briefly, exponentially growing cells were plated in microtiter plates (Corning #3603) and incubated for the indicated times at 37° C. Drugs were added in triplicates at the indicated concentrations, and the plate was incubated for 72 h. Resazurin (55 μM) was added, and the plate read 6 h later using the Analyst GT (Fluorescence intensity mode, excitation 530 nm, emission 580 nm, with 560 nm dichroic mirror). Results were analyzed using the Softmax Pro and the GraphPad Prism softwares. The percentage cell growth inhibition was calculated by comparing fluorescence readings obtained from treated versus control cells. The IC₅₀ was calculated as the drug concentration that inhibits cell growth by 50%.

Quantitative Analysis of Synergy Between mTOR and Hsp90 Inhibitors

To determine the drug interaction between pp242 (mTOR inhibitor) and PU-H71 (Hsp90 inhibitor), the combination index (CI) isobologram method of Chou-Talalay was used as previously described (Chou, 2006; Chou & Talalay, 1984). This method, based on the median-effect principle of the law of mass action, quantifies synergism or antagonism for two or more drug combinations, regardless of the mechanisms of each drug, by computerized simulation. Based on algorithms, the computer software displays median-effect plots, combination index plots and normalized isobolograms (where non constant ratio combinations of 2 drugs are used). PU-H71 (0.5, 0.25, 0.125, 0.0625, 0.03125, 0.0125 μM) and pp242 (0.5, 0.125, 0.03125, 0.0008, 0.002, 0.001 μM) were used as single agents in the concentrations mentioned or combined in a non constant ratio (PU-H71:pp242; 1:1, 1:2, 1:4, 1:7.8, 1:15.6, 1:12.5). The Fa (fraction killed cells) was calculated using the formulae Fa=1-Fu; Fu is the fraction of unaffected cells and was used for a dose effect analysis using the computer software (CompuSyn, Paramus, N.J., USA).

### Flow Cytometry

CD34 isolation—CD34+ cell isolation was performed using CD34 MicroBead Kit and the automated magnetic cell sorter autoMACS according to the manufacturer's instructions (Miltenyi Biotech, Auburn, Calif.). Viability assay—CML cells lines were plated in 48-well plates at the density of 5×10⁵ cells/ml, and treated with indicated doses of PU-H71. Cells were collected every 24 h, stained with Annexin V-V450 (BD Biosciences) and 7-AAD (Invitrogen) in Annexin V buffer (10 mM HEPES/NaOH, 0.14 M NaCl, 2.5 mM CaCl₂). Cell viability was analyzed by flow cytometry (BD Biosciences). For patient samples, primary CML cells were plated in 48-well plates at 2×10⁶ cells/ml, and treated with indicated doses of PU-H71 for up to 96 h. Cells were stained with CD34-APC, CD38-PE-CY7 and CD45-APC-H7 antibodies (BD Biosciences) in FACS buffer (PBS, 0.05% FBS) at 4° C. for 30 min prior to Annexin V/7-AAD staining PU-H71 binding assay—CML cells lines were plated in 48-well plates at the density of 5×10⁵ cells/ml, and treated with 1 μM PU-H71-FITC. At 4 h post treatment, cells were washed twice with FACS buffer. To measure PU-H71-FITC binding in live cells, cells were stained with 7-AAD in FACS buffer at room temperature for 10 min, and analyzed by flow cytometry (BD Biosciences). Alternatively, cells were fixed with fixation buffer (BD Biosciences) at 4° C. for 30 min, permeabilized in Perm Buffer III (BD Biosciences) on ice for 30 min, and then analyzed by flow cytometry. At 96 h post PU-H71-FITC treatment, cells were stained with Annexin V-V450 (BD Biosciences) and 7-AAD in Annexin V buffer, and subjected to flow cytometry to measure viability. To evaluate the binding of PU-H71-FITC to leukemia patient samples, primary CML cells were plated in 48-well plates at 2×10⁶ cells/ml, and treated with 1 μM PU-H71-FITC. At 24 h post treatment, cells were washed twice, and stained with CD34-APC, CD38-PE-CY7 and CD45-APC-H7 antibodies in FACS buffer at 4° C. for 30 min prior to 7-AAD staining At 96 h post treatment, cells were stained with CD34-APC, CD38-PE-CY7 and CD45-APC-H7 antibodies followed by Annexin V-V450 and 7-AAD staining to measure cell viability. For competition test, CML cell lines at the density of 5×10⁵ cells/ml or primary CML samples at the density of 2×10⁶ cells/ml were treated with 1 μM unconjugated PU-H71 for 4 h followed by treatment of 1 μM PU-H71-FITC for 1 h. Cells were collected, washed twice, stained for 7-AAD in FACS buffer, and analyzed by flow cytometry. Hsp90 staining—Cells were fixed with fixation buffer (BD Biosciences) at 4° C. for 30 min, and permeabilized in Perm Buffer III (BD Biosciences) on ice for 30 min. Cells were stained with anti-Hsp90 phycoerythrin conjugate (PE) (F-8 clone, Santa Cruz Biotechnologies; CA) for 60 minutes. Cells were washed and then analyzed by flow cytometry. Normal mouse IgG2a-PE was used as isotype control.

### Statistical Analysis

Unless otherwise indicated, data were analyzed by unpaired 2-tailed t tests as implemented in GraphPad Prism (version 4; GraphPad Software). A P value of less than 0.05 was considered significant. Unless otherwise noted, data are presented as the mean±SD or mean±SEM of duplicate or triplicate replicates. Error bars represent the SD or SEM of the mean. If a single panel is presented, data are representative of 2 or 3 individual experiments.

### Maintenance of the B Cell Receptor Pathway and COP9 Signalosome by Hsp90 Reveals Novel Therapeutic Targets in Diffuse Large B Cell Lymphoma

### Experimental Outline

Heat shock protein 90 (Hsp90) is an abundant molecular chaperone, the substrate proteins of which are involved in cell survival, proliferation and angiogenesis. Hsp90 is expressed constitutively and can also be induced by cellular stress, such as heat shock. Because it can chaperone substrate proteins necessary to maintain a malignant phenotype, Hsp90 is an attractive therapeutic target in cancer. In fact, inhibition of Hsp90 results in degradation of many of its substrate proteins. PUH71, an inhibitor of Hsp90, selectively inhibits the oncogenic Hsp90 complex involved in chaperoning onco-proteins and has potent anti-tumor activity diffuse large B cell lymphomas (DLBCLs). By immobilizing PUH71 on a solid support, Hsp90 complexes can be precipitated and analyzed to identify substrate onco-proteins of Hsp90, revealing known and novel therapeutic targets. Preliminary data using this method identified many components of the B cell receptor (BCR) pathway as substrate proteins of Hsp90 in DLBCL. BCR pathway activation has been implicated in lymphomagenesis and survival of DLBCLs. In addition to this, many components of the COP9 signalosome (CSN) were identified as substrates of Hsp90 in DLBCL. The CSN has been implicated in oncogenesis and activation of NF-κB, a survival mechanism of DLBCL. Based on these findings, we hypothesize that combined inhibition of Hsp90 and BCR pathway components and/or the CSN will synergize in killing DLBCL. Therefore, our specific aims are:

### Specific Aim 1: To Determine Whether Concomitant Modulation of Hsp90 and BCR Pathways Cooperate in Killing DLBCL Cells In Vitro and In Vivo

Immobilized PU-H71 will be used to pull down Hsp90 complexes in DLBCL cell lines to detect interactions between Hsp90 and BCR pathway components. DLBCL cell lines treated with increasing doses of PU-H71 will be analyzed for degradation of BCR pathway components DLBCL cell lines will be treated with inhibitors of BCR pathway components alone and in combination with PU-H71 and assessed for viability. Effective combination treatments will be investigated in DLBCL xenograft mouse models.

### Specific Aim 2: To Evaluate the Role of the CSN in DLBCL

### Subaim 1: To Determine Whether the CSN can be a Therapeutic Target in DLBCL

CPs and treatment with PU-H71 will validate the CSN as a substrate of Hsp90 in DLBCL cell lines. The CSN will be genetically ablated alone and in combination with PU-H71 in DLBCL cell lines to demonstrate DLBCL dependence on the CSN for survival. Mouse xenograft models will be treated with CSN inhibition, alone and in combination with PU-H71, to show effect on tumor growth and animal survival.

### Subaim 2: To Determine the Mechanism of CSN in the Survival of DLBCL

Immunoprecipitations (IPs) of the CSN will be used to demonstrate CSN-CBM interaction. Genetic ablation of the CSN will be used to demonstrate degradation of Bcl10 and ablation of NF-κB activity in DLBCL cell lines.

### Background and Significance

### 1. DLBCL Classification

DLBCL is the most common form of non-Hodgkin's lymphoma. In order to improve diagnosis and treatment of DLBCL, many studies have attempted to classify this molecularly heterogeneous disease. One gene expression profiling study divided DLBCL into two major subtypes (Alizadeh et al., 2000). Germinal center (GC) B cell like (GCB) DLBCL can be characterized by the expression of genes important for germinal center differentiation including BCL6 and CD10, whereas activated B cell like (ABC) DLBCL can be distinguished by a gene expression profile resembling that of activated peripheral blood B cells. The NF-κB pathway is more active and often mutated in ABC DLBCL. Another classification effort using gene expression profiling identified three major classes of DLBCL. OxPhos DLBCL shows significant enrichment of genes involved in oxidative phosphorylation, mitochondrial function, and the electron transport chain. BCR/proliferation DLBCL can be characterized by an increased expression of genes involved in cell-cycle regulation. Host response (HR) DLBCL is identified based on increased expression of multiple components of the T-cell receptor (TCR) and other genes involved in T cell activation (Monti et al., 2005).

These prospective classifications were made using patient samples and have not been the final answer for diagnosis or treatment of patients. Because patient samples are comprised of heterogeneous populations of cells and tumor microenvironment plays a role in the disease, (de Jong and Enblad, 2008), DLBCL cell lines do not classify as well as patient samples. However, well-characterized cell lines can be used as models of the different subtypes of DLBCL in which to investigate the molecular mechanisms behind the disease.

### 2. DLBCL: Need for Novel Therapies

Standard chemotherapy regimens such as the combination of cyclophosphamide, doxorubicin, vincristine, and prednisone (CHOP) cure about 40% of DLBCL patients, with 5-year overall survival rates for GCB and ABC patients of 60% and 30%, respectively (Wright et al., 2003). The addition of rituximab immunotherapy to this treatment schedule (R-CHOP) increases survival of DLBCL patients by 10 to 15% (Coiffier et al., 2002). However, 40% of DLBCL patients do not respond to R-CHOP, and the side effects of this combination chemoimmunotherapy are not well tolerated, emphasizing the need for identifying novel targets and treatments for this disease.

Classification of patient tumors has advanced the understanding of the molecular mechanisms underlying DLBCL to a degree. Until these details are better understood, treatments cannot be individually tailored. Preclinical studies of treatments with new drugs alone and in combination treatments and the investigation of new targets in DLBCL will provide new insight on the molecular mechanisms behind the disease.

### 3. Hsp90: A Promising Target

Hsp90 is an emerging therapeutic target for cancer. The chaperone protein is expressed constitutively, but can also be induced upon cellular stress, such as heat shock. Hsp90 maintains the stability of a wide variety of substrate proteins involved in cellular processes such as survival, proliferation and angiogenesis (Neckers, 2007). Substrate proteins of Hsp90 include oncoproteins such as NPM-ALK in anaplastic large cell lymphoma, and BCR-ACL in chronic myelogenous leukemia (Bonvini et al., 2002; Gorre et al., 2002). Because Hsp90 maintains the stability of oncogenic substrate proteins necessary for disease maintenance, it is an attractive therapeutic target. In fact, inhibition of Hsp90 results in degradation of many of its substrate proteins (Bonvini et al., 2002; Caldas-Lopes et al., 2009; Chiosis et al., 2001; Neckers, 2007; Nimmanapalli et al., 2001). As a result, many inhibitors of Hsp90 have been developed for the clinic (Taldone et al., 2008).

### 4. PU-H71: A Novel Hsp90 Inhibitor

A novel purine scaffold Hsp90 inhibitor, PU-H71, has been shown to have potent anti-tumor effects with an improved pharmacodynamic profile and less toxicity than other Hsp90 inhibitors (Caldas-Lopes et al., 2009; Cerchietti et al., 2010a; Chiosis and Neckers, 2006). Studies from our laboratory have shown that PU-H71 potently kills DLBCL cell lines, xenografts and ex vivo patient samples, in part, through degradation of BCL-6, a transcriptional repressor involved in DLCBL proliferation and survival (Cerchietti et al., 2010a).

A unique property of PU-H71 is its high affinity for tumor related-Hsp90, which explains why the drug been shown to accumulate preferentially in tumors (Caldas-Lopes et al., 2009; Cerchietti et al., 2010a). This property of PU-H71 makes it a useful tool in identifying novel targets for cancer therapy. By immobilizing PU-H71 on a solid support, a chemical precipitation (CP) of tumor-specific Hsp90 complexes can be obtained, and the substrate proteins of Hsp90 can be identified using a proteomics approach. Preliminary experiments using this method in DLBCL cell lines have revealed at least two potential targets that are stabilized by Hsp90 in DLBCL cells: the BCR pathway and the COP9 signalosome (CSN).

### 5. Combination Therapies in Cancer

Identifying rational combination treatments for cancer is essential because single agent therapy is not curative (Table 6). Monotherapy is not effective in cancer because of tumor cell heterogeneity. Although tumors grow from a single cell, their genetic instability produces a heterogeneous population of daughter cells that are often selected for enhanced survival capacity in the form of resistance to apoptosis, reduced dependence on normal growth factors, and higher proliferative capacity (Hanahan and Weinberg, 2000). Because tumors are comprised of heterogeneous populations of cells, a single drug will kill not all cells in a given tumor, and surviving cells cause tumor relapse. Tumor heterogeneity provides an increased number of potential drug targets and therefore, the need for combining treatments.

TABLE 6
Multiple therapeutic agents are required for tumor cure. (Kufe DW, 2003)

| | Number of Agents | Adjuvant or | Number of Agents |
|---|---|---|---|
| Tumor | Required for Cure | Neoadjuvant | Required for Cure |
| Acute lymphoblastic | 4-7 | Wilms | 2-3 |
| leukemia (children) | | | |
| Gestational | | Embryonic Rhabdo | 2-3 |
| Choriocarcinoma^{a} | | | |
| early | 1-3 | OGS | 3 |
| advanced | 2-4 | Soft tissue sarcoma | 3 |
| AML | 3+ | Ovary | 3-4 |
| Testis | 3 | Breast cancer | 2-4 |
| Burkitt^{b} | 1-4 | Colorectal | 2 |
| Hodgkin's disease | 4-5 | Lung non-small-cell carcinoma stage IIIA | 2 |
| DHL | 4-5 | Lung small-cell carcinoma, limited | 2-4 |

^{a}One agent is curative, but a higher cure rate results with two or more.
^{b}One agent cures state 1 African Burkitt, but two or more are better.

Exposure to chemotherapeutics can give rise to resistant populations of tumor cells that can survive in the presence of drug. Avoiding this therapeutic resistance is another important rationale for combination treatments.

Combinations of drugs with non-overlapping side effects can result in additive or synergistic anti-tumor effect at lower doses of each drug, thus lowering side effects. Therefore, the possible favorable outcomes for synergism or potentiation include i) increasing the efficacy of the therapeutic effect, ii) decreasing the dosage but increasing or maintaining the same efficacy to avoid toxicity, iii) minimizing the development of drug resistance, iv) providing selective synergism against a target (or efficacy synergism) versus host. Drug combinations have been widely used to treat complex diseases such as cancer and infectious diseases for these therapeutic benefits.

Because inhibition of Hsp90 kills malignant cells and results in degradation of many of its substrate proteins, identification of tumor-Hsp90 substrate proteins may reveal additional therapeutic targets. In this study, we aim to investigate the BCR pathway and the CSN, substrates of Hsp90, in DLBCL survival as potential targets for combination therapy with Hsp90 inhibition. We predict that combined inhibition of Hsp90 and its substrate proteins will synergize in killing DLBCL, providing increased patient response with decreased toxicity.

### 6. Synergy Between Inhibition of Hsp90 and its Substrate BCL6: Proof of Principle

The transcriptional repressor BCL6, a signature of GCB DLBCL gene expression, is the most commonly involved oncogene in DLBCL. BCL6 forms a transcriptional repressive complex to negatively regulate expression of genes involved in DNA damage response and plasma cell differentiation of GC B cells. BCL6 is required for B cells to undergo immunoglobulin affinity maturation (Ye et al., 1997), and somatic hypermutation in germinal centers. Aberrant constitutive expression of BCL6 (Ye et al., 1993), may lead to DLBCL as shown in animal models. A peptidomimetic inhibitor of BCL6, RIBPI, selectively kills BCL-6-dependent DLBCL cells (Cerchietti et al., 2010a; Cerchietti et al., 2009b) and is under development for the clinic.

CPs using PU-H71 beads reveal that BCL6 is a substrate protein of Hsp90 in DLBCL cell lines, and treatment with PU-H71 induces degradation of BCL6 (Cerchietti et al., 2009a) (FIG. 18). RI-BPI synergizes with PU-H71 treatment to kill DLBCL cell lines and xenografts (Cerchietti et al., 2010b) (FIG. 18). This finding acts as proof of principal that targets in DLBCL can be identified through CPs of tumor-Hsp90 and that combined inhibition of Hsp90 and its substrate proteins synergize in killing DLBCL.

### 7. BCR Signaling

The BCR is a large transmembrane receptor whose ligand-mediated activation leads to an extensive downstream signaling cascade in B cells (outlined in FIG. 19). The extracellular ligand-binding domain of the BCR is a membrane immunoglobulin (mIg), most often mIgM or mIgD, which, like all antibodies, contains two heavy Ig (IgH) chains and two light Ig (IgL) chains. The Igα/Igβ (CD79a/CD79b) heterodimer is associated with the mIg and acts as the signal transduction moiety of the receptor. Ligand binding of the BCR causes aggregation of receptors, inducing phosphorylation of immunoreceptor tyrosine-based activation motifs (ITAMs) found on the cytoplasmic tails of CD79a/CD79b by src family kinases (Lyn, Blk, Fyn). Syk, a cytoplasmic tyrosine kinase is recruited to doubly phosphorylated ITAMs on CD79a/CD79b, where it is activated, resulting in a signaling cascade involving Bruton's tyrosine kinase (BTK), phospholipase Cγ (PLCγ), and protein kinase Cβ (PKC-β). BLNK is an important adaptor molecule that can recruit PLCγ, phosphatidylinositol-3-kinase (PI3-K) and Vav. Activation of these kinases by BCR aggregation results in formation of the BCR signalosome at the membrane, comprised of the BCR, CD79a/CD79b heterodimer, src family kinases, Syk, BTK, BLNK and its associated signaling enzymes. The BCR signalosome mediates signal transduction from the receptor at the membrane to downstream signaling effectors.

Signals from the BCR signalosome are transduced to extracellular signal-related kinase (ERK) family proteins through Ras and to the mitogen activated protein kinase (MAPK) family through Rac/cdc43. Activation of PLCγ causes increases in cellular calcium (Ca²⁺), resulting in activation of Ca²⁺-calmodulin kinase (CamK) and NFAT. Significantly, increased cellular Ca²⁺ activates PKC-β, which phosphorylates Carma1 (CARD11), an adaptor protein that forms a complex with BCL10 and MALT1. This CBM complex activates IκB kinase (IKK), resulting in phosphorylation of IκB, which sequesters NF-κB subunits in the cytosol. Phosphorylated IκB is ubiquitinylated, causing its degradation and localization of NF-κB subunits to the nucleus. Many other downstream effectors in this complex pathway (p38 MAPK, ERK1/2, CaMK) translocate to the nucleus to affect changes in transcription of genes involved in cell survival, proliferation, growth, and differentiation (NF-κB, NFAT). Syk also activates phosphatidylinositol 3-kinase (PI3K), resulting in increased cellular PIP₃. This second messenger activates the acutely transforming retrovirus (Akt)/mammalian target of rapamycin (mTOR) pathway which promotes cell growth and survival (Dal Porto et al., 2004).

### 8. Aberrant BCR Signaling in DLBCL

BCR signaling is necessary for survival and maturation of B cells (Lam et al., 1997), particularly survival signaling through NF-κB. In fact, constitutive NF-κB signaling is a hallmark of ABC DLBCL (Davis et al., 2001). Moreover, mutations in the BCR and its effectors contribute to the enhanced activity of NF-κB in DLBCL, specifically ABC DLBCL.

It has been shown that mutations in the ITAMs of the CD79a/CD79b heterodimer associated with hyperresponsive BCR activation and decreased receptor internalization in DLBCL (Davis et al., 2010). CD79 ITAM mutations also block negative regulation by Lyn kinase. Lyn phosphorylates immunoreceptor tyrosine-based inactivation motifs (ITIMs) on CD22 and the Fc γ-receptor, membrane receptors that communicate with the BCR. After docking on these phosphorylated ITIMs, SHPT dephosphorylates CD79 ITAMs causing downmodulation of BCR signaling. Lyn also phosphorylates Syk at a negative regulatory site, decreasing its activity (Chan et al., 1997). Taken together, mutations in CD79 ITAMs, found in both ABC and GCB DLBCL, result in decreased Lyn kinase activity and increased signaling through the BCR.

Certain mutations in the BCR pathway components directly enhance NF-κB activity. Somatic mutations in the CARD 11 adaptor protein result in constitutive activation of IKK causing enhanced NF-κB activity even in the absence of BCR engagement (Lenz et al., 2008). A20, a ubiquitin-editing enzyme, terminates NF-κB signaling by removing ubiquitin chains from IKK. Inactivating mutations in A20 remove this brake from NF-κB signaling in ABC DLBCL (Compagno et al., 2009).

This constitutive BCR activity in ABC DLBCL has been referred to as “chronic active BCR signaling” to distinguish it from “tonic BCR signaling.” Tonic BCR signaling maintains mature B cells and does not require CARD11 because mice deficient in CBM components have normal numbers of B cells (Thome, 2004). Chronic active BCR signaling, however, requires the CBM complex and is distinguished by prominent BCR clustering, a characteristic of antigen-stimulated B cells and not resting B cells. In fact, knockdown of CARD11, MALT1, and BCL10 is preferentially toxic for ABC as compared to GCB DLBCL cell lines (Ngo et al., 2006). Chronic active BCR signaling is associated mostly with ABC DLBCL, however CARD11 and CD79 ITAM mutations do occur in GCB DLBCL (Davis et al., 2010; Lenz et al., 2008), suggesting that BCR signaling is a potential target across subtypes of DLBCL.

### 9. Targeting the BCR Pathway in DLBCL

Because it promotes cell growth, proliferation and survival, BCR signaling is an obvious target in cancer. Mutations in the BCR pathway in DLBCL (described above) highlight its relevance as a target in the disease. In fact, many components of the BCR have been targeted in DLBCL, and some of these treatments have already translated to patients.

Overexpression of protein tyrosine phosphatase (PTP) receptor-type O truncated (PTPROt), a negative regulator of Syk, inhibits proliferation and induces apoptosis in DLBCL, identifying Syk as a target in DLBCL (Chen et al., 2006). Inhibition of Syk by small molecule fostamatinib disodium (R406) blocks proliferation and induces apoptosis in DLBCL cell lines (Chen et al., 2008). This orally available compound has also shown significant clinical activity with good tolerance in DLBCL patients (Friedberg et al., 2010).

An RNA interference screen revealed Btk as a potential target in DLBCL. Short hairpin RNAs (shRNAs) targeting Btk are highly toxic for DLBCL cell lines, specifically ABC DLBCL. A small molecule irreversible inhibitor of Btk, PCI-32765 (Honigberg et al., 2010), potently kills DLBCL cell lines, specifically ABC DLBCL (Davis et al., 2010). The compound is in clinical trials and has shown efficacy in B cell malignancies with good tolerability (Fowler et al., 2010).

Constitutive activity of NF-κB makes it a rational target in DLBCL. NF-κB can be targeted through different approaches Inhibition of IKK blocks phosphorylation of IκB, preventing release and nuclear translocation of NF-κB subunits. MLX105, a selective IKK inhibitor, potently kills ABC DLBCL cell lines (Lam et al., 2005). NEDD8-activating enzyme (NAE) regulates the CRL1^{βTRCP} ubiquitination of phosphorylated IκB, resulting in its degradation and the release of NF-κB subunits. Inhibition of NAE by small molecules such as MLN4924 induces apoptosis in ABC DLBCL and shows strong tumor burden regression in DLBCL and patient xenograft models. MLN4924 shows more potency in ABC DLBCL, which is expected because of the higher dependence on constitutive NF-κB activity for survival in this subtype (Milhollen et al., 2010). Because it activates IKK, inhibiting PKC-β is another approach to block NF-κB activity. Specific PKC-β inhibitors, such as Ly379196, kill both ABC and GCB DLBCL cell lines, albeit at high doses (Su et al., 2002).

These approaches to targeting NF-κB activity are promising therapies for DLBCL. Inhibition of IKK and NAE is most potent in ABC DLBCL, but less potent effect was also seen in GCB DLBCL. These studies suggest that combining NF-κB activity with other targeted therapies may produce a more robust effect across DLBCL subtypes.

The PI3K/Akt/mTOR pathway is deregulated in many human diseases and is constitutively activated in DLBCL (Gupta et al., 2009). Because malignant cells exploit this pathway to promote cell growth and survival, small molecule inhibitors of the pathway have been heavily researched. Rapamycin (sirolimus), a macrolide antibiotic that targets mTOR, is an FDA approved oral immunosuppressant (Yap et al., 2008). Everolimus, an orally available rapamycin analog, has also been approved as a transplant immunosuppressant (Hudes et al., 2007). These compounds have antitumor activity in DLBCL cell lines and patient samples (Gupta 2009), but their effect is mostly antiproliferative and only narrowly cytotoxic. To achieve cytotoxicity, rapamycin and everolimus have been evaluated in combination with many other therapeutic agents (Ackler et al., 2008; Yap et al., 2008). Phase II clinical studies of everolimus in DLBCL have been moderately successful with an ORR of 35% (Reeder C, 2007). Everolimus has also been shown to sensitize DLBCL cell lines to other cytotoxic agents (Wanner et al., 2006). These findings clearly demonstrate the therapeutic potential of mTOR inhibition in DLBCL, especially in combination therapies.

Inhibition of Akt is also a promising cancer therapy and can be targeted in many ways. Lipid based inhibitors block the PIP3-binding PH domain of Akt to prevent its translocation to the membrane. One such drug, perifosine, has shown antitumor activity both in vitro and in vivo.

Overall, the compound has shown only partial responses, prompting combination with other targeted therapies (Yap et al., 2008). Small molecule inhibitors of Akt, such as GSK690693, cause growth inhibition and apoptosis in lymphomas and leukemias, specifically ALL (Levy et al., 2009), and may be effective in killing DLBCL as a monotherapy or in combination with other targeted therapies.

The MAPK pathway is another interesting target in cancer therapeutics. The oncogene MCT-1 is highly expressed in DLBCL patient samples and is regulated by ERK Inhibition of ERK causes apoptosis in DLBCL xenograft models (Dai et al., 2009). Small molecule inhibitors of ERK and MEK have been developed and demonstrate excellent safety profiles and tumor suppressive activity in the clinic. The response to these drugs, however, has not been robust with four partial patient responses observed and stable disease reported in 22% of patients (Friday and Adjei, 2008) Inhibition of MEK alone may be insufficient to cause cytotoxicity because the upstream regulators of the MAPK pathway, namely Ras and Raf, are most frequently mutated in cancer and may regulate other kinases that maintain cell survival despite MEK inhibition. In the face of these pitfalls, MEK inhibitors such as AZD6244 have entered the clinic. The partial response to MEK inhibition suggests that combinations of these inhibitors with other targeted therapies may reveal a more robust patient response (Friday and Adjei, 2008).

### 10. The CSN: Structure and Function

The CSN was first discovered in Aradopsis in 1996 as a negative regulator of photomorphogenesis (Chamovitz et al., 1996). The complex is highly conserved from yeast to human and is comprised of eight subunits, CSN1-CSN8, numbered in size from largest to smallest (Deng et al., 2000). Most of the CSN subunits are more stable as part of the eight subunit holocomplex, but some smaller complexes, such as the mini-CSN, containing CSN4-7, have been reported (Oron et al., 2002; Tomoda et al., 2002). CSN5, first identified as Junactivation-domain-binding protein (Jab1), functions independently of the holo-CSN, and has been shown to interact with many cellular signaling mediators (Kato and Yoneda-Kato, 2009). The molecular constitution and functionality of these complexes are not yet clearly understood.

CSN5 and CSN6 each contain an MPR1-PAD1-N-terminal (MPN) domain, but only CSN5 contains a JAB 1 MPN domain metalloenzyme motif (JAMM/MPN+ motif). The other six subunits contain a proteasome-COP9 signalosome-initiation factor 3 domain (PCI (or PINT)) (Hofmann and Bucher, 1998). Though the exact function of these domains is not yet fully understood, they bear an extremely similar homology to the lid complex of the proteasome and the eIF3 complex (Hofmann and Bucher, 1998), suggesting that the function of the CSN relates to protein synthesis and degradation.

The best characterized function of the CSN is the regulation of protein stability. The CSN regulates protein degradation by deneddylation of cullins. Cullins are protein scaffolds at the center of the ubiquitin E3 ligase. They also serve as docking sites for ubiquitin E2 conjugating enzymes and protein substrates targeted for degradation. The cullin-RING-E3 ligases (CRLs) are the largest family of ubiquitin ligases. Post-translational modification of the cullin subunit of a CRL by conjugation of Nedd8 is required for CRL activity (Chiba and Tanaka, 2004; Ohh et al., 2002). The CSN5 JAMM motif catalyzes removal of Nedd8 from CRLs; this deneddylation reaction requires an intact CSN holocomplex (Cope et al., 2002; Sharon et al., 2009). Although cullin deneddylation inactivates CRLs, the CSN is required for CRL activation (Schwechheimer and Deng, 2001), and may prevent CRL components from self-destruction by autoubiquitinylation (Peth et al., 2007).

The CSN has many other biological functions, including apoptosis and cell proliferation. Knockout of CSN components 2, 3, 5, and 8 in mice causes early embryonic death due to massive apoptosis with CSN5 knockout exhibiting the most severe phenotype (Lykke-Andersen et al., 2003; Menon et al., 2007; Tomoda et al., 2004; Yan et al., 2003). These functions may be related to the complex's role in protein stability and degradation because the phenotypes in these knockout animals parallel the phenotype of NAE knockout mice (Tateishi et al., 2001) and knockout mice of various cullins (Dealy et al., 1999; Li et al., 2002; Wang et al., 1999).

Ablation of CSN5 in thymocytes results in apoptosis as a result of increased expression of proapoptotic BCL2-associated X protein (Bax) and decreased expression of anti-apoptotic Bcl-xL protein (Panattoni et al., 2008). The interaction of CSN5 with the cyclin-dependent kinase (CDK) inhibitor p27 suggests its role in cell proliferation (Tomoda et al., 1999). CSN5 knockout thymocytes display G2 arrest (Panattoni et al., 2008), while CSN8 plays a role in T cell entry to the cell cycle from quiescence (Menon et al., 2007).

### 11. The CSN and Cancer

The involvement of the CSN in such cellular functions as apoptosis, proliferation and cell cycle regulation suggest that it may play a role in cancer. In fact, overexpression of CSN5 is observed in a variety of tumors (Table 7), and knockdown of CSN5 inhibits the proliferation of tumor cells (Fukumoto et al., 2006). CSN5 is also involved in myc-mediated transcriptional activation of genes involved in cell proliferation, invasion and angiogenesis (Adler et al., 2006). CSN2 and CSN3 are identified as putative tumor suppressors due to their ability to overcome senescence (Leal et al., 2008), and inhibit the proliferation of mouse fibroblasts (Yoneda-Kato et al., 2005), respectively.

TABLE 7
CSN5 Overexpression Correlating Tumor Progression
or Clinical Outcome (Richardson and Zundel, 2005)

| Prognostic | | Increased expression associated with poor clinical |
|---|---|---|
| indicator | Cancer (reference) | outcome |
| CSN5 | Pancreatic ductal adenocarcinoma (101) | Not evaluated |
| CSN5 | Hepatocellular carcinoma (53) | Gene amplification (76%) |
| CSN5 | Hepatocellular carcinoma (102) | Not evaluated |
| CSN5 | Laryngeal squamous cell carcinoma (87) | Indicator of disease-free and overall survival |
| CSN5 | Oral squamous cell carcinoma (103) | Indicator of lymph node metastisis and poor prognosis |
| CSN6 | Lung adenocarcinoma (104) | Indicator of disease state but not clinical outcome |
| CSN6 | Breast ductal carcinoma in situ (105) | Expression is higher in lesions with necrosis |
| CSN6 | Node-negative breast cancer (89) | Associated with tumor size but not disease-free survival |
| CSN5 | Invasive breast carcinoma (89) | Indicator of disease progression and relapse |
| CSN5 | Melanoma (108) | Not evaluated |
| CSN5 | Rhabdomyosarcoma (91) | Not evaluated |
| CSN5 | Pituitary carcinomas (110) | Not evaluated |
| CSN6 | Neuroblastoma (131) | Localization associated with tumor differentiation |
| CSN6 | B-cell non-Hodgkin’s lymphoma (112) | Not evaluated |
| CSN6 | Malignant lymphoma (thyroid, ref. 113) | Predictor of tumor grade and proliferating index |

Knockdown of CSN5 in xenograft models significantly decreases tumor growth (Supriatno et al., 2005). Derivatives of the natural product curcumin inhibit the growth of pancreatic cancer cells by inhibition of CSN5 (Li et al., 2009). Taken together, these findings indicate that the CSN is a good therapeutic target in cancer.

### 12. The CSN and NF-κB Activation: A Role in DLBCL?

The CSN regulates NF-κB activity differently in different cellular contexts. In TNFα-stimulated synviocytes of rheumatoid arthritis patients, knockdown of CSN5 abrogates TNFR1-ligationdependent IκBα degradation and NF-κB activation (Wang et al., 2006). Ablation of CSN subunits in TNFα-stimulated endothelial cells, however, results in stabilization of IκBα and sustained nuclear translocation of NF-κB (Schweitzer and Naumann, 2010).

Studies of the CSN in T cells demonstrate its critical role in T cell development and survival. Thymocytes from CSN5 null mice display cell cycle arrest and increased apoptosis. Importantly, these cells show accumulation of IκBα, reduced nuclear NF-κB accumulation, and decreased expression of anti-apoptotic NF-κB target genes (Panattoni et al., 2008), suggesting that CSN5 regulates T-cell activation. In fact, the CSN interacts with the CBM complex in activated T cells. T-cell activation stimulates interaction of the CSN with MALT1 and CARD11 and with BCL10 through MALT1. CSN2 and CSN5 stabilize the CBM by deubiquitinylating BCL10. Knockdown of either subunit causes rapid degradation of Bcl10 and also blocks IKK activation in TCR-stimulated T cells, suggesting that CSN may regulate NF-κB activity through this mechanism (Welteke et al., 2009).

The exact function of the CSN in NF-κB regulation is not well defined, and may differ depending on cell type. The involvement of the CSN in NF-κB regulation, particularly in T cells and through the stabilization of the CBM, suggests that it may play a role in DLBCL pathology.

### Preliminary Results

CPs were performed in OCI-Ly1 and OCI-Ly7 DLBCL cell lines. Cells were lysed, and cytosolic and nuclear lysates were extracted. Lysates were incubated with either control or agarose beads coated with PUH71 overnight, then washed to remove non-specifically bound proteins. Tightly binding proteins were eluted by boiling in SDS/PAGE loading buffer, separated by SDS/PAGE and visualized by colloidal blue staining Gel lanes were cut into segments and analyzed by mass spectroscopy by our collaborators. Proteins that were highly represented (determined by number of peptides) in PUH71 pulldowns but not control pulldowns are candidate DLBCL-related Hsp90 substrate proteins. After excluding common protein contaminants and the agarose proteome, we obtained 80% overlapping putative client proteins (N=˜200) in both cell lines represented by multiple peptides. One of the pathways highly represented among PU-H71 Hsp90 clients in these experiments is the BCR pathway (23 proteins out of 200, shown in grey in FIG. 19 and FIG. 23). We have begun validating this finding. Preliminary data shows that Syk and Btk are both degraded with increasing PU-H71 and are both pulled down with PU-H71 in CPs of DLBCLs. PU-H71 synergizes with R406, a Syk inhibitor, to kill DLBCL cell lines (FIG. 20).

### Experimental Approach

### AIM1: To Determine Whether Concomitant Modulation of Hsp90 and BCR Pathways Cooperate in Killing DLBCL Cells In Vitro and In Vivo

Our preliminary data identified many components of the BCR pathway as substrate proteins of Hsp90 in DLBCL. The BCR pathway has been implicated in oncogenesis and DLBCL survival. We hypothesize that combined inhibition of Hsp90 and components of the BCR pathway will synergize in killing DLBCL.

### Experimental Design and Expected Outcomes

DLBCL cell lines will be maintained in culture. GCB DLBCL cell lines will include OCI-Ly1, OCI-Ly7, and Toledo. ABC DLBCL cell lines will include OCI-Ly3, OCI-Ly10, HBL-1, TMD8. Cell lines OCI-Ly1, OCI-Ly7, and OCI-Ly10 will be maintained in 90% Iscove's modified medium containing 10% FBS and supplemented with penicillin and streptomycin. Cell lines Toledo, OCI-Ly3, and HBL-1 will be grown in 90% RPMI and 10% FBS supplemented with penicillin and streptomycin, L-glutamine, and HEPES. The TMD8 cell line will be grown in medium containing 90% mem-alpha and 10% FBS supplemented with penicillin and streptomycin.

Components of the BCR pathway were identified as subtrate proteins of Hsp90 in a preliminary experiment of a proteomics analysis of PU-H71 CPs in two DLBCL cell lines. To verify that the components of the BCR pathway are stabilized by Hsp90, CPs will be performed using DLBCL cell lines and analyzed by western blot using commercially available antibodies to BCR pathway components, including CD79a, CD79b, Syk, Btk, PLCγ2, AKT, mTOR, CAMKII, p38 MAPK, p40 ERK1/2, p65, Bcl-XL, Bcl6. CPs will be performed with increasing salt concentrations to show the affinity of Hsp90 for these substrate proteins. Because some proteins are expressed at low levels, nuclear/cytosolic separation of cell lysates will be performed to enrich for Hsp90 substrate proteins that are not readily detected using whole cell lysate.

Hsp90 stabilization of BCR pathway components will also be demonstrated by treatment of DLBCL cell lines with increasing doses of PU-H71. Levels of the substrate proteins listed above will be determined by western blot. Substrate proteins are expected to be degraded by exposure to PU-H71 in a dose-dependent and time-dependent manner.

Viability of DLBCL cell lines will be assessed following treatment with PU-H71 or inhibitors of BCR pathway components (Syk, Btk, PLCγ2, AKT, mTOR, p38 MAPK, p40 ERK1/2, NF-κB). Inhibitors of BCR pathway components will be selected and prioritized based on reported data in DLBCL and use in clinical trials. For example, the Melnick lab has MTAs in place to use PCI-32765 and MLN4924 (described above). Cells will be plated in 96-well plates at concentrations sufficient to keep untreated cells in exponential growth for the duration of drug treatment. Drugs will be administered in 6 different concentrations in triplicate wells for 48 hours. Cell viability will be measured with a fluorometric resazurin reduction method (CellTiter-Blue, Promega).

Fluorescence (560_{excitation}/590ₑₘᵢₛₛᵢₒₙ) will be measured using the Synergy4 microplate reader in the Melnick lab (BioTek). Viability of treated cells will be normalized to appropriate vehicle controls, for example, water, in the case of PU-H71. Dose-effect curves and calculation of the drug concentration that inhibits the growth of the cell line by 50% compared to control (GI50) will be performed using CompuSyn software (Biosoft). Although many of these findings may be confirmatory of published data, instituting effective methods with these inhibitors and determining their dose-responses in our cell lines will be necessary for later combination treatment experiments demonstrating the effect of combined inhibition of Hsp90 and the BCR pathway.

Once individual dose-response curves and GI50s for BCR pathway inhibitors have been established, DLBCL cells will be treated with both PU-H71 and single inhibitors of the BCR pathway to demonstrate the effect of the combination on cell killing. Experiments will be performed in 96-well plates using the conditions described above. Cells will be treated with 6 different concentrations of combination of drugs in constant ratio in triplicate with the highest dose being twice the GI50 of each drug as measured in individual dose-response experiments. Drugs will be administered in different sequences in order to determine the most effective treatment schedule: PU-H71 followed by drug X after 24 hours, drug X followed by PU-H71 after 24 hours, and PU-H71 with drug X. Viability will be determined after 48 hours using the assays mentioned above. Isobologram analysis of cell viability will be performed using Compusyn software.

Combination treatments in DLBCL cell lines proposed above will guide experiments in xenograft models in terms of dose and schedule. The drug schedules that exhibit the best cell killing effect will be translated to xenograft models. DLBCL cell lines will be injected subcutaneously into SCID mice, using two cell lines expected to respond to drug and one cell line expected not to respond as a negative control. Tumor growth will be monitored every other day until palpable (about 75-100 mm³). Animals (n=20) will be randomly divided into the following groups: control, PU-H71, BCR pathway inhibitor (drug X), and PU-H71+drug X with five animals per group. To measure drug effect on tumor growth, tumor volume will be measured with Xenogen IVIS system every other day after drug administration. After ten days, all animals will be sacrificed, and tumors will be assayed for apoptosis by TUNEL. To assess drug effect on survival, a second cohort of animals as specified above will be treated and sacrificed when tumors reach 1000 mm³ in size. Tumors will be analyzed biochemically to demonstrate that the drugs hit their targets, by ELISA for NF-κB activity or phosphorlyation of downstream targets, for example. We will perform toxicity studies established in the Melnick lab (Cerchietti et al., 2009a) in treated mice including physical examination, macro and microscopic tissue examination, serum chemistries and CBCs.

### Alternatives and Pitfalls

If the fluorescence assay used to detect cell viability is incompatible with some cell lines (due to acidity of media, for example,) an ATP-based luminescent method (CellTiter-Glo, Promega) will be used. Also, because some drugs may not kill cells in 48 hours, higher drug doses and longer drug incubations will be performed if necessary to determine optimal drug treatments. It is possible inhibition of some BCR pathway components will not demonstrate an improved effect in killing DLBCL when combined with inhibition of Hsp90, but based on preliminary data shown above, we believe that some combinations will be more effective than either drug alone.

### AIM 2: To Evaluate the Role of the CSN in DLBCL

### Subaim 1: To Determine Whether the CSN can be a Therapeutic Target in DLBCL

Our preliminary data has identified subunits of the CSN as substrate proteins of Hsp90 in DLBCL. The CSN has been implicated in cancer and may play a role in DLBCL survival. We hypothesize that DLBCL requires the CSN for survival and that combined inhibition of Hsp90 and the CSN will synergize in killing DLBCL.

### Experimental Design and Expected Outcomes

Expression of CSN subunits in DLBCL cell lines (described above) will be verified. DLBCL cell lines will be lysed for protein harvest and analyzed by SDS-PAGE and western blotting using commercially available antibodies to the CSN subunits and actin as a loading control.

The CSN was identified as a substrate protein of Hsp90 in a preliminary proteomics analysis of PU-H71 CPs in two DLBCL cell lines. To verify that Hsp90 stabilizes the CSN, CPs will be performed as described above using DLBCL cell lines and analyzed by western blot. Hsp90 stabilization of the CSN will also be demonstrated by treatment of DLBCL cell lines with increasing PU-H71 concentration. Protein levels of CSN subunits will be determined by western blot. CSN subunits are expected to be degraded upon exposure to PU-H71 in a dose-dependent and time-dependent manner.

DLBCL cells lines will be infected with lentiviral pLKO.1 vectors containing short hairpin (sh)RNAs targeting CSN2 or CSN5 and selected by puromycin resistance. These vectors are commercially available through the RNAi Consortium. These subunits will be used because knockdown of one CSN subunit can affect expression of other CSN subunits (Menon et al., 2007; Schweitzer et al., 2007; Schweitzer and Naumann, 2010), and knockdown of CSN2 ablates formation of the CSN holocomplex. CSN5 knockdown will be used because this subunit contains the enzymatic domain of the CSN. A pool of 3 to 5 shRNAs will be tested against each target to obtain the sequence with optimal knockdown of the target protein. Empty vector and scrambled shRNAs will be used as controls. Because we predict that knockdown of CSN subunits will kill DLBCL cells, and we aim to measure cell viability, tetracycline (tet) inducible constructs will be used. This method may also allow us to establish conditions for dose-dependent knockdown of CSN subunits using a titration of shRNA induction. Knockdown efficiency will be assessed by western blot following infection and tet induction. Cells will be assessed for viability using the methods described in Aim 1 following infection. We predict that knockdown the CSN will kill DLBCLs, and ABC DLBCLs are expected to depend on the CSN for survival more than GCB DLBCLs because of the CSN's role in stabilizing the CBM complex.

Following CSN monotherapy experiments in DLBCL, induction of CSN knockdown will be combined with PU-H71 treatment in DLBCL cell lines. shRNA constructs that demonstrate effective dose dependent CSN knock down in 48 hours (as evaluated in earlier experiments) will be used in order to perform 48 hour cell viability experiments. Control shRNAs as described above will be used. Control cells and cells infected with tet-inducible shRNA constructs targeting CSN subunits will be treated with different doses of tet and PU-H71 in constant ratio in triplicate. Drugs will be administered in different sequences in order to determine the most effective treatment schedule: PU-H71 followed by tet, tet followed by PU-H71, and PU-H71 with tet. Cell viability will be measured as described in Aim 1. Combined inhibition of the CSN and Hsp90 is expected to synergize in killing DLBCL, specifically ABC DLBCL.

Combined inhibition of the CSN and Hsp90 in DLBCL cell lines proposed above will guide experiments in xenograft models. The most effective combination of PU-H71 and CSN knockdown from in vitro experiments will be used in xenograft experiments. Control and inducible-knockout-CSN DLBCL cells will be used for xenograft, using two cell lines expected to respond to treatment and one cell line expected not to respond to treatment as a negative control. Animals will be treated with vehicle, PU-H71, or tet, using the dose and schedule of the most effective combination of PU-H71 and tet as determined by in vitro experiments. Tumor growth, animal survival and toxicity will be assayed as described in Aim 1.

### Alternatives and Pitfalls

Accomplishing dose-dependent knockdown of the CSN by titration of tetracycline induction may prove difficult. If this occurs, in order to demonstrate proof of principle, shRNAs with different knockdown efficiencies will be used to simulate increasing inhibition of the CSN as a monotherapy and in combination with different doses of PU-H71.

### Subaim 2: To Determine the Mechanism of DLBCL Dependence on the CSN

Since the CSN has been shown to interact with the CBM complex and activate IKK in stimulated T-cells, we hypothesize that the CSN interacts with the CBM, stabilizes Bcl10, and activates NF-κB in DLBCL.

### Experimental Design and Expected Outcomes

DLBCL cell lysates will be incubated with an antibody to CSN1 that effectively precipitates the whole CSN complex (da Silva Correia et al., 2007; Wei and Deng, 1998). Precipitated CSN1 complexes will be separated by SDS-PAGE and analyzed for interaction with CBM components by western blot using commercially available antibodies to the different components of the CBM: CARD11, BCL10, and MALT1. Based on reported experiments in T cells, we expect the CSN to interact preferentially with CARD11 and MALT1 in ABC DLBCL cell lines as opposed to GCB DLBCL cell lines because of the chronic active BCR signaling in ABC DLBCL.

Because the CSN, specifically CSN5, has been shown to regulate Bcl10 stability and degradation in activated T-cells, we hypothesize that the CSN stabilizes Bcl10 in DLBCL. DLBCL cells lines will be infected with short hairpin (sh)RNAs targeting CSN subunits as described above. Cells will be treated with tet to induce CSN subunit knockdown and Bcl10 protein levels in infected and induced cells will be quantified by western blot. We expect Bcl10 levels to be degraded with CSN subunit knockdown in a dose-dependent and time-dependent manner. To demonstrate that reduction in Bcl10 protein is not a result of cell death, cell viability will be measured by counting viable cells with Trypan blue before cell lysis. CSN subunit knockdown will be combined with proteasome inhibition to demonstrate that Bcl10 degradation is a specific effect of CSN ablation.

Knockdown of CSN2 or CSN5 is expected to abrogate NF-κB activity in DLBCL cell lines. Using DLBCL cell lines infected with control shRNAs or shRNAs to CSN2 or CSN5, control and infected cells will be assayed for NF-κB activity in several ways. First, lysates will be analyzed by western blot to determine levels of IκBα protein. Second, nuclear translocation of the NF-κB subunits p65 and c-Rel will be measured by western blot of nuclear and cytosolic fractions of lysed cells or by plate-based EMSA of nuclei from control and infected cells. Finally, NF-κB target gene expression of these cells will be evaluated at the transcript and protein level by quantitative PCR of cDNA prepared by reverse transcriptase PCR (RT-PCR) and western blot, respectively.

### Alternatives and Pitfalls

Because the CSN was shown to interact with the CBM in TCR-stimulated T cells, we predict that the CSN interacts with the CBM in DLBCL, especially in ABC DLBCL because this subtype exhibits chronic active BCR signaling. If CSN-CBM interaction is not apparent in DLBCL, then cells will be stimulated with IgM in order to activate the BCR pathway and stimulate formation of the CBM. To determine the kinetics of the CSN interaction with the CBM, cellular IPs as described above will be performed over a time course from the point of IgM stimulation. To correlate CSN-CBM interaction with the kinetics of CBM formation, BCL10 IP will be performed to demonstrate BCL10-CARD11 interaction over the same time course.

### Conclusions and Future Directions

The development of PU-H71 as a new therapy for DLBCL is promising, but combination treatments are likely to be more potent and less toxic. PU-H71 can also be used as a tool to identify substrate proteins of Hsp90. In experiments using this method, the BCR pathway and the CSN were identified as substrates of Hsp90 in DLBCL.

The BCR plays a role in DLBCL oncogenesis and survival, and efforts to target components of this pathway have been successful. We predict that combining PU-H71 and inhibition of BCR pathway components will be a more potent and less toxic treatment approach. Identified synergistic combinations in cells and xenograft models will be evaluated for translation to clinical trials, and ultimately advance patient treatment toward rationally designed targeted therapy and away from chemotherapy.

The CSN has been implicated in cancer and NF-κB activation, indicating that it may be a good target in DLBCL. We hypothesize that the CSN stabilizes the CBM complex, promoting NF-κB activation and DLBCL survival. Therefore, we predict that combined inhibition of Hsp90 and the CSN will synergize in killing DLBCL. These studies will act as proof of principle that new therapeutic targets can be identified using the proteomics approach described in this proposal.

Future studies will identify compounds that target the CSN, and ultimately bring CSN inhibitors to the clinic as an innovative therapy for DLBCL. Determining downstream effects of CSN inhibition, such as CBM stabilization and NF-κB activation may reveal new opportunities for additional combinatorial drug regimens of three drugs. Future studies will evaluate combinatorial regimens of three drugs inhibiting Hsp90, the CSN and its downstream targets together.

The most effective drug combinations with PU-H71 found in this study will be performed using other Hsp90 inhibitors in clinical development such as 17-DMAG to demonstrate the broad clinical applicability of identified effective drug combinations.

DLBCL, the most common form of non-Hodgkins lymphoma, is an aggressive disease that remains without cure. The studies proposed herein will advance the understanding of the molecular mechanisms behind DLBCL and improve patient therapy.

Here, we report on the design and synthesis of molecules based on purine, purine-like isoxazole and indazol-4-one chemical classes attached to Affi-Gel® 10 beads (FIGS. 30, 32, 33, 35, 38) and on the synthesis of a biotinylated purine, purine-like, indazol-4-one and isoxazole compounds (FIGS. 31, 36, 37, 39, 40). These are chemical tools to investigate and understand the molecular basis for the distinct behavior of Hsp90 inhibitors. They can be also used to better understand Hsp90 tumor biology by examining bound client proteins and co-chaperones. Understanding the tumor specific clients of Hsp90 most likely to be modulated by each Hsp90 inhibitor could lead to a better choice of pharmacodynamic markers, and thus a better clinical design. Not lastly, understanding the molecular differences among these Hsp90 inhibitors could result in identifying characteristics that could lead to the design of an Hsp90 inhibitor with most favorable clinical profile.

### Methods of Synthesizing of Hsp90 Probes

### 6.1. General

¹H and ¹³C NMR spectra were recorded on a Bruker 500 MHz instrument. Chemical shifts were reported in δ values in ppm downfield from TMS as the internal standard. ¹H data were reported as follows: chemical shift, multiplicity (s=singlet, d=doublet, t=triplet, q=quartet, br=broad, m=multiplet), coupling constant (Hz), integration. ¹³C chemical shifts were reported in δ values in ppm downfield from TMS as the internal standard. Low resolution mass spectra were obtained on a Waters Acquity Ultra Performance LC with electrospray ionization and SQ detector. High-performance liquid chromatography analyses were performed on a Waters Autopurification system with PDA, MicroMass ZQ and ELSD detector and a reversed phase column (Waters X-Bridge C18, 4.6×150 mm, 5 μm) using a gradient of (a) H₂O+0.1% TFA and (b) CH₃CN+0.1% TFA, 5 to 95% b over 10 minutes at 1.2 mL/min. Column chromatography was performed using 230-400 mesh silica gel (EMD). All reactions were performed under argon protection. Affi-Gel® 10 beads were purchased from Bio-Rad (Hercules, Calif.). EZ-Link® Amine-PEO₃-Biotin was purchased from Pierce (Rockford, Ill.). PU-H71 (He et al., 2006) and NVP-AUY922 (Brough et al., 2008) were synthesized according to previously published methods. GM was purchased from Aldrich.

### 6.2. Synthesis

### 6.2.1. 9-(3-Bromopropyl)-8-(6-iodobenzo[d][1,3]dioxol-5-ylthio)-9H-purin-6-amine (2)

1 (He et al., 2006) (0.500 g, 1.21 mmol) was dissolved in DMF (15 mL). Cs₂CO₃ (0.434 g, 1.33 mmol) and 1,3-dibromopropane (1.22 g, 0.617 mL, 6.05 mmol) were added and the mixture was stirred at rt for 45 minutes. Then additional Cs₂CO₃ (0.079 g, 0.242 mmol) was added and the mixture was stirred for 45 minutes. Solvent was removed under reduced pressure and the resulting residue was chromatographed (CH₂Cl₂:MeOH:AcOH, 120:1:0.5 to 80:1:0.5) to give 0.226 g (35%) of 2 as a white solid. ¹H NMR (CDCl₃/MeOH-d₄) δ 8.24 (s, 1H), 7.38 (s, 1H), 7.03 (s, 1H), 6.05 (s, 2H), 4.37 (t, J=7.1 Hz, 2H), 3.45 (t, J=6.6 Hz, 2H), 2.41 (m, 2H); MS (ESI): m/z 534.0/536.0 [M+H]⁺.

### 6.2.2. tert-Butyl 6-aminohexylcarbamate (3) (Hansen et al., 1982)

1,6-diaminohexane (10 g, 0.086 mol) and Et₃N (13.05 g, 18.13 mL, 0.129 mol) were suspended in CH₂Cl₂ (300 mL). A solution of di-tert-butyl dicarbonate (9.39 g, 0.043 mol) in CH₂Cl₂ (100 mL) was added dropwise over 90 minutes at rt and stirring continued for 18 h. The reaction mixture was added to a seperatory funnel and washed with water (100 mL), brine (100 mL), dried over Na₂SO₄ and concentrated under reduced pressure. The resulting residue was chromatographed [CH₂Cl₂:MeOH—NH₃ (7N), 70:1 to 20:1] to give 7.1 g (76%) of 3. ¹H NMR (CDCl₃) δ 4.50 (br s, 1H), 3.11 (br s, 2H), 2.68 (t, J=6.6 Hz, 2H), 1.44 (s, 13H), 1.33 (s, 4H); MS (ESI): m/z 217.2 [M+H]⁺.

### 6.2.3. tert-Butyl 6-(3-(6-amino-8-(6-iodobenzo[d][1,3]dioxol-5-ylthio)-9H-purin-9-yl)propylamino)hexylcarbamate (4)

2 (0.226 g, 0.423 mmol) and 3 (0.915 g, 4.23 mmol) in DMF (7 mL) was stirred at rt for 24 h. The reaction mixture was concentrated and the residue chromatographed [CHCl₃:MeOH:MeOH—NH₃ (7N), 100:7:3] to give 0.255 g (90%) of 4. ¹H NMR (CDCl₃) δ 8.32 (s, 1H), 7.31 (s, 1H), 6.89 (s, 1H), 5.99 (s, 2H), 5.55 (br s, 2H), 4.57 (br s, 1H), 4.30 (t, J=7.0 Hz, 2H), 3.10 (m, 2H), 2.58 (t, J=6.7 Hz, 2H), 2.52 (t, J=7.2 Hz, 2H), 1.99 (m, 2H), 1.44 (s, 13H), 1.30 (s, 4H); ¹³C NMR (125 MHz, CDCl₃) δ 156.0, 154.7, 153.0, 151.6, 149.2, 149.0, 146.3, 127.9, 120.1, 119.2, 112.4, 102.3, 91.3, 79.0, 49.8, 46.5, 41.8, 40.5, 31.4, 29.98, 29.95, 28.4, 27.0, 26.7; HRMS (ESI) m/z [M+H]′ calcd. for C₂₆H₃₇IN₇O₄S, 670.1673. found 670.1670; HPLC: t_{R}=7.02 min.

### 6.2.4. N¹-(3-(6-Amino-8-(6-iodobenzo[d][1,3]dioxol-5-ylthio)-9H-purin-9-yl)propyl)hexane-1,6-diamine (5)

4 (0.310 g, 0.463 mmol) was dissolved in 15 mL of CH₂Cl₂:TFA (4:1) and the solution was stirred at rt for 45 min. Solvent was removed under reduced pressure and the residue chromatographed [CH₂Cl₂:MeOH—NH₃ (7N), 20:1 to 10:1] to give 0.37 g of a white solid. This was dissolved in water (45 mL) and solid Na₂CO₃ added until pH-12. This was extracted with CH₂Cl₂ (4×50 mL) and the combined organic layers were washed with water (50 mL), dried over Na₂SO₄, filtered and concentrated under reduced pressure to give 0.200 g (76%) of 5. ¹H NMR (CDCl₃) δ 8.33 (s, 1H), 7.31 (s, 1H), 6.89 (s, 1H), 5.99 (s, 2H), 5.52 (br s, 2H), 4.30 (t, J=6.3 Hz, 2H), 2.68 (t, J=7.0 Hz, 2H), 2.59 (t, J=6.3 Hz, 2H), 2.53 (t, J=7.1 Hz, 2H), 1.99 (m, 2H), 1.44 (s, 4H), 1.28 (s, 4H); ¹³C NMR (125 MHz, CDCl₃/MeOH-d₄) δ 154.5, 152.6, 151.5, 150.0, 149.6, 147.7, 125.9, 119.7, 119.6, 113.9, 102.8, 94.2, 49.7, 46.2, 41.61, 41.59, 32.9, 29.7, 29.5, 27.3, 26.9; HRMS (ESI) m/z [M+H]′ calcd. for C₂₁H₂₉IN₇O₂S, 570.1148. found 570.1124; HPLC: t_{R}=5.68 min.

### 6.2.5. PU-H71-Affi-Gel 10 beads (6)

4 (0.301 g, 0.45 mmol) was dissolved in 15 mL of CH₂Cl₂:TFA (4:1) and the solution was stirred at rt for 45 min. Solvent was removed under reduced pressure and the residue dried under high vacuum overnight. This was dissolved in DMF (12 mL) and added to 25 mL of Affi-Gel 10 beads (prewashed, 3×50 mL DMF) in a solid phase peptide synthesis vessel. 225 μL of N,N-diisopropylethylamine and several crystals of DMAP were added and this was shaken at rt for 2.5 h. Then 2-methoxyethylamine (0.085 g, 97 μl, 1.13 mmol) was added and shaking was continued for 30 minutes. Then the solvent was removed and the beads washed for 10 minutes each time with CH₂Cl₂:Et₃N (9:1, 4×50 mL), DMF (3×50 mL), Felts buffer (3×50 mL) and i-PrOH (3×50 mL). The beads 6 were stored in i-PrOH (beads: i-PrOH (1:2), v/v) at −80° C.

### 6.2.6. PU-H71-Biotin (7)

2 (4.2 mg, 0.0086 mmol) and EZ-Link® Amine-PEO₃-Biotin (5.4 mg, 0.0129 mmol) in DMF (0.2 mL) was stirred at rt for 24 h. The reaction mixture was concentrated and the residue chromatographed [CHCl₃:MeOH—NH₃ (7N), 5:1] to give 1.1 mg (16%) of 7. ¹H NMR (CDCl₃) δ 8.30 (s, 1H), 8.10 (s, 1H), 7.31 (s, 1H), 6.87 (s, 1H), 6.73 (br s, 1H), 6.36 (br s, 1H), 6.16 (br s, 2H), 6.00 (s, 2H), 4.52 (m, 1H), 4.28-4.37 (m, 3H), 3.58-3.77 (m, 10H), 3.55 (m, 2H), 3.43 (m, 2H), 3.16 (m, 1H), 2.92 (m, 1H), 2.80 (m, 2H), 2.72 (m, 1H), 2.66 (m, 2H), 2.17 (t, J=7.0 Hz, 2H), 2.04 (m, 2H), 1.35-1.80 (m, 6H); MS (ESI): m/z 872.2 [M+H]⁺

### 6.2.7. tert-Butyl 6-(4-(5-(2,4-bis(benzyloxy)-5-isopropylphenyl)-3-(ethylcarbamoyl)isoxazol-4-yl)benzylamino)hexylcarbamate (9)

AcOH (0.26 g, 0.25 mL, 4.35 mmol) was added to a mixture of 8 (Brough et al., 2008) (0.5 g, 0.87 mmol), 3 (0.56 g, 2.61 mmol), NaCNBH₃ (0.11 g, 1.74 mmol), CH₂Cl₂ (21 mL) and 3 Å molecular sieves (3 g). The reaction mixture was stirred for 1 h at rt. It was then concentrated under reduced pressure and chromatographed [CH₂Cl₂:MeOH—NH₃ (7N), 100:1 to 60:1] to give 0.50 g (75%) of 9. ¹H NMR (CDCl₃) δ 7.19-7.40 (m, 12H), 7.12-7.15 (m, 2H), 7.08 (s, 1H), 6.45 (s, 1H), 4.97 (s, 2H), 4.81 (s, 2H), 3.75 (s, 2H), 3.22 (m, 2H), 3.10 (m, 3H), 2.60 (t, J=7.1 Hz, 2H), 1.41-1.52 (m, 13H), 1.28-1.35 (m, 4H), 1.21 (t, J=7.2 Hz, 3H), 1.04 (d, J=6.9 Hz, 6H); MS (ESI): m/z 775.3 [M+H]⁺.

### 6.2.8. 4-(4-((6-Aminohexylamino)methyl)phenyl)-5-(2,4-dihydroxy-5-isopropylphenyl)-N-ethylisoxazole-3-carboxamide (10)

To a solution of 9 (0.5 g, 0.646 mmol) in CH₂Cl₂ (20 mL) was added a solution of BCl₃ (1.8 mL, 1.87 mmol, 1.0 M in CH₂Cl₂) and this was stirred at rt for 10 h. Saturated NaHCO₃ was added and CH₂Cl₂ was evaporated under reduced pressure. The water was carefully decanted and the remaining yellow precipitate was washed a few times with EtOAc and CH₂Cl₂ to give 0.248 g (78%) of 10. ¹H NMR (CDCl₃/MeOH-d₄) δ 7.32 (d, J=8.1 Hz, 2H), 7.24 (d, J=8.1 Hz, 2H), 6.94 (s, 1H), 6.25 (s, 1H), 3.74, (s, 2H), 3.41 (q, J=7.3 Hz, 2H), 3.08 (m, 1H), 2.65 (t, J=7.1 Hz, 2H), 2.60 (t, J=7.1 Hz, 2H), 1.40-1.56 (m, 4H), 1.28-1.35 (m, 4H), 1.21 (t, J=7.3 Hz, 3H), 1.01 (d, J=6.9 Hz, 6H); ¹³C NMR (125 MHz, CDCl₃/MeOH-d₄) δ 168.4, 161.6, 158.4, 157.6, 155.2, 139.0, 130.5, 129.5, 128.71, 128.69, 127.6, 116.0, 105.9, 103.6, 53.7, 49.2, 41.8, 35.0, 32.7, 29.8, 27.6, 27.2, 26.4, 22.8, 14.5; HRMS (ESI) m/z [M+H]⁺ calcd. for C₂₈H₃₉N₄O₄, 495.2971. found 495.2986; HPLC: t_{R}=6.57 min.

### 6.2.9. NVP-AUY922-Affi-Gel 10 Beads (11)

10 (46.4 mg, 0.094 mmol) was dissolved in DMF (2 mL) and added to 5 mL of Affi-Gel 10 beads (prewashed, 3×8 mL DMF) in a solid phase peptide synthesis vessel. 45 μl of N,N-diisopropylethylamine and several crystals of DMAP were added and this was shaken at rt for 2.5 h. Then 2-methoxyethylamine (17.7 mg, 21 μl, 0.235 mmol) was added and shaking was continued for 30 minutes. Then the solvent was removed and the beads washed for 10 minutes each time with CH₂Cl₂ (3×8 mL), DMF (3×8 mL), Felts buffer (3×8 mL) and i-PrOH (3×8 mL). The beads 11 were stored in i-PrOH (beads: i-PrOH, (1:2), v/v) at −80° C.

### 6.2.10. N′-(3,3-Dimethyl-5-oxocyclohexylidene)-4-methylbenzenesulfonohydrazide (14)

### (Hiegel & Burk, 1973)

10.00 g (71.4 mmol) of dimedone (13), 13.8 g (74.2 mmol) of tosyl hydrazide (12) and p-toluene sulfonic acid (0.140 g, 0.736 mmol) were suspended in toluene (600 mL) and this was refluxed with stirring for 1.5 h. While still hot, the reaction mixture was filtered and the solid was washed with toluene (4×100 mL), ice-cold ethyl acetate (2×200 mL) and hexane (2×200 mL) and dried to give 19.58 g (89%) of 14 as a solid. TLC (100% EtOAc) R_{f}=0.23; ¹H NMR (DMSO-d₆) δ 9.76 (s, 1H), 8.65 (br s, 1H), 7.69 (d, J=8.2 Hz, 2H), 7.41 (d, J=8.1 Hz, 2H), 5.05 (s, 1H), 2.39 (s, 3H), 2.07 (s, 2H), 1.92 (s, 2H), 0.90 (s, 6H); MS (ESI): m/z 309.0 [M+H]

### 6.2.11. 6,6-Dimethyl-3-(trifluoromethyl)-6,7-dihydro-1H-indazol-4(5H)-one (15)

To 5.0 g (16.2 mmol) of 14 in THF (90 mL) and Et₃N (30 mL) was added trifluoroacetic anhydride (3.4 g, 2.25 mL, 16.2 mmol) in one portion. The resulting red solution was heated at 55° C. for 3 h. After cooling to rt, methanol (8 mL) and 1M NaOH (8 mL) were added and the solution was stirred for 3 h at rt. The reaction mixture was diluted with 25 mL of saturated NH₄Cl, poured into a seperatory funnel and extracted with EtOAc (3×50 mL). The combined organic layers were washed with brine (3×50 mL), dried over Na₂SO₄ and concentrated under reduced pressure to give a red oily residue which was chromatographed (hexane:EtOAc, 80:20 to 60:40) to give 2.08 g (55%) of 15 as an orange solid. TLC (hexane:EtOAc, 6:4) R_{f}=0.37; ¹H NMR (CDCl₃) δ 2.80 (s, 2H), 2.46 (s, 2H), 1.16 (s, 6H); MS (ESI): m/z 231.0 [M−H]⁻.

### 6.2.12. 2-Bromo-4-(6,6-dimethyl-4-oxo-3-(trifluoromethyl)-4,5,6,7-tetrahydro-1H-indazol-1-yl)benzonitrile (16)

To a mixture of 15 (0.100 g, 0.43 mmol) and NaH (15.5 mg, 0.65 mmol) in DMF (8 mL) was added 2-bromo-4-fluorobenzonitrile (86 mg, 0.43 mmol) and heated at 90° C. for 5 h. The reaction mixture was concentrated under reduced pressure and the residue chromatographed (hexane:EtOAc, 10:1 to 10:2) to give 0.162 g (91%) of 16 as a white solid. ¹H NMR (CDCl₃) δ 7.97 (d, J=2.1 Hz, 1H), 7.85 (d, J=8.4 Hz, 1H), 7.63 (dd, J=8.4, 2.1 Hz, 1H), 2.89 (s, 2H), 2.51 (s, 2H), 1.16 (s, 6H); MS (ESI): m/z 410.0/412.0 [M−H]⁻.

### 6.2.13. 2-(trans-4-Aminocyclohexylamino)-4-(6,6-dimethyl-4-oxo-3-(trifluoromethyl)-4,5,6,7-tetrahydro-1H-indazol-1-yl)benzonitrile (17)

A mixture of 16 (0.200 g, 0.485 mmol), NaOtBu (93.3 mg, 0.9704 mmol), Pd₂(dba)₃ (88.8 mg, 0.097 mmol) and DavePhos (38 mg, 0.097 mmol) in 1,2-dimethoxyethane (15 mL) was degassed and flushed with argon several times. trans-1,4-Diaminocyclohexane (0.166 g, 1.456 mmol) was added and the flask was again degassed and flushed with argon before heating the reaction mixture at 50° C. overnight. The reaction mixture was concentrated under reduced pressure and the residue purified by preparatory TLC (CH₂Cl₂:MeOH—NH₃ (7N), 10:1) to give 52.5 mg (24%) of 17. Additionally, 38.5 mg (17%) of amide 18 was isolated for a total yield of 41%. ¹H NMR (CDCl₃) δ 7.51 (d, J=8.3 Hz, 1H), 6.81 (d, J=1.8 Hz, 1H), 6.70 (dd, J=8.3, 1.8 Hz, 1H), 4.64 (d, J=7.6 Hz, 1H), 3.38 (m, 1H), 2.84 (s, 2H), 2.81 (m, 1H), 2.49 (s, 2H), 2.15 (d, J=11.2 Hz, 2H), 1.99 (d, J=11.0 Hz, 2H), 1.25-1.37 (m, 4H), 1.14 (s, 6H); MS (ESI): m/z 446.3 [M+H]⁺.

### 6.2.14. 2-(trans-4-Aminocyclohexylamino)-4-(6,6-dimethyl-4-oxo-3-(trifluoromethyl)-4,5,6,7-tetrahydro-1H-indazol-1-yl)benzamide (18)

A solution of 17 (80 mg, 0.18 mmol) in DMSO (147 μl), EtOH (590 μl), 5N NaOH (75 μl) and H₂O₂ (88 μl) was stirred at rt for 3 h. The reaction mixture was concentrated under reduced pressure and the residue purified by preparatory TLC [CH₂Cl₂:MeOH—NH₃ (7N), 10:1] to give 64.3 mg (78%) of 18. ¹H NMR (CDCl₃) δ 8.06 (d, J=7.5 Hz, 1H), 7.49 (d, J=8.4 Hz, 1H), 6.74 (d, J=1.9 Hz, 1H), 6.62 (dd, J=8.4, 2.0 Hz, 1H), 5.60 (br s, 2H), 3.29 (m, 1H), 2.85 (s, 2H), 2.77 (m, 1H), 2.49 (s, 2H), 2.13 (d, J=11.9 Hz, 2H), 1.95 (d, J=11.8 Hz, 2H), 1.20-1.42 (m, 4H), 1.14 (s, 6H); MS (ESI): m/z 464.4 [M+H]⁺; HPLC: t_{R}=7.05 min.

### 6.2.15. tert-Butyl 6-(trans-4-(2-carbamoyl-5-(6,6-dimethyl-4-oxo-3-(trifluoromethyl)-4,5,6,7-tetrahydro-1H-indazol-1-yl)phenylamino)cyclohexylamino)-6-oxohexylcarbamate (19)

To a mixture of 18 (30 mg, 0.0647 mmol) in CH₂Cl₂ (1 ml) was added 6-(Boc-amino)caproic acid (29.9 mg, 0.1294 mmol), EDCI (24.8 mg, 0.1294 mmol) and DMAP (0.8 mg, 0.00647 mmol). The reaction mixture was stirred at rt for 2 h then concentrated under reduced pressure and the residue purified by preparatory TLC [hexane:CH₂Cl₂:EtOAc:MeOH—NH₃ (7N), 2:2:1:0.5] to give 40 mg (91%) of 19. ¹H NMR (CDCl₃/MeOH-d₄) δ 7.63 (d, J=8.4 Hz, 1H), 6.75 (d, J=1.7 Hz, 1H), 6.61 (dd, J=8.4, 2.0 Hz, 1H), 3.75 (m, 1H), 3.31 (m, 1H), 3.06 (t, J=7.0 Hz, 2H), 2.88 (s, 2H), 2.50 (s, 2H), 2.15 (m, 4H), 2.03 (d, J=11.5 Hz, 2H), 1.62 (m, 2H), 1.25-1.50 (m, 17H), 1.14 (s, 6H); ¹³C NMR (125 MHz, CDCl₃/MeOH-d₄) δ 191.5, 174.1, 172.3, 157.2, 151.5, 150.3, 141.5, 140.6 (q, J=39.4 Hz), 130.8, 120.7 (q, J=268.0 Hz), 116.2, 114.2, 109.5, 107.3, 79.5, 52.5, 50.7, 48.0, 40.4, 37.3, 36.4, 36.0, 31.6, 31.3, 29.6, 28.5, 28.3, 25.7, 25.4; HRMS (ESI) m/z [M+Na]⁺ calcd. for C₃₄H₄₇F₃N₆O₅Na, 699.3458. found 699.3472; HPLC: t_{R}=9.10 min.

### 6.2.16. 2-(trans-4-(6-Aminohexanamido)cyclohexylamino)-4-(6,6-dimethyl-4-oxo-3-(trifluoromethyl)-4,5,6,7-tetrahydro-1H-indazol-1-yl)benzamide (20)

19 (33 mg, 0.049 mmol) was dissolved in 1 mL of CH₂Cl₂:TFA (4:1) and the solution was stirred at rt for 45 min. Solvent was removed under reduced pressure and the residue purified by preparatory TLC [CH₂Cl₂:MeOH—NH₃ (7N), 6:1] to give 24 mg (86%) of 20. ¹H NMR (CDCl₃/MeOH-d₄) δ 7.69 (d, J=8.4 Hz, 1H), 6.78 (d, J=1.9 Hz, 1H), 6.64 (dd, J=8.4, 1.9 Hz, 1H), 3.74 (m, 1H), 3.36 (m, 1H), 2.92 (t, J=7.5 Hz, 2H), 2.91 (s, 2H), 2.51 (s, 2H), 2.23 (t, J=7.3 Hz, 2H), 2.18 (d, J=10.2 Hz, 2H), 2.00 (d, J=9.1 Hz, 2H), 1.61-1.75 (m, 4H), 1.34-1.50 (m, 6H), 1.15 (s, 6H); ¹³C NMR (125 MHz, MeOH-d₄) δ 191.2, 173.6, 172.2, 151.8, 149.7, 141.2, 139.6 (q, J=39.5 Hz), 130.3, 120.5 (q, J=267.5 Hz), 115.5, 114.1, 109.0, 106.8, 51.6, 50.0, 47.8, 39.0, 36.3, 35.2, 35.1, 31.0, 30.5, 26.8, 26.7, 25.4, 24.8; HRMS (ESI) m/z [M+H]¹ calcd. for C₂₉H₄₀F₃N₆O₃, 577.3114. found 577.3126; HPLC: t_{R}=7.23 min.

### 6.2.17. SNX-2112-Affi-Gel 10 Beads (21)

19 (67 mg, 0.0992 mmol) was dissolved in 3.5 mL of CH₂Cl₂:TFA (4:1) and the solution was stirred at rt for 20 min. Solvent was removed under reduced pressure and the residue dried under high vacuum for two hours. This was dissolved in DMF (2 mL) and added to 5 mL of Affi-Gel 10 beads (prewashed, 3×8 mL DMF) in a solid phase peptide synthesis vessel. 45 μl of N,N-diisopropylethylamine and several crystals of DMAP were added and this was shaken at rt for 2.5 h. Then 2-methoxyethylamine (18.6 mg, 22 μl, 0.248 mmol) was added and shaking was continued for 30 minutes. Then the solvent was removed and the beads washed for 10 minutes each time with CH₂Cl₂ (3×8 mL), DMF (3×8 mL) and i-PrOH (3×8 mL). The beads 21 were stored in i-PrOH (beads: i-PrOH, (1:2), v/v) at −80° C.

### 6.2.18. N-Fmoc-trans-4-aminocyclohexanol (22) (Crestey et al., 2008)

To a solution of trans-4-aminocyclohexanol hydrochloride (2.0 g, 13.2 mmol) in dioxane:water (26:6.5 mL) was added Et₃N (1.08 g, 1.49 mL, 10.7 mmol) and this was stirred for 10 min. Then Fmoc-OSu (3.00 g, 8.91 mmol) was added over five minutes and the resulting suspension was stirred at rt for 2 h. The reaction mixture was concentrated to ˜5 mL, then some CH₂Cl₂ was added. This was filtered and the solid was washed with H₂O (4×40 mL) then dried to give 2.85 g (95%) of 22 as a white solid. Additional 0.100 g (3%) of 22 was obtained by extracting the filtrate with CH₂Cl₂ (2×100 mL), drying over Na₂SO₄, filtering and removing solvent for a combined yield of 98%. TLC (hexane:EtOAc, 20:80) R_{f}=0.42; ¹H NMR (CDCl₃) δ 7.77 (d, J=7.5 Hz, 2H), 7.58 (d, J=7.4 Hz, 2H), 7.40 (t, J=7.4 Hz, 2H), 7.31 (t, J=7.4 Hz, 2H), 4.54 (br s, 1H), 4.40 (d, J=5.6 Hz, 2H), 4.21 (t, J=5.6 Hz, 1H), 3.61 (m, 1H), 3.48 (m, 1H), 1.9-2.1 (m, 4H), 1.32-1.48 (m, 2H), 1.15-1.29 (m, 2H); MS (ESI): m/z 338.0 [M+H]⁺.

### 6.2.19. N-Fmoc-trans-4-aminocyclohexanol tetrahydropyranyl ether (23)

1.03 g (3.05 mmol) of 22 and 0.998 g (1.08 mL, 11.86 mmol) of 3,4-dihydro-2H-pyran (DHP) was suspended in dioxane (10 mL). Pyridinium p-toluenesulfonate (0.153 g, 0.61 mmol) was added and the suspension stirred at rt. After 1 hr additional DHP (1.08 mL, 11.86 mmol) and dioxane (10 mL) were added and stirring continued. After 9 h additional DHP (1.08 mL, 11.86 mmol) was added and stirring continued overnight. The resulting solution was concentrated and the residue chromatographed (hexane:EtOAc, 75:25 to 65:35) to give 1.28 g (100%) of 23 as a white solid. TLC (hexane:EtOAc, 70:30) R_{f}=0.26; ¹H NMR (CDCl₃) δ 7.77 (d, J=7.5 Hz, 2H), 7.58 (d, J=7.5 Hz, 2H), 7.40 (t, J=7.4 Hz, 2H), 7.31 (dt, J=7.5, 1.1 Hz, 2H), 4.70 (m, 1H), 4.56 (m, 1H), 4.40 (d, J=6.0 Hz, 2H), 4.21 (t, J=6.1 Hz, 1H), 3.90 (m, 1H), 3.58 (m, 1H), 3.45-3.53 (m, 2H), 1.10-2.09 (m, 14H); MS (ESI): m/z 422.3 [M+H]⁺.

### 6.2.20. trans-4-Aminocylohexanol tetrahydropyranyl ether (24)

1.28 g (3.0 mmol) of 23 was dissolved in CH₂Cl₂ (20 mL) and piperidine (2 mL) was added and the solution stirred at rt for 5 h. Solvent was removed and the residue was purified by chromatography [CH₂Cl₂:MeOH—NH₃ (7N), 80:1 to 30:1] to give 0.574 g (96%) of 24 as an oily residue which slowly crystallized. ¹H NMR (CDCl₃) δ 4.70 (m, 1H), 3.91 (m, 1H), 3.58 (m, 1H), 3.49 (m, 1H), 2.69 (m, 1H), 1.07-2.05 (m, 14H); MS (ESI): m/z 200.2 [M+H]⁺.

### 6.2.21. 4-(6,6-Dimethyl-4-oxo-3-(trifluoromethyl)-4,5,6,7-tetrahydro-1H-indazol-1-yl)-2-(trans-4-(tetrahydro-2H-pyran-2-yloxy)cyclohexylamino)benzonitrile (25)

A mixture of 16 (0.270 g, 0.655 mmol), NaOtBu (0.126 g, 1.31 mmol), Pd₂(dba)₃ (0.120 g, 0.131 mmol) and DavePhos (0.051 g, 0.131 mmol) in 1,2-dimethoxyethane (20 mL) was degassed and flushed with argon several times. 24 (0.390 g, 1.97 mmol) was added and the flask was again degassed and flushed with argon before heating the reaction mixture at 60° C. for 3.5 h. The reaction mixture was concentrated under reduced pressure and the residue purified by preparatory TLC [hexane:CH₂Cl₂:EtOAc:MeOH—NH₃ (7N), 7:6:3:1.5] to give 97.9 mg (28%) of 25. Additionally, 60.5 mg (17%) of amide 26 was isolated for a total yield of 45%. ¹H NMR (CDCl₃) δ 7.52 (d, J=8.3 Hz, 1H), 6.80 (d, J=1.7 Hz, 1H), 6.72 (dd, J=8.3, 1.8 Hz, 1H), 4.72 (m, 1H), 4.67 (d, J=7.6 Hz, 1H), 3.91 (m, 1H), 3.68 (m, 1H), 3.50 (m, 1H), 3.40 (m, 1H), 2.84 (s, 2H), 2.49 (s, 2H), 2.06-2.21 (m, 4H), 1.30-1.90 (m, 10H), 1.14 (s, 6H); MS (ESI): m/z 529.4 [M−H]⁻.

### 6.2.22. 4-(6,6-Dimethyl-4-oxo-3-(trifluoromethyl)-4,5,6,7-tetrahydro-1H-indazol-1-yl)-2-(trans-4-(tetrahydro-2H-pyran-2-yloxy)cyclohexylamino)benzamide (26)

A solution of 25 (120 mg, 0.2264 mmol) in DMSO (220 μl), EtOH (885 μl), 5N NaOH (112 μl) and H₂O₂ (132 μl) was stirred at rt for 4 h. Then 30 mL of brine was added and this was extracted with EtOAc (5×15 mL), dried over Na₂SO₄, filtered and concentrated under reduced pressure. The residue was purified by preparatory TLC [hexane:CH₂Cl₂:EtOAc:MeOH—NH₃ (7N), 7:6:3:1.5] to give 102 mg (82%) of 26. ¹H NMR (CDCl₃) δ 8.13 (d, J=7.4 Hz, 1H), 7.50 (d, J=8.4 Hz, 1H), 6.74 (d, J=1.9 Hz, 1H), 6.63 (dd, J=8.4, 2.0 Hz, 1H), 5.68 (br s, 2H), 4.72 (m, 1H), 3.91 (m, 1H), 3.70 (m, 1H), 3.50 (m, 1H), 3.34 (m, 1H), 2.85 (s, 2H), 2.49 (s, 2H), 2.05-2.19 (m, 4H), 1.33-1.88 (m, 10H), 1.14 (s, 6H); MS (ESI): m/z 547.4 [M−H]⁻.

### 6.2.23. 4-(6,6-Dimethyl-4-oxo-3-(trifluoromethyl)-4,5,6,7-tetrahydro-1H-indazol-1-yl)-2-(trans-4-hydroxycyclohexylamino)benzamide (SNX-2112)

26 (140 mg, 0.255 mmol) and pyridinium p-toluenesulfonate (6.4 mg, 0.0255 mmol) in EtOH (4.5 mL) was heated at 65° C. for 17 h. The reaction mixture was concentrated under reduced pressure and the residue purified by preparatory TLC [hexane:CH₂Cl₂:EtOAc:MeOH—NH₃ (7N), 2:2:1:0.5] to give 101 mg (85%) of SNX-2112. ¹H NMR (CDCl₃) δ 8.10 (d, J=7.4 Hz, 1H), 7.52 (d, J=8.4 Hz, 1H), 6.75 (d, J=1.3 Hz, 1H), 6.60 (dd, J=8.4, 1.6 Hz, 1H), 5.97 (br s, 2H), 3.73 (m, 1H), 3.35 (m, 1H), 2.85 (s, 2H), 2.48 (s, 2H), 2.14 (d, J=11.8 Hz, 2H), 2.04 (d, J=11.1 Hz, 2H), 1.33-1.52 (m, 4H), 1.13 (s, 6H); ¹³C NMR (125 MHz, CDCl₃/MeOH-d₄) δ 191.0, 171.9, 151.0, 150.0, 141.3, 140.3 (q, J=39.6 Hz), 130.4, 120.3 (q, J=270.2 Hz), 115.9, 113.7, 109.2, 107.1, 69.1, 52.1, 50.2, 40.1, 37.0, 35.6, 33.1, 30.2, 28.0; MS (ESI): m/z 463.3 [M−H]⁻, 465.3 [M+H]⁺; HPLC: t_{R}=7.97 min.

### 6.2.24. Preparation of Control Beads

DMF (8.5 mL) was added to 20 mL of Affi-Gel 10 beads (prewashed, 3×40 mL DMF) in a solid phase peptide synthesis vessel. 2-Methoxyethylamine (113 mg, 129 μL, 1.5 mmol) and several crystals of DMAP were added and this was shaken at rt for 2.5 h. Then the solvent was removed and the beads washed for 10 minutes each time with CH₂Cl₂ (4×35 mL), DMF (3×35 mL), Felts buffer (2×35 mL) and i-PrOH (4×35 mL). The beads were stored in i-PrOH (beads: i-PrOH (1:2), v/v) at −80° C.

### 6.3. Competition Assay

For the competition studies, fluorescence polarization (FP) assays were performed as previously reported (Du et al., 2007). Briefly, FP measurements were performed on an Analyst GT instrument (Molecular Devices, Sunnyvale, Calif.). Measurements were taken in black 96-well microtiter plates (Corning #3650) where both the excitation and the emission occurred from the top of the wells. A stock of 10 μM GM-cy3B was prepared in DMSO and diluted with Felts buffer (20 mM Hepes (K), pH 7.3, 50 mM KCl, 2 mM DTT, 5 mM MgCl₂, 20 mM Na₂MoO₄, and 0.01% NP40 with 0.1 mg/mL BGG). To each 96-well were added 6 nM fluorescent GM (GM-cy3B), 3 μg SKBr3 lysate (total protein), and tested inhibitor (initial stock in DMSO) in a final volume of 100 μL HFB buffer. Drugs were added in triplicate wells. For each assay, background wells (buffer only), tracer controls (free, fluorescent GM only) and bound GM controls (fluorescent GM in the presence of SKBr3 lysate) were included on each assay plate. GM was used as positive control. The assay plate was incubated on a shaker at 4° C. for 24 h and the FP values in mP were measured. The fraction of tracer bound to Hsp90 was correlated to the mP value and plotted against values of competitor concentrations. The inhibitor concentration at which 50% of bound GM was displaced was obtained by fitting the data. All experimental data were analyzed using SOFTmax Pro 4.3.1 and plotted using Prism 4.0 (Graphpad Software Inc., San Diego, Calif.).

### 6.4. Chemical Precipitation, Western Blotting and Flow Cytometry

The leukemia cell lines K562 and MV4-11 and the breast cancer cell line MDA-MB-468 were obtained from the American Type Culture Collection. Cells were cultured in RPMI (K562), in Iscove's modified Dulbecco's media (MV4-11) or in DME/F12 (MDA-MB-468) supplemented with 10% FBS, 1% L-glutamine, 1% penicillin and streptomycin, and maintained in a humidified atmosphere of 5% CO₂ at 37° C. Cells were lysed by collecting them in Felts buffer (HEPES 20 mM, KCl 50 mM, MgCl₂ 5 mM, NP40 0.01%, freshly prepared Na₂MoO₄ 20 mM, pH 7.2-7.3) with added 10 μg/μL of protease inhibitors (leupeptin and aprotinin), followed by three successive freeze (in dry ice) and thaw steps. Total protein concentration was determined using the BCA kit (Pierce) according to the manufacturer's instructions.

Hsp90 inhibitor beads or control beads containing an Hsp90 inactive chemical (2-methoxyethylamine) conjugated to agarose beads were washed three times in lysis buffer. The bead conjugates (80 μL or as indicated) were then incubated overnight at 4° C. with cell lysates (250 μg), and the volume was adjusted to 200-300 μL with lysis buffer. Following incubation, bead conjugates were washed 5 times with the lysis buffer and analyzed by Western blot, as indicated below.

For treatment with PU-H71, cells were grown to 60-70% confluence and treated with inhibitor (5 μM) for 24 h. Protein lysates were prepared in 50 mM Tris pH 7.4, 150 mM NaCl and 1% NP-40 lysis buffer.

For Western blotting, protein lysates (10-50 μg) were electrophoretically resolved by SDS/PAGE, transferred to nitrocellulose membrane and probed with a primary antibody against Hsp90 (1:2000, SMC-107A/B, StressMarq), anti-IGF-IR (1:1000, 3027, Cell Signaling) and anti-c-Kit (1:200, 612318, BD Transduction Laboratories). The membranes were then incubated with a 1:3000 dilution of a corresponding horseradish peroxidase conjugated secondary antibody. Detection was performed using the ECL-Enhanced Chemiluminescence Detection System (Amersham Biosciences) according to manufacturer's instructions.

To detect the binding of PU-H71 to cell surface Hsp90, MV4-11 cells at 500,000 cells/ml were incubated with the indicated concentrations of PU-H71-biotin or D-biotin as control for 2 h at 37° C. followed by staining of phycoerythrin (PE) conjugated streptavidin (SA) (BD Biosciences) in FACS buffer (PBS+0.5% FBS) at 4° C. for 30 min. Cells were then analyzed using the BD-LSRII flow cytometer. Mean fluorescence intensity (MFI) was used to calculate the binding of PU-H71-biotin to cells and values were normalized to the MFI of untreated cells stained with SA-PE.

### 6.5. Docking

Molecular docking computations were carried out on a HP workstation xw8200 with the Ubuntu 8.10 operating system using Glide 5.0 (Schrodinger). The coordinates for the Hsp90α complexes with bound inhibitor PU-H71 (PDB ID: 2FWZ), NVP-AUY922 (PDB ID: 2VCI) and 27 (PDB ID: 3D0B) were downloaded from the RCSB Protein Data Bank. For docking experiments, compounds PU-H71, NVP-AUY922, 5, 10, 20 and 27 were constructed using the fragment dictionary of Maestro 8.5 and geometry-optimized using the Optimized Potentials for Liquid Simulations-All Atom (OPLS-AA) force field (Jorgensen et al., 1996) with the steepest descent followed by truncated Newton conjugate gradient protocol as implemented in Macromodel 9.6, and were further subjected to ligand preparation using default parameters of LigPrep 2.2 utility provided by Schrödinger LLC. Each protein was optimized for subsequent grid generation and docking using the Protein Preparation Wizard provided by Schrödinger LLC. Using this tool, hydrogen atoms were added to the proteins, bond orders were assigned, water molecules of crystallization not deemed to be important for ligand binding were removed, and the entire protein was minimized. Partial atomic charges for the protein were assigned according to the OPLS-2005 force field. Next, grids were prepared using the Receptor Grid Generation tool in Glide. With the respective bound inhibitor in place, the centroid of the workspace ligand was chosen to define the grid box. The option to dock ligands similar in size to the workspace ligand was selected for determining grid sizing.

Next, the extra precision (XP) Glide docking method was used to flexibly dock compounds PU-H71 and 5 (to 2FWZ), NVP-AUY922 and 10 (to 2VCI), and 20 and 27 (to 3D0B) into their respective binding site. Although details on the methodology used by Glide are described elsewhere (Patel et al., 2008; Friesner et al., 2004; Halgren et al., 2004), a short description about parameters used is provided below. The default setting of scale factor for van der Waals radii was applied to those atoms with absolute partial charges less than or equal to 0.15 (scale factor of 0.8) and 0.25 (scale factor of 1.0) electrons for ligand and protein, respectively. No constraints were defined for the docking runs. Upon completion of each docking calculation, at most 100 poses per ligand were allowed to generate. The top-scored docking pose based on the Glide scoring function (Eldridge et al., 1997) was used for our analysis. In order to validate the XP Glide docking procedure the crystallographic bound inhibitor (PU-H71 or NVP-AUY922 or 27) was extracted from the binding site and re-docked into its respective binding site. There was excellent agreement between the localization of the inhibitor upon docking and the crystal structure as evident from the 0.098 Å (2FWZ), 0.313 Å (2VCI) and 0.149 Å (3D0B) root mean square deviations. Thus, the present study suggests the high docking reliability of Glide in reproducing the experimentally observed binding mode for Hsp90 inhibitors and the parameter set for the Glide docking reasonably reproduces the X-ray structure.

TABLE 8
Binding affinity for Hsp90 from SKBr3 cellular extracts.

| Compound | IC₅₀ (nM) |
|---|---|
| GM | 15.4 |
| PU-H71 | 22.4 |
| 5 | 19.8 |
| 7 | 67.1 |
| NVP-AUY922 | 4.1 |
| 10 | 7.0 |
| SNX-2112 | 15.1 |
| 18 | 210.1 |
| 20 | 24.7 |

### REFERENCES

1. Ackler, S., Xiao, Y., Mitten, M. J., Foster, K., Oleksijew, A., Refici, M., Schlessinger, S., Wang, B., Chemburkar, S. R., Bauch, J., et al. (2008). ABT-263 and rapamycin act cooperatively to kill lymphoma cells in vitro and in vivo. Mol Cancer Ther 7, 3265-3274.
2. Adler, A. S., Lin, M., Horlings, H., Nuyten, D. S., van de Vijver, M. J., and Chang, H. Y. (2006). Genetic regulators of large-scale transcriptional signatures in cancer. Nat Genet 38, 421-430.
3. Alizadeh, A. A., Eisen, M. B., Davis, R. E., Ma, C., Lossos, I. S., Rosenwald, A., Boldrick, J. C., Sabet, H., Tran, T., Yu, X., et al. (2000). Distinct types of diffuse large B-cell lymphoma identified by gene expression profiling. Nature 403, 503-511.
4. An, W. G., Schulte, T. W. & Neckers, L. M. (2000). The heat shock protein 90 antagonist geldanamycin alters chaperone association with p210bcr-abl and v-src proteins before their degradation by the proteasome. Cell Growth Differ. 11, 355-360.
5. Andersen, J. N. et al. (2010). Pathway-Based Identification of Biomarkers for Targeted Therapeutics: Personalized Oncology with PI3K Pathway Inhibitors. Sci. Transl. Med. 2, 43ra55.
6. Apsel, B., et al. (2008). Targeted polypharmacology: discovery of dual inhibitors of tyrosine and phosphoinositide kinases. Nature Chem. Biol. 4, 691-699.
7. Ashman, K. & Villar, E. L. (2009). Phosphoproteomics and cancer research. Clin. Transl. Oncol. 11, 356-362.
8. Barril, X.; Beswick, M. C.; Collier, A.; Drysdale, M. J.; Dymock, B. W.; Fink, A.; Grant, K.; Howes, R.; Jordan, A. M.; Massey, A.; Surgenor, A.; Wayne, J.; Workman, P.; Wright, L., Bioorg. Med. Chem. Lett. 2006, 16, 2543-2548.
9. Barta, T. E.; Veal, J. M.; Rice, J. W.; Partridge, J. M.; Fadden, R. P.; Ma, W.; Jenks, M.; Geng, L.; Hanson, G. J.; Huang, K. H.; Barabasz, A. F.; Foley, B. E.; Otto, J.; Hall, S. E., Bioorg. Med. Chem. Lett. 2008, 18, 3517-3521.
10. Bedford, M. T. & Clarke, S. G. (2009). Protein arginine methylation in mammals: who, what, and why. Mol. Cell 33, 1-13.
11. Bonvini, P., Gastaldi, T., Falini, B., and Rosolen, A. (2002). Nucleophosmin-anaplastic lymphoma kinase (NPM-ALK), a novel Hsp90-client tyrosine kinase: down-regulation of NPMALK expression and tyrosine phosphorylation in ALK(+) CD30(+) lymphoma cells by the Hsp90 antagonist 17-allylamino,17-demethoxygeldanamycin. Cancer Res 62, 1559-1566.
12. Brehme, M. et al. (2009). Charting the molecular network of the drug target Bcr-Abl. Proc. Natl. Acad. Sci. USA 106, 7414-7419.
13. Brough, P. A., Aherne, W., Barril, X., Borgognoni, J., Boxall, K., Cansfield, J. E., Cheung, K.-M. J., Collins, I., Davies, N. G. M., Drysdale, M. J., Dymock, B., Eccles, S. A., Finch, H., Fink, A., Hayes, A., Howes, R., Hubbard, R. E., James, K., Jordan, A. M., Lockie, A., Martins, V., Massey, A., Matthews, T. P., McDonald, E., Northfield, C. J., Pearl, L. H., Prodromou, C., Ray, S., Raynaud, F. I., Roughley, S. D., Sharp, S. Y., Surgenor, A., Walmsley, D. L., Webb, P., Wood, M., Workman, P., and Wright, L. (2008). J. Med. Chem. 51, 196-218.
14. Burke, B, A. & Carroll, M. (2010). BCR-ABL: a multi-faceted promoter of DNA mutation in chronic myelogeneous leukemia. Leukemia 24, 1105-1112.
15. Caldas-Lopes, E., Cerchietti, L., Ahn, J. H., Clement, C. C., Robles, A. I., Rodina, A., Moulick, K., Taldone, T., Gozman, A., Guo, Y., et al. (2009). Hsp90 inhibitor PU-H71, a multimodal inhibitor of malignancy, induces complete responses in triple-negative breast cancer models. Proc Natl Acad Sci USA 106, 8368-8373.
16. Carayol, N. et al. (2010). Critical roles for mTORC2- and rapamycin-insensitive mTORC1-complexes in growth and survival of BCR-ABL-expressing leukemic cells. Proc. Natl. Acad. Sci. USA. 107, 12469-12474.
17. Carden, C. P., Sarker, D., Postel-Vinay, S., Yap, T. A., Attard, G., Banerji, U., Garrett, M. D., Thomas, G. V., Workman, P., Kaye, S. B., and de Bono, J. S. (2010). Drug Discov. Today 15, 88-97.
18. Cerchietti, L. C., Ghetu, A. F., Zhu, X., Da Silva, G. F., Zhong, S., Matthews, M., Bunting, K. L., Polo, J. M., Fares, C., Arrowsmith, C. H., et al. (2010a). A small-molecule inhibitor of BCL6 kills DLBCL cells in vitro and in vivo. Cancer Cell 17, 400-411.
19. Cerchietti, L. C., Hatzi, K., Caldas-Lopes, E., Yang, S. N., Figueroa, M. E., Morin, R. D., Hirst, M., Mendez, L., Shaknovich, R., Cole, P. A., et al. (2010b). BCL6 repression of EP300 in human diffuse large B cell lymphoma cells provides a basis for rational combinatorial therapy. J Clin Invest.
20. Cerchietti, L. C., Lopes, E. C., Yang, S. N., Hatzi, K., Bunting, K. L., Tsikitas, L. A., Mallik, A., Robles, A. I., Walling, J., Varticovski, L., et al. (2009a). A purine scaffold Hsp90 inhibitor destabilizes BCL-6 and has specific antitumor activity in BCL-6-dependent B cell lymphomas. Nat Med 15, 1369-1376.
21. Cerchietti, L. C., Yang, S. N., Shaknovich, R., Hatzi, K., Polo, J. M., Chadburn, A., Dowdy, S. F., and Melnick, A. (2009b). A peptomimetic inhibitor of BCL6 with potent antilymphoma effects in vitro and in vivo. Blood 113, 3397-3405.
22. Chamovitz, D. A., Wei, N., Osterlund, M. T., von Arnim, A. G., Staub, J. M., Matsui, M., and Deng, X. W. (1996). The COP9 complex, a novel multisubunit nuclear regulator involved in light control of a plant developmental switch. Cell 86, 115-121.
23. Chan, V. W., Meng, F., Soriano, P., DeFranco, A. L., and Lowell, C. A. (1997). Characterization of the B lymphocyte populations in Lyn-deficient mice and the role of Lyn in signal initiation and down-regulation. Immunity 7, 69-81.
24. Chandarlapaty, S., Sawai, A., Ye, Q., Scott, A., Silinski, M., Huang, K., Fadden, P., Partdrige, J., Hall, S., Steed, P., Norton, L., Rosen, N., and Solit, D. B. (2008). Clin. Cancer Res. 14, 240-248.
25. Chen, L., Juszczynski, P., Takeyama, K., Aguiar, R. C., and Shipp, M. A. (2006). Protein tyrosine phosphatase receptor-type 0 truncated (PTPROt) regulates SYK phosphorylation, proximal Bcell-receptor signaling, and cellular proliferation. Blood 108, 3428-3433.
26. Chen, L., Monti, S., Juszczynski, P., Daley, J., Chen, W., Witzig, T. E., Habermann, T. M., Kutok, J. L., and Shipp, M. A. (2008). SYK-dependent tonic B-cell receptor signaling is a rational treatment target in diffuse large B-cell lymphoma. Blood 111, 2230-2237.
27. Cheung, K. M.; Matthews, T. P.; James, K.; Rowlands, M. G.; Boxall, K. J.; Sharp, S. Y.; Maloney, A.; Roe, S. M.; Prodromou, C.; Pearl, L. H.; Aherne, G. W.; McDonald, E.; Workman, P., Bioorg. Med. Chem. Lett. 2005, 15, 3338-3343.
28. Chiba, T., and Tanaka, K. (2004). Cullin-based ubiquitin ligase and its control by NEDD8-conjugating system. Curr Protein Pept Sci 5, 177-184.
29. Chiosis, G., and Neckers, L. (2006). Tumor selectivity of Hsp90 inhibitors: the explanation remains elusive. ACS Chem Biol 1, 279-284.
30. Chiosis, G., Timaul, M. N., Lucas, B., Munster, P. N., Zheng, F. F., Sepp-Lorenzino, L., and Rosen, N. (2001). A small molecule designed to bind to the adenine nucleotide pocket of Hsp90 causes Her2 degradation and the growth arrest and differentiation of breast cancer cells. Chem Biol 8, 289-299.
31. Chou, T. C. (2006). Theoretical basis, experimental design, and computerized simulation of synergism and antagonism in drug combination studies. Pharmacol. Rev. 58, 621-681.
32. Chou, T. C. & Talalay, P. (1984). Quantitative analysis of dose-effect relationships: the combined effects of multiple drugs or enzyme inhibitors. Adv. Enzyme Regul. 22, 27-55.
33. Claramunt, R. M.; Lopez, C.; Perez-Medina, C.; Pinilla, E.; Torres, M. R.; Elguero, J., Tetrahedron 2006, 62, 11704-11713.
34. Clevenger, R. C.; Raibel, J. M.; Peck, A. M.; Blagg, B. S. J., J. Org. Chem. 2004, 69, 4375-4380.
35. Coiffier, B., Lepage, E., Briere, J., Herbrecht, R., Tilly, H., Bouabdallah, R., Morel, P., Van Den Neste, E., Salles, G., Gaulard, P., et al. (2002). CHOP chemotherapy plus rituximab compared with CHOP alone in elderly patients with diffuse large-B-cell lymphoma. N Engl J Med 346, 235-242.
36. Compagno, M., Lim, W. K., Grunn, A., Nandula, S. V., Brahmachary, M., Shen, Q., Bertoni, F., Ponzoni, M., Scandurra, M., Califano, A., et al. (2009). Mutations of multiple genes cause deregulation of NF-kappaB in diffuse large B-cell lymphoma. Nature 459, 717-721.
37. Cope, G. A., Suh, G. S., Aravind, L., Schwarz, S. E., Zipursky, S. L., Koonin, E. V., and Deshaies, R. J. (2002). Role of predicted metalloprotease motif of Jab1/Csn5 in cleavage of Nedd8 from Cull. Science 298, 608-611.
38. Crestey, F.; Ottesen, L. K.; Jaroszewski, J. W.; Franzyk, H., Tetrahedron Lett. 2008, 49, 5890-5893.
39. da Silva Correia, J., Miranda, Y., Leonard, N., and Ulevitch, R. J. (2007). The subunit CSN6 of the COP9 signalosome is cleaved during apoptosis. J Biol Chem 282, 12557-12565.
40. Dai, B., Zhao, X. F., Hagner, P., Shapiro, P., Mazan-Mamczarz, K., Zhao, S., Natkunam, Y., and Gartenhaus, R. B. (2009). Extracellular signal-regulated kinase positively regulates the oncogenic activity of MCT-1 in diffuse large B-cell lymphoma. Cancer Res 69, 7835-7843.
41. Dal Porto, J. M., Gauld, S. B., Merrell, K. T., Mills, D., Pugh-Bernard, A. E., and Cambier, J. (2004). B cell antigen receptor signaling 101. Mol Immunol 41, 599-613.
42. Davis, R. E., Brown, K. D., Siebenlist, U., and Staudt, L. M. (2001). Constitutive nuclear factor kappaB activity is required for survival of activated B cell-like diffuse large B cell lymphoma cells. J Exp Med 194, 1861-1874.
43. Davis, R. E., Ngo, V. N., Lenz, G., Tolar, P., Young, R. M., Romesser, P. B., Kohlhammer, H., Lamy, L., Zhao, H., Yang, Y., et al. (2010). Chronic active B-cell-receptor signalling in diffuse large B-cell lymphoma. Nature 463, 88-92.
44. de Groot, R. P., Raaijmakers, J. A., Lammers, J. W., Jove, R. & Koenderman, L. (1999). STAT5 activation by BCR-Abl contributes to transformation of K562 leukemia cells. Blood 94, 1108-1112.
45. de Jong, D., and Enblad, G. (2008). Inflammatory cells and immune microenvironment in malignant lymphoma. J Intern Med 264, 528-536.
46. da Rocha Dias, S. et al. (2005). Activated B-RAF is an Hsp90 client protein that is targeted by the anticancer drug 17-allylamino-17-demethoxygeldanamycin. Cancer Res. 65, 10686-10691.
47. Dealy, M. J., Nguyen, K. V., Lo, J., Gstaiger, M., Krek, W., Elson, D., Arbeit, J., Kipreos, E. T., and Johnson, R. S. (1999). Loss of Cull results in early embryonic lethality and dysregulation of cyclin E. Nat Genet 23, 245-248.
48. Deininger, M. W. & Druker, B. J. (2003). Specific targeted therapy of chronic myelogenous leukemia with imatinib. Pharmacol. Rev. 55, 401-423.
49. Deng, X. W., Dubiel, W., Wei, N., Hofmann, K., and Mundt, K. (2000). Unified nomenclature for the COP9 signalosome and its subunits: an essential regulator of development. Trends Genet 16, 289.
50. Dezwaan, D. C. & Freeman, B. C. (2008). HSP90: the Rosetta stone for cellular protein dynamics? Cell Cycle 7, 1006-1012.
51. Dierov, J., Dierova. R. & Carroll, M. (2004). BCR/ABL translocates to the nucleus and disrupts an ATR-dependent intra-S phase checkpoint. Cancer Cell 5, 275-85.
52. Du, Y.; Moulick, K.; Rodina, A.; Aguirre, J.; Felts, S.; Dingledine, R.; Fu, H.; Chiosis, G., J. Biomol. Screen. 2007, 12, 915-924.
53. Dymock, B. W.; Barril, X.; Brough, P. A.; Cansfield, J. E.; Massey, A.; McDonald, E.; Hubbard, R. E.; Surgenor, A.; Roughley, S. D.; Webb, P.; Workman, P.; Wright, L.; Drysdale, M. J., J. Med. Chem. 2005, 48, 4212-4215.
54. Eldridge, M. D.; Murray, C. W.; Auton, T. R.; Paolini, G. V.; Mee, R. P., J. Comput.-Aided Mol. Des. 1997, 11, 425-445.
55. Erdjument-Bromage, H. et al. (1998). Micro-tip reversed-phase liquid chromatographic extraction of peptide pools for mass spectrometric analysis. J. Chromatogr. A 826, 167-181.
56. Fabian, M. A. et al. (2005). A small molecule-kinase interaction map for clinical kinase inhibitors. Nat. Biotechnol. 23, 329-336.
57. Fowler, N., Sharman, J., Smith, S., Boyd, T., Grant, B., Kolibaba, K., Furman, R., Buggy, J., Loury, D., Hamdy, A., et al. (2010). The Btk Inhibitor, PCI-32765, Induces Durable Responses with Minimal Toxicity In Patients with Relapsed/Refractory B-Cell Malignancies: Results From a Phase I Study 52nd ASH Annual Meeting and Exposition.
58. Friday, B. B., and Adjei, A. A. (2008). Advances in targeting the Ras/Raf/MEK/Erk mitogenactivated protein kinase cascade with MEK inhibitors for cancer therapy. Clin Cancer Res 14, 342-346.
59. Friedberg, J. W., Sharman, J., Sweetenham, J., Johnston, P. B., Vose, J. M., Lacasce, A., SchaeferCutillo, J., De Vos, S., Sinha, R., Leonard, J. P., et al. (2010). Inhibition of Syk with fostamatinib disodium has significant clinical activity in non-Hodgkin lymphoma and chronic lymphocytic leukemia. Blood 115, 2578-2585.
60. Friesner, R. A.; Banks, J. L.; Murphy, R. B.; Halgren, T. A.; Klicic, J. J.; Mainz, D. T.; Repasky, M. P.; Knoll, E. H.; Shelley, M.; Perry, J. K.; Shaw, D. E.; Francis, P.; Shenkin, P. S., J. Med. Chem. 2004, 47, 1739-1749.
61. Fukumoto, A., Tomoda, K., Yoneda-Kato, N., Nakajima, Y., and Kato, J. Y. (2006). Depletion of Jab 1 inhibits proliferation of pancreatic cancer cell lines. FEBS Lett 580, 5836-5844.
62. Gorre, M. E., Ellwood-Yen, K., Chiosis, G., Rosen, N., and Sawyers, C. L. (2002). BCR-ABL point mutants isolated from patients with imatinib mesylate-resistant chronic myeloid leukemia remain sensitive to inhibitors of the BCR-ABL chaperone heat shock protein 90. Blood 100, 3041-3044.
63. Grbovic, O. M. et al. (2006). V600E B-Raf requires the Hsp90 chaperone for stability and is degraded in response to Hsp90 inhibitors. Proc. Natl. Acad. Sci. USA 103, 57-62.
64. Gupta, M., Ansell, S. M., Novak, A. J., Kumar, S., Kaufmann, S. H., and Witzig, T. E. (2009) Inhibition of histone deacetylase overcomes rapamycin-mediated resistance in diffuse large Bcell lymphoma by inhibiting Akt signaling through mTORC2. Blood 114, 2926-2935.
65. Hacker, H. & Karin, M. (2006). Regulation and function of IKK and IKK-related kinases. Sci. STKE. (357):rel3.
66. Halgren, T. A.; Murphy, R. B.; Friesner, R. A.; Beard, H. S.; Frye, L. L.; Pollard, W. T.; Banks, J. L., J. Med. Chem. 2004, 47, 1750-1759.
67. Hanahan, D., and Weinberg, R. A. (2000). The hallmarks of cancer. Cell 100, 57-70.
68. Hanash, S. & Taguchi, A. (2010). The grand challenge to decipher the cancer proteome. Nat. Rev. Cancer 10, 652-660.
69. Hansen, J. B.; Nielsen, M. C.; Ehrbar, U.; Buchardt, O., Synthesis 1982, 404-405.
70. He, H. et al. (2006). Identification of potent water soluble purine-scaffold inhibitors of the heat shock protein 90. J. Med. Chem. 49, 381-390.
71. Hendriks, R. W. & Kersseboom, R. (2006). Involvement of SLP-65 and Btk in tumor suppression and malignant transformation of pre-μ cells. Semin. Immunol. 18, 67-76.
72. Hiegel, G. A.; Burk, P., J. Org. Chem. 1973, 38, 3637-3639.
73. Hofmann, K., and Bucher, P. (1998). The PCI domain: a common theme in three multiprotein complexes. Trends Biochem Sci 23, 204-205.
74. Honigberg, L. A., Smith, A. M., Sirisawad, M., Verner, E., Loury, D., Chang, B., Li, S., Pan, Z., Thamm, D. H., Miller, R. A., et al. (2010). The Bruton tyrosine kinase inhibitor PCI-32765 blocks B-cell activation and is efficacious in models of autoimmune disease and B-cell malignancy. Proc Natl Acad Sci USA 107, 13075-13080.
75. Horn, T., Sandmann, T. & Boutros, M. (2010). Design and evaluation of genome-wide libraries for RNA interference screens. Genome Biol. 11(6):R61.
76. Huang, K. H., Veal, J. M., Fadden, R. P., Rice, J. W., Eaves, J., Strachan, J.-P., Barabasz, A. F., Foley, B. E., Barta, T. E., Ma, W., Silinski, M. A., Hu, M., Partridge, J. M., Scott, A., DuBois, L. G., Freed, T., Steed, P. M., Ommen, A. J., Smith, E. D., Hughes, P. F., Woodward, A. R., Hanson, G. J., McCall, W. S., Markworth, C. J., Hinkley, L., Jenks, M., Geng, L., Lewis, M., Otto, J., Pronk, B., Verleysen, K., and Hall, S. E. (2009) J. Med. Chem. 52, 4288-4305.
77. Hudes, G., Carducci, M., Tomczak, P., Dutcher, J., Figlin, R., Kapoor, A., Staroslawska, E., Sosman, J., McDermott, D., Bodrogi, I., et al. (2007). Temsirolimus, interferon alfa, or both for advanced renal-cell carcinoma. N Engl J Med 356, 2271-2281.
78. Hurvitz, S. A.; Finn, R. S., Future Oncol. 2009, 5, 1015-1025.
79. Immormino, R. M.; Kang, Y.; Chiosis, G.; Gewirth, D. T., J. Med. Chem. 2006, 49, 4953-4960.
80. Jaganathan, S., Yue, P. & Turkson, J. (2010). Enhanced sensitivity of pancreatic cancer cells to concurrent inhibition of aberrant signal transducer and activator of transcription 3 and epidermal growth factor receptor or Src. J. Pharmacol. Exp. Ther. 333, 373-381.
81. Janin, Y. L. (2010). ATPase inhibitors of heat-shock protein 90, second season. Drug Discov. Today 15, 342-353.
82. Jorgensen, W. L.; Maxwell, D.; Tirado-Rives, J., J. Am. Chem. Soc. 1996, 118, 11225-11236.
83. Kamal, A. et al. (2003). A high-affinity conformation of Hsp90 confers tumour selectivity on Hsp90 inhibitors, Nature 425, 407-410.
84. Kato, J. Y., and Yoneda-Kato, N. (2009). Mammalian COP9 signalosome. Genes Cells 14, 1209-1225.
85. Katzav, S. (2007). Flesh and blood: the story of Vav1, a gene that signals in hematopoietic cells but can be transforming in human malignancies. Cancer Lett. 255, 241-254.
86. Klejman, A. et al. (2002). The Src family kinase Hck couples BCR/ABL to STAT5 activation in myeloid leukemia cells. EMBO J. 21, 5766-5774.
87. Kolch, W. & Pitt, A. (2010). Functional proteomics to dissect tyrosine kinase signalling pathways in cancer. Nat. Rev. Cancer 10, 618-629.
88. Kufe D W, P. R., Weichselbaum P R, Bast R C, Gansler T S, Holland J F, Frei E (2003). Cancer Medicine 6, Vol Two (Hamilton, BC Decker).
89. Lam, K. P., Kuhn, R., and Rajewsky, K. (1997). In vivo ablation of surface immunoglobulin on mature B cells by inducible gene targeting results in rapid cell death. Cell 90, 1073-1083.
90. Lam, L. T., Davis, R. E., Pierce, J., Hepperle, M., Xu, Y., Hottelet, M., Nong, Y., Wen, D., Adams, J., Dang, L., et al. (2005). Small molecule inhibitors of IkappaB kinase are selectively toxic for subgroups of diffuse large B-cell lymphoma defined by gene expression profiling. Clin Cancer Res 11, 28-40.
91. Law, J. H.; Habibi, G.; Hu, K.; Masoudi, H.; Wang, M. Y.; Stratford, A. L.; Park, E.; Gee, J. M.; Finlay, P.; Jones, H. E.; Nicholson, R. I.; Carboni, J.; Gottardis, M.; Pollak, M.; Dunn, S. E., Cancer Res. 2008, 68, 10238-10246.
92. Le, Y. et al. (2009). FAK silencing inhibits leukemogenesis in BCR/ABL-transformed hematopoietic cells. Am. J. Hematol. 84, 273-278.
93. Leal, J. F., Fominaya, J., Cascon, A., Guijarro, M. V., Blanco-Aparicio, C., Lleonart, M., Castro, M. E., Ramon, Y. C. S., Robledo, M., Beach, D. H., et al. (2008). Cellular senescence bypass screen identifies new putative tumor suppressor genes. Oncogene 27, 1961-1970.
94. Lenz, G., Davis, R. E., Ngo, V. N., Lam, L., George, T. C., Wright, G. W., Dave, S. S., Zhao, H., Xu, W., Rosenwald, A., et al. (2008). Oncogenic CARD11 mutations in human diffuse large B cell lymphoma. Science 319, 1676-1679.
95. Levy, D. S., Kahana, J. A., and Kumar, R. (2009). AKT inhibitor, GSK690693, induces growth inhibition and apoptosis in acute lymphoblastic leukemia cell lines. Blood 113, 1723-1729.
96. Ley, T. J. et al. (2008). DNA sequencing of a cytogenetically normal acute myeloid leukaemia genome. Nature 456, 66-72.
97. Li, B., Ruiz, J. C., and Chun, K. T. (2002). CUL-4A is critical for early embryonic development. Mol Cell Biol 22, 4997-5005.
98. Li, J., Wang, Y., Yang, C., Wang, P., Oelschlager, D. K., Zheng, Y., Tian, D. A., Grizzle, W. E., Buchsbaum, D. J., and Wan, M. (2009). Polyethylene glycosylated curcumin conjugate inhibits pancreatic cancer cell growth through inactivation of Jab1. Mol Pharmacol 76, 81-90.
99. Lim, C. P. & Cao, X. (2006). Structure, function and regulation of STAT proteins. Mol. BioSyst, 2, 536-550.
100. Luo, W.; Dou, F.; Rodina, A.; Chip, S.; Kim, J.; Zhao, Q.; Moulick, K.; Aguirre, J.; Wu, N.; Greengard, P.; Chiosis, G., Proc. Natl. Acad. Sci. USA 2007, 104, 9511-9518.
101. Luo, W.; Rodina, A.; Chiosis, G., BMC Neurosci. 2008, 9(Suppl 2):S7.
102. Luo, W.; Sun, W.; Taldone, T.; Rodina, A.; Chiosis, G., Mol Neurodegener. 2010, 5: 24.
103. Lykke-Andersen, K., Schaefer, L., Menon, S., Deng, X. W., Miller, J. B., and Wei, N. (2003). Disruption of the COP9 signalosome Csn2 subunit in mice causes deficient cell proliferation, accumulation of p53 and cyclin E, and early embryonic death. Mol Cell Biol 23, 6790-6797.
104. Mahajan, S. et al. (2001). Transcription factor STAT5A is a substrate of Bruton's tyrosine kinase in B cells. J. Biol. Chem. 276, 31216-31228.
105. Maloney, A. et al. (2007). Gene and protein expression profiling of human ovarian cancer cells treated with the heat shock protein 90 inhibitor 17-allylamino-17-demethoxygeldanamycin. Cancer Res. 67, 3239-3253.
106. Marubayashi, S.; Koppikar, P.; Taldone, T.; Abdel-Wahab, O.; West, N.; Bhagwat, N.; Lopes-Vazquez, E. C.; Ross, K. N.; Gonen, M.; Gozman, A.; Ahn, J.; Rodina, A.; Ouerfelli, O.; Yang, G.; Hedvat, C.; Bradner, J. E.; Chiosis, G.; Levine, R. L., J. Clin. Invest. 2010, 120, 3578-3593.
107. McClellan, A. J. et al. (2007). Diverse cellular functions of the Hsp90 molecular chaperone uncovered using systems approaches. Cell 131, 121-135.
108. McCubrey, J. A. et al. (2008). Targeting survival cascades induced by activation of Ras/Raf/MEK/ERK, PI3K/PTEN/Akt/mTOR and Jak/STAT pathways for effective leukemia therapy. Leukemia 22, 708-722.
109. Menon, S., Chi, H., Zhang, H., Deng, X. W., Flavell, R. A., and Wei, N. (2007). COP9 signalosome subunit 8 is essential for peripheral T cell homeostasis and antigen receptor-induced entry into the cell cycle from quiescence. Nat Immunol 8, 1236-1245.
110. Mihailovic, T. et al. (2004). Protein kinase D2 mediates activation of nuclear factor kappaB by Bcr-Abl in Bcr-Abl+ human myeloid leukemia cells. Cancer Res. 64, 8939-8944.
111. Milhollen, M. A., Traore, T., Adams-Duffy, J., Thomas, M. P., Berger, A. J., Dang, L., Dick, L. R., Garnsey, J. J., Koenig, E., Langston, S. P., et al. (2010). MLN4924, a NEDD8-activating enzyme inhibitor, is active in diffuse large B-cell lymphoma models: rationale for treatment of NF-{kappa}B-dependent lymphoma. Blood 116, 1515-1523.
112. Moffat, J. et al. (2006). A Lentiviral RNAi Library for Human and Mouse Genes Applied to an Arrayed Viral High-Content Screen. Cell 124, 1283-1298.
113. Monti, S., Savage, K. J., Kutok, J. L., Feuerhake, F., Kurtin, P., Mihm, M., Wu, B., Pasqualucci, L., Neuberg, D., Aguiar, R. C., et al. (2005). Molecular profiling of diffuse large B-cell lymphoma identifies robust subtypes including one characterized by host inflammatory response. Blood 105, 1851-1861.
114. Munday, D. et al. (2010). Quantitative proteomic analysis of A549 cells infected with human respiratory syncytial virus. Mol. Cell Proteomics 9, 2438-2459.
115. Naka, K. et al. (2010). TGF-beta-FOXO signaling maintains leukemia-initiating cells in chronic myeloid leukemia. Nature 463, 676-678.
116. Neckers, L. (2007). Heat shock protein 90: the cancer chaperone. J Biosci 32, 517-530.
117. Ngo, V. N., Davis, R. E., Lamy, L., Yu, X., Zhao, H., Lenz, G., Lam, L. T., Dave, S., Yang, L., Powell, J., et al. (2006). A loss-of-function RNA interference screen for molecular targets in cancer. Nature 441, 106-110.
118. Nimmanapalli, R., O'Bryan, E., and Bhalla, K. (2001). Geldanamycin and its analogue 17-allylamino-17-demethoxygeldanamycin lowers Bcr-Abl levels and induces apoptosis and differentiation of Bcr-Abl-positive human leukemic blasts. Cancer Res 61, 1799-1804.
119. Nishiya, Y.; Shibata, K.; Saito, S.; Yano, K.; Oneyama, C.; Nakano, H.; Sharma, S. V., Anal. Biochem. 2009, 385, 314-320.
120. Nobukuni, T., Kozma, S. C. & Thomas, G. (2007). hvps34, an ancient player, enters a growing game: mTOR Complex1/S6K1 signaling. Curr. Opin. Cell Biol. 19, 135-141.
121. Nomura, D. K., Dix, M. M. & Cravatt, B. F. (2010). Activity-based protein profiling for biochemical pathway discovery in cancer. Nat. Rev. Cancer 10, 630-638.
122. Obermann, W. M. J., Sondermann, H., Russo, A. A., Pavletich, N. P., and Hartl, F. U. (1998). J. Cell Biol. 143, 901-910.
123. Oda, A., Wakao, H. & Fujita, H. (2002). Calpain is a signal transducer and activator of transcription (STAT) 3 and STAT5 protease. Blood 99, 1850-1852.
124. Ohh, M., Kim, W. Y., Moslehi, J. J., Chen, Y., Chau, V., Read, M. A., and Kaelin, W. G., Jr. (2002). An intact NEDD8 pathway is required for Cullin-dependent ubiquitylation in mammalian cells. EMBO Rep 3, 177-182.
125. Old, D. W.; Wolfe, J. P.; Buchwald, S. L., J. Am. Chem. Soc. 1998, 120, 9722-9723.
126. Oron, E., Mannervik, M., Rencus, S., Harari-Steinberg, O., Neuman-Silberberg, S., Segal, D., and Chamovitz, D. A. (2002). COP9 signalosome subunits 4 and 5 regulate multiple pleiotropic pathways in Drosophila melanogaster. Development 129, 4399-4409.
127. Panaretou, B., Prodromou, C., Roe, S. M., O'Brien, R., Ladbury, J. E., Piper, P. W., and Pearl, L. H. (1998). EMBO 17, 4829-4836.
128. Panattoni, M., Sanvito, F., Basso, V., Doglioni, C., Casorati, G., Montini, E., Bender, J. R., Mondino, A., and Pardi, R. (2008). Targeted inactivation of the COP9 signalosome impairs multiple stages of T cell development. J Exp Med 205, 465-477.
129. Parsons, D. W. et al. (2008). An integrated genomic analysis of human glioblastoma multiforme. Science 321, 1807-1812.
130. Patel, P. D.; Patel, M. R.; Kaushik-Basu, N.; Talele, T. T., J. Chem. Inf. Model. 2008, 48, 42-55.
131. Paukku, K. & Silvennoinen, O. (2004). STATs as critical mediators of signal transduction and transcription: lessons learned from STAT5. Cytokine Growth Factor Rev. 15, 435-455.
132. Peth, A., Berndt, C., Henke, W., and Dubiel, W. (2007). Downregulation of COP9 signalosome subunits differentially affects the CSN complex and target protein stability. BMC Biochem 8, 27.
133. Powers, M. V., Clarke, P. A., Workman, P. (2008). Dual targeting of Hsc70 and Hsp72 inhibits Hsp90 function and induces tumor-specific apoptosis. Cancer Cell 14, 250-262.
134. Pratt, W. B., Morishima, Y. & Osawa, Y. (2008). The Hsp90 chaperone machinery regulates signaling by modulating ligand binding clefts. J. Biol. Chem. 283, 22885-22889.
135. Reeder C, G. M., Habermann T, Ansell S, Micallef I, Porrata L, Johnston P, Maurer M, LaPlant B, Kabat B, Inwards D, Colgan J, Call T, Markovic S, Zent C, Zeldenrust S, Tun H, and Witzig T (2007). A Phase II Trial of the Oral mTOR Inhibitor Everolimus (RAD001) in Relapsed Aggressive Non-Hodgkin Lymphoma (NHL). Blood (ASH Annual Meeting Abstracts) 110, 121.
136. Ren, R. (2005). Mechanisms of BCR-ABL in the pathogenesis of chronic myelogenous leukaemia. Nat. Rev. Cancer 5, 172-183.
137. Richardson, K. S., and Zundel, W. (2005). The emerging role of the COP9 signalosome in cancer. Mol Cancer Res 3, 645-653.
138. Rix, U. & Superti-Furga, G. (2009). Target profiling of small molecules by chemical proteomics. Nat. Chem. Biol. 5, 616-624.
139. Roe, S. M.; Prodromou, C.; O'Brien, R.; Ladbury, J. E.; Piper, P. W.; Pearl, L. H., J. Med. Chem. 1999, 42, 260-266.
140. Salgia, R. et al. (1995). Increased tyrosine phosphorylation of focal adhesion proteins in myeloid cell lines expressing p210BCR/ABL. Oncogene 11, 1149-1155.
141. Sawyers, C. L. (1993). The role of myc in transformation by Bcr-Abl. Leuk. Lymphoma. 11, 45-46.
142. Schrodinger, L. L. C., New York.
143. Schwechheimer, C., and Deng, X. W. (2001). COP9 signalosome revisited: a novel mediator of protein degradation. Trends Cell Biol 11, 420-426.
144. Schweitzer, K., Bozko, P. M., Dubiel, W., and Naumann, M. (2007). CSN controls NF-kappaB by deubiquitinylation of IkappaBalpha. EMBO J 26, 1532-1541.
145. Schweitzer, K., and Naumann, M. (2010). Control of NF-kappaB activation by the COP9 signalosome. Biochem Soc Trans 38, 156-161.
146. Serenex; Huang, K.; Ommen, A. J.; Barta, T. E.; Hughes, P. F.; Veal, J. M.; Ma, W.; Smith, E. D.; Woodward, A. R.; McCall, W. S., WO-2008130879A2 2008.
147. Serenex; Huang, K.; Ommen, A. J.; Barta, T. E.; Hughes, P. F.; Veal, J. M.; Ma, W.; Smith, E. D.; Woodward, A. R.; McCall, W. S., US-20080269193A1 2008.
148. Sharon, M., Mao, H., Boeri Erba, E., Stephens, E., Zheng, N., and Robinson, C. V. (2009). Symmetrical modularity of the COP9 signalosome complex suggests its multifunctionality. Structure 17, 31-40.
149. Si, J. & Collins, S. J. (2008). Activated Ca2+/calmodulin-dependent protein kinase IIgamma is a critical regulator of myeloid leukemia cell proliferation. Cancer Res. 68, 3733-3742.
150. Sidera, K.; Patsavoudi, E., Cell Cycle 2008, 7, 1564-1568.
151. Solit, D. B., Zheng, F. F., Drobnjak, M., Munster, P. N., Higgins, B., Verbel, D., Heller, G., Tong, W., Cardon-Cardo, C., Agus, D. B., Scher, H. I., and Rosen, N. (2002). Clin. Cancer Res. 8, 986-993.
152. Stebbins, C. E.; Russo, A. A.; Schneider, C.; Rosen, N.; Hartl, F. U.; Pavletich, N. P., Cell 1997, 89, 239-250.
153. Su, T. T., Guo, B., Kawakami, Y., Sommer, K., Chae, K., Humphries, L. A., Kato, R. M., Kang, S., Patrone, L., Wall, R., et al. (2002). PKC-beta controls I kappa B kinase lipid raft recruitment and activation in response to BCR signaling. Nat Immunol 3, 780-786.
154. Supriatno, Harada, K., Yoshida, H., and Sato, M. (2005). Basic investigation on the development of molecular targeting therapy against cyclin-dependent kinase inhibitor p27Kip1 in head and neck cancer cells. Int J Oncol 27, 627-635.
155. Taldone, T. & Chiosis, G. (2009). Purine-scaffold hsp90 inhibitors. Curr. Top. Med. Chem. 9, 1436-1446.
156. Taldone, T., Gozman, A., Maharaj, R., and Chiosis, G. (2008). Targeting Hsp90: small-molecule inhibitors and their clinical development. Curr Opin Pharmacol 8, 370-374.
157. Taldone, T., Sun, W., Chiosis, G. (2009). Bioorg. Med. Chem. 17, 2225-2235.
158. Taldone T, Zatorska D, Patel P D, Zong H, Rodina A, Ahn J H, Moulick K, Guzman M L, Chiosis G. Design, synthesis, and evaluation of small molecule Hsp90 probes. Bioorg Med Chem. 2011 Apr. 15; 19(8):2603-14. Epub 2011 Mar. 12. PMID: 21459002
159. Taldone T, Gomes-DaGama E M, Zong H, Sen S, Alpaugh M L, Zatorska D, Alonso-Sabadell R, Guzman M L, Chiosis G. Synthesis of purine-scaffold fluorescent probes for heat shock protein 90 with use in flow cytometry and fluorescence microscopy. Bioorg Med Chem Lett. 2011 Sep. 15; 21(18):5347-52. Epub 2011 Jul. 14. PMID: 21802945
160. Tateishi, K., Omata, M., Tanaka, K., and Chiba, T. (2001). The NEDD8 system is essential for cell cycle progression and morphogenetic pathway in mice. J Cell Biol 155, 571-579.
161. Thome, M. (2004). CARMA1, BCL-10 and MALT1 in lymphocyte development and activation. Nat Rev Immunol 4, 348-359.
162. Tomoda, K., Kubota, Y., Arata, Y., Mori, S., Maeda, M., Tanaka, T., Yoshida, M., Yoneda-Kato, N., and Kato, J. Y. (2002). The cytoplasmic shuttling and subsequent degradation of p27Kip1 mediated by Jab1/CSN5 and the COP9 signalosome complex. J Biol Chem 277, 2302-2310.
163. Tomoda, K., Kubota, Y., and Kato, J. (1999). Degradation of the cyclin-dependent-kinase inhibitor p27Kip1 is instigated by Jab1. Nature 398, 160-165.
164. Tomoda, K., Yoneda-Kato, N., Fukumoto, A., Yamanaka, S., and Kato, J. Y. (2004). Multiple functions of Jab1 are required for early embryonic development and growth potential in mice. J Biol Chem 279, 43013-43018.
165. Trinkle-Mulcahy, L., et al. (2008). Identifying specific protein interaction partners using quantitative mass spectrometry and bead proteomes. J. Cell. Biol. 183, 223-239.
166. Tsaytler, P. A., Krijgsveld, J., Goerdayal, S. S., Rudiger, S. & Egmond, M. R. (2009). Novel Hsp90 partners discovered using complementary proteomic approaches. Cell Stress Chaperones 14, 629-638.
167. Wang, J., Li, C., Liu, Y., Mei, W., Yu, S., Liu, C., Zhang, L., Cao, X., Kimberly, R. P., Grizzle, W., et al. (2006). JAB1 determines the response of rheumatoid arthritis synovial fibroblasts to tumor necrosis factor-alpha. Am J Pathol 169, 889-902.
168. Wang, Y., Penfold, S., Tang, X., Hattori, N., Riley, P., Harper, J. W., Cross, J. C., and Tyers, M. (1999). Deletion of the Cull gene in mice causes arrest in early embryogenesis and accumulation of cyclin E. Curr Biol 9, 1191-1194.
169. Wanner, K., Hipp, S., Oelsner, M., Ringshausen, I., Bogner, C., Peschel, C., and Decker, T. (2006). Mammalian target of rapamycin inhibition induces cell cycle arrest in diffuse large B cell lymphoma (DLBCL) cells and sensitises DLBCL cells to rituximab. Br J Haematol 134, 475-484.
170. Wei, N., and Deng, X. W. (1998). Characterization and purification of the mammalian COP9 complex, a conserved nuclear regulator initially identified as a repressor of photomorphogenesis in higher plants. Photochem Photobiol 68, 237-241.
171. Welteke, V., Eitelhuber, A., Duwel, M., Schweitzer, K., Naumann, M., and Krappmann, D. (2009). COP9 signalosome controls the Carma1-Bcl10-Malt1 complex upon T-cell stimulation. EMBO Rep 10, 642-648.
172. Whitesell, L. & Lindquist, S. L. (2005). Nat. Rev. Cancer 5, 761-772.
173. Whitesell, L., Mimnaugh, E. G., De Costa, B., Myers, C. E., and Neckers, L. M. (1994). Proc. Natl. Acad. Sci. USA 91, 8324-8328.
174. Winkler, G S. et al. (2002). Isolation and mass spectrometry of transcription factor complexes. Methods 26, 260-269.
175. Workman, P., Burrows, F., Neckers, L. & Rosen, N. (2007). Drugging the cancer chaperone HSP90: combinatorial therapeutic exploitation of oncogene addiction and tumor stress. Ann. N. Y Acad. Sci. 1113, 202-216.
176. Wright, G., Tan, B., Rosenwald, A., Hurt, E. H., Wiestner, A., and Staudt, L. M. (2003). A gene expression-based method to diagnose clinically distinct subgroups of diffuse large B cell lymphoma. Proc Natl Acad Sci USA 100, 9991-9996.
177. Xu, D. & Qu, C. K. (2008). Protein tyrosine phosphatases in the JAK/STAT pathway. Front. Biosci. 13, 4925-4932.
178. Yan, J., Walz, K., Nakamura, H., Carattini-Rivera, S., Zhao, Q., Vogel, H., Wei, N., Justice, M. J., Bradley, A., and Lupski, J. R. (2003). COP9 signalosome subunit 3 is essential for maintenance of cell proliferation in the mouse embryonic epiblast. Mol Cell Biol 23, 6798-6808.
179. Yap, T. A., Garrett, M. D., Walton, M. I., Raynaud, F., de Bono, J. S., and Workman, P. (2008). Targeting the PI3K-AKT-mTOR pathway: progress, pitfalls, and promises. Curr Opin Pharmacol 8, 393-412.
180. Ye, B. H., Cattoretti, G., Shen, Q., Zhang, J., Hawe, N., de Waard, R., Leung, C., Nouri-Shirazi, M., Orazi, A., Chaganti, R. S., et al. (1997). The BCL-6 proto-oncogene controls germinal-centre formation and Th2-type inflammation. Nat Genet 16, 161-170.
181. Ye, B. H., Lista, F., Lo Coco, F., Knowles, D. M., Offit, K., Chaganti, R. S., and Dalla-Favera, R. (1993). Alterations of a zinc finger-encoding gene, BCL-6, in diffuse large-cell lymphoma. Science 262, 747-750.
182. Yoneda-Kato, N., Tomoda, K., Umehara, M., Arata, Y., and Kato, J. Y. (2005). Myeloid leukemia factor 1 regulates p53 by suppressing COP1 via COP9 signalosome subunit 3. EMBO J 24, 1739-1749.
183. Zhao, X. et al. (2009). Methylation of RUNX1 by PRMT1 abrogates SIN3A binding and potentiates its transcriptional activity. Genes Dev. 22, 640-653.
184. Zuehlke, A. & Johnson, J. L. (2010). Hsp90 and co-chaperones twist the functions of diverse client proteins. Biopolymers 93, 211-217.

1. A method for selecting an inhibitor of a cancer-implicated pathway, or of a component of a cancer-implicated pathway, for coadministration with an inhibitor of Hsp90, to a subject suffering from a cancer which comprises the following steps:
(a) contacting a sample containing cancer cells from the subject with (i) an inhibitor of Hsp90 which binds to Hsp90 when such Hsp90 is bound to cancer pathway components present in the sample; or (ii) an analog, homolog, or derivative of such Hsp90 inhibitor which binds to Hsp90 when such Hsp90 is bound to such cancer pathway components in the sample;
(b) detecting pathway components bound to Hsp90;
(c) analyzing the pathway components detected in step (b) so as to identify a pathway which includes the components detected in step (b) and additional components of such pathway; and
(d) selecting an inhibitor of the pathway or of a pathway component identified in step (c).

2. A method of claim 1, wherein the cancer-implicated pathway is a pathway involved in metabolism, genetic information processing, environmental information processing, cellular processes, or organismal systems.

3. A method of claim 2, wherein the cancer-implicated pathway is a pathway listed in Table 1.

4. A method of claim 1, wherein the cancer-implicated pathway or the component of the cancer-implicated pathway is involved with a cancer selected from the group consisting of colorectal cancer, pancreatic cancer, thyroid cancer, a leukemia including acute myeloid leukemia and chronic myeloid leukemia, basal cell carcinoma, melanoma, renal cell carcinoma, bladder cancer, prostate cancer, a lung cancer including small cell lung cancer and non-small cell lung cancer, breast cancer, neuroblastoma, myeloproliferative disorders, gastrointestinal cancers including gastrointestinal stromal tumors, esophageal cancer, stomach cancer, liver cancer, gallbladder cancer, anal cancer, brain tumors including gliomas, lymphomas including follicular lymphoma and diffuse large B-cell lymphoma, and gynecologic cancers including ovarian, cervical, and endometrial cancers.

5. (canceled)

6. A method of claim 1, wherein in step (a) the subject is the same subject to whom the inhibitor of the cancer-implicated pathway or the component of the cancer-implicated pathway is to be administered.

7. A method of claim 1, wherein in step (a) the subject is a cancer reference subject.

8. A method of claim 1, wherein in step (a) the sample comprises a tumor tissue.

9. A method of claim 1, wherein in step (a) the sample comprises a biological fluid.

10. A method of claim 9, wherein the biological fluid is blood.

11. A method of claim 1, wherein in step (a) the sample comprises disrupted cancer cells.

12. A method of claim 11, wherein the disrupted cancer cells are lysed cancer cells.

13. A method of claim 11, wherein the disrupted cancer cells are sonicated cancer cells.

14-45. (canceled)

46. A method of treating a subject suffering from a chronic myelogenous leukemia (CML) which comprises administering to the subject an inhibitor of CAPM1.

47. A method for identifying a cancer-implicated pathway or one or more components of a cancer-implicated pathway in a subject suffering from cancer which comprises:
(a) contacting a sample containing cancer cells from the subject with (i) an inhibitor of Hsp90 which binds to Hsp90 when such Hsp90 is bound to cancer pathway components present in the sample; or (ii) an analog, homolog, or derivative of such Hsp90 inhibitor which hinds to Hsp90 when such Hsp90 is bound to such cancer pathway components in the sample;
(b) detecting pathway components bound to Hsp90;
so as to thereby identify the cancer-implicated pathway or said one or more pathway components.

48. A method of claim 47, wherein the cancer-implicated pathway or the component of the cancer-implicated pathway is involved with a cancer selected from the group consisting of colorectal cancer, pancreatic cancer, thyroid cancer, a leukemia including acute myeloid leukemia and chronic myeloid leukemia, basal cell carcinoma, melanoma, renal cell carcinoma, bladder cancer, prostate cancer, a lung cancer including small cell lung cancer and non-small cell lung cancer, breast cancer, neuroblastoma, myeloproliferative disorders, gastrointestinal cancers including gastrointestinal stromal tumors, esophageal cancer, stomach cancer, liver cancer, gallbladder cancer, anal cancer, brain tumors including gliomas, lymphomas including follicular lymphoma and diffuse large B-cell lymphoma, and gynecologic cancers including ovarian, cervical, and endometrial cancers.

49. A method of claim 47, wherein in step (a) the sample comprises a tumor tissue.

50. A method of claim 47, wherein in step (a) the sample comprises a biological fluid.

51. A method of claim 50, wherein the biological fluid is blood.

52. A method of claim 47, wherein in step (a) the sample comprises disrupted cancer cells.

53. A method of claim 52, wherein the disrupted cancer cells are lysed cancer cells.

54. A method of claim 52, wherein the disrupted cancer cells are sonicated cancer cells.

55-67. (canceled)

68. The method for selecting an inhibitor of a cancer-implicated pathway or a component of a cancer-implicated pathway which comprises identifying the cancer-implicated pathway or one or more component of such pathway according to the method of claim 44 and then selecting an inhibitor of such pathway or such component.

69. The method of treating a subject comprising selecting an inhibitor according to the method of claim 68 and administering the inhibitor to the subject.

70. The method of claim 69, further comprising administering to the subject said inhibitor and an inhibitor of Hsp90.

71. The method of claim 69, wherein said administering is effected repeatedly.

72-77. (canceled)


SEQUENCE LISTING (27 sequences)

SEQ ID NO: 1 - DNA, 22 bp, Artificial Sequence
  CCND2 primer
gttgttctggtccctttaatcg

SEQ ID NO: 2 - DNA, 18 bp, Artificial Sequence
  CCND2 primer
acctcgcatacccagaga

SEQ ID NO: 3 - DNA, 20 bp, Artificial Sequence
  MYC primer
atgcgttgctgggttatttt

SEQ ID NO: 4 - DNA, 20 bp, Artificial Sequence
  MYC primer
cagagcgtgggatgttagtg

SEQ ID NO: 5 - DNA, 20 bp, Artificial Sequence
  Intergenic control region primer
ccacctgagtctgcaatgag

SEQ ID NO: 6 - DNA, 20 bp, Artificial Sequence
  Intergenic control region primer
cagtctccagcctttgttcc

SEQ ID NO: 7 - DNA, 20 bp, Artificial Sequence
  MYC primer
agaagagcatcttccgcatc

SEQ ID NO: 8 - DNA, 20 bp, Artificial Sequence
  MYC primer
cctttaaacagtgcccaagc

SEQ ID NO: 9 - DNA, 20 bp, Artificial Sequence
  CCND2 primer
tgagctgctggctaagatca

SEQ ID NO: 10 - DNA, 20 bp, Artificial Sequence
  CCND2 primer
acggtactgctgcaggctat

SEQ ID NO: 11 - DNA, 24 bp, Artificial Sequence
  BCL-XL primer
cttttgtggaactctatgggaaca

SEQ ID NO: 12 - DNA, 19 bp, Artificial Sequence
  BCL-XL primer
cagcggttgaagcgttcct

SEQ ID NO: 13 - DNA, 19 bp, Artificial Sequence
  MCL1 primer
agaccttacgacgggttgg

SEQ ID NO: 14 - DNA, 20 bp, Artificial Sequence
  MCL1 primer
acattcctgatgccaccttc

SEQ ID NO: 15 - DNA, 20 bp, Artificial Sequence
  CCND1 primer
cctgtcctactaccgcctca

SEQ ID NO: 16 - DNA, 18 bp, Artificial Sequence
  CCND1 primer
ggcttcgatctgctcctg

SEQ ID NO: 17 - DNA, 21 bp, Artificial Sequence
  HPRT primer
cgtcttgctcgagatgtgatg

SEQ ID NO: 18 - DNA, 22 bp, Artificial Sequence
  HPRT primer
gcacacagagggctacaatgtg

SEQ ID NO: 19 - DNA, 20 bp, Artificial Sequence
  GAPDH primer
cgaccactttgtcaagctca

SEQ ID NO: 20 - DNA, 20 bp, Artificial Sequence
  GAPDH primer
ccctgttgctgtagccaaat

SEQ ID NO: 21 - DNA, 21 bp, Artificial Sequence
  RPL13A primer
tgagtgaaagggagccagaag

SEQ ID NO: 22 - DNA, 20 bp, Artificial Sequence
  RPL13A primer
cagatgccccactcacaaga

SEQ ID NO: 23 - RNA, 19 nt, Artificial Sequence
  Active sequence against Hsp70 (Hsp70A)
ggacgaguuugagcacaag

SEQ ID NO: 24 - RNA, 19 nt, Artificial Sequence
  Active sequence against Hsp70 (Hsp70B)
ccaagcagacgcagaucuu

SEQ ID NO: 25 - RNA, 19 nt, Artificial Sequence
  Inverted control siRNA sequence (Hsp70C)
ggacgaguuguagcacaag

SEQ ID NO: 26 - DNA, 20 bp, Artificial Sequence
  CARM1 forward primer
tgatggccaagtctgtcaag

SEQ ID NO: 27 - DNA, 20 bp, Artificial Sequence
  CARM1 reverse primer
tgaaagcaacgtcaaaccag